(19) United States
(12) Patent Application Publication
(10) Pub. No.: US 2021/0022676 A1
(43) Pub. Date: Jan. 28, 2021

(54) MONITORING DEVICES AND METHODS

(71) Applicant: True Wearables, Inc., Rancho Santa Margarita, CA (US)

(72) Inventors: Marcelo Malini LAMEGO, Mission Viejo, CA (US); Tatiana Buticosky LAMEGO, Mission Viejo, CA (US); Isadora Buticosky LAMEGO, Mission Viejo, CA (US); Larissa Buticosky LAMEGO, Mission Viejo, CA (US)

(73) Assignee: True Wearables, Inc., Rancho Santa Margarita, CA (US)

(21) Appl. No.: 16/939,925

(22) Filed: Jul. 27, 2020

Related U.S. Application Data
(63) Continuation-in-part of application No. 16/551,437, filed on Aug. 26, 2019.
(60) Provisional application No. 62/878,670, filed on Jul. 25, 2019.
(60) Provisional application No. 62/884,578, filed on Aug. 8, 2019.
(60) Provisional application No. 62/932,394, filed on Nov. 7, 2019.
(60) Provisional application No. 62/951,651, filed on Dec. 20, 2019.

(51) Int. Cl. A61B 5/00; G08C 15/06; G08C 17/02; A42B 1/24; A41D 1/00

(52) U.S. Cl. CPC A61B 5/6803; G08C 15/06; A61B 5/0017; A42B 1/242; A41D 1/002; G08C 17/02

(57) ABSTRACT

Monitoring devices and monitoring technology are disclosed for a number of applications. A monitoring device that attaches to a measurement site includes at least one of an optical sensor, a temperature sensor, or first and second electrical contact sensors. A monitoring device or a smart garment can be powered by one or more bio-batteries that is each formed by electrodes in contact with the user's body (e.g., skin) or an animal. Various method and algorithms can be used to process signals received from the optical sensor, a temperature sensor, and/or first and second electrical contact sensors. The signals received from the optical sensor, a temperature sensor, and/or first and second electrical contact sensors can be transmitted to a host device. An application program on a host device can process the signals to compute one or more physiological parameters, waveform data, trend data, and/or one or more reports.

### CROSS-REFERENCE TO RELATED APPLICATION(S)

This application is a continuation-in-part of, and claims priority to, U.S. application Ser. No. 16/551,437 entitled “Monitoring Devices and Methods”, filed on Aug. 26, 2019, which claims priority to U.S. Provisional Application No. 62/723,290 entitled “Monitoring Devices and Methods”, filed on Aug. 27, 2018, and to U.S. Provisional Application No. 62/722,676 entitled “Monitoring Devices and Methods”, filed on Aug. 24, 2018, of which each entire disclosure is hereby incorporated by reference in its entirety. This application also claims priority to U.S. Provisional Application 62/878,670 entitled “Software and Firmware For Medical, Fitness, and Wellness Real-Time Applications”, filed on Jul. 25, 2019; to U.S. Provisional Application 62/884,578 entitled “Software and Firmware For Medical, Fitness, and Wellness Real-Time Applications and with Extended Ranges,” filed on Aug. 8, 2019; to U.S. Provisional Application 62/932,394 entitled “Software and Firmware For Medical, Fitness, and Wellness Real-Time Applications and with Extended Ranges,” filed on Nov. 7, 2019; and to U.S. Provisional Application No. 62/951,651 entitled “Software and Firmware For Medical, Fitness, and Wellness Real-Time Applications and with Extended Ranges,” filed on Dec. 20, 2019, of which each entire disclosure is hereby incorporated by reference in its entirety.

### BACKGROUND

Devices that determine various health parameters are used regularly by consumers and medical personnel. For example, the measurement of blood oxygen saturation (SpO2), pulse rate (PR), and perfusion index (PI) are health parameters consumers and medical personnel monitor to receive feedback on the user's health and/or fitness.

### SUMMARY

Embodiments disclosed herein enable clinical-grade monitoring technology in a number of applications where low-cost, wireless, multi-parameter, single-use and multi-use medical and fitness and/or wellness devices are useful and beneficial. Wireless characteristics enable convenience, comfort, and/or freedom of movement for the user. The interplay between single-use and multi-use designs enables use-case flexibility. In medical applications, single-use designs reduce the risks of cross contamination and healthcare associated infections, simplifies workflow, and eliminates failures due to equipment wear and tear. In fitness and wellness applications, multi-use designs enable more affordable solutions for personal use. Embodiments of the monitoring technology can be applied to a number of clinical settings and fitness and wellness applications, including pulse oximetry for COPD, anesthesia, aviation and sports, apnea detection, and oxygen therapy, non-invasive continuous blood glucose monitoring for diabetes disease management, continuous body temperature monitoring, ECG spot-check monitoring, electroencephalogram (EEG) continuous monitoring, non-invasive monitoring of water in blood for body hydration management, non-invasive total hemoglobin monitoring for anemia and/or blood transfusion management, continuous dyshemoglobinemia monitoring, among many others.

The monitoring device can be a distinct monitoring device that attaches to a measurement site of a user or and animal. Alternatively, one or more monitoring devices may be included in a wearable monitoring device or a smart garment. A wearable monitoring device includes, but is not limited to, eyewear, jewelry, and other technologies that are worn on or in the body of a user or an animal. In some embodiments, a wearable monitoring device or smart garment is powered by one or more bio-batteries that is each formed by a cathode electrode and an anode electrode in partial or complete contact with the body (e.g., skin) of the user or the animal.

A monitoring device, a wearable monitoring device, or a smart garment can include first circuitry comprising an authentication and/or storage memory or memories operably connected to a processing device (e.g., microprocessor(s)), wireless communication device(s), input/output (IO) interface circuitry, and power management circuitry. The monitoring device, wearable monitoring device, or smart garment may further include second circuitry that comprises an optical frontend operably connected to optical sensor(s), an electrical frontend operably connected to electrical sensor(s), an ultrasound frontend operably connected to ultrasound sensor(s), a temperature frontend operably connected to temperature sensor(s), a motion and/or posture frontend operably connected to motion and/or posture sensor(s), and/or a location and/or environmental frontend operably connected to location and/or environmental sensors. The electrical sensor(s), ultrasound sensor(s), temperature sensor(s), motion and/or posture sensor(s), and location/environmental sensor(s) are auxiliary sensors. In one embodiment, the optical frontend, the electrical frontend, the ultrasound frontend, the temperature frontend, the motion and/or posture frontend, and/or the location and/or environmental frontend control the transmission of data that is captured by the optical sensor(s), the electrical sensor(s), the ultrasound sensor(s), the temperature sensor(s), the motion and/or posture sensor(s), and/or the location and/or environmental sensors, respectively.

In one aspect, a monitoring device, wearable monitoring device, or smart garment includes an optical sensor, a temperature sensor, a first electrical contact sensor, and a second electrical contact sensor within an enclosure of the monitoring device. The optical sensor includes a light source and a photodetector positioned adjacent a first surface of the enclosure. The light source is operable to emit light towards the measurement site and the photodetector is operable to receive light reflected from the measurement site when the first surface is in contact with a measurement site of a first body part of a user. The temperature sensor is positioned adjacent the first surface of the enclosure and is operable to measure a temperature at the measurement site when the first surface is in contact with the measurement site. The first electrical contact sensor is positioned adjacent the first surface of the enclosure to be in contact with the measurement site when the first surface is in contact with the measurement site. The second electrical contact sensor is positioned adjacent a second surface of the enclosure. The first and the second electrical contact sensors detect heart signals when a different second body part of the user contacts the second electrical contact sensor. The monitoring device also includes a wireless communication device that is operable to transmit signals received from the photodetector, temperature measurements, and the heart signals to an application program on a host device.

In some aspects, the temperature of a user (e.g., skin temperature) is measured at one or more extremities of the user. The temperature can be can be used as a proxy for the user's “comfort level”. Other measurements can be used in combination with the continuous temperature measurements to determine the user's “comfort level”. For example, other physiological measurements such as heart rate, perfusion index, pulse amplitude, respiration rate, oxygen saturation, and the like can be combined with the continuous temperature measurements.

The monitoring device, wearable monitoring device, or smart garment can be operably connected to a host device, a computing system, and/or a cloud computing system. The host device and the computing system can each be in the same location (e.g., room) as the monitoring device, wearable monitoring device, or smart garment. Alternatively, the monitoring device, wearable monitoring device, or smart garment along with the host device and/or the computing system can form a distributed system where the monitoring device, wearable monitoring device, or smart garment is in one location and the host device and/or the computing system is in another (different) location.

The monitoring device can obtain measurements for various physiological parameters continuously or at select times and transmit the measurements to the host device, the computing system, and/or the cloud computing system. An application program on the host device can display a measurement gauge for one or more physiological parameters, a body temperature gauge, one or more waveforms, and/or trend waveforms or charts in a user interface screen of the application program. The application program can generate an alarm when a physiological parameter exceeds an upper limit and/or is below a lower limit. The upper and lower limits can be set in a settings user interface screen of the application program

In some aspects, the battery life of the battery in the monitoring device can be estimated by the application program on the host device, by the monitoring device, or in a distributed process using both the host device and the monitoring device. The computations in are performed in both a closed loop and an open loop via different functions. In the closed loop, the battery voltage and/or other available parameters of interest (e.g., ambient temperature, circuitry loads, etc.) is used to estimate the battery charge directly. In the open loop, the battery charge is estimated indirectly using one or more counters and/or other available parameters of interest (e.g., ambient temperature, circuitry loads, etc.).

In some embodiments, an authentication method includes the host device receiving a first identifier from the monitoring device. The host device authenticates the monitoring device based on the first identifier. In a non-limiting example, the first identifier is created when the monitoring device is manufactured using a unique factory-programmed identifier(s) stored in the processing device or the memory (e.g., a secure memory) in the monitoring device. If the authentication is successful the host device sends a second identifier to the monitoring device. In one embodiment, the second identifier is calculated from the authenticated first identifier. The monitoring device receives the second identifier and compares the second identifier with an internally calculated or stored third identifier. In one embodiment, the second identifier is a second hash code computed by the host device and the third identifier is a third hash code calculated by the monitoring device from the same data stored in the first identifier. When the second and the third identifiers match, the monitoring device is authenticated and a connection (e.g., a wireless connection) is established between the monitoring device and the host device.

In one aspect, a wearable monitoring device or garment are made so that the biological tissue of a user (e.g., the skin) acts as the electrolytes of a battery, and circuitry in the wearable monitoring device or garment provides the electrodes (i.e., cathode(s) and anode(s)) of the battery.

In some embodiments, a distributed fuel gauge system is used to measure the battery life of the monitoring device. The distributed fuel gauge system relies on measurements of the battery and circuitry (e.g., temperatures, clock frequencies, currents, voltages, loads, etc.) that are taken by the monitoring device and transmitted to the host device. In addition, the monitoring device can maintain one or more non-volatile counters that log the energy consumed by the monitoring device in each of the states of the monitoring device. In one example, the monitoring device can have a standby counter that is active whenever the monitoring device is in standby and is not connected to the host device. Additionally or alternatively, the monitoring device may have a usage counter that is active whenever the monitoring device is running and/or is connected to the host device. A connection timer (watchdog timer) can also be used.

Example operations that can be performed while a monitoring device is running and connected to a host device include acquiring data streams and demodulating and demultiplexing the data streams to produce individual data streams. Auxiliary data streams can also be demodulated and/or demultiplexed. For instance, the monitoring device can also produce a time-multiplexed data stream where each time slot provides a value that can be from an electrical current, a resistance, or a voltage variable, a temperature variable, a digital counter variable, or a diagnostic variable. The auxiliary data stream is demultiplexed to produce a number of data streams, one for each time multiplexed variable. The demultiplexed data streams are then operated on by a decimation process, where the sampling rate and the frequency bandwidth of the demultiplexed data streams are reduced to minimize the signal processing requirements, to reduce power consumption, and/or to increase the signal-to-noise ratio through averaging by the decimation filters. The auxiliary data streams can be decimated or interpolated depending on the desired sampling frequency. Additional calculations can be performed, where the additional calculations include operations for converting scales of data streams and variables, electronic saturation detection, or any other computation performed by or for the device firmware in the monitoring device. The demodulated, demultiplexed, and decimated data streams and auxiliary data streams (if any), along with other optional parameters and variables are sent to a host device. The data streams are received in real-time by the host device, and in one embodiment are further processed by the host device.

In another aspect, a hybrid system that employs state space representation for the measurement algorithms. A hybrid system is a system that exhibits both continuous and discrete dynamic behavior. In some embodiments, data in the hybrid system is processed in a discrete-time periods. In such embodiments, data processing is performed for each epoch (e.g., each discrete time period). The method includes initializing one or more constants and the input, output, and current state variables (i.e., state variables defined in current epoch) using the one or more initialized constants and/or one or more default values. Real-time data is received by the host device from the monitoring device and the next states and outputs are calculated from the one or more inputs, the current states, and the one or more constants. The current states are then updated from the calculated next states. The data that is displayed, shared, and/or stored is updated, along with any alarms, fuel gauges (e.g., battery icon), and/or notifications.

In an example embodiment, an optical sensor is positioned within an enclosure of a monitoring device. The optical sensor includes a light source and a photodetector, where the light source is operable to emit light towards a measurement site and the photodetector is operable to receive light reflected from the measurement site when the first surface is in contact with a measurement site of a first body part of a user. The monitoring device also includes within the enclosure an auxiliary sensor, a wireless communication device, a memory, and a processing device operably connected to the optical sensor, the auxiliary sensor, the memory, and the wireless communication device. The memory stores instructions, that when executed by the processing device, cause operations to be performed. The operations include receiving a data stream from the optical sensor and receiving an auxiliary data stream from the auxiliary sensor. The data stream is demodulated and demulitplexed to produce at least two individual data streams. The at least two individual data streams are decimated to produce at least two decimated individual data streams. The auxiliary data stream is demodulated and decimated to produce a time-multiplexed data stream. The at least two decimated individual data streams and the time-multiplexed data stream are transmitted to a computing device using the wireless communication device.

In one aspect, a method includes receiving a data stream from the optical sensor; receiving an auxiliary data stream from the auxiliary sensor; demodulating and demultiplexing the data stream to produce at least two individual data streams; decimating the at least two individual data streams to produce at least two decimated individual data steams; demodulating and demultiplexing the auxiliary data stream to produce a time-multiplexed data stream; and transmitting, using the wireless communication device, the at least two decimated individual data streams and the time-multiplexed data stream to a computing device.

### BRIEF DESCRIPTION OF THE DRAWINGS

Non-limiting, and non-exhaustive examples are described with reference to the following Figures. The elements of the drawings are not necessarily to scale relative to each other. Identical reference numerals have been used, where possible, to designate identical features that are common to the figures.

FIG. 1 is a block diagram illustrating a first example of a monitoring device connected to a computing device;

FIGS. 2A-2I illustrate example measurement sites for a monitoring device;

FIG. 3 is a block diagram illustrating a second example of a monitoring device;

FIG. 4 is a block diagram illustrating a third example of a monitoring device;

FIGS. 5A-5C are schematic diagrams depicting an example monitoring device;

FIG. 6 depicts a cross-section view of an example monitoring device;

FIGS. 7A-7D illustrate example embodiments of a monitoring device;

FIG. 7E depicts frontend circuitry for the temperature sensor and the electrical sensor;

FIGS. 8A-8B illustrate different contact surface configurations of a monitoring device;

FIG. 9 depicts a first example distributed system suitable for processing signals produced by a monitoring device and a first example modulation scheme that is suitable for use with a monitoring device;

FIG. 10A illustrates a second example modulation scheme that is suitable for use with a monitoring device;

FIG. 10B depicts a third example modulation scheme that is suitable for use with a monitoring device;

FIG. 11A illustrates a flowchart of a first example method of determining a modulation scheme to use in a monitoring device;

FIG. 11B depicts a flowchart of a second example method of determining a modulation scheme to use in a monitoring device;

FIG. 12A illustrates a second example of a distributed system suitable for processing signals produced by a monitoring device;

FIG. 12B depicts an integrated device that includes a monitoring device and a host device;

FIG. 13 illustrates a third example of a distributed system suitable for processing signals produced by a monitoring device;

FIG. 14 depicts a fourth example of a distributed system suitable for processing signals produced by a monitoring device;

FIG. 15 illustrates a fifth example of a distributed system suitable for processing signals produced by a monitoring device;

FIG. 16 depicts a sixth example of a distributed system suitable for processing signals produced by a monitoring device;

FIG. 17 illustrates a seventh example of a distributed system suitable for processing signals produced by a monitoring device;

FIG. 18 illustrates an eighth example of a distributed system suitable for processing signals produced by a monitoring device;

FIG. 19 depicts a ninth example of a distributed system suitable for processing signals produced by a monitoring device;

FIG. 20 illustrates the steps to activate a monitoring device;

FIG. 21 depicts a flowchart of an example method of an authentication process for connecting the monitoring device to a host device;

FIG. 22 illustrates a first example method of determining an active noise floor of a monitoring device;

FIG. 23 depicts a second example method of determining an active noise floor of a monitoring device;

FIG. 24 illustrates a flowchart of a first example method of operating a monitoring device;

FIG. 25 depicts an example state diagram for a host device;

FIG. 26 illustrates an example state diagram for a monitoring device;

FIG. 27 depicts a flowchart of an example sensor calibration process that is suitable for use by a monitoring device;

FIG. 28 illustrates an example sensor calibration system that is suitable to execute the sensor calibration process shown in FIG. 27;

FIG. 29 depicts example technical specifications of a monitoring device when used in combination with a host device;

FIG. 30 shows an example scatter plot and an example Bland-Altman plot illustrating a typical SpO2 clinical performance for a reflectance-based pulse oximetry system;

FIG. 31 illustrates example experimental points of a typical calibration curve for a pulse oximetry system;

FIG. 32 depicts a flowchart of an example method of providing SpO2 values that exceed one hundred percent;

FIG. 33 illustrates a flowchart of example operations that are performed while a monitoring device is running and connected to a host device;

FIG. 34 depicts a flow chart illustrating an example method of processing measurement data received from a monitoring device;

FIG. 35 illustrates a flowchart of an example method of computing the pulse amplitude (PA) log-volatility or perfusion index (PI) log-volatility of the measurement data;

FIG. 36 depicts a flowchart of an example method of computing the pulse rate (PR) volatility of the measurement data;

FIG. 37 illustrates a flowchart of an example method of computing the Ln log-volatility of the measurement data;

FIG. 38 depicts a flowchart of a method of computing the Ln volatility of measurement data;

FIG. 39 depicts a flowchart that is used to describe a hybrid system employing state space representation for the measurement algorithms;

FIG. 40 illustrates a flowchart of example operations that can be performed in block 3900 shown in FIG. 39;

FIG. 41A-41H depict example data flow diagrams;

FIG. 42 is a block diagram that illustrates how the example data flow diagrams shown in FIGS. 41A-41H can be connected together and processed at each epoch for a series of indexed models (i.e., i=1, 2, . . . , I), and a series of reference models (i.e., j=1, 2, . . . , J);

FIG. 43 depicts a block diagram of an alternate embodiment of how the example data flow diagrams shown in FIGS. 41A-41H can be connected together and processed at each epoch for a series of indexed models (i.e., i=1, 2, . . . , I), and a series of reference models (i.e., j=1, 2, . . . , J);

FIG. 44 illustrates a flowchart of example operations that are suitable for use in block 3906 shown in FIG. 39;

FIG. 45 depicts a flowchart of example operations that are suitable for use in block 4400 shown in FIG. 44;

FIG. 46 illustrates a flowchart of example operations that are suitable for use in blocks 4402 and 4404 shown in FIG. 44;

FIG. 47 depicts a flowchart of a method of operating a distributed fuel gauge system that measures the battery life of the monitoring device;

FIG. 48 illustrates an example fuel gauge indicator that considers hysteresis to prevent jittering;

FIG. 49A depicts a flowchart of a method of initializing and restarting a monitoring device based on one or more counters associated with battery energy;

FIG. 49B illustrates a flowchart of a method of operating a watchdog timer;

FIG. 50 depicts an example battery discharge curve;

FIG. 51 illustrates a flowchart of a method of estimating the life of the battery in the monitoring device based on the battery discharge curve shown in FIG. 50;

FIG. 52 illustrates a flowchart of an anti-tampering method for the monitoring device;

FIG. 53 depicts the example battery discharge curve that is shown in FIG. 50

FIG. 54 illustrates an example method of estimating the battery life of the battery in the monitoring device;

FIG. 55 depicts a flowchart of an alternate example method of estimating the battery life of the battery in the monitoring device;

FIG. 56 illustrates a graphic representation of the host application installed in a host device that works with a monitoring device;

FIGS. 57-58 depict example operations to start the host application in a host device

FIGS. 59-61 illustrate example operations to connect the monitoring device to the host device;

FIGS. 62-64 depict an example user interface screen that may be displayed at a host device;

FIG. 65 illustrates several user interface screens that can be displayed on a host device;

FIGS. 66A-66B depict a workflow of a host device sharing the data received from the monitoring device;

FIGS. 66C-66N show example user interface screens that are suitable for display on a host device or for inclusion in data output as a report;

FIG. 67 illustrates an example screen of identification parameters produced by the host application on the host device;

FIG. 68 depicts an example screen of hardware diagnostics parameters produced by the host application;

FIG. 69 illustrates an example embodiment on sharing the measurement data with a technical support team of the monitoring device;

FIG. 70 depicts an example embodiment of an alarm/warning system;

FIG. 71 illustrates an example settings screen of the host application executing on the host device;

FIG. 72 depicts a battery icon and example battery states;

FIG. 73 illustrates a flowchart of an example method of providing SpO2 measurement values that exceed one hundred percent;

FIG. 74 depicts a user interface screen of the host application that is suitable to use for displaying one or more measurement values over one hundred percent;

FIG. 75 illustrates an example settings user interface screen of the host application that can be to display extended measurement values over one hundred percent;

FIG. 76 depicts an example user interface screen of a host application;

FIGS. 77A and 77B illustrate a package that includes a monitoring device and example accessories;

FIG. 78 depicts a workflow showing processes for attaching a monitoring device to a measurement site on a finger;

FIGS. 79A-79F illustrate example tape layouts suitable for use with a monitoring device;

FIGS. 80-82 depict a monitoring device attached the forehead of a user;

FIG. 83 illustrates a monitoring device attached to the posterior auricle (ear) of a user;

FIG. 84 depicts an example wearable hat that includes a monitoring device;

FIG. 85 illustrates several example configurations for the wearable hat shown in FIG. 84;

FIG. 86 depicts an example wearable patch positioned on a user;

FIGS. 87-88 illustrate example embodiments for veterinary applications using a monitoring device, a host device, and a computing system as shown in FIG. 12A;

FIG. 89 depicts a flowchart of an example first method of providing SpO2 values that exceed one hundred percent in veterinary uses;

FIG. 90 illustrates a flowchart of an example second method of providing SpO2 values that exceed one hundred percent in veterinary uses;

FIG. 91 depicts an example embodiment for space applications using a monitoring device, a host device, a computing system, and a cloud computing system as shown in FIG. 12A;

FIG. 92 illustrates another example method of using a monitoring device;

FIG. 93 depicts a flowchart of an example method for disease diagnosis and/or screening, and efficacy evaluation of disease treatment on human or veterinary users;

FIGS. 94A-94E illustrate different placements of a monitoring device on a user;

FIG. 95 depicts an example first method of determining a comfort level of a user;

FIG. 96 illustrates an example second method of determining a comfort level of a user;

FIG. 97 depicts an example first method of determining a calorie count using temperature measured at least one extremity of a user;

FIG. 98 illustrates an example second method of determining a calorie count using temperature measured at least one extremity of a user;

FIG. 99 depicts an example first method of using pulse oximetry and thermometry data to determine a sensor is placed correctly on a valid measurement site;

FIG. 100 illustrates an example second method of using pulse oximetry and thermometry data to determine a sensor is placed correctly on a valid measurement site;

FIG. 10A depicts example user interface screens that are suitable for display on a host device;

FIG. 101 illustrates optical data and thermometry data collected by a monitoring device;

FIGS. 102-103 illustrate various aspects to consider when designing an accurate thermometry system;

FIGS. 104A-104C illustrate an example monitoring device that includes built-in thermometry and pulse oximetry;

FIG. 105 illustrates example circuitry that is suitable for use as a built-in temperature sensor in a monitoring device;

FIG. 106 depicts an example embodiment that is used to describe how to apply the embodiments described herein to implement a distributed biosensing system with a wristwatch and a forehead patch both worn by a user;

FIG. 107 illustrates a flowchart of an example first method of operating a wearable monitoring device that includes one or more bio-batteries;

FIG. 108 depicts a flowchart of an example second method of operating a wearable monitoring device that includes one or more bio-batteries;

FIG. 109 illustrates a flowchart of an example third method of operating a wearable monitoring device that includes one or more bio-batteries;

FIG. 110 depicts a flowchart of an example method of operating a wearable monitoring device that includes reusable circuitry and disposable component(s) with at least one bio-battery;

FIG. 111 illustrates a cross-section of a measurement site and cross-section and top views of an example wearable monitoring device that is powered by a bio-battery;

FIG. 112 depicts the bio-battery of the wearable monitoring device shown in FIG. 111;

FIG. 113 is a chart illustrating an example operation of the wearable monitoring device shown in FIG. 111;

FIG. 114 depicts a flowchart of an example method of operating a garment that includes one or more bio-batteries;

FIG. 115 illustrates a flowchart of an example first method of operating a garment that includes one or more bio-batteries and one or more monitoring devices;

FIG. 116 depicts a flowchart of an example second method of operating a garment that includes one or more bio-batteries and one or more monitoring devices;

FIG. 117 illustrates a flowchart of an example method of operating a disposable smart garment that includes reusable circuitry, at least one bio-battery, and at least one monitoring device;

FIG. 118 depicts a flowchart of an example first method of operating a washable smart garment;

FIG. 119 illustrates a flowchart of an example second method of operating a washable smart garment;

FIGS. 120-122 depict example smart garments that have the cathode and anode electrodes of bio-batteries distributed throughout the smart garment;

FIG. 123 illustrates a network of bio-batteries that is suitable for use in a smart garment;

FIG. 124 depicts example first circuitry that is suitable for use with the network of bio-batteries shown in FIG. 123;

FIG. 125 illustrates example second circuitry that is suitable for use with the network of bio-batteries shown in FIG. 123;

FIG. 126 depicts a front and a back view of an example shirt, where the shirt includes two continuous areas that serve as the cathode and the anode electrodes of a bio-battery;

FIG. 127 illustrates example circuitry that is suitable for use with the shirt shown in FIG. 126; and

FIG. 128 depicts an example user interface screen that is suitable for displaying measurements calculated from data received from the garments shown in FIGS. 120-122 and FIG. 126.

### DETAILED DESCRIPTION

As used herein, the term “optimal” is intended to be construed broadly and is intended to cover values and models that provide best, substantially best, and acceptable values and models. As used herein, the term “data stream” refers to data sequentially indexed by an exogenous quantity (e.g., time, space, etc.). For instance, data streams that are function of time, are assumed to be indexed by time such as in a discrete-time system. Data streams that are function of space, are assumed to be indexed by space. Depending on the application, data streams can be indexed by quantities that have physical meaning or that are abstract in nature. The embodiments disclosed herein can be applied to any data stream regardless of its indexing or sampling method.

As used herein, variables and functions can have implicit or explicit arguments in equations, drawings, and text. This is used in order to simplify notation. For instance, whenever possible, a variable or function x(t, i) can have some of its arguments dropped (e.g., x(i), x(t)) in a particular equation, figure, or text in order to make the description shorter and more easily readable. For instance, if a variable or function x(t, i) is written as x(i), it means that “t” is an implicit argument and “i” is an explicit argument. In addition, depending on the context, x(t) can represent a variable or function in time, a set of discrete-time points of a variable or function, or a continuous range of values for a variable or function in time. The term “epoch” is used to define time or time periodicity for a particular embodiment. For instance, a data stream s(t) can be processed every time “epoch”, which means that a sample, or a finite set of samples, or a range of values, or a continuous range of values was processed every certain amount of time, which can be a whole or a fraction of the elapsed time interval. The same is applicable to a set of data streams, sₖ(t), where “k”, or some other variable, indexes each data stream in the set. The time intervals between epochs can be constant or variable depending on the context and application. Hence, epochs “t”, “(t+1)”, . . . , can indicate the evolution of time represented by constant or variable time intervals. The calculations, processing, and manipulations that occur at each epoch “t”, “(t+1)”, . . . , can refer to a single point in time, several points in time, several points in time between adjacent, consecutive, or disjoint epochs, or a time interval, depending on the application and/or context. In a particular equation, figure, or text, the notation x(t, i) can represent a single point in time for a single instance of “x” indexed by a particular “i”, a single point in time for all instances of “x” indexed by “i”, several points in time for a single instance of “x” indexed by a particular “i”, several points in time for all instances of “x” indexed by “i”, etc. The notation x(t, i) can also represent an instance or several instances of a multi-dimensional variable or function indexed by “i”. In this case, a point becomes a vector, array, matrix, or a set of values. In the case of continuous systems, several points or a set of values in time are replaced with ranges of continuous values or sets of continuous values in time.

As used herein, a series of functions, variables, data streams, data sets, models, reference models, etc. are indexed by variables so as to represent the entire series, an instance of series, or several instances of series. For instance, m_{k,i}(t) or ƒ_{k,i}(t) represent a series of data streams or data sets indexed by “i”, with elements indexed by “k”, where each element comprises of a stream with a single value or a plurality of values as function of time epoch “t”. Variables such as r_{k,j}, Corrₖ(i), and w_{k,i}, for example, represent a series of parameters or metrics indexed by “j” or “i”, where each element “k” comprises of a single value or a plurality of values. The argument “t” has been dropped from r_{k,i}, Corrₖ(i), and w_{k,i} to simplify notation. However, they are also evaluated at each time epoch “t”, and as result, they implicitly are functions of time or some other exogenous component quantity that can be used to sequence events or dynamic behavior.

As used herein, the symbols “*” and “+” can be used as a superscript to denote an optimal solution or optimal values for a particular variable or function. For instance, g_{k,i}* represents the optimal value for parameter g_{k,i}, or the optimal values for series of parameters g_{k,i} indexed by “i”, where each parameter comprises of a set of values indexed by “k”. Even though the argument “t” has been dropped from g_{k,i}*, its value or values are evaluated at each time epoch “t”, and as result, g_{k,i}* is implicitly function of time. Series of indexed measurements such as PR(i), SpO2(i), and PI(i) are indexed by “i” and are computed at each time epoch “t”. The corresponding measurements PR(i⁺), SpO2(i⁺), and PI(i⁺) are selected at each time epoch “t” via optimal index “i⁺” value that has been determinate by means of computation algorithms (e.g., classification, optimization, search, etc.).

In general, the term “firmware” is used in conjunction with a monitoring device to distinguish from the program instructions in a host device, and the terms “software” and “application” are used in conjunction with a host device to distinguish from the program instructions in a monitoring device. However, the terms “firmware”, “software”, and “application” are to be construed broadly and are intended to cover software, firmware, and combinations thereof. The use of the term “firmware” does not limit embodiments to only firmware in a monitoring device. Similarly, the use of the terms “software” and “application” do not limit embodiments to only software and/or an application in a host device.

A user can be a layperson, a patient in a medical setting or under medical care, an athlete, an animal, or another person who uses a monitoring device as described herein. A caregiver can be a doctor, a nurse, another medical professional, an at-home care provider, a family or friend, or any other person assisting, monitoring, or otherwise associated with the care of a user.

Reference will now be made in detail to representative embodiments illustrated in the accompanying drawings. It should be understood that the following descriptions are not intended to limit the embodiments to the described example embodiments. A monitoring device is an electronic fitness or monitoring device that measures, tracks, and/or reports data regarding a comfort level of a user and/or measurements of one or more physiological parameters, including, but not limited to, heart rate, blood perfusion, oxygen saturation, body temperature, calorie count, and the like. The measurements or data relating to the measurements of the one or more physiological parameters can be sent to a computing device for further processing. For example, oxygen saturation (SpO2), pulse rate (PR), perfusion index (PI), and/or pulse amplitude (PA) can be estimated on a computing device.

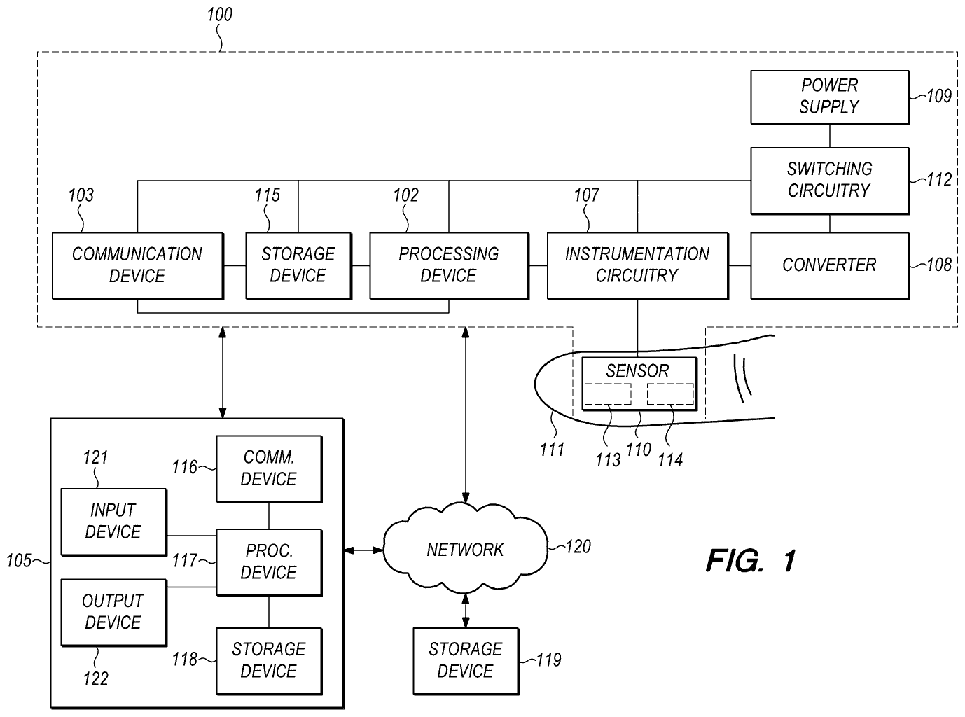

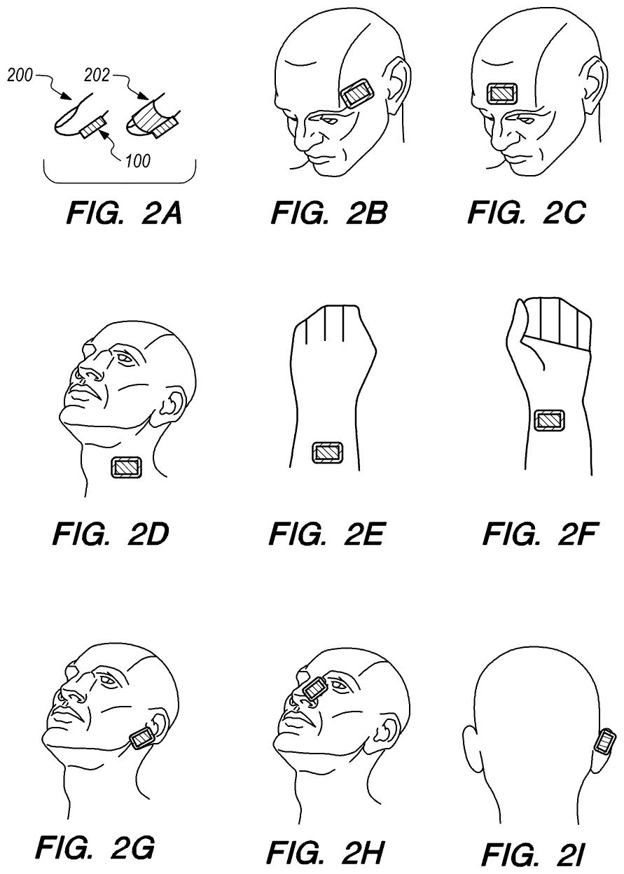

FIG. 1 is a block diagram illustrating a first example of a monitoring device connected to a computing device and network. A monitoring device 100 is attached to one or more measurement sites from which the sensor can readily access blood perfusion information. In the illustrated embodiment, the measurement site 111 is a finger or digit (see also FIG. 2A). Other example measurement sites include, but are not limited to, a user's temple (FIG. 2B), forehead (FIG. 2C), neck (FIG. 2D), arm (FIGS. 2E and 2F), ear or ear lobe (FIG. 2G), nose (FIG. 2H), and/or the posterior auricle of an ear (FIG. 2I).

In one embodiment, the monitoring device 100 includes a processing device 102, instrumentation circuitry 107, a communication device 103, and a storage device 115. The instrumentation circuitry 107, the communication device 103, and the storage device 115 are connected to the processing device(s) 102. A converter 108 is connected to the instrumentation circuitry 107 and switching circuitry 112. The communication device 103, the storage device 115, the processing device 102, and the instrumentation circuitry 107 along with a power supply 109 are connected to the switching circuitry 112. The monitoring device 100 may also include adhesive tape to attach the monitoring device 100 to a measurement site.

The monitoring device 100 may be turned on using the switching circuitry 112. In one instance, the switching circuitry 112 is a single-use, conductive tape-switch. The instrumentation circuitry 107 may include one or more light sources, such as a light-emitting diode (LED), control circuitry and logic, and one or more photodetectors, such as a photodiode. The communication device 103 can be any suitable type of communication device, including, but not limited to, a wireless low energy radio (examples of which include, but are not limited to, BLE, ANT, Zigbee, etc.). Wireless connection and authentication (when required) between communication devices 116 and 103 can be accomplished through standard pairing methods (i.e., Just Works, etc.) and out-of-band methods, such as Near-Field Communication (NFC), barcode/image scanning, or via an optical link between the optical sensor 110 and camera (or optical sensor) housed in the host device 105. Depending on the configuration of the monitoring device 100, the storage device 115 may comprise, but is not limited to, volatile storage (e.g., random access memory), non-volatile storage (e.g., read-only memory), flash memory, or any combination of such memories.

The monitoring device 100 can be communicatively coupled to a host device 105, such as a smart phone, a tablet computing device, a desktop or laptop computer, a wireless computing and/or data aggregator appliance device, a bedside monitor, or a similar computing device through a wired or wireless connection. The host device 105 can include a communication device 116 and a storage device 118 connected to a processing device 117. The monitoring device 100 transmits, via the communication device 103, measurement data to the host device 105 (via the communication device 116) to be processed, displayed and/or stored. The measurement data can be used for alarms, for electronic medical record data transfer, for data sharing, and/or for other uses of the data.

The host device 105 may further include one or more input devices (represented by input device 121) and/or one or more output devices (represented by output device 122). The input and output devices 121, 122 are connected to the processing device 117. The input device 121 can be implemented as any suitable input device, such as a keyboard (physical or virtual), a mouse, a trackball, a microphone (for voice recognition), an image capture device, and/or a touchscreen or touch display, or any other computer-generated perceptual input information. The output device 122 may be implemented as any suitable output device, such as a display, one or more speakers, and/or a printer, or any other computer-generated perceptual output information. In some embodiments, the measurement data or data representative of the measurement data can be provided to the output device 122. For example, the measurement data or data representative of the measurement data may be displayed on a display.

In some embodiments, the host device 105 and/or the monitoring device 100 can access an external storage device 119 through one or more networks (represented by network 120) to store and/or retrieve measurement data. In one or more embodiments, the network 120 is illustrative of any suitable type of network, for example, an intranet, and/or a distributed computing network (e.g., the Internet) over which the computing device and/or the monitoring device 100 may communicate with other computing devices.

As will be described in more detail later, the measurement data produced by the monitoring device 100 can be processed to determine or estimate one or more physiological parameters (e.g., pulse rate, blood oxygen saturation, calorie count). As part of the processing, one or more signals are processed with a numerical solver device. The numerical solver device can be implemented with one or more circuits (circuitry), a software algorithm or program executed by one or more processing devices (e.g., processing device 102 and/or processing device 117), or a combination of circuitry and a software algorithm.

For example, in one embodiment, the storage device 115 in the monitoring device 100 can include a number of software programs or algorithms and data files, including a numerical solver device. While executing on the processing device 102, the numerical solver device may perform and/or cause to be performed processes including, but not limited to, the aspects as described herein. In another embodiment, the storage device 118 in the host device 105 can include a number of software programs or algorithms and data files, including a numerical solver device. While executing on the processing device 117, the numerical solver device may perform and/or cause to be performed processes including, but not limited to, the aspects as described herein. In yet other embodiments, the operations of the numerical solver device are distributed such that some of the operations are performed by the processing device 102 and some of the operations are performed by the processing device 117.

Small Footprint

Embodiments of the monitoring device can provide a relatively small footprint (size). Among other aspects, a smaller size can require less material in manufacturing, improved ease of use, less room required for storage, less costs for transport, and a less intrusive device and instrument for users' increased comfort and mobility when using the monitoring device. In one embodiment, the monitoring device 100 may include a printed circuit board (PCB) comprising the processing device 102 with an integrated communication device 103, a compact integrated circuit that includes the instrumentation circuitry 107 for signal conditioning and LED current driving, the power supply 109, and the converter 108. The power supply 109 combined with the converter 108 provide the required higher voltage to drive the light sources 113 and/or a photodetector 114 of the optical sensor 110. In one embodiment, the converter 108 is a single DC-DC switched converter, the power supply 109 is a disposable battery, the light sources 113 are LEDs, and the photodetector 114 is a silicon photodiode.

The processing device 102 and the instrumentation circuitry 107 can be powered directly by the power supply 109. The optical sensor 110 may be encapsulated with the PCB by any one of a wide variety of suitable apparatus and methods, including (by way of example) by attaching flexible adhesive tapes of various types (optionally combined with PTFE) to the PCB. Persons of ordinary skill in the art will understand that the PCB may be rigid or flexible, or be in the form of a substrate where some or all the components are die attached and wire bonded to the substrate, and encapsulated for protection using epoxy or some other encapsulation material. Further, the optical sensor 110 may be attached to a measurement site 111 using any of a wide variety of suitable apparatus and methods, including (by way of example) by using the adhesive tapes that are part of the optical sensor 110 encapsulation structure (as described herein).

Low Power Consumption

In some aspects, the processing device 102 is a low-power ARM processor with dual functionality for controlling a wireless low energy radio (communication device 103) and instrumentation circuitry 107. The optical sensor 110 can include high efficiency LEDs and at least one silicon photodiode that are arranged in a reflective configuration such that the LEDs and the at least one silicon photodiode are physically separated from each other to minimize the required LED currents and frontend gains in the instrumentation electronics. The instrumentation circuitry 107 may have very low bias currents and operate at low voltages. In one embodiment, ambient light interferences may be avoided or at least reduced by modulating and time-multiplexing the LEDs' currents at a higher frequency to shift the spectral content of the generated and detected optical signals to a range in the spectrum where ambient light interferences are less likely to occur.

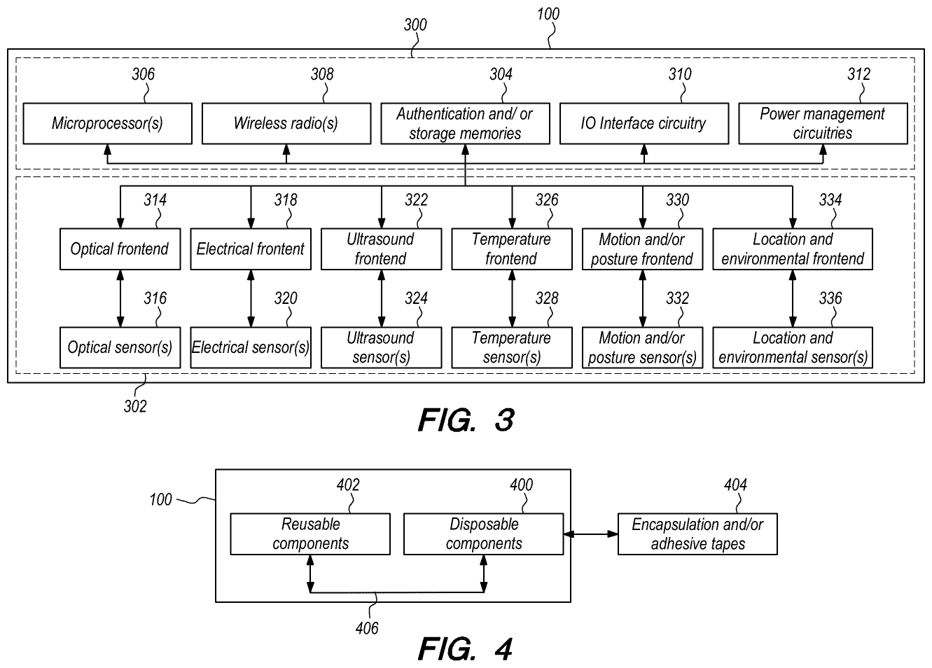

FIG. 3 is a block diagram illustrating a second example of a monitoring device. The monitoring device 100 includes a first section 300 operably connected to a second section 302. The first section 300 includes an authentication and/or storage memory or memories 304 operably connected to microprocessor(s) 306, wireless communication device(s) 308, input/output (IO) interface circuitry 310, and power management circuitry 312. The second section 302 may include an optical frontend 314 operably connected to optical sensor(s) 316, an electrical frontend 318 operably connected to electrical sensor(s) 320, an ultrasound frontend 322 operably connected to ultrasound sensor(s) 324, a temperature frontend 326 operably connected to temperature sensor(s) 328, a motion and/or posture frontend 330 operably connected to motion and/or posture sensor(s) 332, and/or a location and/or environmental frontend 334 operably connected to location and/or environmental sensors 336. In one embodiment, the optical frontend 314, the electrical frontend 318, the ultrasound frontend 322, the temperature frontend 326, the motion and/or posture frontend 330, and the location and/or environmental frontend 334 control the transmission of sensor data from the respective sensors to the first section 300. The frontends 314, 318, 322, 326, 330, 334 are used to interface with the sensors 316, 320, 324, 328, 332, 336, respectively to provide control, communication, and power signals to the respective sensors 316, 320, 324, 328, 332, 336 to enable sensor correct operations. The sensor correct operations include, but are not limited to, electrical voltage level and current level translations, analog-to-digital conversions, digital-to-analog conversions, electronic gain selections, saturation detection, error detection, modulation, demodulation, filtering, decimation, and/or time and/or frequency multiplexing and demultiplexing.

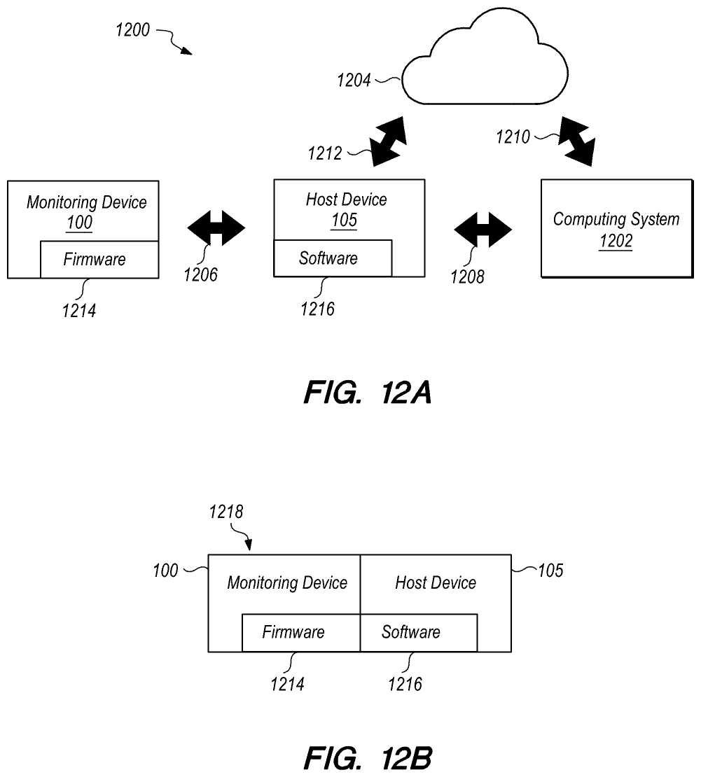

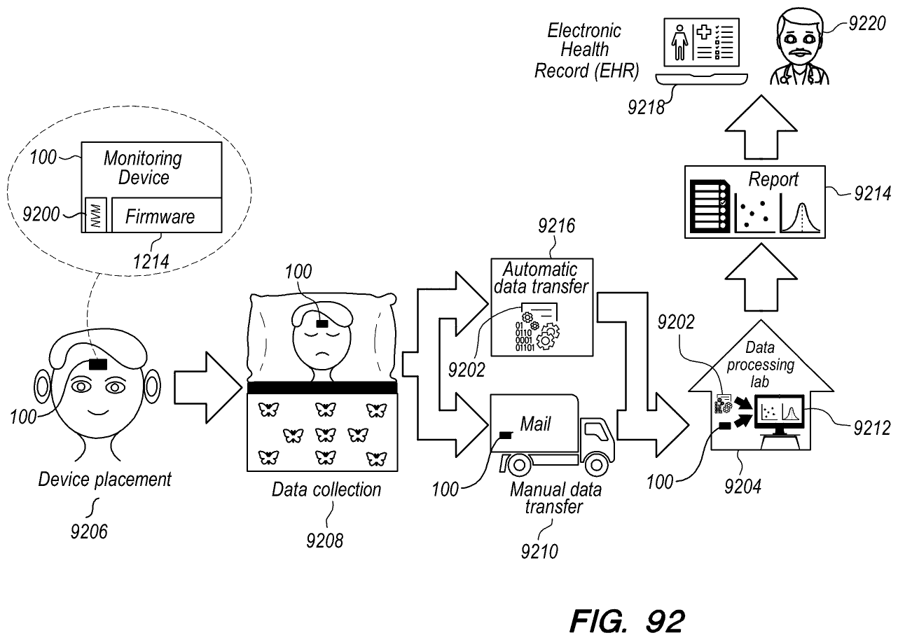

In one embodiment, all of the components shown in FIG. 3 are included in the monitoring device 100. In another embodiment, only a portion of the components are included in the monitoring device 100, depending on the application and measurement site. For instance, in the example embodiments depicted in FIG. 92 and FIGS. 94B-94E, the monitoring device 100 includes the microprocessor(s) 306, the wireless communication device(s) 308, the authentication and/or storage memory or memories 304, the power management circuitry 312, the optical frontend 314, the optical sensor(s) 316, the temperature frontend 326, the temperature sensor(s) 328, the electrical frontend 318, and the electrical sensor(s) 320. The power management circuitry 312 can comprise of linear, boost and buck regulators, energy harvesting circuitries, rechargeable and/or non-rechargeable batteries and/or battery fuel gauge circuitries. The microprocessor(s) 306 can run firmware (e.g., firmware 118 in FIG. 12A) that controls monitoring device 100. The wireless communication device 308 can be used to transfer data from the monitoring device 100 to a processing entity (e.g., a data processing laboratory or business and/or a host device (e.g., host device 105 in FIG. 1)). The authentication and storage memory or memories 304 can be used to store data collected by the monitoring device 100 (e.g., waveforms, measurement trends, etc.) before the collected data is transferred to a processing entity (e.g., a data processing lab and/or a host device). The authentication and storage memory or memories 304 can also be used to prevent the monitoring device 100 (or any disposable assemblies from the memory device 100) from being counterfeited and/or connected to unauthorized third-parties. Additionally or alternatively, storing the collected data in the authentication and storage memory or memories 304 can ensure the collected data does not exceed a maximum amount of usage (e.g., maximum number of transitions, maximum usage time, etc.).

Electroencephalogram data changes when a user (e.g., a patient) is asleep or awake, and the brainwave pattern evolution can augment the detection of sleep modes (e.g., REM sleep and non-REM sleep). For applications where the electrical sensor(s) are placed on a measurement site on the user's upper or lower chest or the upper back, the electrical sensor(s) can be used to collect electrocardiogram (ECG) data. The ECG data can be used for the screening or diagnosis of cardiovascular diseases, as well as used in conjunction with optical data obtained by the optical sensor(s) to estimate pulse transit time and blood pressure. The ultrasound frontend 322 and the ultrasound sensor(s) 324 can be used to probe the measurement site for information related to geometry, tissue layer, and mechanical properties to augment and account for measurement site variabilities when measuring physiological parameters such as blood pressure, glucose, etc. The motion and/or posture frontend 330 and the motion and/or posture sensor(s) 332 can be used to measure, for instance, the user's physical motion and posture during sleep to improve the detection of the sleep modes. The location and environmental frontend 334 and the location and/or environmental sensor(s) 336 can be employed to measure the user's physical location, and/or environmental altitude, pressure, humidity, and/or temperature to account for measurement errors caused by changes in physiology due to environmental factors.

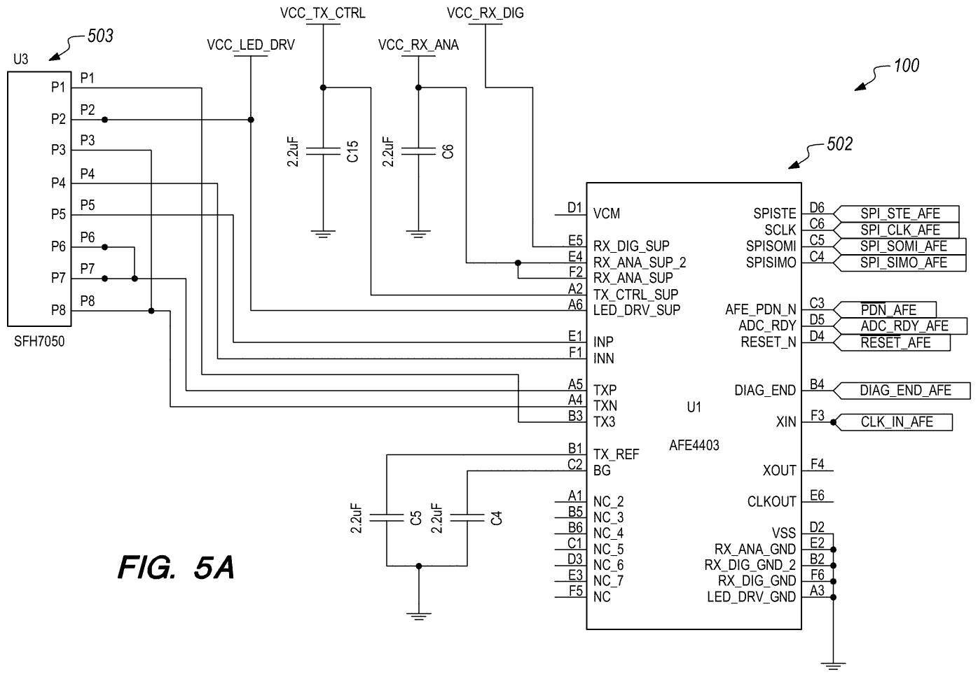

FIG. 4 is a block diagram illustrating a third example of a monitoring device.

Depending on the application, the monitoring device 100 can be constructed so as to have disposable components 400 and/or reusable components 402. The components that are in contact with the user's skin (e.g., electrode(s), electrical sensor(s), optical sensor, etc.) can be designed to be single-use (disposable) to minimize the risks of cross contamination among users (e.g., patients). The electronics and the substrates can be made fully disposable or reusable (or partially reusable) to reduce the final cost per user of the monitoring device. Encapsulation and/or adhesive tapes 404 can be employed to optimize the attachment of the monitoring device 100 to a measurement site when the application requires prolonged use. The encapsulation can be reusable, partially reusable, or fully disposable depending on costs, risks, and regulations, and made out of foams, plastics, carbon fibers, organic and/or recyclable materials, paper, etc. Adhesive tapes can be designed to be disposable single- or multi-piece, with thermally and/or electrically conductive tapes, low-cost connectors and/or cables, low-cost self-aligning magnetic connectors to enable electrical, optical, thermal, and mechanical connections 406. In a medical setting, such as a hospital or a doctor's office, recharging the battery of the monitoring device 100 prior to use may be challenging given the complex scheduling, lack of resources, the number of users to examine that day, and/or the procedures to be performed. To simplify the workflow, the battery of the monitoring device can be made non-rechargeable and part of a single-use assembly with adhesive tapes, encapsulations, and/or other electronic components (e.g., low-cost anti-tamper memory part of the authentication and/or storage memory/memories 304 that prevents counterfeiting, and monitors device usage).

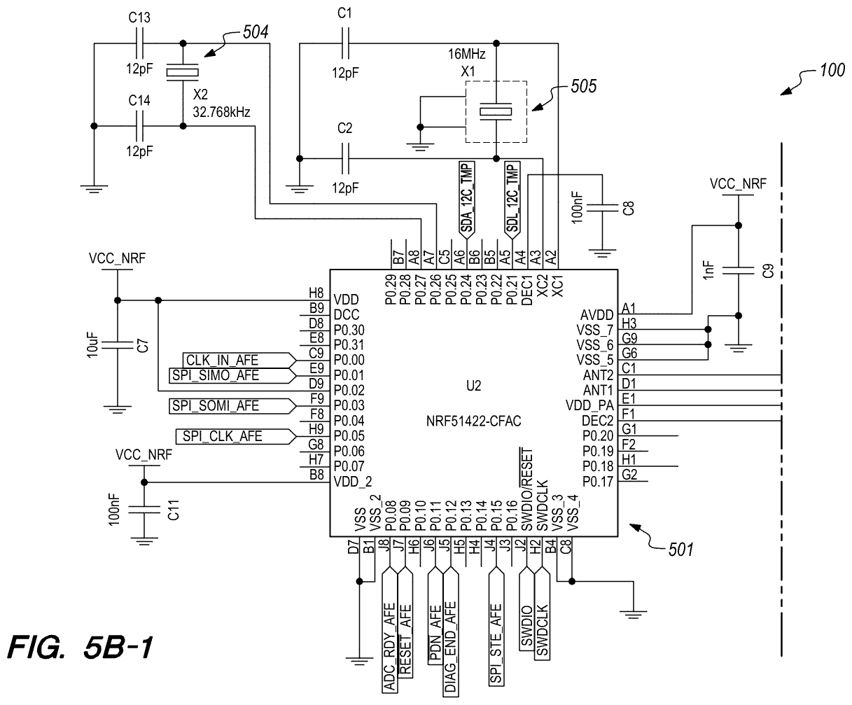

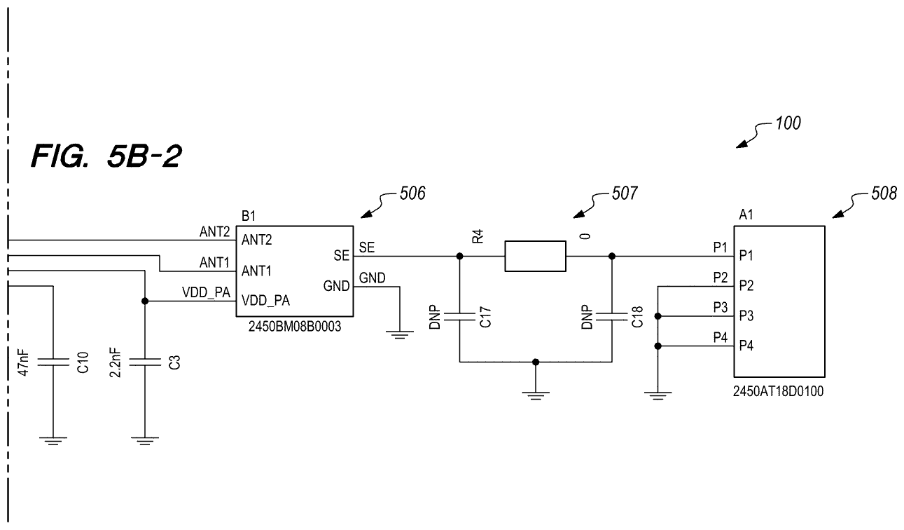

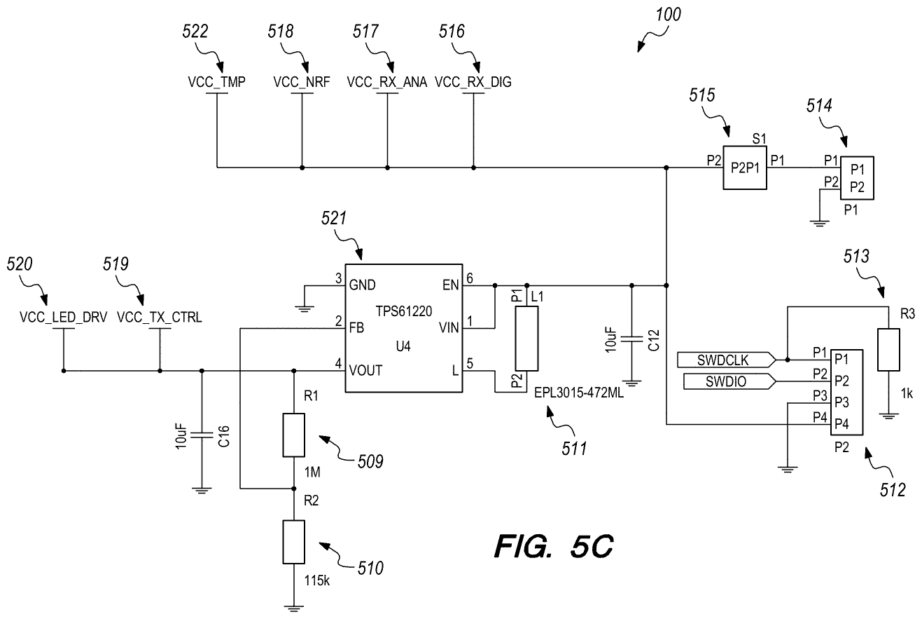

FIGS. 5A-5C are schematic diagrams illustrating an example monitoring device. The components depicted in FIGS. 5A-5C, and the corresponding descriptions of FIGS. 5A-5C, are for purposes of illustration only and are not intended to limit embodiments to a particular sequence of steps or a particular combination of hardware or software components.

The monitoring device 100 may include an integrated circuit 502 (FIG. 5A), such as the AFE4403 or the AFE4490 circuits by Texas Instruments, including a photodiode frontend, LED drivers, and control logic. An optical sensor 503 (FIG. 5A), such as the SFH7050 sensor by OSRAM, may include green, red, and near-infrared LEDs and a silicon photodiode. The monitoring device 100 may include a main processing device 501 (FIG. 5B), such as the ARM Cortex M0 processor available from Nordic Semiconductors. Further, the monitoring device 100 may include a 16 MHz crystal oscillator 505, a 32.768 kHz crystal oscillator 504 (when ANT low-energy radio is used), a 2.45 GHz impedance balloon filter (single to differential) 506, a matching impedance circuit 507, and an antenna 508 (FIG. 5B). A power management circuit of the monitoring device 100 is shown in FIG. 5C and may include: a boost converter 521 such as TPS61220 from Texas Instruments, a ferrite inductor 511, boost converter voltage setting resistors 509, 510, debug pads 512 for the main processing device 501, noise rejection pull down resistor 513, battery voltage terminals 514, ON switch pads 515, and voltages 516, 517, 518, 519, 520, 522 for the main processing device 501 (FIG. 5B) and integrated circuits. In one embodiment, the ON switch pads 515 are single use pads.

As should be appreciated, the components depicted in FIGS. 5A-5C, and the corresponding descriptions of FIGS. 5A-5C, are for purposes of illustration only and are not intended to limit embodiments to a particular sequence of steps or a particular combination of hardware or software components.

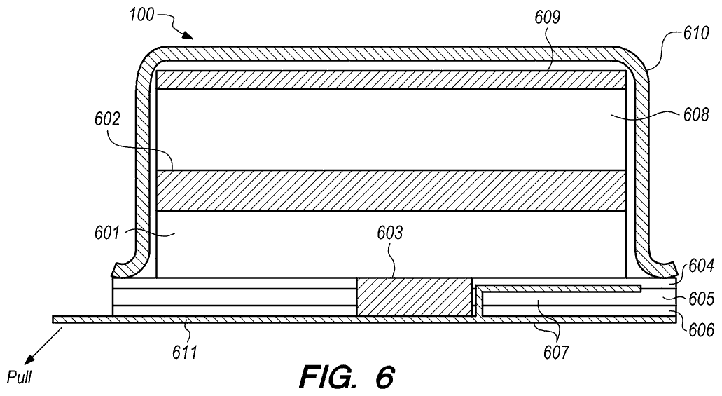

FIG. 6 depicts a cross-section view of an example monitoring device. FIG. 6 depicts one of the many ways to fabricate a stack-up of the various components of the monitoring device 100. A PTFE encapsulation pocket or an origami made out of biocompatible tape 610 may house the components of the monitoring device 100. Beginning at the top surface 610 and moving down to the bottom surface 611, the illustrated monitoring device 100 includes: an antenna 609, a battery 608, a printed circuit board (PCB) 601 and PCB circuitry 602, and an optical sensor 603. For attachment to a user's measurement site such as a fingertip, the monitoring device 100 includes a PCB-to-skin adhesive layer 606. A contact layer 605 is made of electrically conductive tape (such as an isotropically conductive pressure sensitive tape), and the contact layer 604 contains electrical contacts that (when closed) supply power to the PCB 601. A release liner 607 may be disposed between the contact layers 604 and 605, and on adhesive layer 606 such that when the release liner 607 is pulled, the optical sensor 603 and the adhesive layer 606 for attachment to a measurement site are exposed. Also exposed are the connecting layers 604 and 605 to turn on the monitoring device 100.

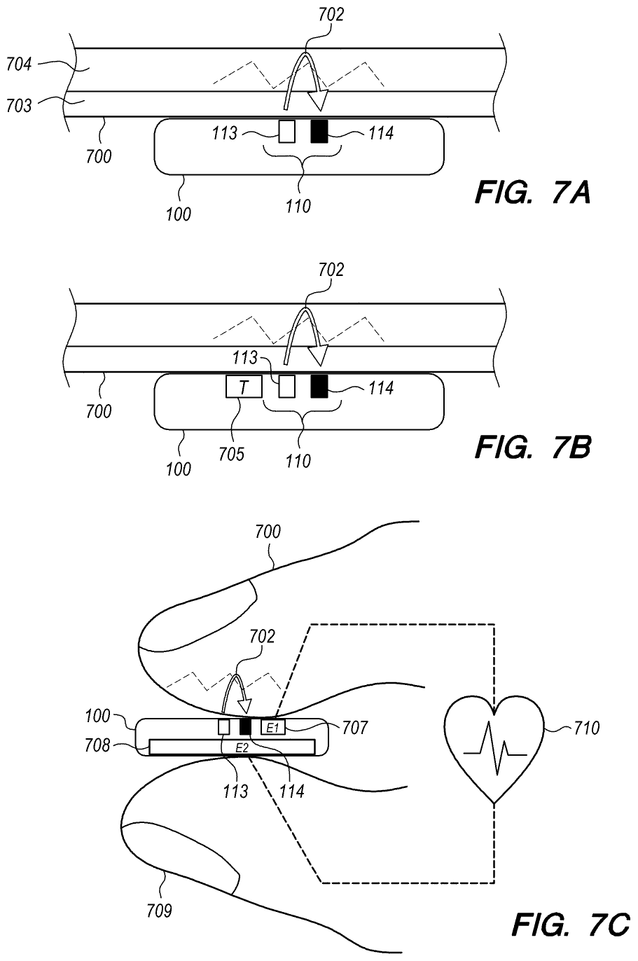

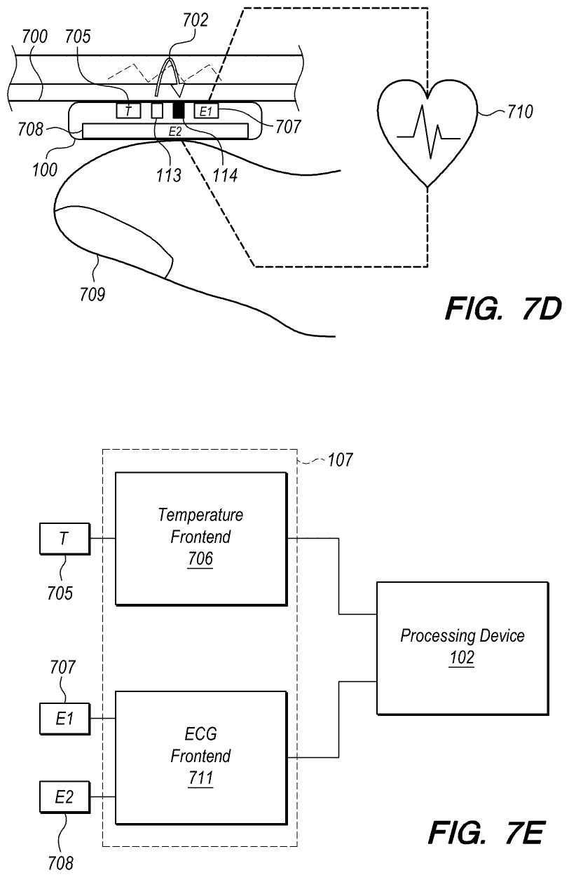

FIGS. 7A-7E depict example embodiments of a monitoring device. In FIG. 7A, the monitoring device 100 obtains signals from an optical sensor (e.g., photoplethysmographs). The monitoring device 100 is affixed to a measurement site 700 and includes an optical sensor 110. As described previously, the optical sensor 110 includes one or more light sources 113 and one or more photodetectors 114. The one or more light sources 113 emit light 702 towards the measurement site 700 that penetrates the epidermis 703 and the blood perfused dermis 704 and interacts with the heart's pulsatile signal to create optical pulsatile signals. The dashed line in FIGS. 7A-7D represents changes in blood volume that occur at the blood perfused dermis 704 caused by the heart activity (plethysmographs). The optical pulsatile signals (photoplethysmographs) are captured by a frontend circuitry (e.g., instrumentation circuitry 107 and processing device 102 in FIG. 1) connected to the one or more photodetectors 114. The frontend circuitry filters, conditions, and/or converts the optical pulsatile signals to digital signals. These digital signals can be transmitted wirelessly to a host device (e.g., host device 105 using the communication device 103 in FIG. 1) for further processing and analysis, real-time measurement displaying, warning (alarm) generation, and/or storage of the subject's SpO2, PR, PI, etc.

In FIG. 7B, the monitoring device 100 obtains both the signals from the optical sensor as well as signals from a temperature sensor. The monitoring device 100 includes the optical sensor 110 and one or more temperature sensors (represented by temperature sensor (T) 705) in contact with the measurement site 700. The temperature sensor 705 is used to measure the core body temperature. For instance, the monitoring device 100 can be attached to the forehead, the ear (posterior auricle), or some other location on the user's body with superficial temperature correlated to the body's core temperature. The temperature sensor 705 can be connected to temperature frontend circuitry 706 (depicted in FIG. 7E) that conditions, filters, and/or converts the temperature signal to a digital temperature signal. The digital temperature signal can then be transmitted wirelessly to a host device (e.g., host device 105 using communication device 103 in FIG. 1) for further processing and analysis, real-time measurement displaying, warning (alarm) generation, and/or storage of the body's core temperature. In one embodiment, the temperature frontend circuitry 706 can be included in the instrumentation circuitry 107 that interfaces (for data collection) with the processing device 102 shown in FIG. 1.

The monitoring device 100 in FIG. 7C obtains the signals from the optical sensor as well as signals from an electrical sensor. The monitoring device 100 includes a first electrical contact sensor (E1) 707 and a second electrical contact sensor (E2) 708. The example embodiment can be used on measurement sites where either the temperature sensor (T) 705 is not needed, or the superficial temperatures at the measurement sites are not correlated to the body's core temperature. In the illustrated embodiment, the monitoring device 100 is attached to the measurement site 700 (e.g., the user's finger). The first electrical contact sensor (E1) 707 is positioned on an interior side of a first surface of the monitoring device 100, where the exterior side of the first surface is in contact with the measurement site 700 when the monitoring device 100 is attached to the measurement site 700. Accordingly, the first electrical contact sensor (E1) 707 is in constant contact (or near constant contact) with the measurement site 700 when the monitoring device 100 is positioned on the measurement site 700.

The second electrical contact sensor (E2) 708 is positioned on an interior side of a second surface of the monitoring device 100. In FIG. 7C, the second surface is opposite the first surface, although other embodiments are not limited to this configuration. The second electrical contact sensor (E2) 708 can be located on a side of any surface such that the second electrical contact sensor (E2) 708 is not in contact with the measurement site 700 when the monitoring device 100 is attached to the measurement site 700.

When the monitoring device 100 is placed or attached on the measurement site 700, the user can touch the electrical contact sensor 708 with a different body part (e.g., thumb 709 of the opposite hand) to create a closed electrical path to the user's heart 710 and enable the measurement of the user's heart electrical activity (e.g., an ECG). The first and the second electrical contact sensors 707, 708 can be connected to the ECG frontend circuitry 711 (depicted in FIG. 7E). The ECG frontend circuitry 711 conditions, filters, and/or converts the ECG signal to an ECG digital signal. The ECG digital signal can be transmitted wirelessly to a host device (e.g., host device 105 using communication device 103 in FIG. 1) for further processing and analysis, real-time displaying of measurements, warning (alarm) generation, and/or storage of the ECG signal. In one embodiment, the ECG frontend circuitry 711 is part of the instrumentation circuitry 107 and includes the processing device 102 shown in FIG. 1.

With reference to FIG. 7D, the monitoring device obtains the signals from the optical sensor, the signals from the temperature sensor, and the signals from the electrical sensor. In the example embodiment, the monitoring device 100 includes the optical sensor 110 from FIGS. 7A-C (represented by elements 113 and 114 in FIG. 7D), the temperature sensor (T) 705, and the first and the second electrical contact sensors 707, 708. In the illustrated embodiment, the measurement site 700 is assumed to have a superficial temperature that is correlated to the body's core temperature (i.e., a forehead, ear (posterior auricle), armpit, etc.) or have a temperature that can be mapped to correlate with the body's core temperature (i.e., finger, etc.). Like FIG. 7C, the user creates a closed path to the user's heart 710 when the monitoring device 100 is placed on the measurement site 700 such that the first electrical contact sensor (E1) 707 contacts the measurement site 700 and the user touches the second electrical contact sensor (E2) 708 with a different body part (e.g., the thumb 709 or any other body part) that creates a closed electrical path to the user's heart 710. The first and the second electrical contact sensors 707, 708 depicted in FIGS. 7C and 7D can be replaced with capacitive contact sensors in embodiments where electrical isolation between the monitoring device 100 and the user's body is preferred. In such embodiments, the ECG frontend circuitry 711 can have a high input impedance to enable the acquisition of the heart's ECG without any low-frequency harmonic distortions (attenuation) that can be created by the capacitive contact sensors.

FIG. 7E depicts additional frontend circuitries 706 and 711 that, in addition to the optical frontend 314 depicted in FIG. 3, can be part of the instrumentation circuitry 107 to enable thermometry and electrical activity (e.g., ECG) monitoring. A comprehensive list of possible frontends (i.e., frontends 314, 316, 322, 326, 330, 334 in FIG. 3) and sensors that can be built into a monitoring device 100 are described in conjunction with FIG. 3.

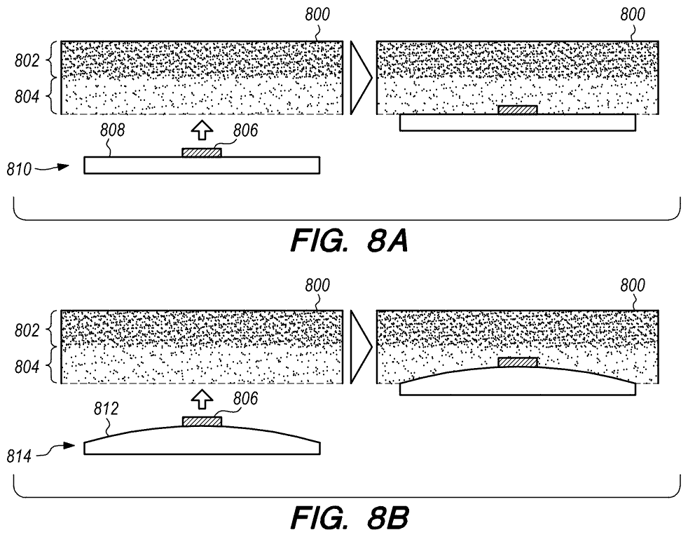

FIGS. 8A-8B illustrate different contact surface configurations of a monitoring device. In a non-limiting example, the contact surface configurations can be used in veterinary applications or human applications. A measurement site 800 of an animal patient or a user is shown in FIGS. 8A and 8B with a gradient filling. The darker region 802 represents tissue having a higher peripheral blood circulation (dermis), or higher blood perfusion. The lighter region 804, closer to the optical sensor 806 (e.g., optical sensor 110 in FIG. 1), represents tissue having a lower peripheral blood circulation (epidermis), or lower blood perfusion. In FIG. 8A, the surface 808 of the monitoring device 100 is depicted in a flat-surface configuration 810 with the optical sensor 806 is attached to the surface 808. When the monitoring device 100 in FIG. 8A is attached to the measurement site 800, additional placement pressure may be needed to improve the optical compliance of the optical sensor 806 and the blood perfused tissue. This can be accomplished by using any suitable technique (not shown). For example, a tape may be wrapped around the monitoring device 100 and the measurement site 800 whenever possible. Additionally or alternatively, an adhesive bandage can be applied over the monitoring device 100 and the measurement site 800.

In FIG. 8B, the surface 812 of the monitoring device 100 is depicted as a convex surface in a convex-surface configuration 814. With the convex-surface configuration 814, attaching only the monitoring device 100 to the measurement site 800 creates additional placement pressure on the tissue around the location of the optical sensor 806, given that the convex surface 812 bulges out and compresses the tissue. The convex-surface configuration 814 can improve the optical compliance of the optical sensor 806 and the measurement site 800, as well as increase the peripheral blood circulation around the location of the optical sensor 806. In the embodiment shown in FIG. 8B, the use of a tape, adhesive bandage, or another attachment mechanism to apply additional placement pressure between the monitoring device 100 and the measurement site 800 is optional or unnecessary based on the convex surface 812 of the monitoring device 100. The adhesive layer in the monitoring device 100 that attaches the monitoring device to the measurement site 800 may be sufficient.

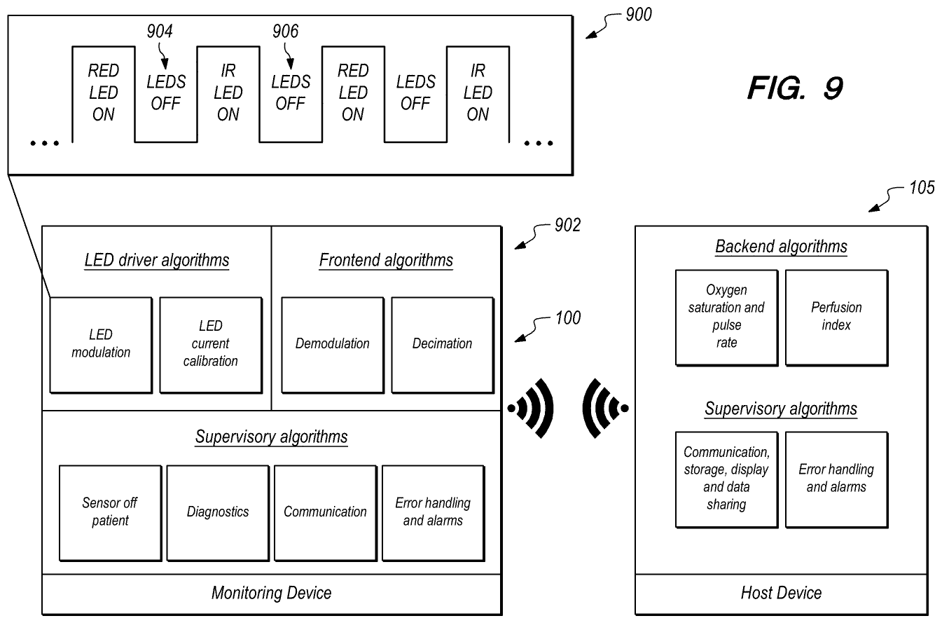

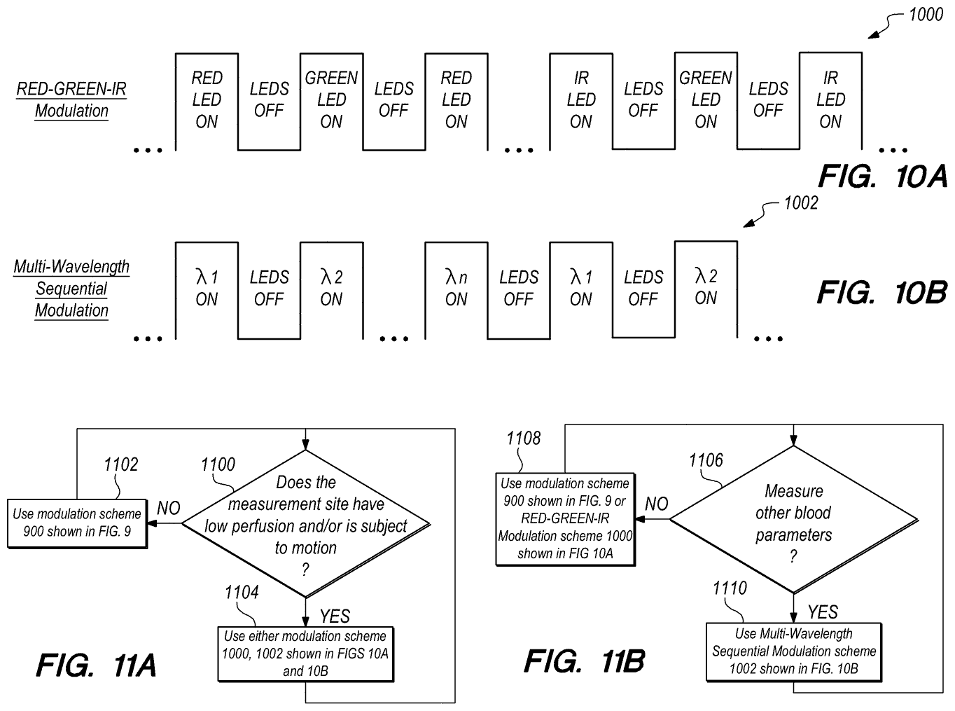

FIG. 9 depicts a distributed system suitable for processing signals produced by a monitoring device and an example modulation scheme that is suitable for use with a monitoring device. The modulation scheme 900 may reduce the complexity of the demodulation, decimation, LED current calibration, sensor off user, error handling and alarms, diagnostics, and/or communication algorithms shown in the algorithm block diagram 902. Some or all of the blocks in the algorithm block diagram 902 are included in a monitoring device 100. The LED driver algorithms, the frontend algorithms, and the supervisory algorithms can each be software programs that are stored in a storage device (e.g., storage device 115 in FIG. 1).

In the modulation scheme depicted in FIG. 9, each LED (light source 113) is turned on for approximately 25% of the modulation time cycle (LED duty cycle). In other embodiments, smaller LED duty cycles can be used to reduce overall power consumption. The LEDs can be turned off for approximately 50% of the modulation time cycle. The intervals where the LEDs are turned off can also be increased if the LED duty cycles are to be reduced and if the modulation frequency is kept the same. The two slots 904, 906 in the waveform represent times when all of the LEDs are turned off. The two slots 904, 906 can be used to probe and cancel the effects of ambient light. Modulation frequencies as low as one Kilohertz (1 KHz) can be adopted with signal-to-noise ratio figures similar to medical-grade pulse oximeters in embodiments that perform sophisticated filtering and signal processing in the demodulation scheme to recover the optical signals generated by the interplay of the LEDs optical signals and the attenuation caused by the measurement site's blood perfused tissue.

In some embodiments, a distributed computing architecture may be used to calculate one or more physiological parameters such as blood oxygen saturation (SpO2), pulse rate (PR), pulse amplitude (PA), and/or perfusion index (PI). For example, the SpO2, PR, PA, and PI are estimated on the host device 105 (e.g., a mobile phone or laptop) to increase the battery life of the monitoring device 100. In one embodiment, one or more numerical solver devices can be included in the backend algorithms of the host device 105. For example, a numerical solver device can be included in the oxygen saturation and pulse rate algorithm and in the perfusion index algorithm. In another example, one or more numerical solver devices can be a separate algorithm that is called by the oxygen saturation and pulse rate algorithm and by the perfusion index algorithm.

In other embodiments, one or more numerical solver devices can be included in the monitoring device 100. For example, one or more numerical solver devices may be implemented in the frontend algorithms, such as, for example, the demodulation algorithm.

A processing device in the monitoring device 100 (e.g., processing device 102 in FIG. 1) may execute the time critical, high frequency, low latency and low complexity tasks. Data processed by the processing device in the monitoring device 100 may be reduced in bandwidth by decimation algorithms, and sent wirelessly to the host device 105 (e.g., to a processing device 117 in the host device 105). In one embodiment, the host device 105 may execute more complex, high latency tasks to calculate and continuously display the measurement values for SpO2, PR and PI.

In an example embodiment, the monitoring device frontend from Texas Instruments (AFE4403) can be used as the instrumentation circuitry 107 in FIG. 1. In such embodiments, the monitoring device frontend may be programmed to generate and control directly the required LED modulation scheme without the need for additional resources from a processing device in the monitoring device 100 (e.g., the processing device 102 in FIG. 1).

Other example modulation schemes are shown in FIGS. 10A and 10B. The RED-GREEN-IR Modulation scheme 1000 shown in FIG. 10A and/or the Multi-Wavelength Sequential Modulation scheme 1002 depicted in FIG. 10B can be used with measurement sites that have low perfusion and/or are subject to excessive motion. FIG. 11A and FIG. 11B depict flowcharts of example methods of determining which modulation scheme to use. FIGS. 11A-11B show example scenarios where a particular type of modulation can be advantageous. In the method shown in FIG. 11A, the adopted modulation scheme depends on the aforementioned factors (e.g., low perfusion and/or subject to excessive motion). Initially, as shown in block 1100, a determination is made as to whether the measurement site has low perfusion and/or is subject to motion. If not, the process passes to block 1102 where the modulation scheme 900 shown in FIG. 9 can be used. When the measurement site has low perfusion and/or is subject to motion, the method continues at block 1104 where the RED-GREEN-IR Modulation scheme 1000 (FIG. 10A) or the Multi-Wavelength Sequential Modulation scheme 1002 (FIG. 10B) may be used.

In the method shown in FIG. 11B, a determination is made at block 1106 as to whether one or more measurements of other blood parameters are to be obtained or determined. The blood parameters can include, but are not limited to, glucose, water, and hemoglobin. If one or more measurements of other blood parameters is to be determined, the process passes to block 1108 where the modulation scheme 900 shown in FIG. 9 or the RED-GREEN-IR Modulation scheme 1000 shown in FIG. 10A can be used. If one or more measurements of other blood parameters will not be determined, the method continues at block 1110 where the Multi-Wavelength Modulation scheme 1002 (FIG. 10B) may be used.

For the RED-GREEN-R Modulation scheme 1000 shown in FIG. 10A, green and red LEDs are activated and modulated for a period of time according to the on-off pattern described, and then, the red LED (RED) is replaced with the near-infrared LED (IR) and also modulated for a period of time. This sequence of events repeats itself while the measurement site is subject to motion and/or low perfusion levels. Applying light in the wavelength range between violet and yellow (i.e., between 400 to 590 nm approximately) to a blood-perfused measurement site, the higher light scattering and absorption seen in this region, create photoplethysmographs that are much larger in amplitude when compared to the photoplethysmographs in the red and near-infrared wavelength regions. Typically, the green wavelength is used because LEDs in this range offer good efficiency and reliability as well as lower cost when compared to other wavelengths in the violet-yellow range. Also, the optical properties of blood in the green region are desirable in terms of scattering and absorption levels. The photoplethysmograph associated with the green LED can be used to improve detection of the heart rate and/or the detection of the red and near-infrared true photoplethysmograph amplitudes and waveforms, which are required for an accurate measurement of the blood's oxygen saturation under low-perfusion and motion conditions.

The Multi-Wavelength Sequential Modulation scheme 1002 shown in FIG. 10B can be used in some embodiments when the parameters of interest require other wavelengths in addition to the red and near-infrared LEDs. Examples include the non-invasive measurement of other blood constituents (parameters), such as glucose, for diabetes disease management, water, for body hydration management, total hemoglobin, for anemia and/or blood transfusion management, etc. As shown in FIG. 10B, a number of light sources of various centroid wavelengths (i.e., λ1, λ2, . . . , λn LEDs) are turned on and off sequentially over time. In the case of the non-invasive measurement of glucose, multiple LEDs in the range of 900 nm to 1700 nm can be adopted. In the case of the non-invasive measurement of total hemoglobin and/or water, wavelengths in the range of 600 nm to 1350 nm should be sufficient. The spectral ranges defined are sufficient because blood and bloodless components at the measurement site have spectral features that are typically quite distinctive depending on the wavelength sub-range under consideration. For instance, water and glucose have higher absorption in the 1550 nm to 1700 nm range than the other components, the hemoglobin species have pronounced features in the 600 nm to 1350 nm, fat has in general pronounced scattering properties throughout the whole range when compared to other blood components, and so on and so forth. The modulation schemes 900, 1000, 1002 shown in FIG. 9, FIG. 10A, and FIG. OB, respectively, can be switched over time depending on the particular application and/or measurement conditions.

In some embodiments, the method shown in FIG. 11B can be used in a multi-parameter monitoring device that continuously measures SpO2, PR, PA, and PI using the modulation scheme 900 shown in FIG. 9 and/or the RED-GREEN-IR Modulation scheme 1000 shown in FIG. 10A. Embodiments of the monitoring device described herein comprise a multi-parameter monitoring device. The multi-parameter monitoring device can perform lower-frequency periodic spot-check measurements of other blood parameters, such as the ones previously mentioned (i.e., glucose, water, etc.) using the Multi-Wavelength Modulation scheme 1002 (FIG. 10B). Such lower-frequency periodic spot-check measurements are possible because typically water, glucose, hemoglobin, etc. concentrations in blood vary slower when compared to SpO2, PR, PA and PI. Because typical measurement periodicity for the said parameters (e.g., glucose, water, total hemoglobin, etc.) is in general much longer (i.e., once every minute, few, or several minutes, once an hour, once every few hours, etc.), the increase in the power consumption of the multi-parameter monitoring device is not significant. In some instances, the additional LEDs and photodetector technologies (i.e., silicon and indium gallium arsenide photodiodes for the 600 nm to 1700 nm wavelength measurement range) that can be used in the multi-parameter monitoring device result in a small incremental cost and a negligible increase in the optical sensor footprint.

The Multi-Wavelength Modulation scheme 1002 shown in FIG. 10B can also be used to measure SpO2, PR, PA and PI. In this embodiment, the red and near-infrared LEDs are combined with other wavelengths to create “n” photoplethysmographs that can be used to improve SpO2 accuracy or motion performance. Accuracy is improved at least in part because additional LEDs throughout the visible and near-infrared range enable estimation algorithms to counter the optical interference effects of other blood and bloodless components not needed in the measurement of oxygen saturation, pulse rate and/or perfusion. Operation under motion is improved because the effects of motion acceleration on the venous and capillary blood creates optical interferences in the measurement site that have distinct morphological features depending on the wavelength range, and hence are more likely to be eliminated from the photoplethysmographs through advanced signal processing such as the numerical solver device described herein.

Persons of ordinary skill in the art will understand that the wavelengths and other measurements and ranges discussed herein are generally intended to be representative of certain embodiments of the inventions, and not as limiting as to the many ways in which the inventions can be practiced.

As described earlier, a distributed computing architecture may be used to compute SpO2, PR, PA, and/or PI, where SpO2, PR, PA and/or PI are estimated on a host device 105 to increase the battery life of the monitoring device 100. The processing device in the monitoring device 100 (e.g., processing device 102 in FIG. 1) may execute the time critical, high frequency, low latency and low complexity tasks. Data processed by the processing device in the monitoring device 100 may be reduced in bandwidth by decimation algorithms, and sent wirelessly to the host device 105. In one embodiment, one or more processing devices in the host device (e.g., processing device 117 in FIG. 1) may execute more complex, high latency tasks to calculate and continuously display the measurement values for SpO2, PR, PA, and/or PI.

FIG. 12A depicts a second example of a distributed system suitable for processing signals produced by a monitoring device. The system 1200 includes a monitoring device 100, a host device 105, a computing system 1202, and a cloud computing system 1204. The monitoring device 100 can be a single-use or reusable monitoring device that employs biosensing technology for applications such as medical, fitness, and wellness applications. The monitoring device 100 can be, for example, a pulse oximeter, a pulse co-oximeter, an ECG patch, an ultrasound sensor, an imaging sensor, a non-invasive glucometer, an activity tracker, a wristwatch, a ring, a smart garment, and other suitable implementations.

The host device 105 can be a mobile phone, a tablet computer, a desktop or laptop computer, a wall appliance, a dedicated monitor, a multi-parameter monitor, several wall appliances and or/computer devices working together in a distributed architecture to increase functionalities and wireless coverage range, etc. The host device 105 can be wireless or wired connected to the monitoring device 100 via the connection 1206. The host device 105 receives spot-check or continuous data from the monitoring device 100 and processes, stores, and presents visual, audible, olfactory, or tactile information that convey measurements, analysis, results, and alarms based on the data received from the monitoring device 100.

The computing system 1202 can be a mobile phone, a tablet computer, a desktop or laptop computer, a wall appliance, a dedicated monitor, a multi-parameter monitor, several wall appliances and or/computer devices working together in a distributed architecture to increase functionalities and wireless coverage range, etc. In one embodiment, the computing system 1202 can be connected to the host device 105 via the wired or wireless connection 1208. The computing system 1202 and the host device 105 may be connected to the cloud computing system 1204 via the wired or wireless connections 1210 and 1212, respectively, to provide additional functionalities, such as electronic health record (EHR) connectivity, distributed alarm, measurement, warning, and notification systems, etc.

The biosensing and supporting functionalities described herein are enabled by firmware 1214 in the monitoring device 100 and software 1216 in the host device 105. In one embodiment, the firmware 1214 and the software 1216 implement real-time systems. Real-time systems ensure that all tasks are executed substantially on time in applications that require immediate or timely responses. In another embodiment, the firmware 1214 and the software 1216 implement hybrid configurations where real-time systems are combined with non-time-critical systems. The hybrid configurations enable more flexible and efficient computing architectures where real-time tasks can take precedence over non-time-critical ones. One non-limiting example of a real-time system would be a discrete-time data collection system, where data samples are to be collected and processed with very low latency at consecutive time intervals. A non-limiting example of a non-time-critical system would be a monitor that calculates and displays measurements over time, in which the measurement refresh rate requirements and the data buffering capabilities allow for a variable latency time without compromising the safety and efficacy of the system.

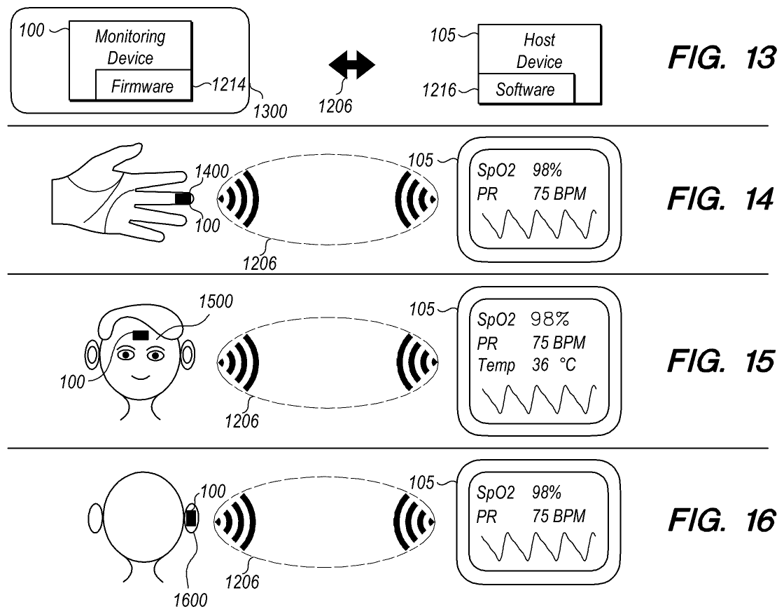

In some instances, the monitoring device 100 and the host device 105 can be integrated into a single physical unit 1218 (FIG. 12B), such as a wristwatch with biosensing functionalities, a display for viewing user interface screens, storage, processing, and connectivity functionalities. Additionally or alternatively, the computing system 1202 may be integrated into the host device 105. In non-limiting examples, the host device 105 shown in FIGS. 13-16 may be a host device 105 with the computing system 1202 integrated therein. In some embodiments, the computing system 1202 and the cloud computing system 1204 can be integrated into a single device. For example, the cloud computing system 1204 and the computing system 1202 may be integrated into the cloud computing system 1204.

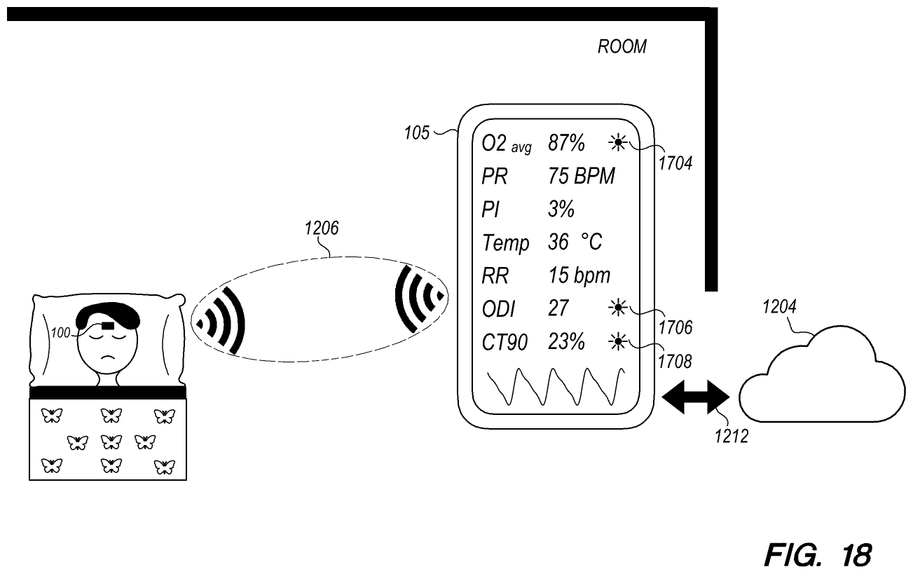

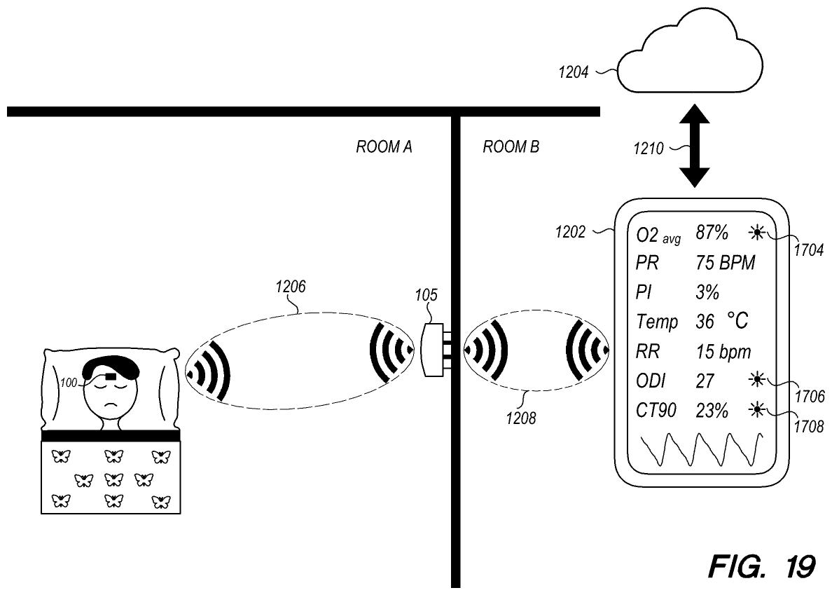

FIGS. 13-19 illustrate other example embodiments of a distributed system suitable for processing signals produced by a monitoring device. The monitoring device 100 can be placed on the user's skin 1300 (FIG. 13), digit 1400 (FIG. 14), forehead 1500 (FIG. 15, FIG. 18, and FIG. 19), or behind the user's ear 1600 (e.g., posterior auricle) (FIG. 16). The monitoring device 100 in FIGS. 13-15, 18, and 19 transmits data to the host device 105 using the connection 1206. As shown in FIGS. 14-16, the host device 105 can be configured (via software 1216) to display measurement values such as SpO2, PR, and/or Temperature. In FIGS. 14-16 and in FIG. 18, the host device 105 can be a multi-parameter monitor, mobile device, desktop, laptop, tablet computer, or other computing device.

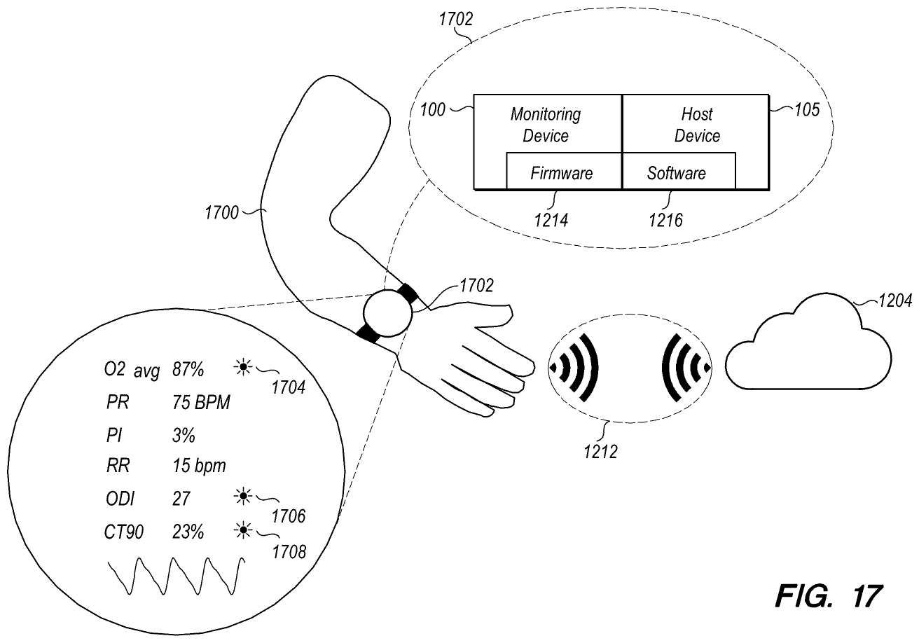

In FIG. 17, the monitoring device is positioned on the user's arm 1700. The wristwatch 1702 is an example embodiment where the monitoring device 100 and the host device 105 are integrated into a single physical unit (e.g., unit 1218 in FIG. 12B) that communicates with the cloud computing system 1204 via the connection 1212. The wristwatch 1702 can use one or more optical sensors to continuously and noninvasively measure SpO2, PR, PI, PA or signal strength, interstitial and blood glucose concentrations, respiratory rate (RR), total hemoglobin, concentrations of dyshemoglobins, among several other optical-based measurements depending on the optical sensor design and signal processing. However, when compared to a fingertip or a forehead, for instance, the wrist as a measurement site has larger anatomical differences (function of race, gender, weight, etc.) between users along with relatively lower peripheral blood perfusion. Additionally, the signals from the optical sensor(s) are affected by motion artifacts due to internal movements of the long tendons that deliver motion from the muscles to the finger joints. In some instances, these factors can make the measurement of SpO2 on the wrist challenging and subject to larger measurement errors. An alternative to directly displaying SpO2 as a measurement is to compute and display long-term measurements, metrics, and scores derived from SpO2 instantaneous measurements (i.e., O2_{avg}, ODI, CT90, etc.). For instance, in the case of O2_{avg} and CT90, longer averaging time (30 minutes, 1 hour, 8 hours, etc.) can improve the measurement accuracy whenever SpO2 instantaneous measurements have larger errors but are unbiased over longer periods of time. In the case of ODI, the accuracy can be substantially improved, even if SpO2 instantaneous measurements are biased over long periods of time. This is because the computation of ODI involves the long-term scoring of the amplitude of desaturation events, which requires subtractions of SpO2 instantaneous measurements at different times within relatively short time intervals in order to compute the desaturation amplitudes. The subtraction operations remove biases, and long-term averages reduce measurement errors. The same rationale can be applied to other instantaneous measurements (e.g., PR, PI, RR, glucose concentration, etc.), and their long-term derivatives measured by a wristwatch or a patch sensor placed on the wrist.

FIGS. 18 and 19 show the monitoring device 100 attached to the user's forehead. In FIG. 18, both the monitoring device 100 and the host device 105 are located in the same location (e.g., a Room). The monitoring device 100 transmits data to the host device 105 via the connection 1216, and the host device 105 communicates (e.g., transmits data) to the cloud computing system 1204 via the connection 1212.

In FIG. 19, the monitoring device 100 and the host device 105 are located in the same location (e.g., Room A) while the computing system 1202 is located in a different location (e.g., Room B). The monitoring device 100 communications with (e.g., transmits data to) the host device 105 via the connection 1216. As shown, the host device 105 is a wall appliance that is plugged into a power outlet on a wall. In some aspects, the software 1216 in the host device 105 may not provide the user interface functionalities. Instead, the user interface functionalities can be provided by the computing system 1202 (in ROOM B) in the form of a multi-parameter monitor that receives measurements and data from the host device 102 via the connection 1208.

The embodiments in FIGS. 13-19 can be used for wireless, continuous, noninvasive monitoring of fractional and functional oxygen saturation (SpO2), pulse rate (PR) or heart rate, perfusion index (PI), pulse amplitude (PA) or signal strength, respiration rate (RR), interstitial and blood glucose concentrations, total hemoglobin, concentrations of dyshemoglobins, oxygen desaturation index (ODI), cumulative percentages of time spent at oxygen saturations below 90% (CT90), mean oxygen saturation level (O2_{avg}), user motion or activity, etc. Noninvasive continuous monitoring of the user's body core temperature (Temp) is also possible when the monitoring device 100 is placed on the user's forehead (FIG. 15, FIG. 18, and FIG. 19). The embodiments in FIGS. 13-17 can be used in fitness and wellness monitoring applications such as sports, aviation, etc. The embodiments shown in FIGS. 13-19 can be used in clinical monitoring applications (home and hospital settings) such as anesthesia, narcotic administration for pain management with opioids through subcutaneous injection or infusion pump, user transport, user recovery, evaluation of home oxygen therapy, analgesia for dentistry (general, orthodontics, endodontics), sleep monitoring studies, analgesia for short-term procedures, such as burned or fractured users, renal colic management using morphine (subcutaneous injection or infusion pump), cancer patients under terminal conditions, gastrointestinal procedures (endoscopy, colonoscopy), etc.

The embodiments in FIGS. 17-19 can also be utilized to confirm previously diagnosed obstructive sleep apnea (OSA) or rapidly recognize it in sleep monitoring studies. In the case of low-cost, wireless, continuous, single-use or reusable implementations for the monitoring device 100, the embodiments in FIGS. 17-19 can expedite the treatment of more severe OSA users in waiting list for expensive and complex Polysomnography (PSG) monitoring solutions. The embodiments in FIGS. 17-19 can offer relatively low-cost solutions that calculate scores and metrics over single or multiple sleep cycles, such as O2_{avg}, ODI, CT90, etc. in order to aid OSA diagnosis. In FIGS. 17-19, element 1704 depicts an example embodiment of an alarm event during a sleep study, where O2_{avg} reached an abnormally low value while monitoring a severe OSA user. Elements 1706, 1708 in FIGS. 17-19 depict examples of alarm events during a sleep study, where ODI and CT90 reached abnormally high values.

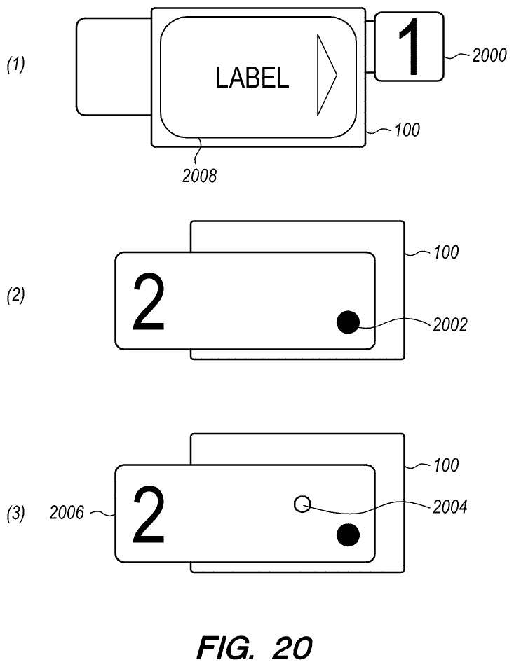

FIG. 20 illustrates the steps to activate a monitoring device. In step one, a first tab 2000 labeled “1” is removed to close an electrical contact and turn on the monitoring device 100. In one embodiment, the first tab 2000 is made of a conductive tape. In step two, an indicator 2002 (e.g., a circle or dot) on the exterior surface of the monitoring device 100 is pressed by the user to confirm the electrical contact that turns on the monitoring device is closed. When the monitoring device 100 is turned on (step three), a light 2004 is turned on and indicates the monitoring device 100 is able to be operably connected to the host device. In one instance, the light 2004 is produced by at least one of the one or more light sources 113 in the optical sensor (e.g., a green light). Using s light source in the optical sensor can reduce costs and the footprint of the monitoring device.

Continuing with step three, a second tab 2006 labeled “2” is removed to expose an adhesive tape that is used to attach the monitoring device 100 to a measurement site. For example, the adhesive tape can be a biocompatible adhesive tape. In one embodiment, a second electrical contact sensor (e.g., 708 in FIG. 7D) is the label 2008 depicted in step one of FIG. 9D. The label 2008 can be made out of a metallic adhesive tape so as to have the dual function of identifying the monitoring device 100 and functioning as the second electrical contact sensor. Additionally or alternatively, the first electrical contact sensor (e.g., 707 in FIG. 7D) and the temperature sensor (e.g., 705 in FIG. 7D) can be part of the adhesive tape that is exposed when the second tab 2006 is removed. The adhesive tape, as part of the outer shell or enclosure of the monitoring device, can be designed to have good adherence to the subject's skin, as well as good electrical and thermal properties to enable a more accurate measurement of the body's core temperature and the heart's ECG.

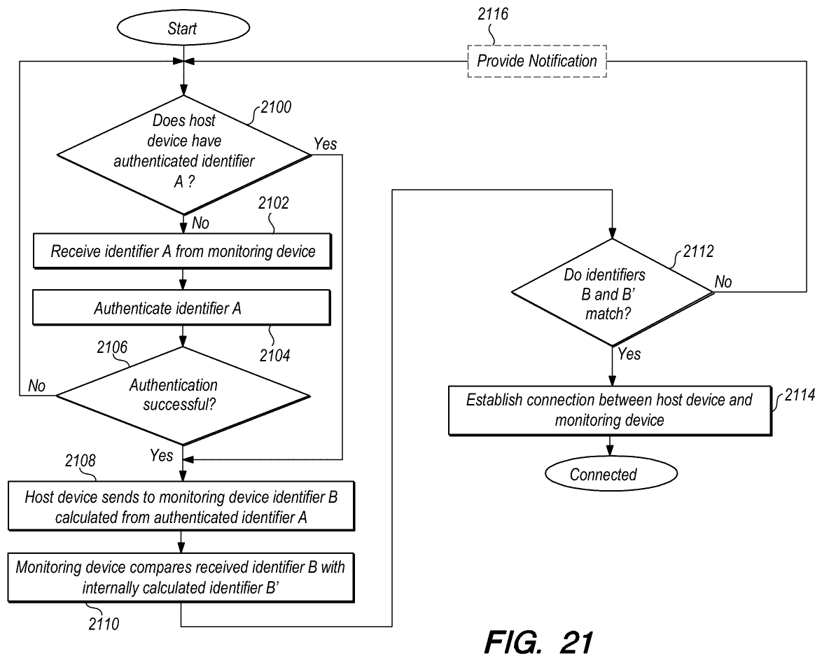

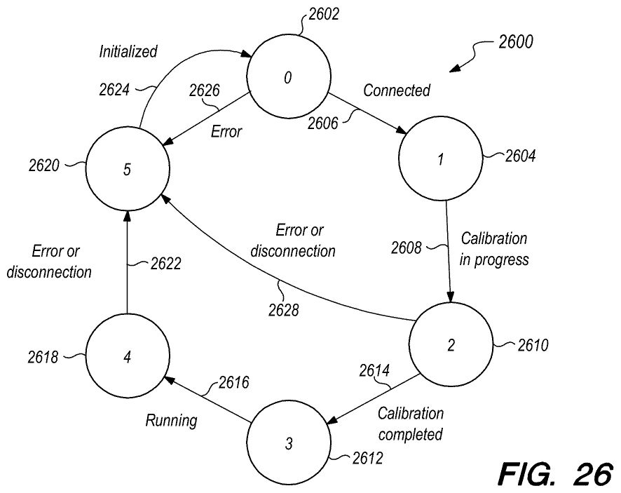

Once the monitoring device is activated, the monitoring device can be connected to a host device. FIG. 21 depicts a flowchart of an example method of an authentication process for connecting the monitoring device to a host device. In one embodiment, the authentication process is performed when the current state of the host application in the host device is state 0 (state 2502 depicted in FIG. 25) and the current state of the firmware in the monitoring device is state 0 (state 2602 shown in FIG. 26). Other embodiments can perform the authentication process when the current state of the host device and/or the monitoring device is at one or more different states.

Initially, a decision is made at block 2100 as to whether the host device has an authenticated first identifier (e.g., identifier A) for the monitoring device from a previous authentication process. If the host device does not have a previously authenticated first identifier, the method passes to block 2102 where the host device receives the first identifier from the monitoring device. The host device then authenticates the monitoring device using the first identifier (block 2104). In one embodiment, the first identifier is created when the monitoring device is manufactured using a unique factory-programmed identifier(s) stored in the processing device or the memory (e.g., a secure memory) in the monitoring device. Other information from the manufacturing process (e.g., lot number, expiration date, etc.) can be stored in the monitoring device along with the first identifier. The first identifier can be printed or transferred to a label, internal circuitry, or product packaging of the monitoring device during the manufacturing process.

In a non-limiting example, the first identifier is received by host device via barcode scanning of the monitoring device's label or product packaging. The host device perform the scanning by Radio-Frequency Identification, by optical identification, by means of optical sensors, or by any other technique that enables the transfer of identifiers. An example authentication process can include the host device (e.g., software running on the host device) computing a hash code (e.g., a first hash code) based on information stored in the first identifier and comparing the computed hash code to a hash code stored in the first identifier. The authentication of the monitoring device (e.g., the authentication of the first identifier) is successful when the calculated hash code matches the stored hash code.

Next, as shown in block 2106, a determination is made as to whether the authentication is successful or not. If the authentication fails (is not successful), the method returns to block 2100 and blocks 2100, 2102, 2104, and 2106 repeat. In one embodiment, blocks 2100, 2102, 2104, 2106 repeat for a given period of time. At the end of the period of time, and if the authentication is still unsuccessful, a notification and/or alert may be provided to the monitoring device and/or to the host device regarding the unsuccessful authentication. For example, the host device can display a notification and/or issue an audible alert.

When the authentication is successful at block 2106, or when the host device has a previously authenticated first identifier (block 2100), the process passes to block 2108. In block 2108, the host device sends a second identifier (e.g., identifier B) to the monitoring device. In one embodiment, the second identifier is calculated from the authenticated first identifier. For example, some or all of the data that is stored in the first identifier can be sent to the monitoring device or the host device can calculate a second hash code using data stored in the first identifier.

The monitoring device receives the second identifier and compares the second identifier with an internally calculated or stored third identifier (e.g., identifier B′) at block 2110. In one embodiment, the second identifier is a second hash code computed by the host device and the third identifier is a third hash code calculated by the monitoring device from the same data stored in the first identifier (e.g., the first unique factory-programmed identifier(s)) stored in the processing device or memory (e.g., a secure memory) of the monitoring device).

A determination is then made at block 2112 as to whether the second and the third identifiers match. If not, the method returns to block 2100 and blocks 2100, 2102, 2104, and 2106 repeat. In one embodiment, blocks 2100, 2102, 2104, 2106 repeat for a given period of time. At the end of the period of time, and if the authentication is still unsuccessful, a notification and/or alert may be provided to the monitoring device and/or to the host device regarding the unsuccessful authentication (block 2116). For example, the host device can display a notification and/or issue an audible alert.

When the second and the third identifiers match at block 2112, the monitoring device is authenticated and a connection (e.g., wireless connection) is established between the monitoring device and the host device (block 2114). In some embodiments, the authentication process can repeat a finite number of times before the authentication process ends. When the authentication process ends, other security mechanisms (e.g., recovery password, verification code etc.) can be performed to allow the authentication process to continue.

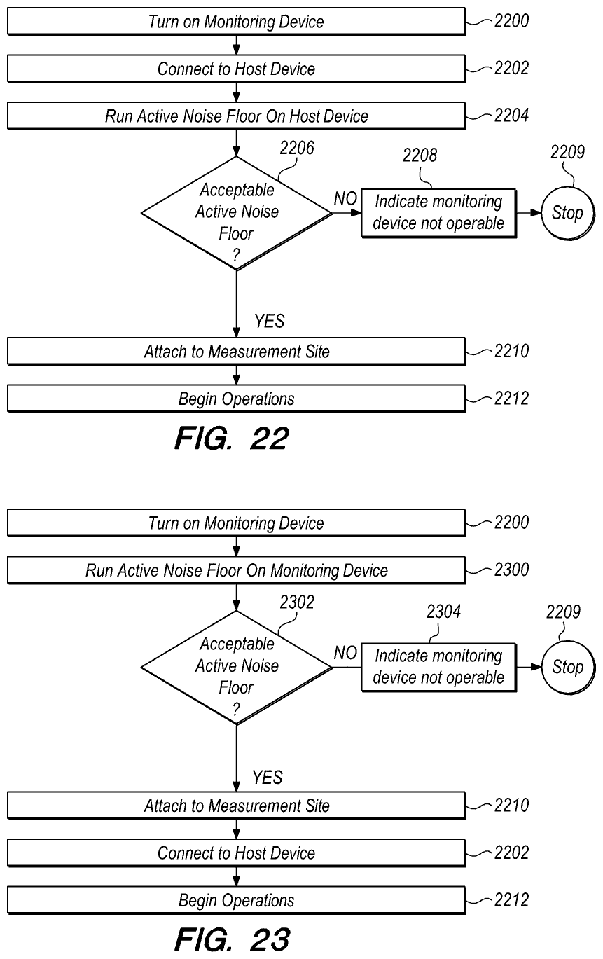

FIG. 22 depicts a first example method of determining an active noise floor of a monitoring device. Initially, the monitoring device is turned on at block 2200. For example, in one embodiment, the monitoring device is turned on by removing the first tab and pressing the indicator (e.g., first tab 2000 and indicator 2002 in FIG. 20). In block 2202, the monitoring device is authenticated and connected (e.g., wirelessly connected) to the host device. In an example embodiment, the monitoring device is paired with the host device using BLUETOOTH.

Once the connection between the monitoring device and the host device is established, an active noise floor measurement is performed on the host device (block 2204). In a non-limiting example, the monitoring device is placed in a dark environment (e.g., limited or no ambient light) for the active noise floor measurement. In another non-limiting example, the monitoring device modulates the light source signals and/or demodulates the received optical signals in order to enable active noise floor measurements under ambient light and/or electromagnetic interferences. The modulation and demodulation algorithms prevent ambient light signals and/or electromagnetic interferences from interfering with the active noise floor measurement. The noise floor measurement is used to quantify the signal-to-noise ratios and metrics that quantify the performance of the monitoring device prior to placing the monitoring device on a measurement site. The noise floor measurement is useful in applications that the manufacturing, storage, shipping and/or handling processes may cause the monitoring device to become out of specifications due to unforeseeable events. In an example embodiment, the noise floor measurement is generated by activating the one or more light sources (e.g., 113 in FIG. 1) in the monitoring device as if the monitoring device is in normal operation. The light will diffuse through the material in the second tab 2006 in FIG. 20 and reach the one or more photodetectors via diffusive and/or specular reflectance. The signals produced by the photodetector(s) (e.g., 114 in FIG. 1) are collected by the monitoring device and are demodulated, filtered, processed and sent to the host device for computation of the signal-to-noise ratios and metrics.

In block 2206, a determination is made as to whether the computed signal-to-noise ratios and/or the metrics are acceptable (e.g., greater than a predefined threshold). If not, the host device indicates to the user that the monitoring is non-operational or defective and operations stop (blocks 2208 and 2209). For example, the host device can produce a visual notification and/or an audible alert. In an alternate embodiment, multiple noise floor runs can be acquired and processed before block 2206 is performed.

When the computed signal-to-noise ratios and/or the metrics are acceptable, the process passes to block 2210 where the monitoring device is attached to a measurement site. For example, the second tab 2006 in FIG. 20 is removed to expose the adhesive material and attach the monitoring device to a measurement site. The monitoring device and the host device then enter normal monitoring operations (block 2212).

FIG. 23 illustrates a second example method of determining an active noise floor of a monitoring device. In the illustrated embodiment, the noise floor calculations are performed by the monitoring device prior to connecting with host device. Initially, the monitoring device is turned on at block 2200. An active noise floor measurement is then obtained by the monitoring device at block 2300. The noise floor measurement is determined by activating the one or more light sources in the monitoring device as if the monitoring device is in normal operations (e.g., light source(s) 113 in FIG. 1). The light will diffuse through the material in the second tab 2006 in FIG. 20 and be detected by the one or more photodetectors via diffusive and/or specular reflectance (e.g., photodetector(s) 114 in FIG. 1). The signals produced by the photodetector(s) are demodulated, filtered, and processed by the monitoring device to compute the signal-to-noise ratios and metrics.

A determination is then made at block 2302 as to whether the computed signal-to-noise ratios and/or the metrics are acceptable (e.g., greater than predefined thresholds). If the computed signal-to-noise ratios and/or the metrics are not acceptable, the monitoring device indicates to the user that the monitoring device is non-operational or defective and the operations end (blocks 2304 and 2209, respectively). For example, the monitoring device can produce a flashing light(s) in a particular pattern or mode using at least one light source in the monitoring device. In an alternate embodiment, multiple noise floor runs can be acquired and processed before block 2202 is performed.

When the computed signal-to-noise ratios and/or the metrics are acceptable, the process passes to block 2110 where the monitoring device is attached to a measurement site. The monitoring device is then connected (e.g., wirelessly connected) to the host device and the monitoring device and the host device enter normal monitoring operations (blocks 2102 and 2112, respectively).

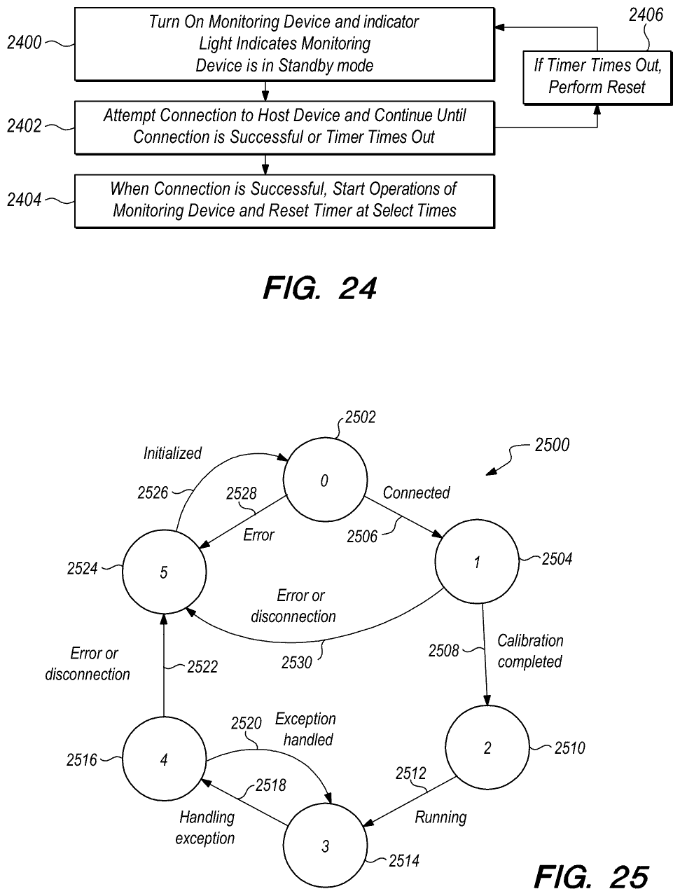

FIG. 24 illustrates a flowchart of a first example method of operating a monitoring device. Initially, as shown in block 2400, the monitoring device is turned on and an indicator light indicates the monitoring device is turned on and in standby mode. The standby mode represents a mode in which the monitoring device is waiting to be operably connected to a host device (e.g., wirelessly connected to the host device). In one embodiment, a connection timer begins when the monitoring device is turned on, where the connection timer monitors (e.g., counts down) a given time period in which the monitoring device attempts to connect to a host device.

The monitoring device then attempts to connect to a host device at block 2402. In one embodiment, the monitoring device repeatedly attempts to connect to the host device until the monitoring device either successfully connects to the host device or until the connection timer times out (e.g., expires). When the monitoring device successfully connects to the host device, the process passes to block 2404 where the monitoring device begins operations. In some embodiments, the connection timer is a watchdog timer that is reinitialized at select times or periodically by the monitoring device (e.g., the firmware 1214 in FIG. 12A) whenever the monitoring device is in normal operation. Reinitializing the connection timer ensures that the processor and circuitry in the monitoring device are only reset if a software and/or hardware fault occurs and the connection timer (i.e., watchdog timer) times out. If the monitoring device is in standby mode, then the connection timer (i.e., watchdog timer) will time out at select times or periodically, forcing the monitoring device to reset itself. This ensures that the monitoring device will be always operational (i.e., in standby or connected) and eliminates completely the need for reset and/or power switches in the monitoring device circuitry in order to make unnecessary manual resets or power up sequences by the user in the event of a software or hardware fault.

When the monitoring device is not able to connect to the host device at block 2402 within the given time period or the monitoring device loses connection with the host device in block 2404 due to a hardware or a software fault, the connection timer (i.e., watchdog timer) times out, and the method continues at block 2406 where a reset operation on the monitoring device is performed. The processor and circuitry in the monitoring device are reset, and the indicator light is turned off. After a given period of time, the method returns to block 2400 where the processor and circuitry in the monitoring device are reinitialized (e.g., turned off and on) and the indicator light turned on. Blocks 2402 and 2404 or blocks 2402 and 2406 repeat.

FIG. 25 depicts an example state diagram for a host device. The state diagram 2500 is used to describe the logical behavior of the software (“host software”) in the host device (e.g., software 1216 in FIG. 12A). In the illustrated example, the host software works in conjunction with the firmware (“device firmware”) in the monitoring device (e.g., firmware 1214 in FIG. 12A). When the current state of the host software is state 0 (state 2502), the host device is initialized, disconnected from the monitoring device, and ready to be connected to the monitoring device.

The state transitions to state 1 (state 2504) when the event “Connected” 2506 occurs after a successful authentication process between the host device and the monitoring device 100 over a wireless or a wired connection (e.g., connection 1206 in FIG. 12A), or internally if the host and the monitoring devices are integrated into a single physical unit (e.g., unit 1218 in FIG. 12B). In state 1, the host device is waiting for the monitoring device to complete a calibration process. When the event “Calibration completed” 2508 occurs, the state of the host software transitions from state 1 to state 2 (state 2510).

State 2 signals that the calibration process in the monitoring device is successfully completed. The event “Running” 2512, which causes the host software to transition from state 2 to state 3 (state 2514), indicates that real-time data transfer is taking place from the monitoring device to the host device and that the host software can start real-time data processing to store and share visual, audible, olfactory, or tactile information that convey measurements, analysis, and results. A transition from state 3 to state 4 (state 2516) occurs whenever a “Handling exception” event 2518 transpires and indicates that an exception or an error has occurred. Example exceptions include, but are not limited to, measurement alarms and warnings, an unreliable connection between the monitoring device and the host device, low or empty battery on the monitoring device, low battery on the host device, and errors including lost or invalid biosignal, lost connection between the host device and the monitoring device, software faults, and the like. The host software returns to state 3 (state 2514) once the exception has been handled. For instance, a measurement alarm may become inactive after the user state returns to normal.

When the exception has been handled, the event “Exception handled” 2520 occurs and the host software transitions to state 3 (state 2514). In the case of an ‘Error or disconnection” event 2522, the host software transitions to state 5 (state 2524), where some or all of the host software services, libraries, variables, and parameters are reinitialized. Once reinitialization is complete, the event “Initialized” 2526 occurs and the host software transitions to state 0 (state 2502). While at state 0 (state 2502), any “Error” event 2528 causes the host software to transition to state 5 (state 2524) for reinitialization, critical error handling, and/or critical error handling. While at state 1 (state 2504), any “Error or disconnection” event 2530 also causes the host software to transition to state 5 (state 2524) for reinitialization, critical error handling, and/or critical error handling.

FIG. 26 illustrates an example state diagram for a monitoring device. The state diagram 2600 is used to describe the logical behavior of the device firmware (e.g., firmware 1214 in FIG. 12A). In this example, the device firmware works in conjunction with the host software. When the current state of the device firmware is 0 (state 2602), the monitoring device is initialized, disconnected from the host device, and ready to be connected to the host device.

The state of the device firmware transitions to state 1 (state 2604) when the event “Connected” 2606 occurs after a successful authentication process between the host device and the monitoring device over a wireless or a wired connection (e.g., connection 1206 in FIG. 12A), or internally if the host and the monitoring devices are integrated into a single physical unit (e.g., unit 1218 in FIG. 12B). When the “Calibration in progress” event 2608 transpires, the state transitions from state 1 to state 2 (state 2610).

State 2 signals that the calibration process of the monitoring device is in progress. The calibration process configures the monitoring device's dynamic range, sensing, and data collection settings for favorable or optimal compliance with the physical and physiological properties of the measurement site. The device firmware transitions from state 2 to state 3 (state 2612) after the “Calibration completed” event 2614 occurs and indicates that the calibration process of the monitoring device is complete.

The “Running” event 2616, which causes the device firmware to transition from state 3 to state 4 (state 2618), indicates that the monitoring device is sending data with favorable or optimal dynamic range, sensing, and data collection settings to the host device. A transition from state 4 to state 5 (state 2620) occurs whenever the “Error or disconnection” event 2622 transpires, which indicates that an error has occurred or that the connection between the monitoring device and the host device has become inoperable. Example errors include, but are not limited to, frontend electronic or optical saturation caused by changes in the measurement site, external environmental factors, interferences, faulty sensors, and the like. At state 5 some or all of the software services, libraries, variables, and parameters of the device firmware are reinitialized.

Once reinitialization is completed, the event “Initialized” 2624 occurs and the device firmware returns to state 0 (state 2602). While at state 0, any “Error” event 2626 causes the device firmware to transition from state 0 to state 5 for reinitialization. Similarly, while at state 2, any “Error or disconnection” event 2628 causes the device firmware to transition from state 2 to state 5 for reinitialization

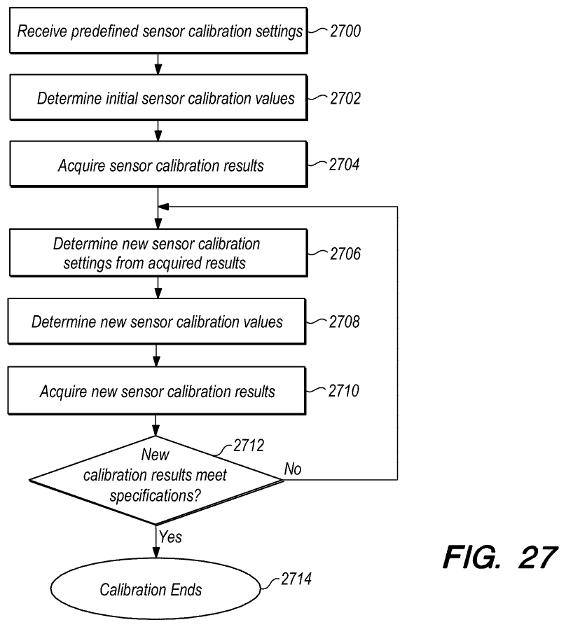

FIG. 27 illustrates and flowchart of an example sensor calibration process that is suitable for use by a monitoring device. In one embodiment, the sensor calibration process is performed when the device firmware is in state 2 (state 2610) of the state diagram shown in FIG. 26. In general, the monitoring device performs biosensing functionalities through one or more sensors that apply and/or measure energy in the measurement site (e.g., optical, electrical, mechanical, etc.). The sensor(s) typically require some form of real-time calibration before the sensor(s) can be used by the device firmware to collect biosensing data.

Initially, the sensor calibration process receives and uses initial predefined sensor calibration settings in the form of a table, a database, or other form that is stored in the device firmware (block 2700). Initial sensor calibration values are set in block 2702 that result in the application of energy signals (e.g., light) to the measurement site. The energy signals interact with the measurement site to produce initial sensor calibration results that are acquired at block 2704. The initial sensor calibration results are then used to calculate new sensor calibration settings (block 2706).

The new sensor calibration settings are used to determined new sensor calibration values (block 2708). The new sensor calibration values result in the application of new energy signals to the measurement site, which in turn produce new sensor calibration results that are acquired in block 2710. The new sensor calibration results are tested or compared against predefined sensor calibration specifications and a determination is made at block 2712 as to whether the new sensor calibration results meet the predefined sensor calibration specifications. If the predefined sensor calibration specifications are met, the sensor calibration process passes to block 2714 where the sensor calibration process ends.

When the predefined sensor calibration specifications are not met, the method returns to block 2706. Blocks 2706, 2708, 2710, and 2712 repeat until the predefined sensor calibration specifications are met. Alternatively, in other embodiments, the loop with blocks 2706, 2708, 2710, and 2712 repeat for a maximum number of iterations or until the sensor calibration specifications are met. The monitoring device (via the device firmware) can then decide whether to inform the host device of the successful or unsuccessful calibration of the sensor(s). If the calibration process in unsuccessful, the monitoring device (via the device firmware) can decide to repeat (or not) the sensor calibration process.

In some embodiments, flexibility can be increased by storing the initial predefined sensor calibration settings (block 2700) and the new sensor calibration settings (block 2706) in the host device and transferring the initial predefined sensor calibration settings and the new sensor calibration settings to and from the monitoring device. In addition, the sensor calibration process can be executed in a distributed system, where blocks 2700, 2702, 2704, 2708, and 2710 are executed by the device firmware in the monitoring device, and block 2706 and block 2712 are executed by the software in the host device. Using a distributed system to execute the sensor calibration process enables the host to use more complex algorithms for the calculation of favorable or optimal sensor calibration settings and sensor calibration specifications. The distributed system can be useful when the monitoring device is a low-cost, single use, and fully disposable monitoring device with limited computation resources and battery life. The more advanced and faster computation resources in the host device can be leveraged to enable complex algorithms for the calculation of sensor calibration settings and sensor calibration specifications without sacrificing the computational resources and battery life in the monitoring device.

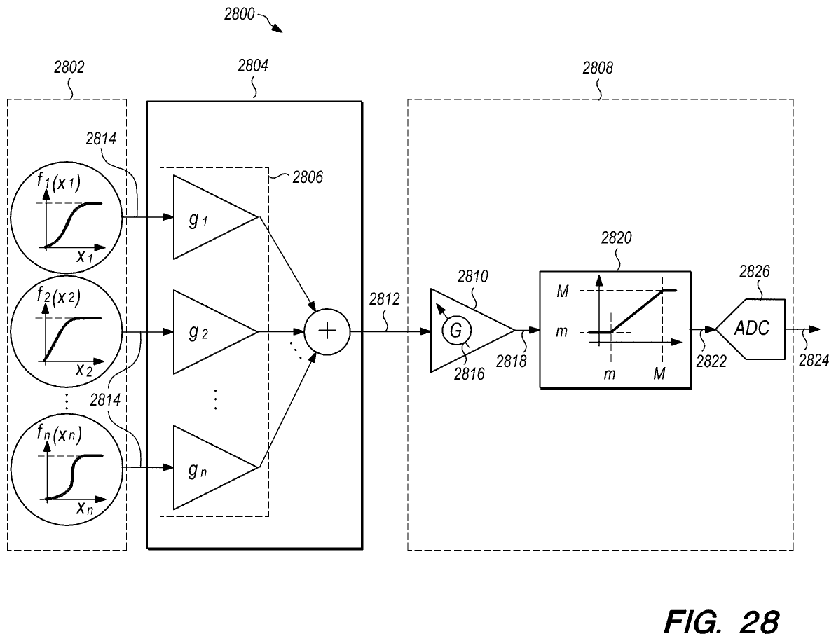

FIG. 28 illustrates an example sensor calibration system that is suitable to execute the sensor calibration process shown in FIG. 27. In the illustrated embodiment, the sensor calibration system 2800 can be used to calibrate a sensor in a monitoring device that has multiple energy sources 2802 that are time-multiplexed and applied to a measurement site 2804. The measurement site 2804 has unknown transfer functions 2806 with properties (gains or attenuations) that depend on the physical properties of the energy sources 2802 and the constituents of the measurement site 2804. Example constituents of the measurement site include, but are not limited to, blood, fat, and bone. In one example embodiment, if the energy sources 2802 are light sources, such as Light-Emitting Diodes (LEDs) or lasers, then the transfer functions 2806 of the measurement site 2804 depend on the optical properties of the constituents of the measurement site 2804 and the optical wavelength emissions of the light sources.

Each energy source 2802 is assumed to have an unknown nonlinear functional relationship between its input excitation and its output power. For example, in the case of LEDs and lasers, the electrical current of excitation and the output optical power have a nonlinear relationship that resembles an S-shaped curve. For very low excitation currents, the output optical powers change very little with changes in excitation currents. For excitation currents in the LEDs' normal operating ranges, the relationship between excitation currents and output optical powers are affine or monotone increasing, and as a result, the output optical powers change almost linearly with changes in excitation currents. For very high values of excitation currents, the output optical powers saturate, and no longer vary with changes in the excitation currents. The S-shaped curve can change from one LED unit to another, and depend on the design and manufacturing technologies, manufacturing process variabilities, operating conditions, and ambient temperature.

The frontend 2808 includes a frontend amplifier 2810 that collects a time-multiplexed energy signals 2812 after the signals 2814 are attenuated by the unknown transfer functions 2806 using a transducer or detector (or a plurality of transducers or detectors). In the case of optical energy, a photodetector (e.g., a photodiode) or a photomultiplier, which is part of the frontend amplifier 2812, can be used to collect and convert the time-multiplexed attenuated optical energy signals 2810 into electrical signals. The electrical signals are then amplified by the frontend amplifier adjustable gain 2816 to produce amplified signals 2818. The frontend amplifier 2810 can convert and amplify energy signals with bounded amplitudes. The allowed input-output dynamic range is represented by the saturation element 2820. The signals 2822 are identical, or substantially identical, to the amplified signals 2818 provided that the signals 2812 are within the allowed dynamic range defined by the saturation element 2820 and the adjustable gain 2816. The signals 2822 are converted into digital signals 2824 by means of an analog-to-digital converter (ADC) 2826.

For the sensor calibration process shown in FIG. 27, the input excitations for the energy sources 2802 are assumed to be known and adjustable via the device firmware in the monitoring device, so that the digital signal 2824 can also be read by the device firmware. If a single energy source k is activated while keeping the other energy sources turned off, then the relationship between the adjustable input excitation xₖ in 2802 and the digital signal y (digital signal 2824) can be described by the equation:

yₖ(i)=gGgₖƒₖ(xₖ(i)) Equation 1

where gₖ is the gain (or attenuation) for the unknown transfer function 2806 relative to the energy source k, and ƒₖ is the unknown nonlinear function for the energy source k, for k=1, 2, . . . , n, G is the frontend amplifier adjustable gain 2816, and g is a unknown and constant conversion factor that accounts for other attenuations, gains, and scale conversion factors in the signal path that produces the digital signal yₖ 2824. The index “i” refers to calibration instances or values. Hence, xₖ(i) refers to the i^{th} calibration value for input excitation xₖ, and yₖ(i) refers to the corresponding i^{th} calibration result for digital signal yₖ. For convenience, the unknown terms in Equation 1 are joined together in order to produce equation,

yₖ(i)=Ghₖ(xₖ(i)) Equation 2

where hₖ(xₖ(i)) ggₖfk(xₖ(i)). Each unknown function hₖ can be approximated by a polynomial or some other parametrized nonlinear function. For an example polynomial approximation,

hₖ(xₖ(i))=Σₚ₌₀^{P}α_{p,k}xₖ(i)^{p} Equation 3

where coefficients α_{p,k}, for k=1, 2, . . . , n, and p=0, 1, . . . , P, are unknown. The following Equation 4 can be obtained by combining Equations 2 and 3,

yₖ(i)GΣₚ₌₀^{P}α_{p,k}xₖ(i)^{p} Equation 4

Equation 4 maps, via a polynomial function k of order P, the i^{th} calibration value for input excitation xₖ into a corresponding i^{th} calibration result for the digital signal yₖ.

Equation 4 can be applied to the sensor calibration process shown in FIG. 27 together with the example sensor calibration system depicted in FIG. 28. The following steps describe the example application.

1. Block 2700 in FIG. 27: Assuming that the functional relationship between the input excitation xₖ and the digital signal yₖ can be approximated by a P^{th} order polynomial as described in Equation 4, then the device firmware in the monitoring device reads the initial predefined sensor calibration settings for each energy source k with at least (P+1) calibration values for xₖ (i.e., {xₖ(i): i=1, 2, . . . , (P+1), (P+2), . . . }, and predefined gain value G.

2. Blocks 2702 and 2704 in FIG. 27: A predefined gain value G for the frontend amplifier adjustable gain 2816 (FIG. 28) is set by the device firmware in the monitoring device. Then, for each energy source k, the device firmware sets corresponding initial sensor calibration values and acquires corresponding sensor calibration results for yₖ (i.e., {yₖ(i): i=1, 2, . . . , (P+1), (P+2), . . . }.

3. Block 2706 in FIG. 27: The device firmware in the monitoring device determines the new sensor calibration settings from the acquired sensor calibration results. The determination can be accomplished by solving for each Aₖ the following systems of linear equations,

$$\begin{matrix}{Y}_{k} = {GX}_{k} {A}_{k} & Equation 5 \\ where , & \\ {Y}_{k} = [ \begin{matrix}{y}_{k} ( 1 ) \\ {y}_{k} ( 2 ) \\ ⋮ \\ {y}_{k} ( P + 1 ) \\ {y}_{k} ( P + 2 ) \\ ⋮\end{matrix} ] & \\ {Y}_{k} = [ \begin{matrix}1 & {x}_{k} ( 1 ) & {{x}_{k} ( 1 )}^{2} & … & {{x}_{k} ( 1 )}^{P} \\ 1 & {x}_{k} ( 2 ) & {{x}_{k} ( 2 )}^{2} & … & {{x}_{k} ( 2 )}^{P} \\ ⋮ & ⋮ & ⋮ & ⋱ & ⋮ \\ 1 & {x}_{k} ( P + 1 ) & {{x}_{k} ( P + 1 )}^{2} & … & {{x}_{k} ( P + 1 )}^{P} \\ 1 & {x}_{k} ( P + 2 ) & {{x}_{k} ( P + 2 )}^{2} & … & {{x}_{k} ( P + 2 )}^{P} \\ ⋮ & ⋮ & ⋮ & ⋱ & ⋮\end{matrix} ] & \\ and & \\ {A}_{k} = [ \begin{matrix}{α}_{0 , k} \\ {α}_{1 , k} \\ {α}_{2 , k} \\ ⋮ \\ {α}_{P , k}\end{matrix} ] & \end{matrix}$$

where Yₖ is the acquired sensor calibration results in vector form, Xₖ is the sensor calibration values in matrix form (polynomial representation), and Aₖ are the polynomial coefficients in vector form for each energy source k. The optimal least-squares solution for each energy source k can be computed through the following equation,

$$\begin{matrix}{\hat{A}}_{k} = ( 1 / G ) {( {X}_{k}^{T} {X}_{k} )}^{- 1} {X}_{k}^{T} {Y}_{k} \\ where & Equation 6 \\ {\hat{A}}_{k} = [ \begin{matrix}{\hat{α}}_{0 , k} \\ {\hat{α}}_{1 , k} \\ {\hat{α}}_{2 , k} \\ ⋮ \\ {\hat{α}}_{P , k}\end{matrix} ] & \end{matrix}$$

Superscripts “−1” and “T” denote the matrix inverse and transpose operators. If the matrix Xₖ is square, then (Xₖ)⁻¹=(Xₖ^{T}Xₖ)⁻¹Xₖ^{T}. In an alternative embodiment, the technique and/or system disclosed in U.S. patent application Ser. No. ______, entitled “Method and System for Processing Data Streams” can be used to calculate Âₖ. This alternate technique provides an efficient numerical calculation of a global optimal solution for Âₖ while being insensitive to outliers that can be present in the acquired sensor calibration results Yₖ, due to circuitry and/or optical saturations, nonlinearities, and other factors. With the values for Âₖ calculated for each energy source k, the new sensor calibration settings can be calculated. First, the device firmware creates or retrieves a set of desired sensor calibration results (that are within dynamic range limits defined by the saturation element 2820 in FIG. 28) for each energy source,

$${\hat{Y}}_{k} = [ \begin{matrix}{\hat{y}}_{k} ( 1 ) \\ {\hat{y}}_{k} ( 2 ) \\ ⋮ \\ {\hat{y}}_{k} ( P + 1 ) \\ {\hat{y}}_{k} ( P + 2 ) \\ ⋮\end{matrix} ] \qquad Equation 7$$

Vector Ŷₖ can be the sensor calibration results for the digital signal 2824 around a main target sensor calibration result. For instance, if the main target sensor calibration result for the digital signal 2824 is ŷₖ(i^{TARGET})=50% of the full dynamic range defined in saturation element 2820, then the desired sensor calibration results can be distinct values between 40% and 60% of the full dynamic range in the saturation element 2820. Second, for each energy source k=1, 2, . . . , n, the device firmware in the monitoring device computes the new sensor calibration values,

{\hat{x}ₖ(i): i=1, 2, . . . , (P+1), (P+2), . . . } Equation 8

and the new gain value Ĝ for the frontend amplifier adjustable gain 2816 by solving the polynomials in Equation 4 for xₖ(i) and G, with yₖ) ŷₖ(i) and α_{p,k}=\hat{α}_{p,k} specified in order to obtain \hat{x}ₖ(i) and Ĝ. This computation can be accomplished by polynomial root analysis and root-finding methods, numerical optimization techniques, or by creating two-dimensional tables of points, one for each polynomial k of order P, that map yₖ and G values into xₖ, and then selecting the new sensor calibration values \hat{x}ᵢ(i) (for Equation 8) and the new gain value Ĝ that produce the closest results to the desired sensor calibration results ŷₖ(i) (in Equation 7) without violating the dynamic range limits in the saturation element 2820. The new sensor calibration settings in block 2706 are the new sensor calibration values in Equation 8, and the new gain value Ĝ. Another technique that can be used to compute the new sensor calibration values and the new gain value for the frontend amplifier adjustable gain 2816 is to solve Equation 4 for xₖ(i^{TARGET}) and G, with yₖ(i^{TARGET})=ŷₖ(i^{TARGET}) and α_{p,k}=\hat{α}_{p,k} specified to obtain \hat{x}ₖ(i^{TARGET}) and Ĝ. This computation results in asystem of n equations coupled by gain G, with (n+1) variables,

ŷₖ(i^{TARGET})=GΣₚ₌₀^{P}\hat{α}_{p,k}xₖ(i^{TARGET})^{p} Equation 9

for k=1, 2, . . . , n. The solution for xₖ(i^{TARGET}) and G in Equation 9 can be determined by polynomial root analysis and root-finding methods, numerical optimization techniques, or by creating two-dimensional tables of points, one for each polynomial k of order P, that map ŷₖ and G values into xₖ, and then selecting the target sensor calibration values \hat{x}ₖ(i^{TARGET}) and the new gain value G that produce the closest target sensor calibration results ŷₖ(i^{TARGET}) without violating the dynamic range limits in the saturation element 2820. To calculate the desired sensor calibration results and the new sensor calibration values so as to complete the vector entries in Equation 7, and complete the set elements in Equation 8, values for \hat{x}ₖ (i) are generated around the calculated \hat{x}ₖ(i^{TARGET}), and for each value, the corresponding ŷₖ(i) is computed directly via Equation 4 with G=Ĝ. In other words, the polynomials in Equation 4 are solved for yₖ(i), with xₖ(i)=\hat{x}ₖ(i), G=Ĝ, and α_{p,k}=\hat{α}_{p,k} specified to obtain ŷₖ(i) without violating the dynamic range limits in the saturation element 2820. The new sensor calibration settings are the new sensor calibration values in Equation 8, and the new gain value Ĝ for the frontend amplifier adjustable gain 2816.

4. Blocks 2708 and 2710 in FIG. 27: For each energy source k, the device firmware in the monitoring device determines the new sensor calibration values \hat{x}ₖ(i) (Equation 8) and the new gain value Ĝ for the frontend amplifier adjustable gain 2816 and acquires corresponding new sensor calibration results.

5. Block 2712 in FIG. 27: If the new sensor calibration results are close (e.g., within a given difference) to the desired sensor calibration results determined from the Equation 7 via computation of metrics (i.e., max absolute error, root mean square error, etc.) and do not violate the dynamic range limits in the saturation element 2820, then the sensor calibration process is completed, with the favorable or optimal sensor calibration settings given by the new gain value G for the frontend amplifier adjustable gain 2816 and the new sensor calibration values \hat{x}ₖ(i^{TARGET}) for each energy source k (i.e., {\hat{x}ₖ(i^{TARGET}): k=1, 2, . . . , n}). Otherwise, the device firmware in the monitoring device repeats the sensor calibration process starting from numbered paragraph 3 (Block 2706 in FIG. 27) using the new gain value calculated in the numbered paragraph 3 and the sensor calibration values calculated in the numbered paragraph 3 to assemble the matrices Xₖ, and the new sensor calibration results acquired in the numbered paragraph 4 (Blocks 2708 and 2710 in FIG. 27) to assemble vector Yₖ.

In some embodiments, sensor calibration process shown in FIG. 27 is iterative. The iterative sensor calibration process can increase flexibility and allow for simpler approximations (e.g., first-order or second-order polynomial) of the unknown nonlinear functions for the energy sources 2802. With the right predefined sensor calibration settings, the iterative version of the algorithm shown in FIG. 27 can improve the calculation of the sensor calibration settings at each iteration. A few iterations are typically performed to reach the favorable or optimal sensor calibration settings. In some embodiments, when calculating the new sensor calibration settings in the numbered paragraph 3 (Block 2706 in FIG. 27) using Equation 9, there can be multiple solutions for xₖ(i^{TARET}) and for G for each polynomial k in Equation 9. For example, consider P=1 and n=2, and (P+1) sensor calibration values with i^{TARGET}=1. Then Equation 9 becomes a system of two equations,

ŷ₁(1)=G(\hat{α}_{0,1}+\hat{α}_{1,1}x₁(1))

ŷ₂(1)=G(\hat{α}_{0,2}+\hat{α}_{1,2}x₂ Equations 10

with three variables (e.g., x₁(1), x₂(1), and G). This underdetermined system of equations (e.g., number of variables greater than the number of independent equations) allows for multiple feasible solutions whenever the calculated solutions fall within feasible dynamic ranges. The chosen solution can be the one that produces the smallest value for G, the highest value for G, or the solution that produces a G value closest to a predefined target value, or some other criteria that involve feasible values and/or ranges of x₁(1), x₂(1), and/or G in order to maximize signal-to-noise ratio, minimize power consumption, maintain power consumption and/or signal-to-noise ratio at predefined target values, etc.

The device firmware in the monitoring device can pick the smallest allowed gain value, the largest allowed gain value, or any allowed new gain value for G (the frontend amplifier adjustable gain 2816) that produces a feasible target sensor calibration value \hat{x}₁(1) for energy source 1, and \hat{x}₂(1) for energy source 2. The allowed values for the gain G (the frontend amplifier adjustable gain 2816) are typically functions of a programmable-gain amplifier (PGA) that typically has a finite number of settable gain values (e.g., 8, 16, 32, 64, 128, etc.). Once a value for the gain G is picked (i.e., C), the remaining sensor calibration values and the desired sensor calibration results can be calculated by setting x₁(2) to \hat{x}₁(2), a slightly higher or lower value than \hat{x}₁(1) (e.g., +/−10, +/−20, etc.), setting x₂(2) to \hat{x}₂(2), a slightly higher or lower value than \hat{x}₂(1) (e.g., +/−10, +/−20, etc.), and solving for ŷ₁(2) and ŷ₂(2) the following equations,

ŷ₁(2)=Ĝ(\hat{α}_{0,1}+\hat{α}_{1,1}\hat{x}₁(2))

ŷ₂(2)=Ĝ(\hat{α}_{0,2}+\hat{α}_{1,2}\hat{x}₂(2)) Equations 11

to complete the process for obtaining the desired sensor calibration results (i.e., ŷ₁(1), ŷ₂(1), ŷ₁(2), and ŷ₂(2)), the new sensor calibration values (i.e., \hat{x}₁(1), \hat{x}₂(1), \hat{x}₁(2), and \hat{x}₂(2)), and new gain value G for the frontend amplifier adjustable gain 2816.

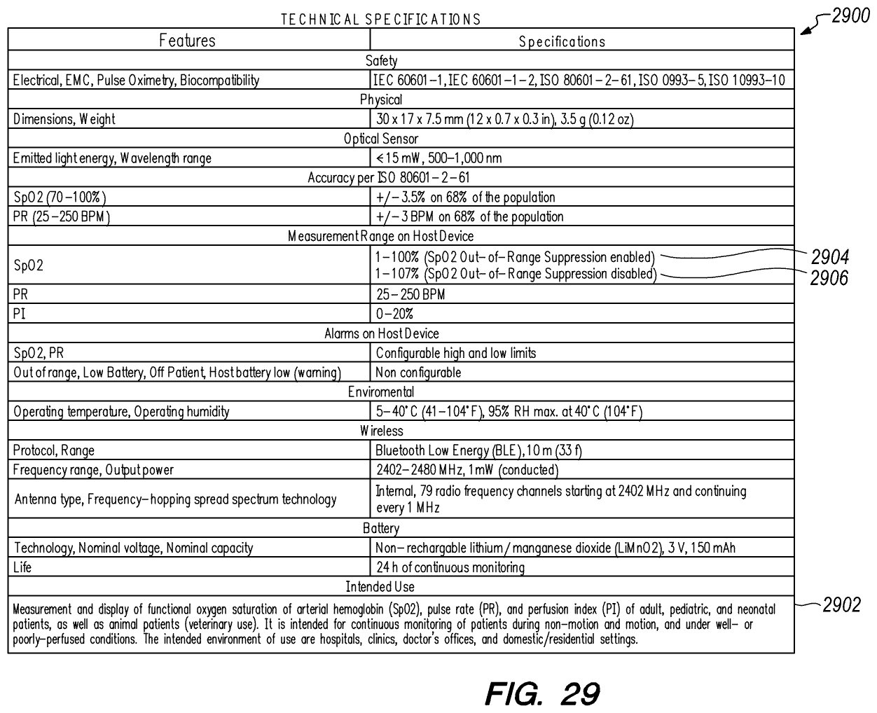

In one embodiment, the monitoring device, the host device, and the associated methods and apparatuses described herein can represent a clinical-grade pulse oximetry system for human or veterinary use. FIG. 29 depicts example technical specifications of a monitoring device when used in combination with a host device. Other embodiments are not limited to any of the specifications shown in FIG. 29.

In one aspect, the example specifications 2900 can be obtained by design and through biocompatibility tests, EMC and electrical safety tests, environmental tests, bench tests, and/or clinical tests. A pulse oximetry system, for instance, is typically certified to comply with several electrical equipment safety standards, electromagnetic compatibility, and FCC standards. Example standards may include, but are not limited to: (i) IEC 60601-1-2:2014 Medical Electrical Equipment—Part1-2: General requirements for basic safety and essential performance—Collateral Standard: Electromagnetic Compatibility—Requirements and Test. Safety Requirements for Electrical Equipment for Measurement, Control and Laboratory Use—Part 1: General Requirements; (ii) ANSI/AAMI ES 60601-1:2005/@ 2012 Medical electrical equipment—Part 1: General requirement for basic safety and essential performance; and (iii) 47 CFR Part 15 Subpart B Class B Devices and Innovation, Science; (iv) FCC Part 15, Subpart C and IC RSS-247, Issue 1, May 2015.

An example intended use 2902 can indicate uses such as measuring and displaying functional oxygen saturation of arterial hemoglobin (SpO2), pulse rate (PR), pulse amplitude (PA), and/or perfusion rate (PI) of adult, pediatric, and neonatal patients, as well as animal patients (veterinary use). The pulse oximetry system can be intended for continuous monitoring of human users (e.g., patients) and animal patients during non-motion and motion, and/or under well-perfused or poorly-perfused conditions. Example intended environments of use can be hospitals, clinics, doctor's offices, and domestic/residential settings, as well as confined areas, grazing lands, and outdoors.

A clinical-grade pulse oximetry system is commonly subjected to clinical testing to certify whether the pulse oximetry system meets the intended use 2902. For instance, in the case of a SpO2 accuracy clinical study recognized by the Food and Drug Administration (FDA), the pulse oximetry system may be tested in accordance to the Code of Federal Regulations (CFR) for Non-Significant Risk (NSR) investigational studies, following ISO 14155:2011 as appropriate, and the applicable sections of the pulse oximetry guidelines of ISO 80601-2-61:2011, and Pulse Oximeters—Premarket Notifications Submissions [510(k)s] Guidance For Industry and Food and Drug Administration Staff (issued: Mar. 4, 2013). The purpose of such a clinical study is to evaluate the SpO2 accuracy and performance during desaturations with medical devices placed on human subjects (fingers, forehead, temple, ear, etc.) over the range of 70-100% SaO2, arterial blood samples, assessed by CO-Oximetry. The objective is to show the SpO2 accuracy performance of the pulse oximetry system under test. Typically, a population of a given number of subjects (e.g., at least ten healthy adult subjects (male and female)), ranging in pigmentation from light to dark is enrolled into such a desaturation study. During the clinical tests, each subject is in a reclined position and connected to a breathing circuit for administering medical grade oxygen and nitrogen. The gas flow delivery is adjusted for subject comfort. The gas mixture is controlled to various levels of induced hypoxia resulting in stable oxygen saturation plateaus between 100% and 70% SaO2. Arterial blood samples are drawn during simultaneous data collection from the pulse oximetry system under test. The arterial blood samples are immediately analyzed by Reference CO-Oximetry providing functional SaO2 for the basis of the SpO2 accuracy comparison.

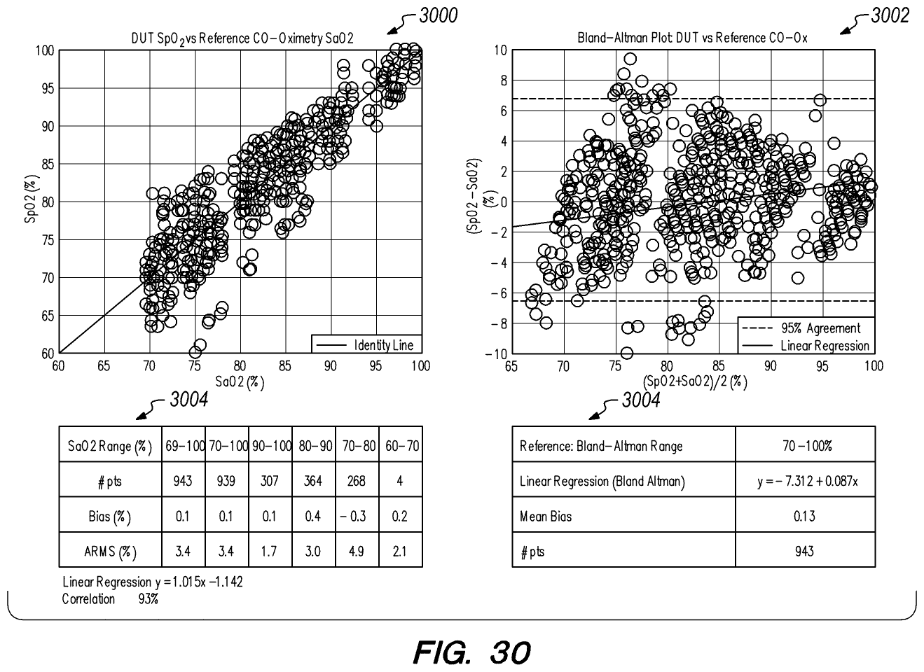

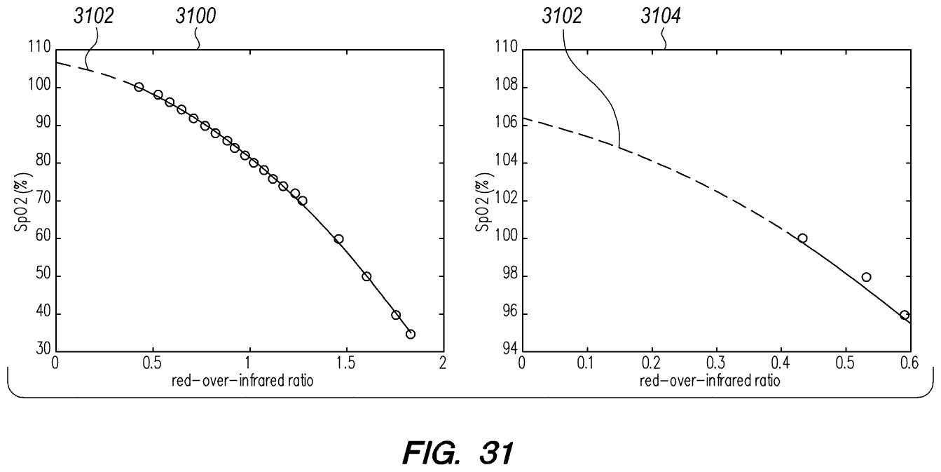

FIG. 30 shows an example scatter plot and an example Bland-Altman plot illustrating a typical SpO2 clinical performance for a reflectance-based pulse oximetry system. In the illustrated embodiment, the scatter plot 3000 and the Bland-Altman plot 3002 depicts the typical SpO2 clinical performance for a reflectance-based pulse oximetry system is in the 70-100% Sa02 range. The example performance metrics 3004 are in general the Accuracy Root Mean Square (Arms), bias, linear regression, correlation, etc. computed over a number of paired samples (SaO2, SpO2). For the scatter plot 3000 and the Bland-Altman plot 3002 shown in FIG. 30, nine hundred and forty-three (943) paired samples, evenly spaced over the 70-100% Sa02 range, were used to compute the performance metrics 3004.

There are medical, fitness, and wellness applications where users are given a high oxygen concentration by means of a nasal cannula or a facemask, or inside a hyperbaric chamber. These applications are generally referred to as oxygen therapy. Oxygen therapy is the use of oxygen as a medical or wellness treatment and is applied to carbon monoxide toxicity treatment, headache treatment, oxygen concentration supply during anesthesia procedures, and treatment of COPD (cystic fibrosis, etc.) in users with chronically low oxygen saturation. Aviators may use a nasal oxygen cannula or wear an oxygen facemask when piloting airplanes with non-pressurized cabins at high altitudes, or during emergencies to keep their blood oxygen saturation at acceptable levels. Athletes may use nasal oxygen cannulas to increase performance, or stay in pressurized oxygen chambers for a period of time to recover more quickly after workouts.

Typically, medical devices are calibrated via the aforementioned clinical studies, where adult patients are subjected to oxygen desaturations over the range of 70-100% SaO2. These patients are typically healthy adults with normal cardiorespiratory systems. Typically, healthy adult patients will have a measured SaO2 as high as 97-100% at normal arterial oxygen partial pressure. However, patients still have a percentage of hemoglobin with some of their oxygen-binding sites still empty at normal arterial oxygen partial pressure. This implies that, if such patients or users are exposed to a higher concentration of oxygen (such as during oxygen therapy), additional hemoglobin sites will bind to oxygen molecules. However, this increase cannot be captured by conventional medical devices because they are calibrated to measure 100% SpO2 at normal arterial oxygen partial pressures. Higher values are typically truncated to 100%, because the clinical community and regulatory agencies have adopted the convention that SpO2 should be limited to 100%, since it is a saturation (concentration) measure. What may be missed is the fact that the 100% saturation value is the result of instrument calibration by means of desaturation studies, and is applicable to patients, users, and animal patients with normal cardiorespiratory systems and not subjected to oxygen therapy. For patients under oxygen therapy, the ability to read oxygen saturation values higher than 100% is very useful, because it may guide physicians and/or caregivers in terms of oxygen partial pressure settings to be administered to a particular patient (based on SpO2 readings above 100%), and also to serve as a metric for treatment of patients with compromised cardiorespiratory systems (under oxygen therapy). Controlling oxygen partial pressure settings, through the monitoring of SpO2 in patients and users, also minimizes the effects of oxygen toxicity (i.e., oxidative damage to cell membranes, retinal detachment, etc.), and reduces oxygen consumption and its associated monetary costs. Oxygen toxicity is a concern for premature (neonatal) babies, pilots, astronauts in space, underwater divers, and patients undergoing treatment in a hyperbaric oxygen chamber

Co-oximeters (such as the ABL90 FLEX from Radiometer) provide a range of indication for SaO2 (e.g., the arterial oxygen saturation in the blood measured by a co-oximeter) above 100% (102% for the ABL90 FLEX). Typically, the user has the option to activate the “Out-of-range suppression” option and prevent the co-oximeter from reporting values higher than 100%. These in-vitro instruments have SaO2 accuracy better than 1%. The range of indication above 100% is built-in so as to account for measurement errors, as well as to provide clinicians with SaO2 reading values above 100% for patients under oxygen therapy. The inconvenience of such in-vitro instruments (co-oximeters) relates to the inability to provide continuous measurements, as well as the need for arterial blood samples. Therefore, a non-invasive pulse oximetry system, such as the monitoring device disclosed herein operating in combination with a host device that can also monitor SpO2 continuously for values above 100%, can be invaluable in clinical and non-clinical settings where oxygen therapy is required or adopted, or where oxygen toxicity is a concern.

FIG. 31 illustrates example experimental points (circles) of a typical calibration curve (calcurve) for a pulse oximetry system. The calcurve 3100 in FIG. 31 maps the optical ratios (red over infrared) produced by a pulse oximetry system (e.g., the monitoring device and the host device) into SpO2 values. Such a calcurve is usually obtained experimentally by means of clinical studies, such as the ones aforementioned, where users are subjected to oxygen desaturations, via optical and physiological modeling using photon-diffusion methods (e.g., Boltzmann's Transport Equation), or via a combination of modeling and experimental data. To enable a monitoring device and a host device to measure and display SpO2 readings above 100%, the calcurve is extrapolated for SaO2 values above 100%. In the illustrated experimental calcurve 3100, a red-over-infrared ratio of 0.42 corresponds to 100% SpO2. To extrapolate the calcurve 3100, a curve can be fit to the experimental points and then the resulting curve's expression can be used to extrapolate for red-over-infrared ratios below 0.42 (i.e., SpO2 values above 100%). The dashed line 3102 represents a smooth extrapolation after the experimental points are fit to a quadratic polynomial. In some embodiments, the resulting calcurve polynomial is defined by the following equation,

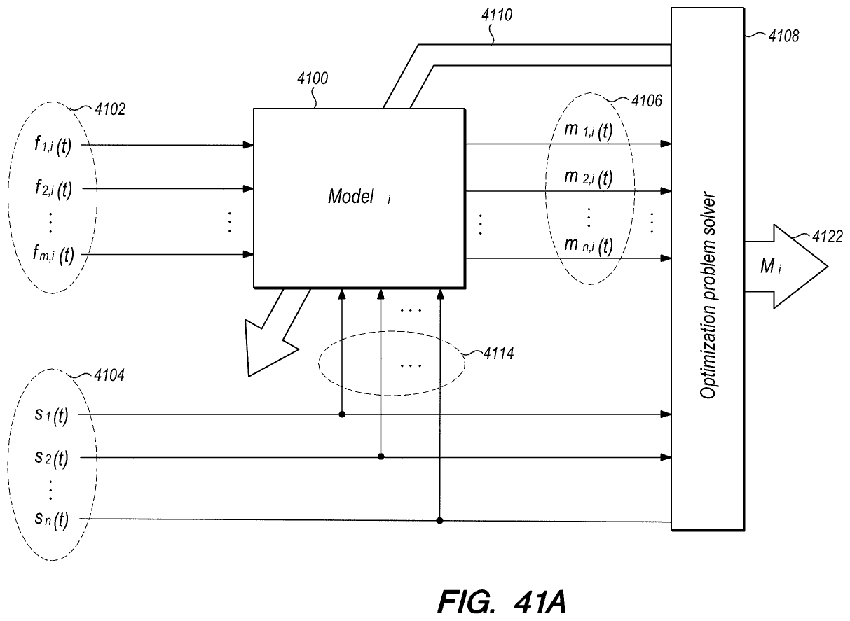

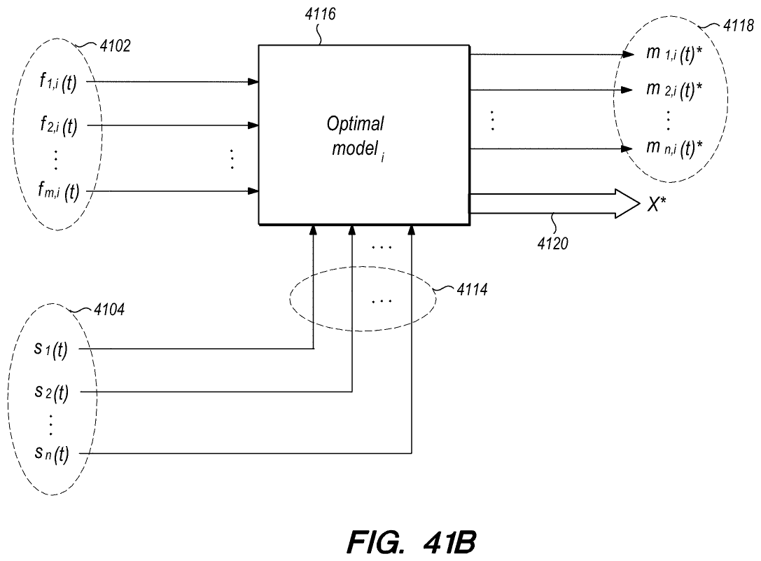

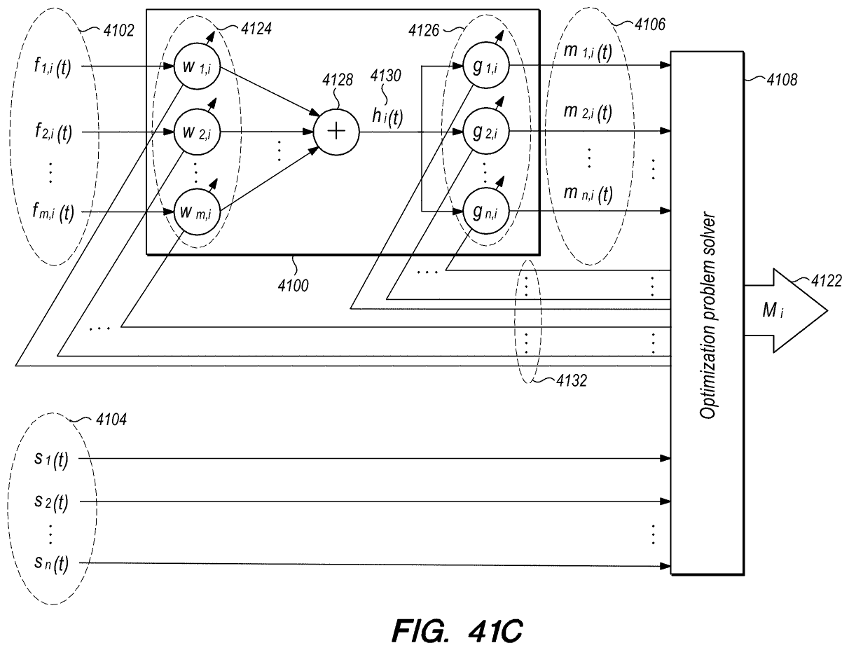

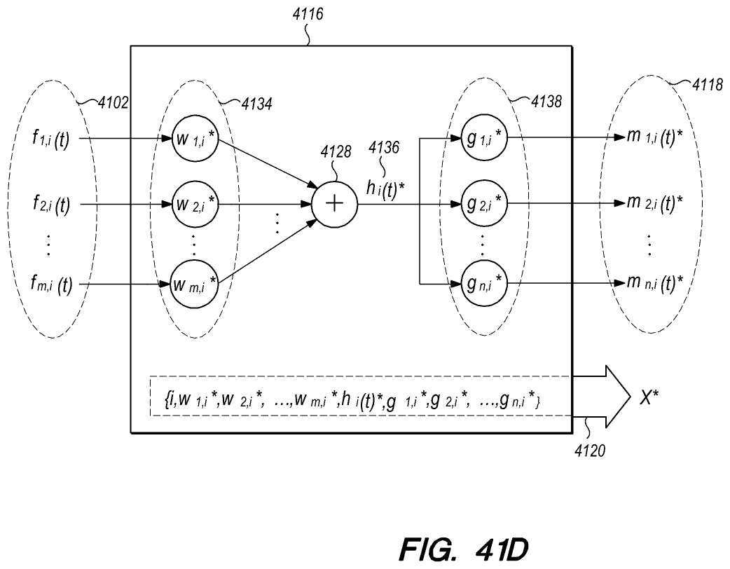

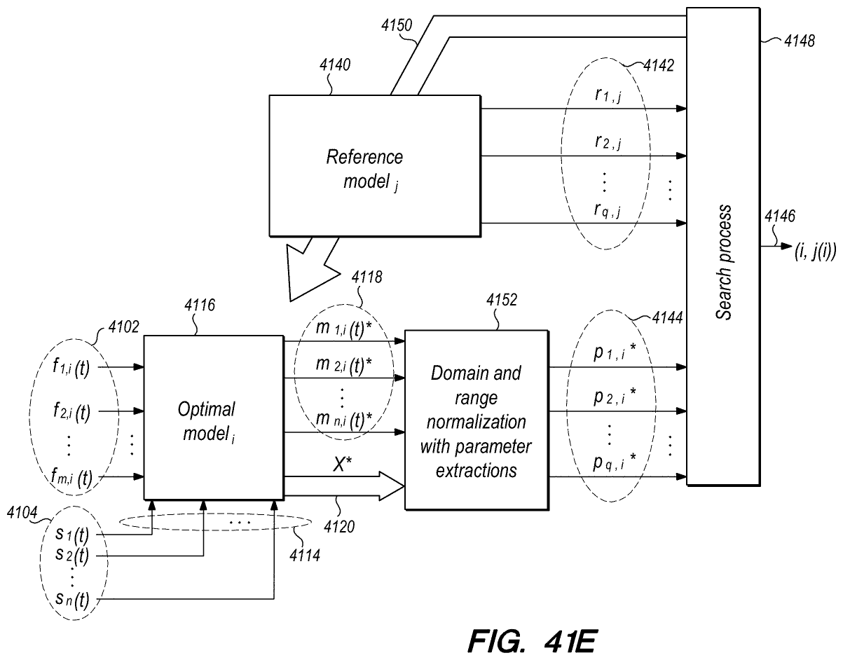

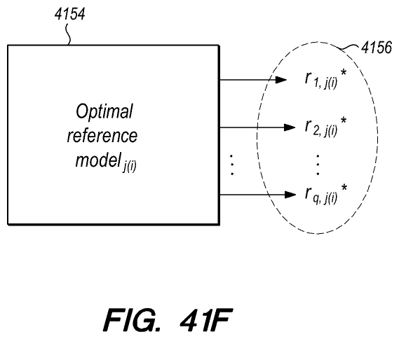

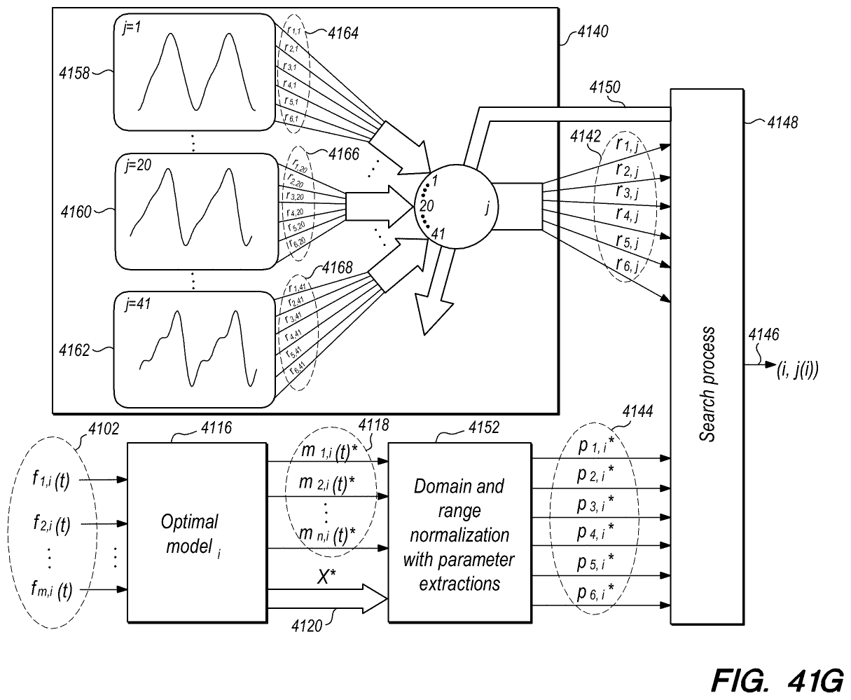

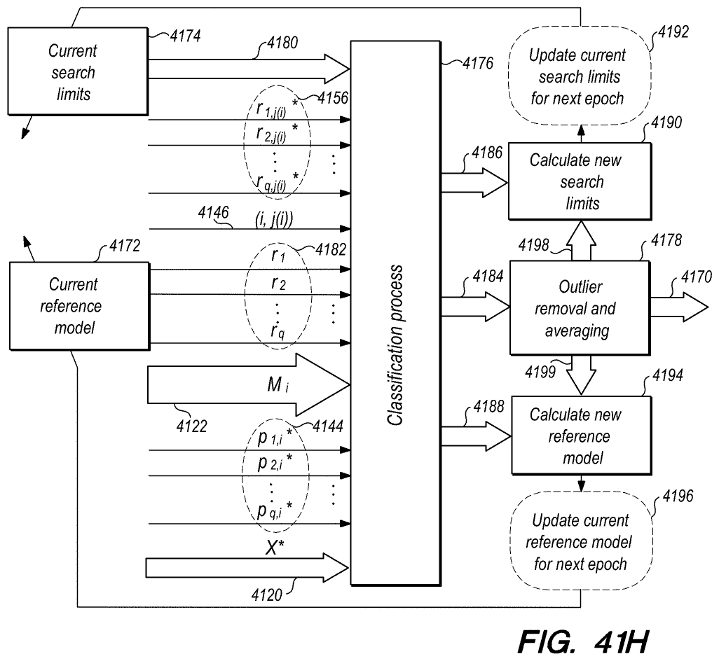

y=−16.79x²−8.19x+106.43 Equation 12

where x is the red-over-infrared ratio value and y is the SpO2 value. The “red-over-infrared” ratio is not necessarily obtained or calculated by dividing two quantities (the red photoplethysmograph over the infrared photoplethysmograph). Because of numerical stability, noise, and interferences, the division may be replaced with iterative methods that find the best linear relationship between the two quantities of interest, such as the embodiments described herein (i.e., FIGS. 41A-41H, 42, and 43). The calculated linear relationship will be an estimate for the ratio and can be used as a value for x in Equation 12. The “red-over-infrared” ratio can also represent more complex ratiometric relations between red, infrared, and other wavelength photoplethysmograph amplitudes such as the functional ratiometric form shown in Equation 32, or some other functional form with algebraic loops that is solved through iterative methods.

Other fittings such as cubic, exponential, etc. can be used to yield similar results. In addition, constrained optimization (fitting) combined with photon-diffusion models can be applied to find an extrapolated curve that satisfies both the calcurve experimental (model) data and the hill's equation (with a cubic term for better accuracy, such as the one disclosed by John W. Severinghaus, in Simple, Accurate Equations for Human Blood O2 Dissociation Computations. J. Appl. Physiol: Respirat. Environ. Exercise Physiol. 46(3):599-602, 1979. Revisions, 1999, 2002, 2007) for a given nominal partial pressure of oxygen. FIG. 31 also depicts a magnified view 3104 of the extrapolated SpO2 calcurve 3100.

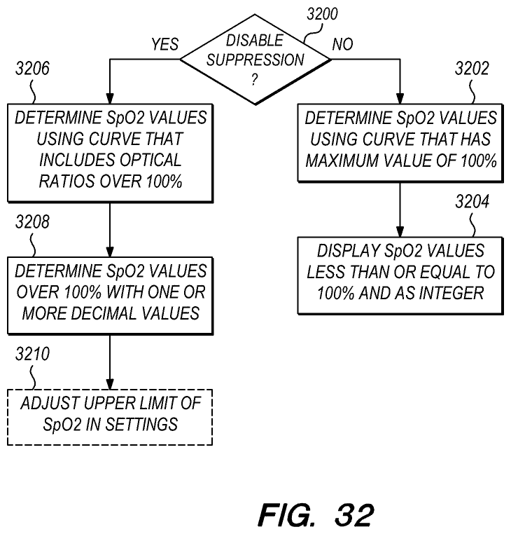

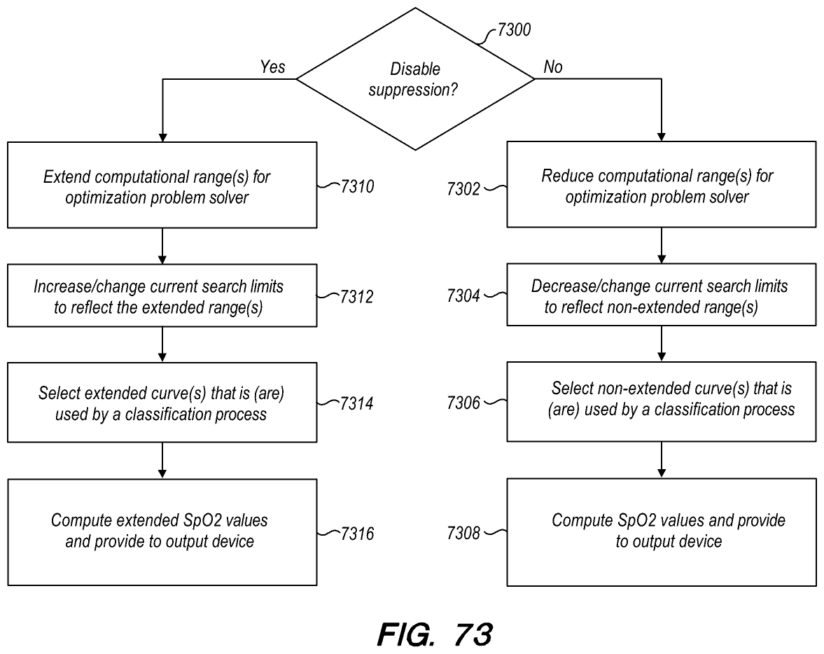

Returning to FIG. 29, the measurement ranges for SpO2 are shown with the “SpO2 out-of-range suppression” enabled 2904 and disabled 2906. In one embodiment, the maximum value of the extended SpO2 range can be 107%, which can be obtained by rounding up the SpO2 value computed from the extended calcurve (e.g., calcurve 3102 in FIG. 31) for a red-over-infrared ratio equals to zero. Other lower or higher maximum values can be used depending on the adopted calcurve, the user population, and/or the application. An example method for providing SpO2 values over 100% is shown and discussed in more detail in conjunction with FIGS. 32 and 73.

Another aspect of implementing the extrapolated SpO2 calcurve 3102 in FIG. 31 with the magnified view 3104 relates to the recommendations described in ISO 80601-2-61:2011, the standard for basic safety and essential performance of pulse oximeter equipment. According to ISO 80601-2-61:2011 (section EE.2.3.4 Data analysis), an invasive controlled desaturation study data analysis should be performed as follows: “for pulse oximeter monitors that place an upper limit on displayed SpO2 (e.g. 99% or 100%), a means that does not bias the Arms result should be used. EXAMPLE 1—Include only observations where SpO2 readings are less than the upper display limit; EXAMPLE 2—Statistically down-weight those values with SpO2=100% (e.g. treat observations of 100% as censored, as is done in the analysis of survival data); EXAMPLE 3—Configure the data-collection system to record values of SpO2>100%.” Therefore, “EXAMPLE 3” enables SpO2 data collected above 100% by the pulse oximetry system under test (during a controlled desaturation clinical study) to compute its SpO2 Arms accuracy. No special considerations or exclusions, such as in “EXAMPLE 1” and “EXAMPLE 2”, are needed. “EXAMPLE 3” is enabled by means of an extrapolated SpO2 calcurve (e.g., calcurve 3102), where a “SpO2 Out-of-Range Suppression” setting is disabled.

There are other pulse oximetry solutions that display ad hoc proprietary indexes to monitor users under oxygen therapy. In these implementations, indexes typically vary from zero to one, where the zero value corresponds to an arbitrary lower limit, and one to an arbitrary upper limit. This approach can be confusing, and not very practical for clinicians. Clinicians are typically trained to interpret values of SpO2 in the normal scale unit and range. Displaying SpO2 values above 100%, in the same scale as the values of SpO2 in the standard range (0-100%), is not only natural, but also enables physicians and other health care providers to use their clinical experience (as far as interpreting SpO2 readings in the standard scale) to make more accurate clinical decisions on users under oxygen therapy. The use of an index is not based on scientific principles. The use of an index can be a strategy to enable medical device manufacturers to claim monitoring capabilities during oxygen therapy without providing accuracy claims or clinical validations based on scientific methods. In addition, because indexes use unit-less scales, manufacturers have the freedom to disclose measurement principles that are in line with their strategy, and not necessarily in agreement with scientific methods and principles. The extrapolated SpO2 calcurve, such as the calcurve 3102, is a model-based and data-based approach to measure and display SpO2 values above 100% from users under oxygen therapy, and from users in general that have their SpO2 baseline shifted upwards because of particular physiological and/or environmental conditions. The extrapolated SpO2 calcurve provides a scientific-based alternative to arbitrary and ad hoc indexes currently available in the marketplace.

FIG. 32 depicts a flowchart of an example method of providing SpO2 values that exceed one hundred percent. Initially, a determination is made at block 3200 as to whether the “SpO2 Out-of-Range Suppression” is disabled. In one embodiment, the out-of-range suppression is disabled using a setting in a user interface screen (e.g., setting 7502 in FIG. 75). If the out-of-range suppression is enabled (e.g., the out-of-range suppression is not disabled), the process passes to block 3202 where the SpO2 value(s) is determined using a calcurve or plot that has a maximum SpO2 value of 100%. Any SpO2 values that exceed 100% may be rounded down to 100%. The SpO2 value or values are then provided to an output device (e.g., a display) as an integer number that is equal to or less than 100% (block 3204).

When a determination is made at block 3200 that the out-or-range suppression is disabled, the method continues at block 3206 where the SpO2 value(s) is determined using a calcurve or plot that has a maximum value greater than 100%. For example, as described earlier, the maximum value can be 107%. The calcurve includes or represents optical ratios (red over infrared) over 100%. In some instances, a curve can be fit to the values and then the resulting curve's expression can be used to extrapolate SpO2 values above 100%.

At block 3208, the SpO2 value(s) is provided to an output device (e.g., a display) and represented by a whole number and optionally one or more decimal place values (e.g., tenths place value, hundredths place value, etc.). Optionally, the upper limit of the SpO2 value for an alarm can be automatically adjusted at block 3210 to a value over 100% (e.g., the maximum value over 100%). For example, as shown in FIG. 75, the upper limit in the SpO2 Alarm Limits setting 7504 can be automatically adjusted to a value higher than 100%.

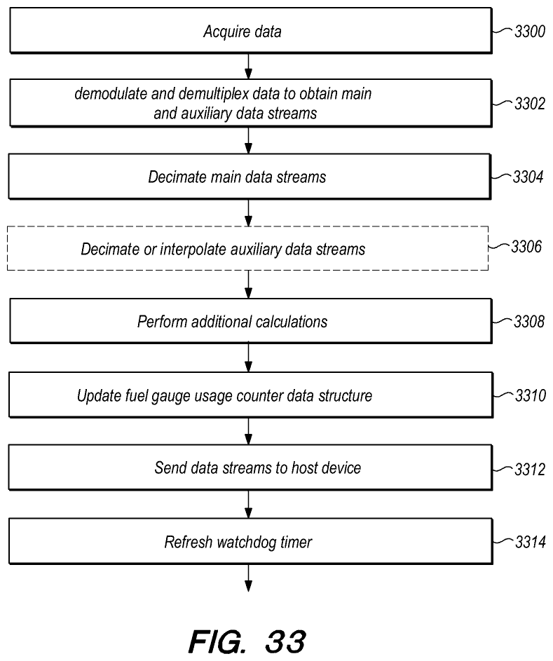

FIG. 33 illustrates a flowchart of example operations that are performed while a monitoring device is running and connected to a host device. Initially, data streams are acquired from the monitoring device (block 3300). The data streams, depending on the signal-to-noise ratio requirements, can be acquired at a relatively high sampling frequency (e.g., 4 kHz, 20 kHz, etc.), when compared to the data streams' inherent frequency bandwidths (e.g., 10 Hz, 20 Hz, 100 Hz, etc.), to enable averaging through decimation processes. The relatively high sampling frequency of the data acquisition can also enable the correct demodulation and demultiplexing of data streams that can be time multiplexed and modulated by energy sources (optical, electrical, mechanical, etc.).

In an example embodiment, a monitoring device uses red and infrared light sources (e.g., light sources 113 in FIG. 1) to measure oxygen saturation and pulse rate. Time multiplexed and modulated red and infrared lights are generated by the monitoring device (e.g., the frontend circuitry) and applied to the measurement site. The red and infrared lights penetrate the blood perfused dermis and interact with the heart pulsatile signals in order to create time multiplexed and modulated red and infrared optical signals with pulsatile components (photoplethysmographs). The red and infrared optical signals are captured by one or more photodetectors (e.g., photodetector 114 in FIG. 1) in the monitoring device (e.g., the frontend circuitry) and converted to discrete-time digital samples through an analog-to-digital converter. The sequence of discrete-time digital samples forms a data stream that is acquired in block 3300.

The data stream or data streams are demodulated and demultiplexed to produce individual data streams at block 3302. In a non-limiting example, the demultiplexing and demodulation process produces two data streams, one for the red optical signal, and another for the infrared optical signal. Auxiliary data streams can also be demodulated and/or demultiplexed. For instance, the monitoring device can also produce a time-multiplexed data stream where each time slot provides a value that can be from an electrical current, a resistance, or a voltage variable, a temperature variable, a digital counter variable, or a diagnostic variable. The auxiliary data stream is demultiplexed in block 3302 to produce a number of data streams, one for each time multiplexed variable.

Next, as shown in block 3304, the demultiplexed data streams are operated on by a decimation process, where the sampling rate and the frequency bandwidth of the demultiplexed data streams are reduced to minimize the signal processing requirements, to reduce power consumption, and/or to increase the signal-to-noise ratio through averaging by the decimation filters. In blocks 3302 and 3304, any suitable demodulation technique and decimation technique can be used. For example, in one embodiment, the demodulation and decimation techniques and/or system disclosed in U.S. patent application Ser. No. ______, entitled “Method And System For Symmetric Square-Wave Demodulation” can be used in blocks 3302 and 3304 to demodulate, demultiplex, and decimate the individual data streams produced by the monitoring device in block 3300.

The auxiliary data streams can be decimated or interpolated depending on the desired sampling frequency at block 3306. For example, the temperature in the measurement site (related to the user's core body temperature when the monitoring device is placed on forehead), can be collected at a sampling rate of 1 kHz and decimated to 5 Hz, given that the user's core body temperature frequency bandwidth is typically much less than 5 Hz. Depending on the type of auxiliary data streams, interpolation may be performed to increase the sampling rate to make all of the auxiliary data streams have the same sampling frequency. Interpolation can also be used prior to decimation to achieve an overall rational decimation factor.

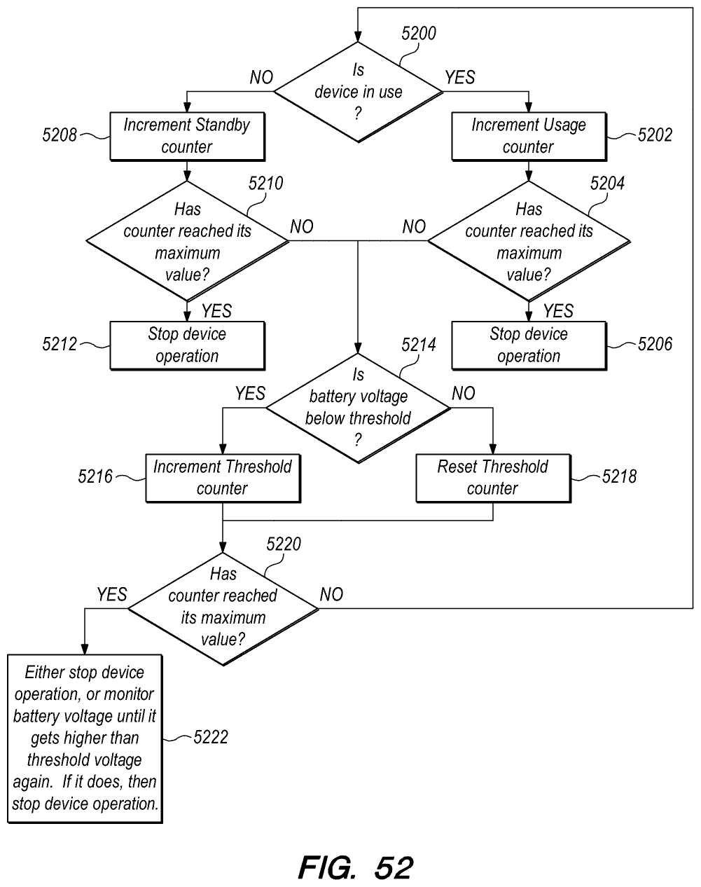

In block 3308, additional calculations are performed. The additional calculations can be operations for converting scales of data streams and variables, electronic saturation detection, or any other computation performed by or for the device firmware in the monitoring device. A fuel gauge usage counter data structure is updated in block 3310 with an increment (or decrement) to reflect a decrease in the battery's energy. An example usage counter for a fuel gauge is described in more detail in conjunction with FIG. 52.

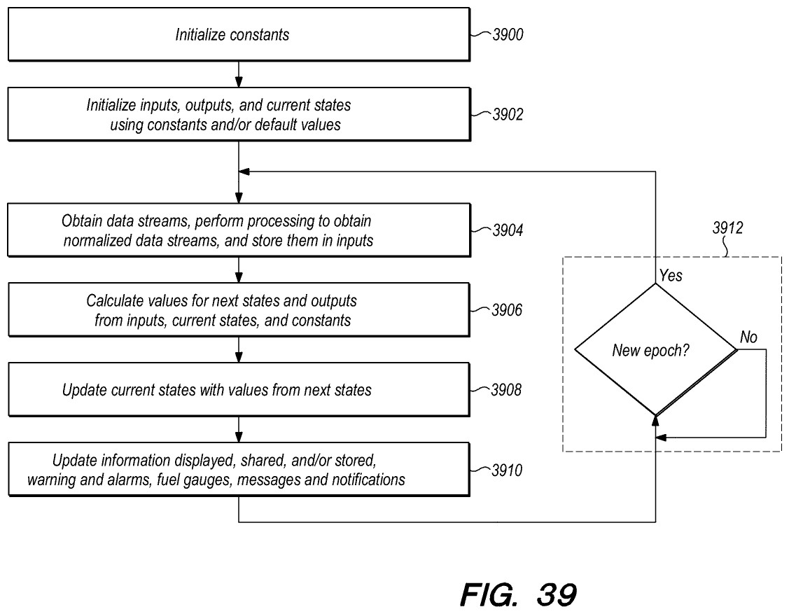

The demodulated, demultiplexed, and decimated data streams and auxiliary data streams (if any), along with other optional parameters and variables are sent to a host device at block 3312. The data streams are received in real-time by the host device, and in one embodiment are further processed by the host device. In an example embodiment, such as depicted in block 3904 (FIG. 39), the data streams are passed through a non-linear (such as a log, hyperbolic, exponential, polynomial function, etc.), linear, or affine function and the resulting data streams are fed into linear or non-linear low-pass, band-pass, or high-pass filter(s) to obtain normalized data streams. The normalized data streams are used by the host software in the host device, as shown in the embodiments of FIGS. 41A-41H, 42, and 43.

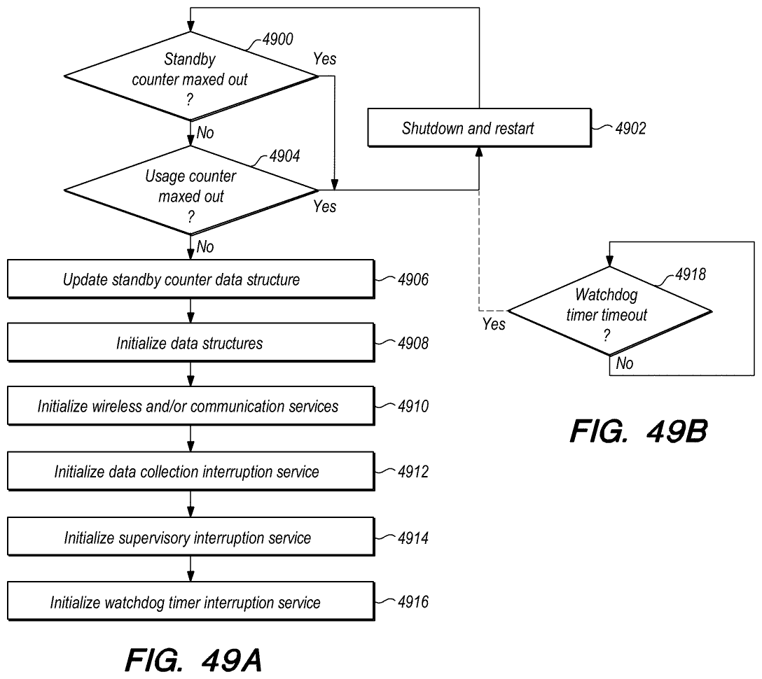

Next, as shown in block 3314, a watchdog timer is refreshed to prevent a watchdog timer interruption from becoming active and stopping the normal operation of the monitoring device. An example watchdog timer is described in more detail in conjunction with FIG. 49B.

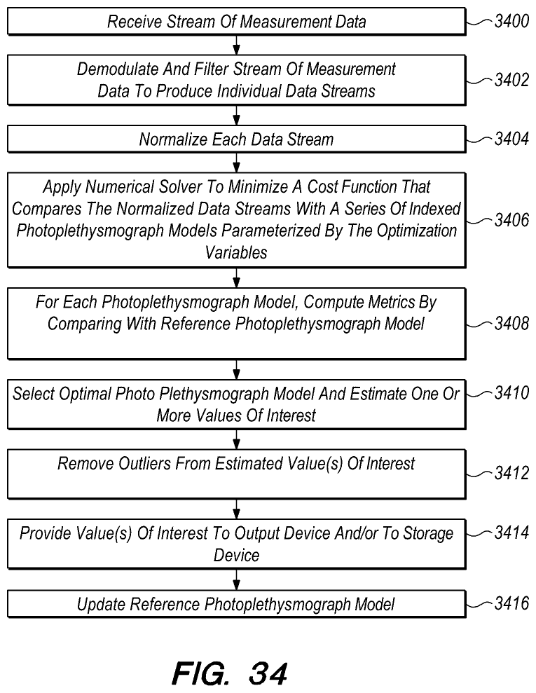

FIGS. 34-38 depict different example methods of processing measurement data that can be executed by a host device. FIG. 34 depicts a flow chart illustrating an example method of processing measurement data received from a monitoring device. The illustrated method fits measurement data to a model and based on the model, determines one or more physiological parameters (e.g., PR, SpO2, PI). Depending on the application, the method of FIG. 34 is performed once, or the method repeats for a given number of times. For example, while working in combination with a monitoring device, the method shown in FIG. 34 can repeat as long as one or more streams of measurement data is received. In a non-limited example, the method of FIG. 34 repeats substantially every 0.75 seconds.

Initially, as shown in block 3400, a stream of measurement data is received. In one embodiment, the stream of measurement data is a time-multiplexed and modulated digital stream of measurement data. In a monitoring device embodiment, the stream of measurement data represents any suitable number of measurement samples that are captured by the monitoring device at a given sampling frequency (e.g., 4 kHz). In one embodiment, the stream of measurement data is captured continuously by the monitoring device, although other embodiments are not limited to this implementation.

The stream of measurement data is then demodulated and filtered at block 3402 to produce individual data streams for each wavelength channel (e.g., red, infrared, etc.). Any suitable demodulation technique can be used. In a non-limiting example embodiment, a demodulation system can include a multi-channel symmetric square wave demodulator device operably connected to a filter device, as disclosed in co-pending U.S. application Ser. No. 16/198,550 filed on Nov. 21, 2018. The filter device can be implemented as a single stage or a multi-stage filter device. In some embodiments, the demodulator device and/or the filter device perform decimation, where the sampling frequency is reduced to a lower value (e.g., from 4 kHz to 1 kHz, from 1 kHz to 50 Hz) to reduce signal processing requirements, wireless bandwidth, and/or power consumption. Additionally or alternatively, the demodulation system and techniques are capable of removing most or substantially all interference signals within a pre-defined continuous frequency range (i.e., 0 Hz to 800 Hz).

In some aspects, each individual data stream is a photoplethysmograph data stream. At block 3404, each individual data stream is normalized. In one embodiment, a log of each data stream is taken and bandpass filtered to produce a photoplethysmograph data stream for each wavelength channel.

Next, at block 3406, the photoplethysmograph data streams are processed by a numerical solver device to calculate or estimate optimization variables that minimize a cost function to produce one or more photoplethysmograph models. In one embodiment, the photoplethysmograph data streams are processed in data batches of any size that is suitable for a particular application. For example, with a monitoring device, the data batch size can be the equivalent of a few seconds of data (e.g., 250 samples collected over 5 seconds) and updated in real-time every certain time interval (e.g., 0.75 seconds).

In one aspect, the numerical solver can compare the data streams with a series of indexed photoplethysmograph models parameterized by the optimization variables. For example, for each Pulse Rate (PR) value, from 25 to 250 BPM in steps of 1 BPM, the numerical solver device calculates values for the optimization variables that minimize a cost function to produce the best photoplethysmograph model for the given data streams. As disclosed in co-pending U.S. application Ser. No. 16/198,504 filed on Nov. 21, 2018, in one embodiment, the cost function can be defined by the following equation:

J(x,z)=Σᵢ₌₁ⁿ(zᵢAx−bᵢ)^{T}(zᵢAx−bᵢ), Equation 13

where A∈R^{k×m}, k≥m, is a constant matrix, bᵢ∈R^{k}, i=1, 2, . . . n are constant vectors, x∈R^{m} and z=[z₁ z₂ . . . zₙ]T∈Rⁿ are the optimization variable vectors, and the T superscript is the transpose operator.

Each photoplethysmograph model produced based on the numerical solver device and its corresponding PR value is considered a data point (pair). As a result, in this example, the photoplethysmograph models are indexed by the PR values. If the cost function is given by Equation 13, then each photoplethysmograph model is represented by the vector Ax and scaling factors zᵢ, and the photoplethysmograph data streams are represented by the vectors bᵢ. The optimization variables are the vector x and scaling factors zᵢ. Each column in Matrix A provides information regarding the underlying application or phenomenon. In one embodiment, Matrix A is indexed by (function of) the PR values. As a result, the entries in Matrix A change for each PR value, which in turn change the optimal solutions for x and scaling factors z that minimize the cost function in Equation 1.

Next, as shown in block 3408, the one or more metrics are computed for each photoplethysmograph model indexed by a PR value by comparing the photoplethysmograph models with a reference photoplethysmograph model. The reference photoplethysmograph model represents the best or a selected photoplethysmograph model for a user associated with the measurement data. In one embodiment, the one or more metrics are associated with the photoplethysmograph model. Example metrics include, but are not limited to, root mean square accuracy (Arms), correlation, L2 norm, L1 norm, Linf norm, power, correlation, and harmonic and morphology analysis matching. Because the one or more metrics are calculated from photoplethysmograph models that are indexed by PR values, the one or more metrics are also indexed by the same PR values.

For example, in some embodiments, the one or more computed metrics are compared with corresponding metrics associated with the reference photoplethysmograph model (reference metric(s)) to determine how close or similar the one or more computed metrics are to the corresponding reference metric(s). Additionally or alternatively, the shape of each photoplethysmograph model is compared with a shape of the reference photoplethysmograph model to determine how similar or dissimilar each photoplethysmograph model is to the reference photoplethysmograph model. In some embodiments, metrics such as the root mean square accuracy (Arms), correlation, L2 norm, L1 norm, Linf norm, correlation, and harmonic and morphology analysis matching can be used to access the degree of compliance (shape similarity) between photoplethysmograph model and reference photoplethysmograph model.

At block 3410, an optimal photoplethysmograph model is selected or determined for each wavelength channel and one or more values of interest are estimated or computed. The values of interest can include values of interest for physiological parameter such as SpO2, PR, PA, PI, and/or other physiological parameters of interest. The values of interest are computed by applying classification criteria (algorithms) to the computed metrics (i.e., maximum value, minimum value, a ratio of values, linear and non-linear classification algorithms, etc.). For instance, the best estimate for PR, for given red and infrared data streams, may be obtained by picking the PR value that produces the photoplethysmograph model with the largest normalized power, provided that the corresponding photoplethysmograph model Arms error value (when compared to the most current reference photoplethysmograph model) is less than a specified threshold. The best estimate for SpO2 and PI may be calculated via the scaling factors (red and infrared amplitudes) from the photoplethysmograph model that produced the best estimate for PR.

One or more outliers are then removed from the estimated values of interest to produce a subset of values of interest. In some embodiments, average estimates of the values of interest are produced at block 3412. Any suitable technique can be used to remove the outliers.

The subset of values of interest are then provided to a storage device (e.g., memory) and/or an output device (block 3414). For example, the one or more values of interest can be displayed on a display. Next, as shown in block 3416, the reference photoplethysmograph model is updated based on the subset of values of interest and/or the optimal photoplethysmograph model (e.g., the associated optimization variables). In one embodiment, the reference photoplethysmograph model is updated via an update rule that produces a weighted average of the current reference photoplethysmograph model and the optimal photoplethysmograph model

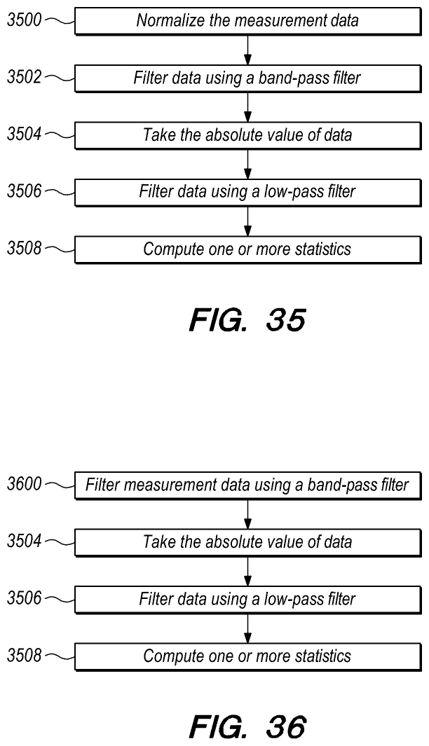

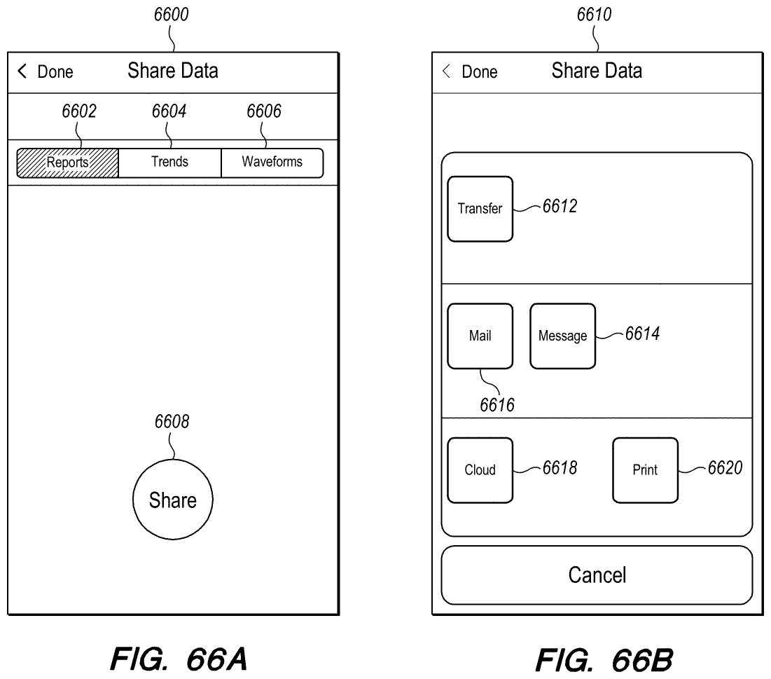

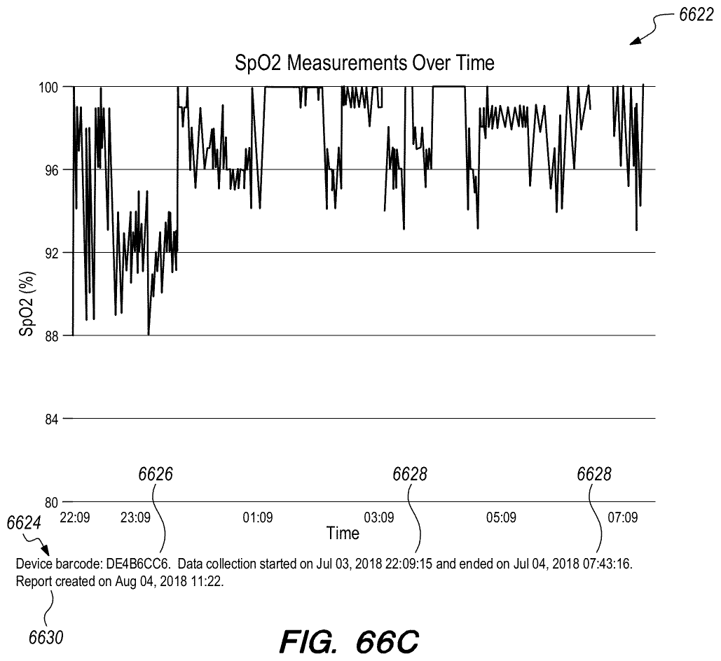

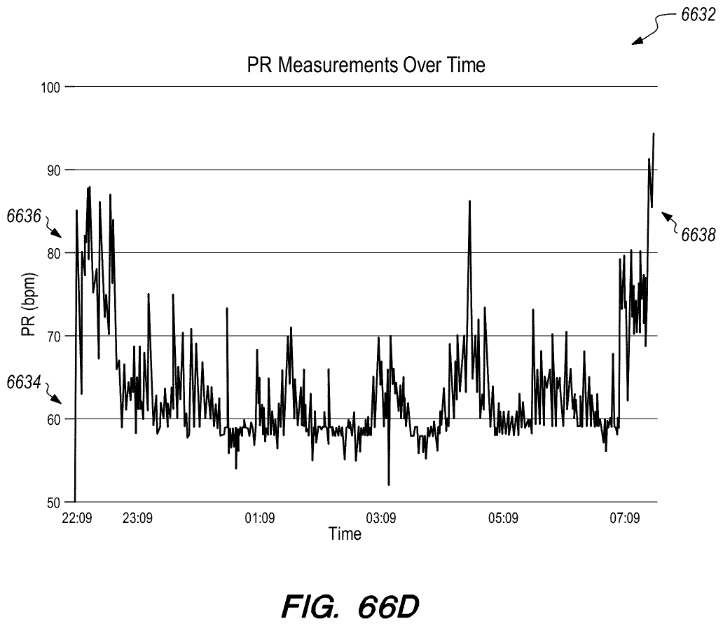

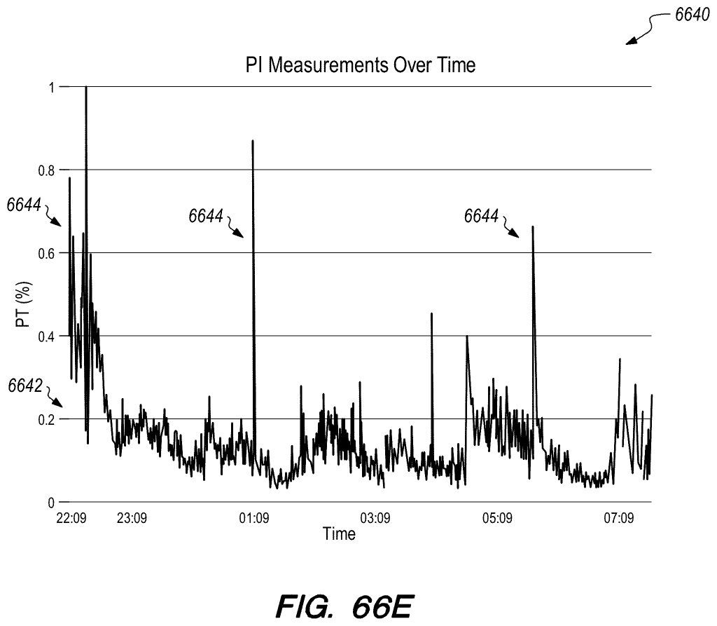

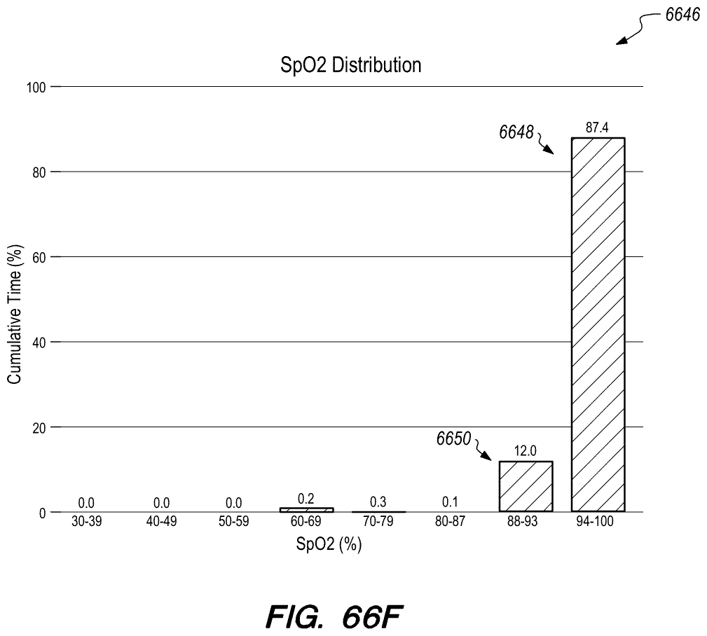

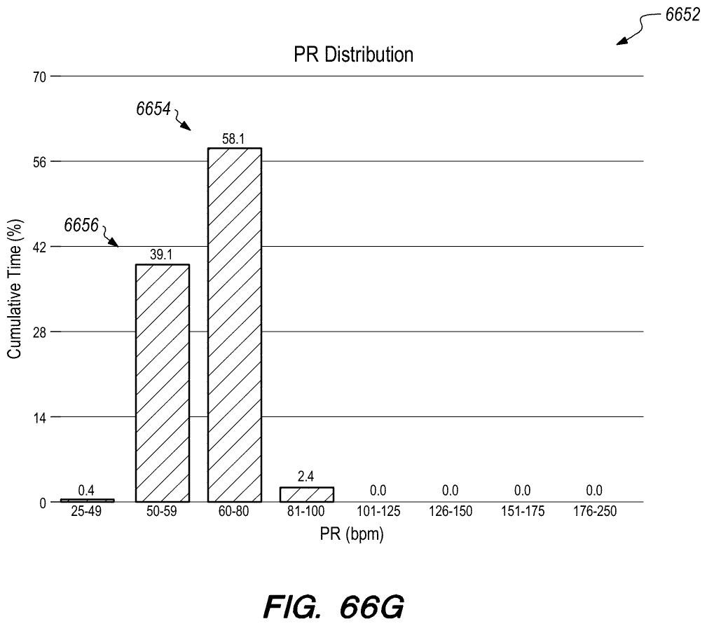

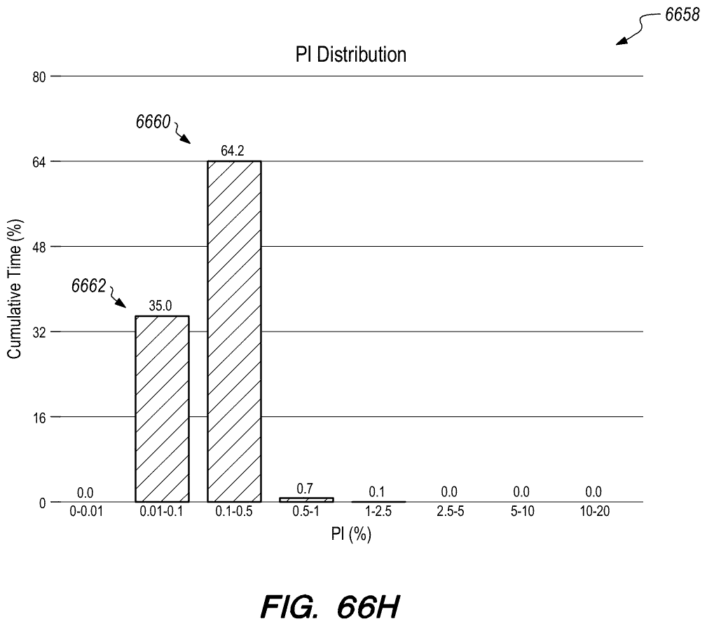

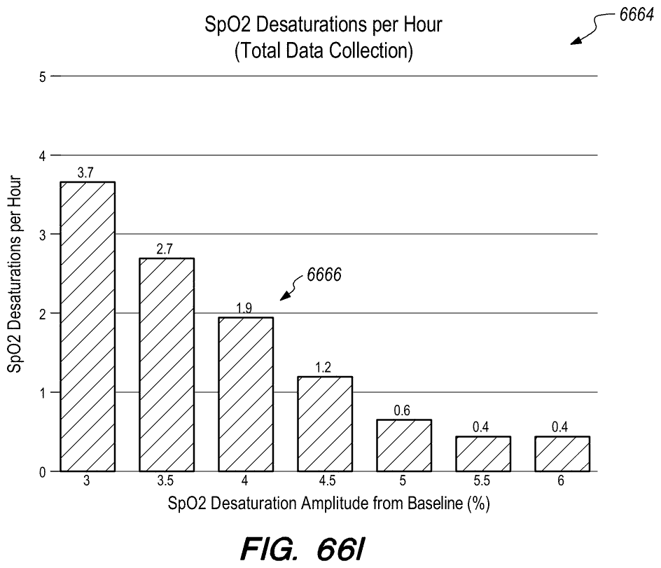

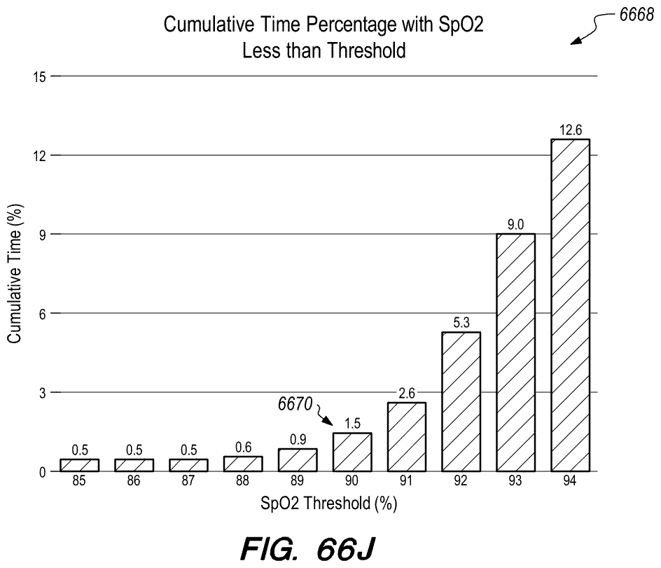

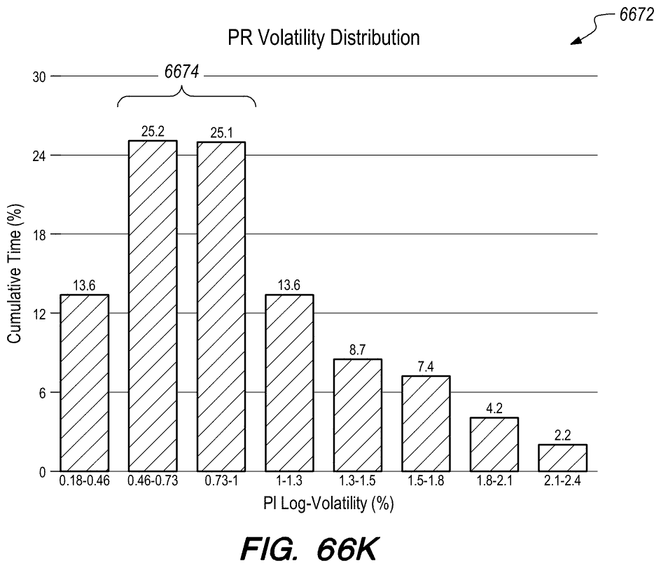

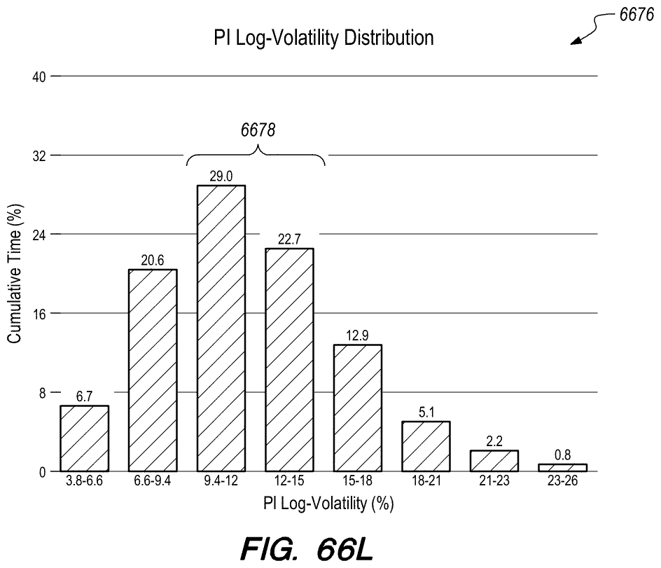

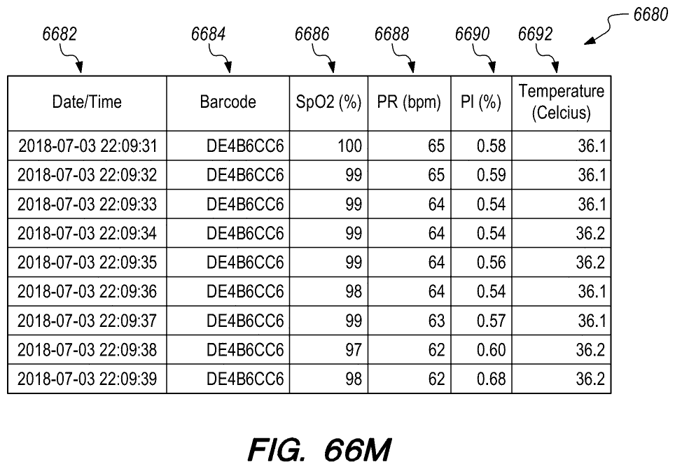

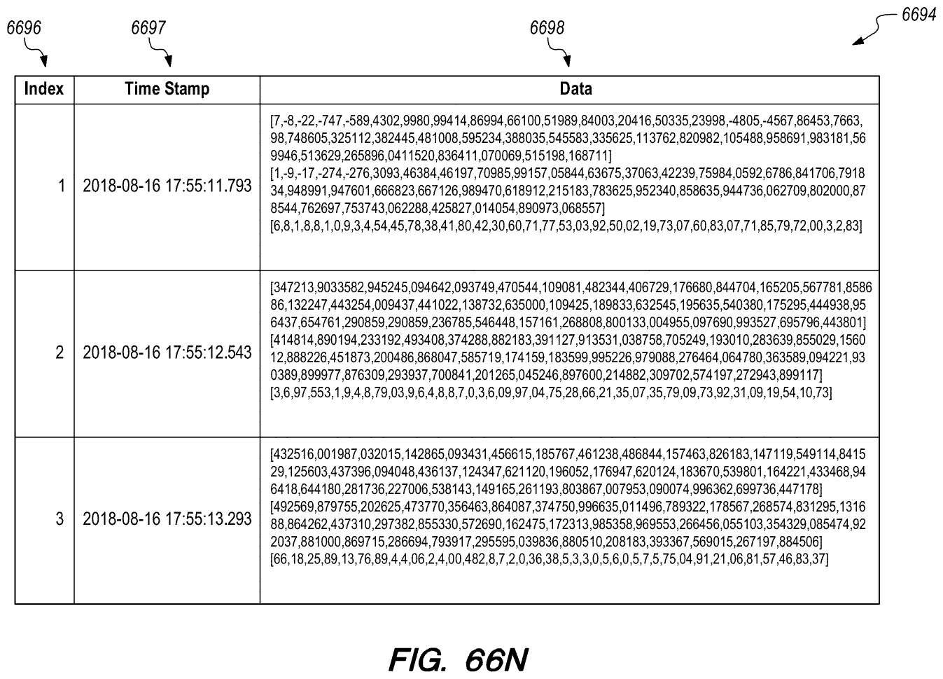

FIG. 35 illustrates a flowchart of an example method of computing the pulse amplitude (PA) log-volatility or perfusion index (PI) log-volatility of the measurement data. The PA or PI Log-Volatility expresses the short-term variability in the pulse amplitude or perfusion index. The process shown in FIG. 66O, for example, can produce the PI Log-Volatility Distribution depicted in FIG. 66L.

Initially, as shown in block 3500, the measurement data in a measurement data stream is normalized. In a non-limiting example, the measurement data is normalized by determining the natural logarithm of the measurement data. For instance, the measurement data stream can be the PA, PI, PR, Temperature, or the SpO2 measurement data streams.

Next, at block 3502, the normalized measurement data is filtered to produce a symmetric or asymmetric data stream (around the origin) representing the normalized variability of the original measurement data stream. In one embodiment, a band-pass filter is used to filter the normalized measurement data. At block 3504, the absolute value of the measurement data is determined to turn all of the variability values positive. The measurement data is filtered at block 3506 to obtain the Log-Volatility data stream. In one embodiment, the measurement data is filtered by a low-pass filter for averaging purposes. The resulting measurement data represents the Log-Volatility over time of the original measurement data stream. Statistical analyses (block 3508) can be applied to the resulting measurement data to determine, for example, volatility metrics and/or probability distributions.

FIG. 36 depicts a flowchart of an example method of computing the pulse rate (PR) volatility of the measurement data. The PR Volatility expresses the short-term variability of the heart rate (or pulse rate). The method depicted in FIG. 36, for example, can produce the PR Volatility Distribution depicted in FIG. 66K.

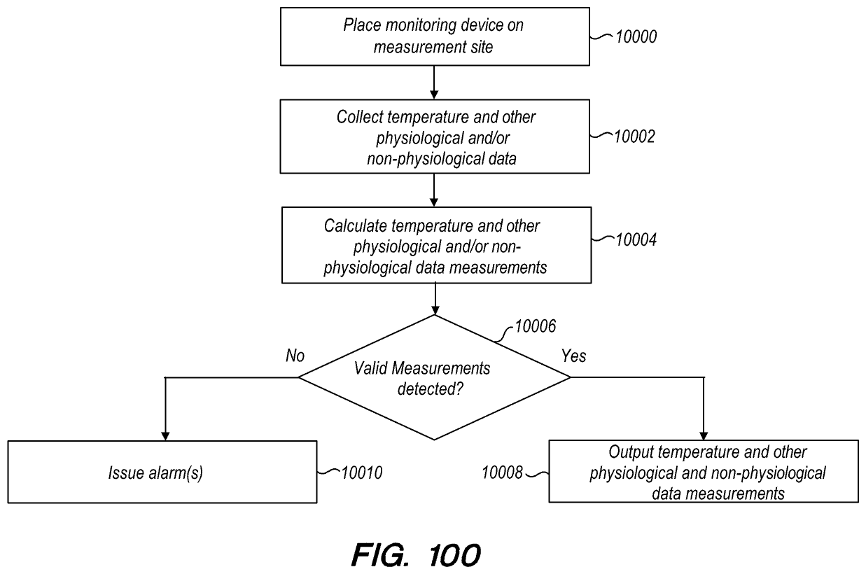

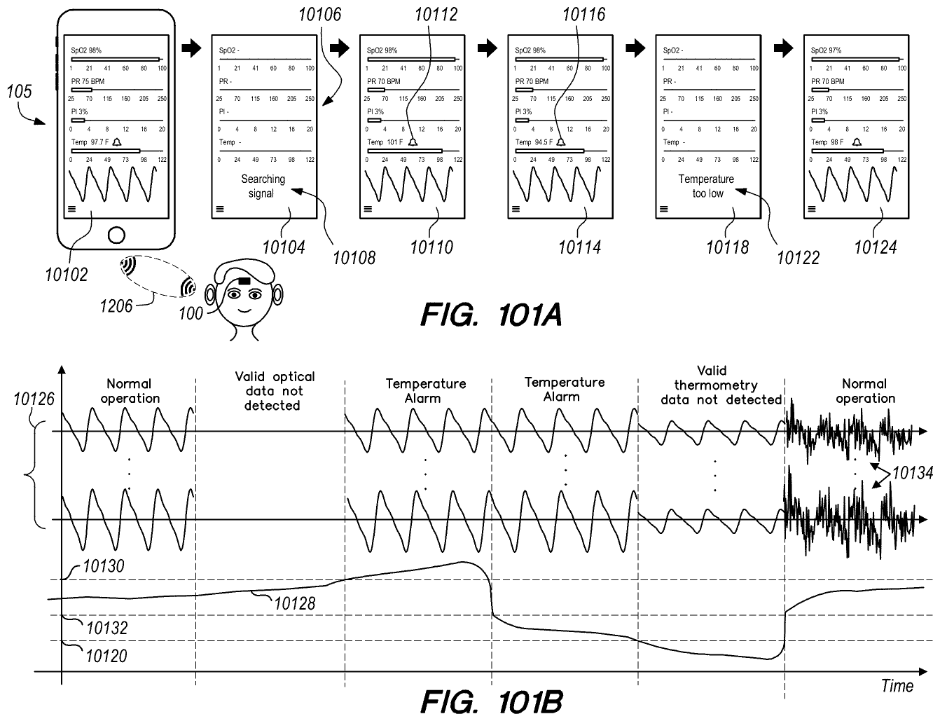

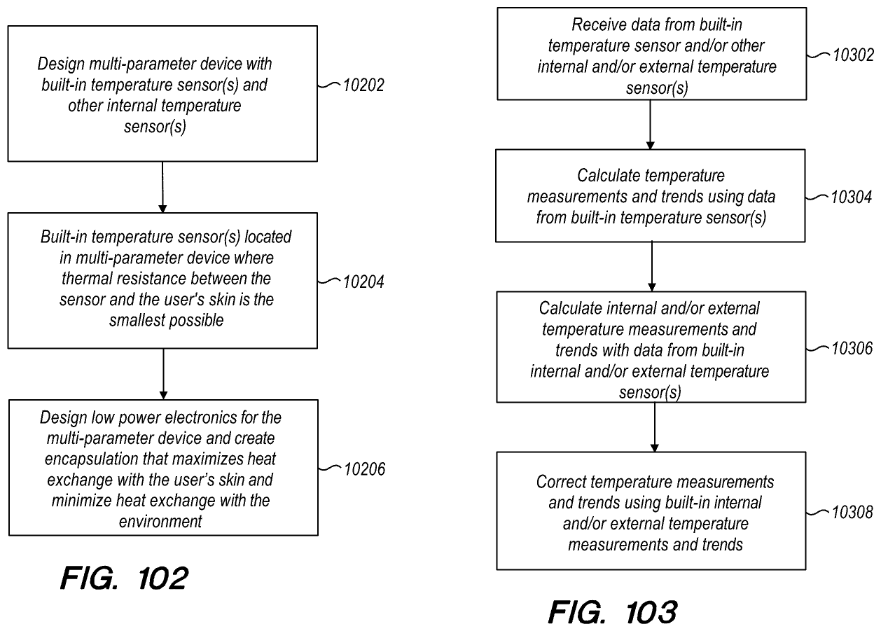

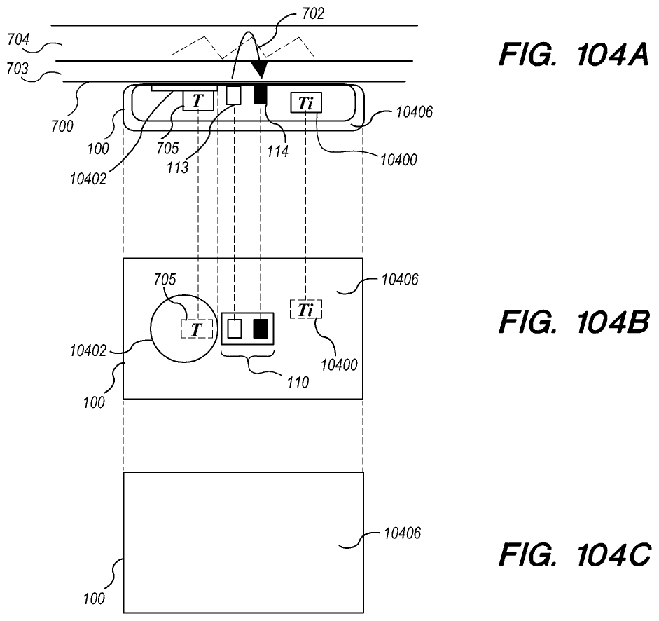

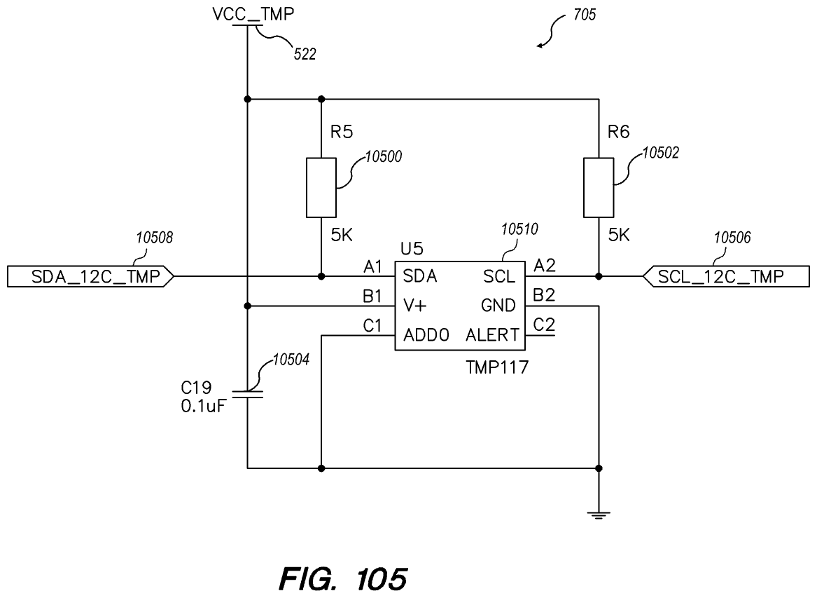

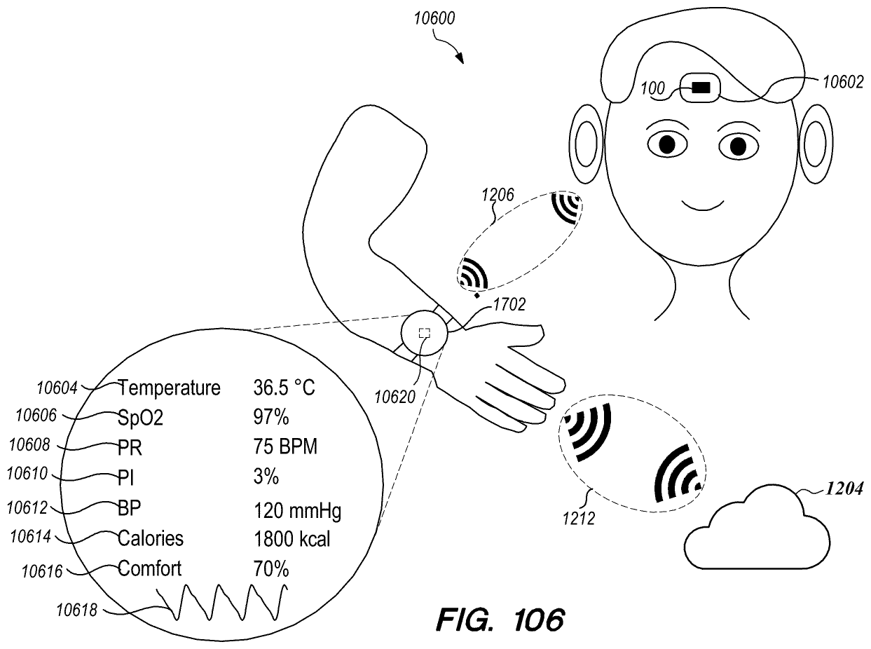

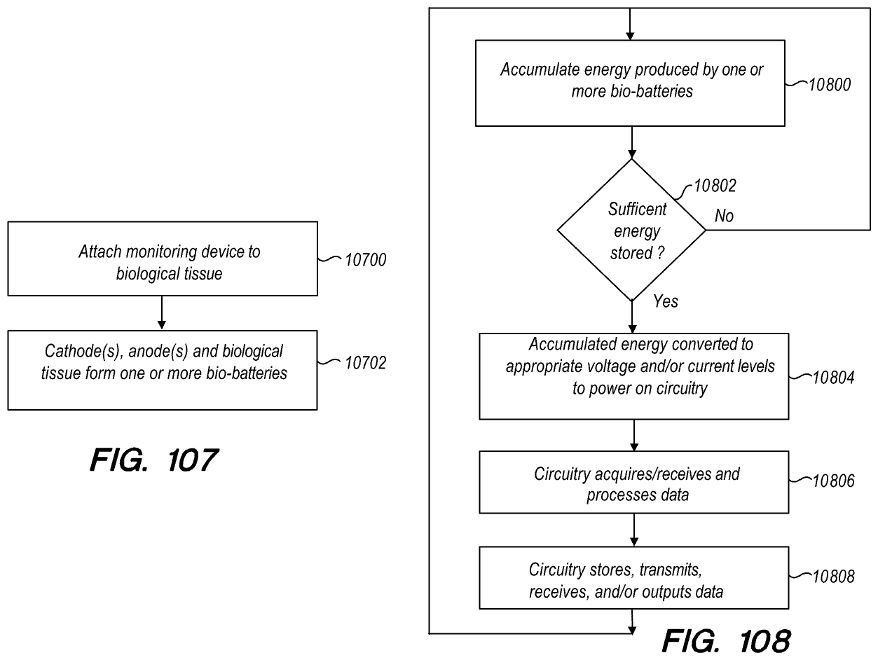

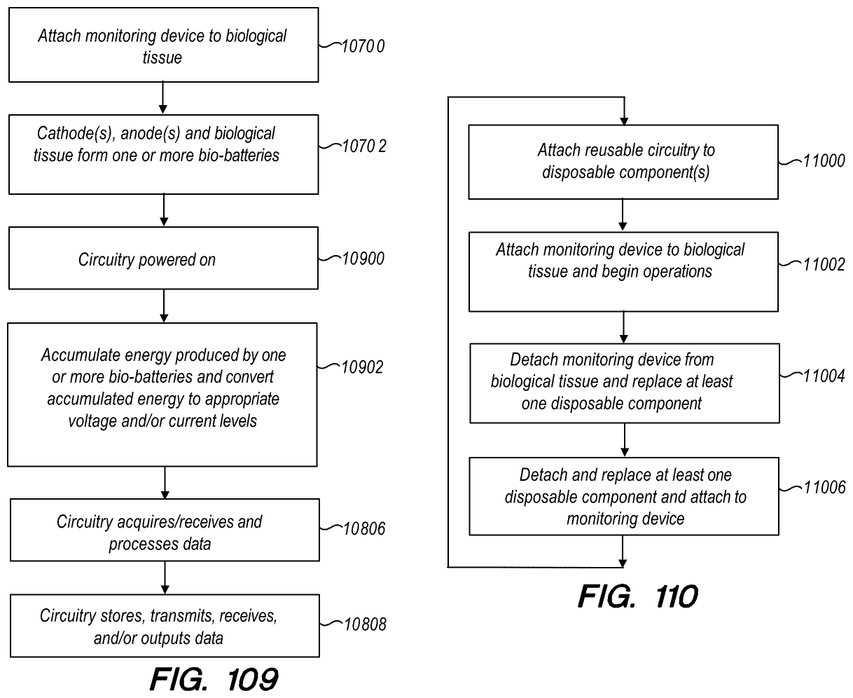

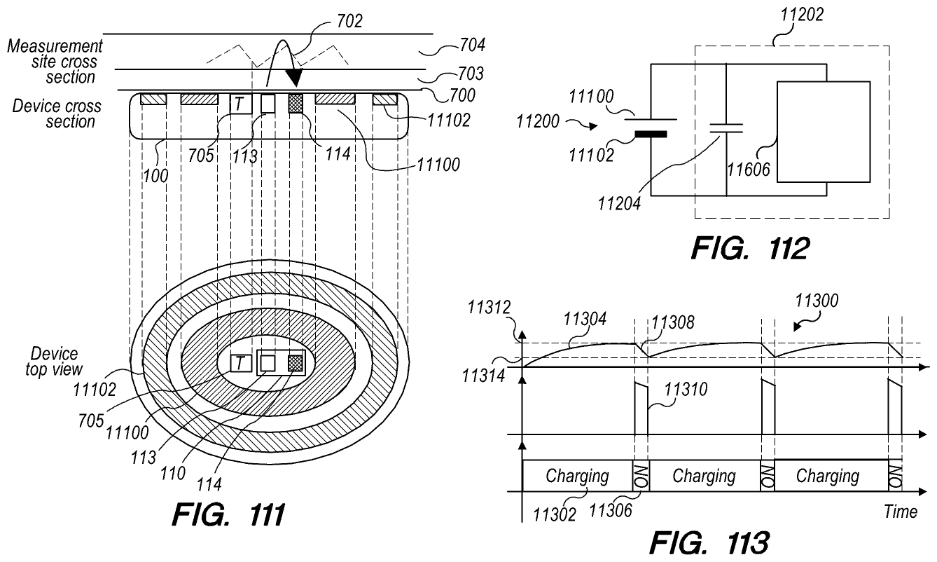

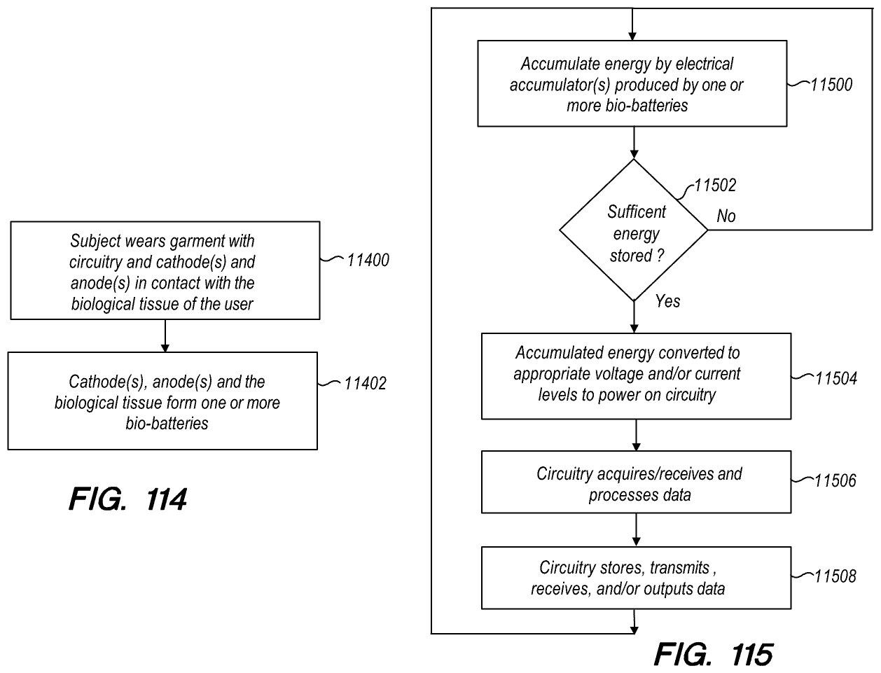

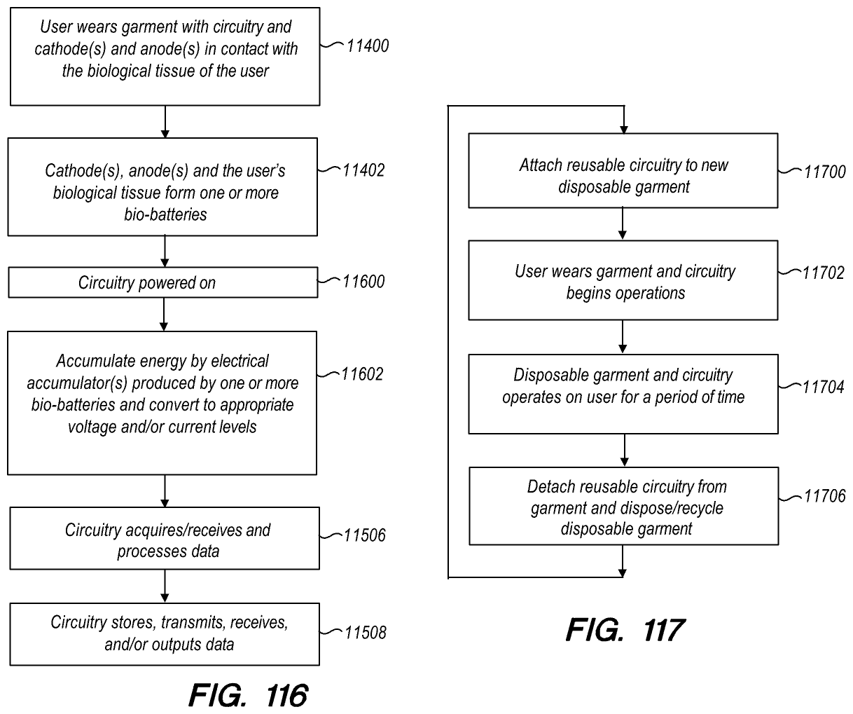

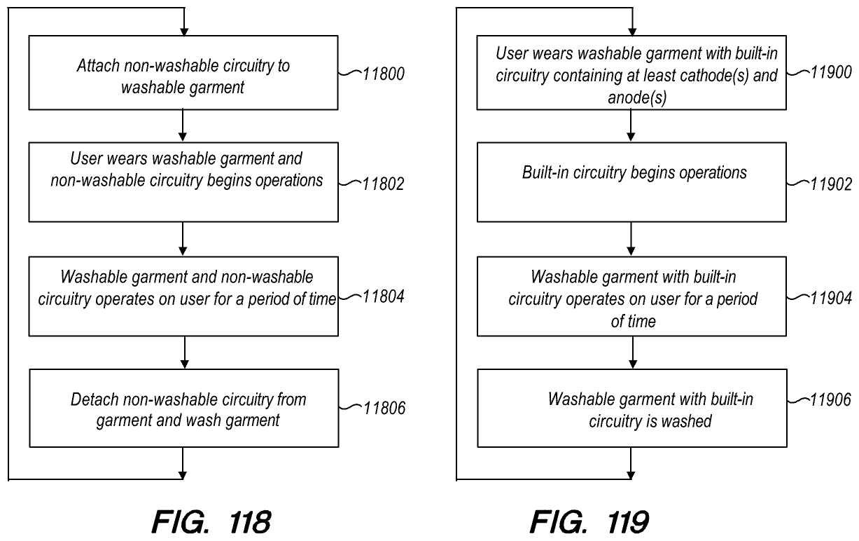

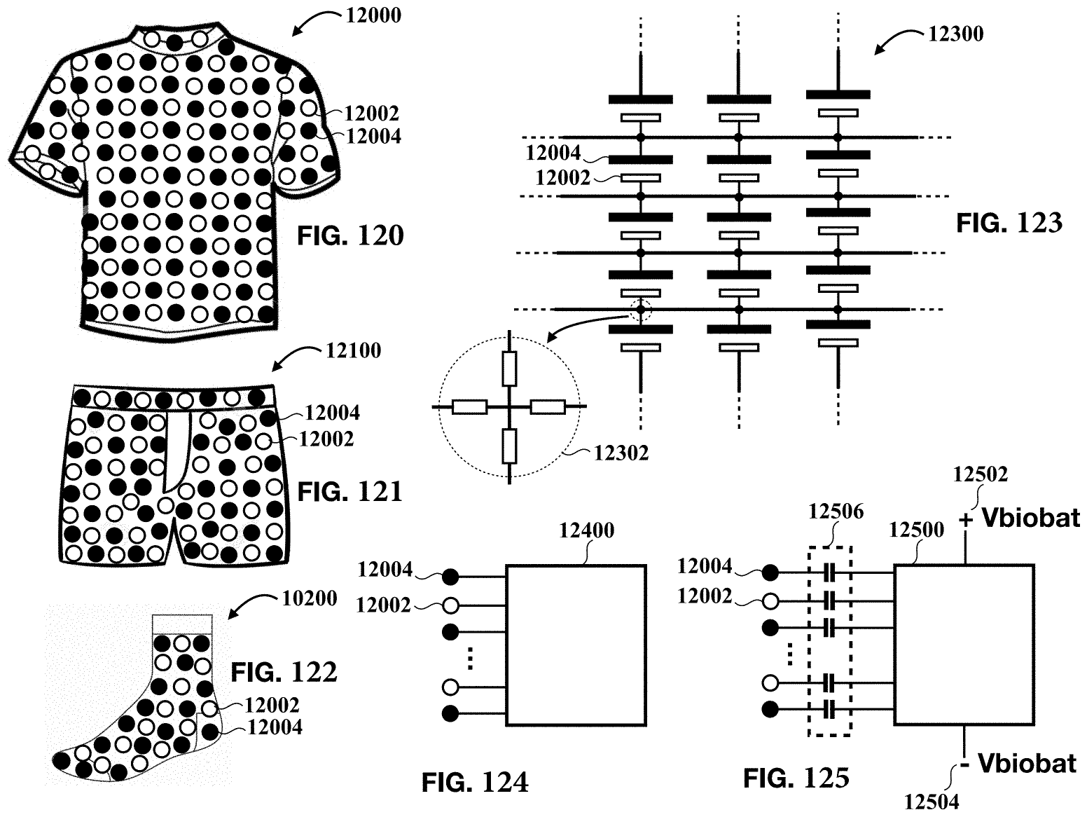

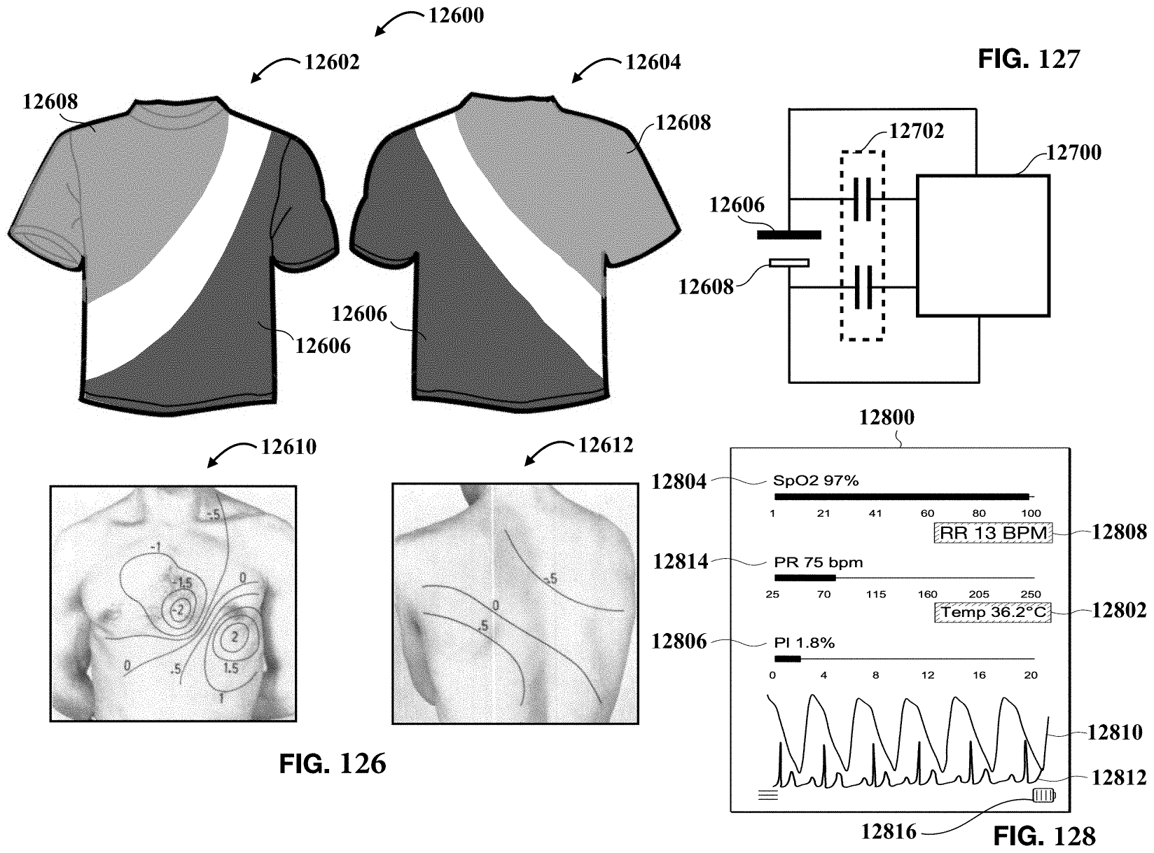

Unlike the resulting data produced by the method of FIG. 100, the PR volatility measures, produced by the process shown in FIG. 36, have the same unit as the original measurement data stream. The method of FIG. 36 is the same process as the method of FIG. 35 except for block 3500, which is omitted in FIG. 36. Thus, non-normalized measurement data is filtered at block 3600. In some embodiments, blocks 3504 and 3506 can be omitted from the method in FIG. 36, and statistical analyses (block 3508) is performed after block 3600.

In embodiments where blocks 3504 and 3506 are omitted, the data may have positive and negative values. The resulting statistics are then computed in block 3508 taking into account that the interlining probability distributions are symmetric or asymmetric around the origin. For instance, in some embodiments, the mean value may be zero while the second (e.g., variance, standard deviation), or higher moments may not because block 3504 is not used. Therefore, for embodiments where blocks 3504 and 3506 are omitted, second or higher order moments may be more appropriated to represent the underling volatility or variability of the data. Also, in some instances, The PR Volatility Distribution and the PI Log-Volatility Distribution depicted in FIG. 66K and FIG. 66L respectively, can be replaced with reports depicting the PR Volatility and the PI Log-Volatility over time.

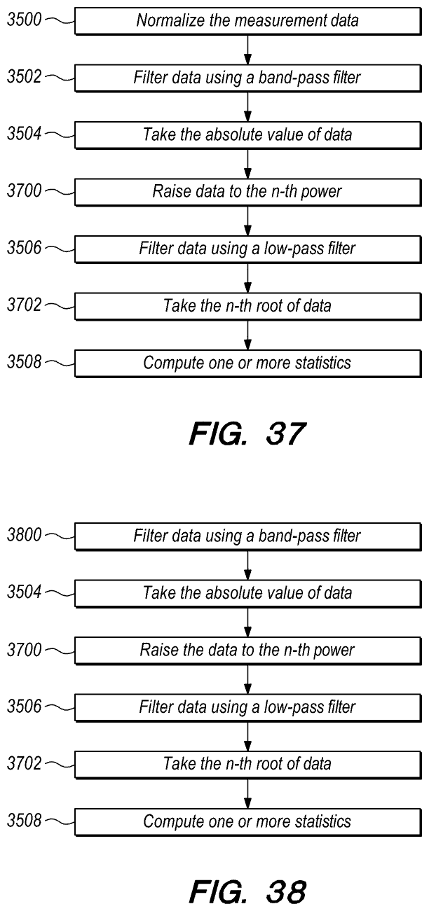

FIG. 37 illustrates a flowchart of an example method of computing the Ln log-volatility of the measurement data. The illustrated method is a generalization of the methods shown in FIG. 35. As such, some of the blocks in FIG. 35 are in the process shown in FIG. 37 and are not described in more detail for brevity.

Initially, as shown in block 3500, the measurement data in a measurement data stream is normalized. In a non-limiting example, the measurement data is normalized by determining the natural logarithm of the measurement data. Next, as shown in block 3502, the normalized measurement data is filtered to produce a symmetrical data stream (around the origin) representing the normalized variability of the original measurement data stream. At block 3504, the absolute value of the measurement data is determined to change all of the variability values positive.

Next, as shown in block 3700, the measurement data is raised to the n-th power. The number n can be any positive number. For n=1, algorithms described in FIG. 37 become identical to the algorithms described in FIG. 35. The measurement data is then filtered at block 3506. Next, the n-th root of the data is determined to provide a higher or a lower weight for each measurement data sample, based on the amplitude of the measurement data (block 3702). Block 3702 converts the measurement data back to the same scale as the original measurement data in the measurement data stream. In one embodiment, blocks 3506 and 3700 are performed concurrently. Statistical analyses (block 3508) can be applied to the resulting measurement data to determine, for example, volatility metrics and/or probability distributions.

FIG. 38 depicts a flowchart of a method of computing the Ln volatility of measurement data. The method of FIG. 38 is the same process as the method of FIG. 37 except for block 3500, which is omitted in FIG. 38. Thus, non-normalized measurement data is filtered at block 3800. In some embodiments, the method of FIG. 38 can be used to compute the volatility of data streams when the unit of the computed volatility values is the same as the measurement data (no normalization), and when higher or lower weighting via block 3702 is required for each measurement data sample, based on the amplitude of the measurement.

FIGS. 39-46 depict embodiments of real-time measurement algorithms for processing the data streams produced by the monitoring device. Real-time data is received from the monitoring device and fed into the measurement algorithms by the host device. In one embodiment, the algorithms are executed by the host software when the host software is in state 3 (state 2514), and by the device firmware in state 4 (state 2618).

FIG. 39 is used to describe a hybrid system that employs state space representation for the measurement algorithms. A hybrid system is a system that exhibits both continuous and discrete dynamic behavior. The hybrid system has inputs, outputs, states, and constants in the form of floating-point, fixed-point, and integer numbers, discrete values, literals. Example inputs into the host software include, but are not limited to, data streams and waveforms, data stream and waveform batches, measurement site's temperature, pressure, humidity, height, weight, and body rates, images, movies, acceleration, velocity, location, time, and date, battery voltage and temperature, microprocessor's clock frequency and temperature, circuitry loads, current, voltage, ambient light, counter, timer, electronic gain, and optical gain values, visual, audible, olfactory, and/or tactile information. Non-limiting example outputs from or by the host software are measurement values, trends, rate of changes, higher-order rate of changes, ratios, products, and other derivatives of measurement values, performance, forecast, and diagnostic metrics, model parameters, gauges, charts, data structures with images, movies, messages, and notifications, and visual, audible, olfactory, and tactile information, etc. Outputs are completely specified by knowledge of states, inputs, and/or constants.

Example states (i.e., set of variables and/or parameters that completely specify all possible configurations and/or behaviors of a system) of the host software include, but are not limited to, arrays, vectors, matrices, structures, classes, multi-dimensional data structures, data streams and waveforms, data stream and waveform batches, data structures with images and movies, buffers and indexed buffers, such as normalized power, correlation, root mean square accuracy, model parameters, measurements, limits, maximum, mean, median, and model values, models, reference models, optimal models, search intervals, elapsed time, signal power measures, vector and matrix norms, correlations, variances, covariances, standard deviations, probability distributions, higher-order probability distribution moments, accuracies, errors, measurement values and trends, rate of changes, higher-order rate of changes, ratios, products, and other derivatives of measurements and intermediate values, performance, forecast, and diagnostic metrics, visual, audible, olfactory, and tactile information, filter buffers such as filter states, inputs and outputs, counters, timers, temperature, pressure, humidity, height, weight, and body rates, current, voltage, resistance, elapsed time, ambient light, and intensity values, and/or any other variable or intermediate value used to specify completely the dynamic behavior of the host software at each epoch (e.g., each given period of time) provided that the inputs and the constants are known.

Non-limiting example constants in the host software data transfer block sizes, filter constants, discrete and continuous ranges, model constants in the form of values, arrays, vectors, matrices, structures and classes, limit constants, thresholds, averaging time constants, time constants, timeout constants, calibration curves, ranges, thresholds, limits, number of harmonics, and/or bases in general, averaging time in epochs or samples, sampling frequency, windowing functions and parameters, indexed buffers, arrays, vectors, matrices, or multi-dimensional data structures containing model and reference model constants, parameter refresh, display, and timeout rates, filter parameters, tables and data structures for data authentication, codification and decodification, and/or encryption and decryption.

FIG. 39 depicts a flowchart of example state representation operations performed by a host device. In some embodiments, data in the hybrid system is processed in a discrete-time periods. In such embodiments, the illustrated process is performed for each epoch (e.g., each discrete time period). In other embodiments, an epoch can refer to other exogenous variables (location, velocity, acceleration, temperature, pressure, computer iteration, etc.) that can also define the dynamic behavior and evolution of a hybrid system.

Initially, one or more constants are initialized and the input, output, and current state variables (i.e., state variables defined in current epoch) are initialized using the one or more initialized constants and/or one or more default values (blocks 3900 and 3902, respectively). Real-time data is received by the host device from the monitoring device (block 3904). In some embodiments, the real-time data is processed to filter and/or normalize the data streams. One example of a normalization process feeds the data streams through a non-linear (e.g., a log, hyperbolic, exponential, polynomial function, etc.), linear, or affine function, and then feeding the resulting data streams into a linear or non-linear low pass, band pass, or high pass filter(s) to obtain the normalized data streams.

Next, as shown in block 3906, the next states and outputs are calculated from the one or more inputs, the current states, and the one or more constants. The current states are then updated from the calculated next states (block 3908). The data that is displayed, shared, and/or stored is updated, along with any alarms, fuel gauges (e.g., battery icon), and/or notifications (block 3910).

As noted earlier, the method shown in FIG. 39 can be performed for each epoch (e.g., each time period). Block 3912 represents the detection of an event signaling that a certain amount of time (an epoch) has elapsed and that the process can return to block 3904 for the next iteration. At block 3912, a determination is made as to whether there is a new epoch that has data for processing. If so, the method returns to block 3904 and blocks 3904, 3906, 3908, 3910, and 3912 repeat. When a determination is made at block 3912 that there is no new epoch, the process waits at block 3912 until there is a new epoch.

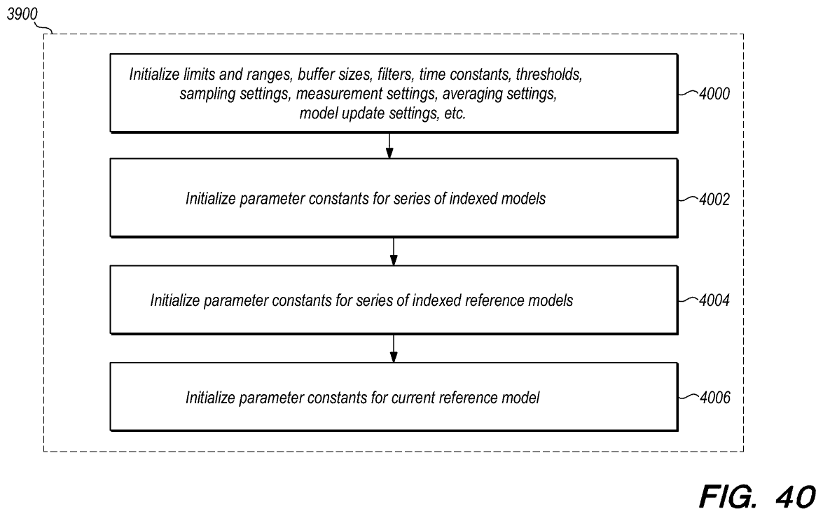

FIG. 40 illustrates a flowchart of example operations that can be performed in block 3900 shown in FIG. 39. Initially, as shown in block 4000, one or more limits and ranges, buffer sizes, filters, time constants, thresholds, sampling settings, measurement settings, averaging settings, and/or model update settings are initialized with values suitable for a particular medical and/or fitness and wellness monitoring application or set of applications. Next, one or more parameter constants for a series of indexed models are initialized at block 4002. The parameter constants for the series of indexed models can define dimensions, number of equations, type of equations, equation parameters, operating regions, variable sizes and types, limits, thresholds, topology, architecture, algorithm configurations, and/or constants from the mathematical operator(s) from the indexed models that is used for initialization and calculation of the series of indexed models.

In block 4002, the term “series of indexed models” is used. A series of indexed models is a set of equations parametrized by one or more adjustable parameters or optimization variables. The optimization variables have to be calculated for a given data set so as to select and adjust a model that optimally reproduces the data set. Whenever some of the model's optimization variables cannot be calculated through an efficient algorithm or optimization problem, then the optimization variables have to be tested (varied) in steps within a certain range to determine values that best fit a model to the specified data set. In FIG. 40, such optimization variables are called indexes for the model because the optimization variables are varied within a certain number of discrete values or discrete sub-ranges, and such values or discrete sub-ranges affect (index) the model. For instance, the waveforms of a pulse oximeter, pulse co-oximeter, ECG monitor, or non-invasive optical glucometer monitor that are obtained from the heart activity of a user can be expressed by mathematical equations or models. The waveform model can be indexed by the heart rate because the heart rate is variable within a certain range of heart beats (e.g., 25 beats per minute (BPM) to 250 BPM). An implied heart rate is typically difficult to calculate from a given waveform data set with efficient algorithms or optimization problems when the data set is subject to noise, motion artifacts, or interferences.

In one example embodiment, a series of 226 models indexed by the heart rate from 25 BPM to 250 BPM in steps of 1 BPM can be defined. For a given data set, a series of 226 indexed models are compared to the data set by means of algorithms or optimization problems with associated cost functions or metrics, and the heart rate that produces the model with the best performance (e.g., smallest cost function value, highest profit function value, etc.) is selected. During this selection process, the algorithm or optimization problem can involve the calculation of other optimization variables. For instance, the complete series of indexed models for the waveforms of a pulse oximeter, pulse co-oximeter, ECG monitor, or a non-invasive glucometer monitor for human or veterinary applications can be conveniently represented by 226 equations of weighted sums of sinusoidal functions with frequencies that are indexed by the heart rate from 25 BPM to 250 BPM in steps of 1 BPM. As a result, 226 optimization problems are solved to find the best (optimal) model from the series of 226 indexed models, where each optimization problem determines the optimal weights that best fit the data set for a given heart rate. The heart rate and associated weights with the best overall performance define the optimal model. The heart rate and weights are the optimization variables.

However, only the heart rate indexes the series of indexed models in this example, given that the other optimization variables (i.e., weights) can be efficiently calculated by algorithms or optimization problems for a given heart rate. Similar analysis can be applied to a respiration rate monitor if one assumes that the respiration rate indexes the series of indexed models. A more complex series of indexed models representing the photoplethysmographs of a pulse oximeter that also calculates the respiration rate of the user can be two-dimensionally indexed by the respiration rate and heart rate values.

In one embodiment, a series of 9,718 indexed models are two-dimensionally indexed by the respiration rate from 3 breaths per minute to 45 breaths per minute in steps of 1 breath per minute, and by the heart rate from 25 BPM to 250 BPM in steps of 1 BPM. Depending on the size of the series of indexed models, the number of algorithms or optimization problems to be solved to determine the optimal model may become prohibitive for real-time applications. In such embodiments, the optimization variables that index the series of indexed models can be solved together with the other optimization variables using local models by means of efficient algorithms and optimization problems that are defined within each sub-range of the optimization variables that act as indexes.

For example, the complete series of indexed models for the respiration rate calculated by a pulse oximeter can be conveniently represented by 130 equations of weighted sums of products of sinusoidal functions with frequencies that are two-dimensionally indexed by the heart rate sub-ranges from 25 BPM to 250 BPM such as [25, 30), [30, 36), [36, 43), [43, 51), [51, 61), [61, 72), [72, 86), [86, 103), [103, 123), [123, 147), [147, 175), [175, 209), [209, 250], and the respiration rate sub-ranges from 3 breaths per minute to 45 breaths per minute such as [3, 4), [4, 5), [5, 7), [7, 9), [9, 12), [12, 15), [15, 20), [20, 26), [26, 34), [34, 45]. To find the best (optimal) model from the series of 130 indexed models, 130 optimization problems are solved, where each optimization problem determines the optimal weights, heart rate, and respiration rate values that best fit the specified data set for a given heart rate and respiration rate two-dimensional sub-range (e.g., [25, 30)×[3, 4), [25, 30)×[4, 5), . . . , [25, 30)×[34, 45], . . . , [209, 250)×[3, 4), [209, 250)×[4, 5), . . . , [209, 250]×[34, 45]). In the case of the two-dimensional sub-range [25, 30)×[3, 4), for instance, the optimization problem efficiently calculates the weights, heart rate, and respiration rate values that best fit the model for heart rate values between 25 BPM and 30 BPM, and respiration rate between 3 breaths per minute and 4 breaths per minute. The same process is applied to the remaining 129 two-dimensional sub-ranges in order to obtain the optimal models from the series of 130 indexed models.

Next, as shown in block 4004, the parameter constants for a series of indexed reference models are then initialized. The parameter constants for the series of indexed reference models can define dimensions, number of equations, type of equations, equation parameters, operating regions, variable sizes and types, limits, thresholds, topology, architecture, algorithm configurations, and/or constants from the mathematical operator(s) in the reference models that is used for initialization and calculation of the series of indexed reference models. In block 4006, one or more parameter constants for a current reference model is initialized. The parameter constants for the current reference model can define dimensions, number of equations, type of equations, equation parameters, operating regions, variable sizes and types, limits, thresholds, topology, architecture, and algorithm configurations, and/or constants from the mathematical operators in the current reference model that is used for initialization and calculation of the series of current reference models.

FIG. 41A depicts a data flow diagram of an example method of detailing the optimization process involving a series of indexed models. Element 4100 is an indexed model (modeli). Element 4100 is indexed by the index variable “i”, which transforms a sequence of discrete values, sub-ranges, n-tuples of discrete values, or n-tuples of sub-range values used to index a particular series of indexed models into an integer sequence that also indexes the same series of indexed models. The index variable “i” provides a convenient way of generalizing the notion of indexing. For instance, multidimensional sub-ranges or grids can be easily mapped into a sequence of integer values represented by the index variable “i”.

Element 4102 represents “i” sequences of “m” real or complex functions that are used by the indexed models to span a series of dynamics or behaviors in the form of data streams or data sets that are suitable for a particular application. The functions in the element 4102 can be transcendental, polynomial, outputs of recursive filters and systems, outputs of FIR and IIR filters, outputs of Neural Networks, and/or the like.

Element 4104 represents the data set or a sequence of “n” data streams, which can be real or complex valued. The data set of the sequence of “n” data streams represent the real-time data collected by the application and available for data processing and analysis. For instance, a sequence of “n” discrete-time photoplethysmographs can be represented by the element 4104.

Element 4106 represents the behavior simulated by the element 4100 (e.g., the indexed model). The element 4106 represents “i” data sets or “i” sequences of “n” real or complex data streams produced by the series of indexed models. The elements 4102, 4104, and 4106 are indexed by the exogenous variable “t”, which can represent time, space, velocity, location, etc. For instance, data streams that are a function of time are assumed to be indexed by time, such as in a discrete-time system. Data streams that are a function of space are assumed to be indexed by space. Depending on the application, data sets or data streams can be indexed by quantities that have physical meaning or that are abstract in nature. As used herein, the term data streams or data set refer to data sequentially indexed by “t”.

Element 4108 is the optimization problem solver. For a given “i”, the element 4108 calculates the values for the model parameters (e.g., the optimization variables) that best reproduce in the element 4106 the behavior observed in the element 4104 for a given element 4102. The process of varying the optimization variables until the behavior of the element 4100 is optimal is represented by the element 4110. In some applications, the indexed models can have different operating regions that are defined by processing the element 4104 (e.g., the data streams). The different operating regions are represented by the element 4114, which feeds the element 4104 into the element 4100 for processing, classification, and/or selection of an operating region.

The overall behavior of the embodiment shown in FIG. 41A can be described as follows:

1. A monitoring device collects and processes real-time data through the device firmware in the monitoring device to produce data streams that are sent to the host device.

2. The host device receives the data streams and the host software in the host device produces the element 4104 (e.g., the data streams or data set). In some embodiments, the host device, via the host software, processes the data streams received from the host device using non-linear (such as a log, hyperbolic, exponential, polynomial function, etc.), linear, or affine functions, and then feeds the results into linear or non-linear low-pass, band-pass, or high-pass filter(s) in order to obtain normalized data streams or data sets.

3. The element 4108 (e.g., the optimization problem solver) is implemented in the host software. The element 4108 calculates, for each indexed model “i” (e.g., element 4100), the optimal values for the optimization variables that best reproduce in the element 4106 the behavior observed in the element 4104 for a given element 4102. The functions or data streams represented by the element 4102 are generated internally by the host software in the host device.

4. Each indexed model “i” represented by the element 4100 produces an indexed optimal model “i” that is represented by element 4116 in FIG. 41B. The optimal behavior for the element 4106 (e.g., the “i” data sets or “i” sequences of “n” real or complex data streams produced by the series of indexed models) is represented by the element 4118 in FIG. 41B. Element 4120 represents all parameters (e.g., optimal values for the optimization variables or the model's parameters, etc.) and internal/intermediate signals and variables other than the “i” data sets or “i” sequences of “n” real or complex data streams represented by the element 4118 from an indexed optimal model “i” represented by the element 4116. Depending on the application and the information to be produced by the indexed optimal model “i” represented by the element 4116, the element 4120 can be in the form of a class, set, array, matrix, vector, structure, list, or any other representation suitable for a particular application.

5. The optimization problem solver 4108, after numerical convergence, also outputs the optimal parameters (optimal values for the optimization variables) for each optimal model “i” represented by element 4116, and depending on the application, series of metrics, parameters, and results in scalar, array, n-tuple, range, sub-range, grid, vector, matrix, list, structure, and/or class forms that are all represented by the element 4122.

Element 4100 can be any topology, system, or circuitry with parameters to be determined via optimization problem solver 4108. Element 4100 can be a multi-layer neural network, a radial basis network, a rule-based or Bayesian network expert system, a Fuzzy Logic expert system, a hybrid system with discrete and continuous variables, parameters, weights, and/or internal memory, and any fusion or combination thereof. In all these possible embodiments, the inputs are represented by elements 4102 and 4104, and the outputs by element 4106. The process of calculating the network's or system's optimal parameters (i.e., optimization variables, parameters, weights, etc.) via the optimization problem solver 4108 (to determine indexed optimal models 4116) is represented by element 4110.

FIG. 41C illustrates an example embodiment that includes a series of indexed models that involve the product of optimization variables. The indexed models in the series of indexed models can be efficiently applied to data sets that comprise several data streams that share the same underlining waveform with different amplitudes, and that can be corrupted by noise, interferences, and/or motion artifacts, which in turn make the perceived data stream waveforms different from each other. For instance, the waveforms (photoplethysmographs) from a measurement site gathered by a pulse oximeter, pulse co-oximeter, or an optical non-invasive glucometer at different wavelengths are created from a single source (the heart's activity or pulsation). The photoplethysmograph amplitudes are a function of the measurement site physical properties at the excitation wavelengths. On one hand, the photoplethysmographs will have similar waveforms with different amplitudes whenever the noise, interferences, and/or motion artifacts are not present. On the other hand, if the interferences, noise, and/or motion artifacts are present, the acquired data streams can have significantly different waveforms even though the data stream components generated by the heart's activity still share the same underlining noiseless waveforms with different amplitudes. The embodiment described in FIG. 41C can be used to obtain accurate estimates of the underlining noiseless waveforms and their associated amplitudes from the data streams that are subjected to noise, interferences, and/or motions artifacts.

The overall behavior of the embodiment shown in FIG. 41C can be described as follows:

1. A monitoring device collects and processes real-time data through the device firmware in the monitoring device to produce data streams that are sent to the host device.

2. The host device receives the data streams and the host software in the host device produces the element 4104 (e.g., the data streams or data set). In some embodiments, the host device, via the host software, processes the data streams received from the host device using non-linear (such as a log, hyperbolic, exponential, polynomial function, etc.), linear, or affine functions, and then feeds the results into linear or non-linear low-pass, band-pass, or high-pass filter(s) in order to obtain normalized data streams or data sets.

3. The host device, via the host software, calculates, for each indexed model “i” represented by the element 4100, the optimal values for the optimization variables represented by the adjustable weights 4124, 4126 that best reproduce in the element 4106 the behavior observed in the element 4104 for a given element 4102. Each indexed model “i” represented by the element 4100 is represented by the adjustable weights 4124, 4126 and the elements 4128, 4130. The element 4128 sums the signals from the element 4102 after the signals are multiplied by the adjustable weights 4124 to produce a waveform represented by the element 4130. The waveform represented by the element 4130 is then multiplied by the adjustable weights 4126 to produce the signals represented by the element 4106, which are scaled waveforms that can reproduce to a certain degree the signals in the data streams represented by 4104 that are corrupted by noise, motion artifacts, and/or interferences. The optimization problem solver 4108 calculates the adjustable weights 4124, 4126 through the element 4132. Element 4132 represents the process of calculating optimal values (solutions) for optimization variables and parameters in element 4100 via the optimization problem solver 4108 so as to produce a series of indexed optimal models (indexed by “i”) in FIG. 41D represented by the element 4116. In this example embodiment, the indexed models represented by the element 4100 have only a single operating region, and as a result, the data streams represented by the element 4104 are not fed into the indexed models (represented by the element 4100) via the element 4114 as depicted in FIG. 41A for a multi-operating region case. In an alternate embodiment, if the indexed models represented by element 4100 have multi-operating regions, then element 4114 can be fed into element 4100 so as to enable the selection of applicable operating region(s) and corresponding set of optimization variables and parameters, which in turn have their optimal values calculated by optimization problem solver 4108.

4. Each indexed model “i” represented by the element 4100 produces an indexed optimal model “i” represented by the element 4116 depicted in FIG. 41D. The optimal behavior for the element 4106 is represented by the element 4134 in FIG. 41D. The optimal behavior represented by the element 4134 is created by summing the signals from the functions represented by the element 4102 after the signals are multiplied by the optimal weights 4134 to produce the optimal waveform 4136. The optimal waveform 4136 is then multiplied by the optimal weights represented by the element 4138 to produce the optimal signals represented by the element 4118. The optimal signals represented by the element 4118 are optimal scaled waveforms that are optimal estimates of the signals in the data streams represented by the element 4104 that are corrupted by noise, motion artifacts, and/or interferences. In this example embodiment, the indexed optimal models represented by the element 4116 have only a single operating region, and as a result, the data streams represented by the element 4104 are not fed into the indexed model represented by the element 4100 via the element 4114 as depicted in FIG. 41B for the multi-operating region general case. In an alternate embodiment, if the indexed models represented by element 4100 have multi-operating regions, then element 4114 can be fed into element 4100 so as to enable the selection of applicable operating region(s) and corresponding set of optimization variables and parameters, which in turn have their optimal values calculated by optimization problem solver 4108.

5. The optimization problem solver 4108, after numerical convergence, outputs for each optimal model “i” represented by the element 4116 the optimal parameters (optimal values for the optimization variables) and depending on the application, one or more series of metrics, parameters, and results in scalar, array, n-tuple, range, sub-range, grid, vector, matrix, list, structure, and/or class forms that are all represented by the element 4122 (e.g., correlation, cross correlation, covariances, standard deviations, histograms, probability density functions, probability mass functions, joint probability density functions, joint probability mass functions, root mean square accuracies, maxima, minima, means, medians, modes, phases, amplitudes, frequencies, spectral, temporal, and morphological measurements, etc.). The optimization problem solver 4108, after numerical convergence, produces a series of indexed optimal models represented by the element 4116 (indexed by “i”) with the data set or data streams represented by the element 4118, and the 4120 that represents all parameters (e.g., optimal values for the optimization variables or model's parameters, etc.) and internal/intermediate signals and variables other than the data set or data streams represented by the element 4118.

In FIG. 41C, the optimization problem solver 4108 adjusts the optimization variables (e.g., the adjustable weights 4124, 4126) through the element 4132. The optimization process may converge or not to an optimal solution depending on the numerical solver (e.g., the technique) employed to adjust or calculate the adjustable weights 4124, 4126. This is because the problems that involve weighted sums of products of optimization variables (weights) are not convex in nature, and if not correctly solved by means of an efficient numerical solver may lead to solutions that are not globally optimal (i.e., local optimal solution) and lack the desired performance. In one embodiment, the technique and/or system disclosed in U.S. patent application Ser. No. ______, entitled “Method and System for Processing Data Streams” can be used calculate the adjustable weights 4124, 4126. This technique produces by design the efficient numerical calculation of global optimal solutions for the adjustable weights 4124, 4126 (i.e., the optimal weights 4134, 4138 in FIG. 41D) for the functions represented by the element 4102 and data sets or data streams represented by the element 4104.

Block 4004 in FIG. 40 uses the term “series of indexed reference models.” A series of indexed reference models is an indexed set of parameters, data streams, and specifications that represent ideal temporal, spectral, dynamical or morphological behaviors for the data set or data streams represented by 4118. FIG. 41E depicts the process of searching an indexed reference model “j” 4140 with a set of normalized parameters represented by the element 4142 that best represents an indexed optimal model “i” 4116 with a set of normalized parameters represented by the element 4144. The optimal representation “j” for each “i”, “j(i)” in index pair “(i, j(i))” on output 4146, is determined by a search process 4148 that uses metrics, scores, and rules to compare the set of normalized parameters represented by the element 4144 with the set of normalized parameters represented by the element 4142. The element 4150 symbolizes the process of searching for the best reference model “j” 4140 for a given optimal model “i” 4116. The element 4152 provides one or more domain and range normalizations followed by one or more parameter extractions from each optimal model “i” 4116. The domain and range normalization(s) are performed prior to the parameter extraction(s) in some applications, where the reference model 4140 is agnostic to the parameter or set of parameters that require indexing “i”.

In a non-limiting example, consider the reference model 4140 for photoplethysmographs measured by a pulse oximeter, pulse co-oximeter, optical-based fitness tracker, or non-invasive optical-based glucometer. The idealized behavior of the photoplethysmographs is not dependent on the heart rate values. For two different heart rates from the same user, if the photoplethysmographs are normalized in time or frequency (domain) and amplitude or power (range), then the photoplethysmographs should become very similar to each other. If “i” represents the indexing of model “i” 4100 (FIGS. 41A and 41C) via the heart rate values, then the reference model “j” 4140 can be agnostic to “i”. Note that being agnostic to “i” does not necessarily imply being agnostic to the indexed optimal models “i” 4116. The indexed optimal models 4116 can have other parameters (optimization variables) with optimal values that change with each model instance “i”, and such gamma of optimal values and behaviors are to be tested against idealized optimal values and behaviors captured by the indexed reference models “j” 4140. For example, the index “j” can index reference models that are a weighted sum of sinusoidal functions with normalized amplitudes and frequencies. The weighs (parameters) of the weighted sum can differ for each reference model “j” 4140 to reproduce ideal behaviors of the indexed optimal models “i” 4116. Each reference model “j” can represent the resulting sinusoidal expansion of a photoplethysmograph, in which a given point in its normalized cycle defines the occurrence of its maximum normalized amplitude value. For instance, “j=1” could correspond to a photoplethysmograph that begins its normalized cycle at a minimum value, and reaches a maximum amplitude value at 60% of the normalized cycle for the photoplethysmograph. The index variable “j”, for j varying from 1 to 41, can represent 41 photoplethysmographs, where the first (j=1) has maximum value at 60% of its normalized cycle, the second (j=2) at 61%, the third (j=3) at 62%, . . . , and the last (j=41) at 100%.

The overall behavior of the embodiment depicted in FIG. 41E can be described as follows:

1. The element 4152 takes the data set or data streams represented by the element 4118 from the indexed optimal model “i” 4116 and normalizes the data set or the data streams in the domain and the range to obtain a set of parameters represented by the element 4144. In some embodiments, the set of parameters represented by the element 4144 can be obtained more efficiently or directly from the optimal values (model optimal parameters) of the optimization variables that comprise each indexed optimal model “i” 4116. The model optimal parameters can be obtained from the element 4120.

2. For each indexed model “i” 4100, the search process 4148 determines the reference model “j” 4140 that best reproduces the indexed optimal model “i” 4116 through metrics that compare the parameter set in element 4144 with the parameter set in element 4142.

3. After numerical convergence, the search process 4148 outputs the index pair “(i,j(i))” that indexes the reference model “j(i)” that best reproduces the indexed optimal model “i” 4116. FIG. 41F shows series of indexed optimal reference models indexed by “j(i)” 4154 with an optimal set of parameters represented by the element 4156. As a result of the search process 4148, each indexed optimal model “i” 4116 has a corresponding indexed optimal reference model “j(i)” 4154.

Element 4140 can be any topology, system, or circuitry with search process defined by element 4148. Element 4140 can be a multi-layer neural network, a radial basis network, a rule-based or Bayesian network expert system, a Fuzzy Logic expert system, a hybrid system with discrete and continuous variables, exogenous functions and signals, parameters, weights, and/or internal memory, and any fusion or combination thereof. In all these possible embodiments, the outputs are represented by element 4142. The process of searching for indexed optimal reference models 4154 via search process 4148 is represented by element 4150.

FIG. 41G depicts an example data flow diagram for the general data flow diagram shown in FIG. 41E. The illustrated embodiment can be applied to noninvasive optical-based pulse oximeters, hemoglobin meters, fitness trackers, glucometers, and the like. The following describes the example operations shown in FIG. 41G:

1. The indexed reference model (j) 4140 is a series of forty-one indexed reference models “j” 4140. Indexed reference model 4158 (j=1), the indexed reference model 4160 (j=20), and the indexed reference model 4162 (j=41) represent three instances of the forty-one indexed reference models 4140. Each reference model “j” has waveform defined by the following equation parametrized by the set of normalized parameters represented by the element 4142,

Σₖ₌₁^{q/2}r_{k,j} sin kθ+Σ_{k=1+q/2}^{q}r_{k,j} cos(k−q/2)θ Equation 14

where θ represents normalized time, and q is even. For q=6 (as depicted in FIG. 41G), Equation 14 is a 3-harmonic sinusoidal approximation of a triangular wave representing the reference behaviors of a photoplethysmograph, in which a given point in its normalized cycle defines the occurrence of its maximum normalized amplitude value. For instance, the indexed reference model 4158 (j=1) approximates a triangular wave with maximum value at 60% of its normalized cycle, which leads to the following values (4 decimals) for the parameters in the element 4164: r_{1,1=0.0000}, r_{2,1}=0.1611, r_{3,1}=0.0012, r_{4,1}=1.0000, r_{5,1}=0.0091, r_{6,1}=0.0747. The indexed reference model 4160 (j=20) approximates a triangular wave with maximum value at 79% of its normalized cycle, which leads to the following values (4 decimals) for the parameters in the element 4166: r_{1,20}=0.0000, r_{2,20}=0.3998, r_{3,20}=0.0100, r_{4,20}=1.0000, r_{5,20}=0.0224, r_{6,20}=−0.1585. The indexed reference model 4162 (j=41) approximates a triangular wave with maximum value at 100% of its normalized cycle, which leads to the following values (4 decimals) for the parameters in the element 4168: r_{1,41}=0.0000, r_{2,41}=0.4997, r_{3,41}=0.0189, r_{4,41}=1.0000, r_{5,41}=0.0283, r_{6,41}=−0.3335. The search process 4148 selects through the element 4150, the parameters (e.g., in elements 4164, 4166, 4168) of an indexed reference model instance “j” (e.g., indexed reference models 4158, 4160, 4162) and transfers the parameters to the set of parameters in the element 4142. The same rational is valid for any number of harmonics, or other equations that can represent photoplethysmographs (e.g., Volterra, Taylor, and/or Fourier series, polynomial and/or transcendental series, complex exponential series, etc.) with an arbitrary number of indexed reference models. In addition, instead of photoplethysmographs, the waveforms can be waveforms that are sensed, for example, by ultrasound, thermal, resistive, capacitive, inductive, pressure, acceleration, velocity, or position transducer array systems, or the pixels of an optical camera.

2. The element 4152 performs a domain and range normalization on the optimal models (i) 4116 to perform parameter extractions. Assuming that the series of indexed optimal models “i” 4116 is implemented as shown in the example embodiment of FIG. 41D, the element 4102 has the following function components:

ƒ_{k,i}(t)=sin(2(i+24)(1/60)(1/Fs)kt) Equation 15

for k=1, 2, . . . , m/2

ƒ_{k,i}(t)=cos(2π(i+24)(1/60)(1/Fs)(k−m/2)t) Equation 16

for k=m/2+1, m/2+2 . . . , m, with m even, and for i=1, 2, . . . , 226. The constant Fs is the sampling frequency in Hertz (e.g., 50, 60, 100 Hz, etc.). The variable t is the normalized time (t=1, 2, 3, . . . ). For q=6 (as depicted in FIG. 41G), the element 4102 represent six discrete-time sinusoidal waveforms. The index “i” indexes the series of optimal models 4116 for heart rate values varying from 25 BPM to 250 BPM in steps of 1 BPM. For each epoch (e.g., normalized time interval defined by a number of samples), the indexed models “i” 4100 as depicted in FIG. 41C are solved for a certain sample size (e.g., 250, 400, 500 samples etc.) for the data streams or data set in the element 4104 and the functions in the element 4102, collected and generated at the same sampling frequency Fs, to produce the indexed optimal models “i” 4116 as shown in FIG. 41D. For each indexed optimal model “i” 4116, the domain and range normalization in the element 4152 can be the process of transforming through trigonometric identities the signal 4136,

hᵢ(t)*=Σₖ₌₁^{m/2}w_{k,i}*sin(2π(i+24)(1/60)(1/Fs)kt)+Σ_{k=1+m/2}^{m}w_{k,i}*cos(2π(i+24)(1/60)(1/Fs)(k−m/2)t) Equation 17

into a normalized signal,

Pₖ₌₁^{q/2}p_{k,i}*sin(kθ)+Σ_{k=1+q/2}^{q}p_{k,i}*cos((k−q/2)θ) Equation 18
For m and q equal to 6, the parameter extractions in the element 4152 occur by outputting the weights that multiply each normalized sinusoidal signal as parameters in the element 4144 (e.g., p_{1,i}*, p_{2,i}*, p_{3,i}*, p_{4,i}*, p_{5,i*}, and p_{6,i}*) in FIG. 41G. During the normalization process, the parameter p_{1,i}* can be set to zero, and p_{4,i}* to unity in order to be compliant with r_{1,j} and r_{4,j}.

3. The search process 4148 selects the best indexed reference model “j” 4140 for each indexed optimal model “i” 4116. In one embodiment, the selection can be accomplished by computing metrics that relate to the parameters in the elements 4142, 4144. For instance, for each indexed model “i” 4116, the indexed reference model “j” 4140 that produces the smallest covariance (Cov),

$$Cov ( j , i ) = {\sum }_{k = 1}^{q} \frac{{( {p}_{k , i}^{*} )}^{2}}{2} + \frac{{( {r}_{k , j} )}^{2}}{2} - ( {p}_{k , i}^{*} ) ( {r}_{k , j} ) \qquad Equation 19$$

or the smallest root mean square accuracy (Arms),

Arms(j,i)=\sqrt{Cov(j,i)} Equation 20

can be selected to be the indexed optimal reference model for “i”

$$( i . e . , j ( i ) = {arg min}_{j} Cov ( j , i ) or j ( i ) = {arg min}_{j} Arms ( j , i ) ) .$$

Alternatively, for each indexed model “i” 4100, the indexed reference model “j” 4140 that produces the largest correlation (Corr),

$$Corr ( j , i ) = \frac{{\sum }_{k = 1}^{q} ( {p}_{k , i}^{*} ) ( {r}_{k , j} )}{\sqrt{{\sum }_{k = 1}^{q} {( {p}_{k , i}^{*} )}^{2}} \sqrt{{\sum }_{k = 1}^{q} {( {r}_{k , j} )}^{2}}} \qquad Equation 21$$

can be selected to be the indexed optimal reference model for “i”

$$( i . e . , j ( i ) = {arg max}_{j} Corr ( j , i ) ) .$$

For this particular example, q in Equations 19 and 21 is equal to 6. Larger or
smaller values for q can be adopted depending on the number of sinusoidal functions being used. It is also possible to select the indexed reference model “j(i)” 4140 for “i” that produces the smallest ratio Arms/Corr, the largest ratio Corr/Arms, the smallest specified weighted sum of cos⁻¹(Corr) and Arms, or any other expression or composition of expressions that compute a particular metric or metrics of interest.

Block 4006 in FIG. 40 uses the term “current reference model.” A current reference model is an instance from the series of indexed optimal reference models 4116. The current reference model is obtained via the example data flow diagram shown in FIG. 41H, which calculates output measurements 4170, and updates the current reference model 4172 and the current search limits 4174 by means of a classification process 4176, and an outlier removal and averaging process 4178. The embodiment shown in FIG. 41H uses as inputs the elements 4120, 4122 obtained in the embodiments of FIGS. 41A-41D, the elements 4144, 4156 obtained in the embodiments of FIGS. 41E-41G, and the element 4180 obtained from the current search limits 4174 and the parameters in the element 4182 that are obtained from the current reference model 4172 to calculate the output measurements 4170 and to update the current reference model 4172 and the current search limits 4174. The following describes the example operations in FIG. 7H:

1. The element 4122 is calculated in the embodiments shown in FIG. 41A and in FIG. 41C by the optimization problem solver 4108 prior to being input into the classification process 4176. In an example embodiment, the element 4122 can be a structure or a container that stores, for each indexed optimal model “i” 4116, the power, sampled correlation, and sampled root mean square accuracy arrays. For each indexed optimal model “i” 4116 (FIG. 41D), the corresponding entry “i” of the power array can be computed from the optimal weights 4134 using the following equation,

Power(i)=\sqrt{(w_{1,i}*)²+(w_{2,i}*)²+ . . . +(w_{m,i}*)} Equation 22

Similarly, the corresponding entry “i” of the sampled correlation array and the corresponding entry “i” of the sampled root mean square accuracy array can be computed from the data streams or data set in the optimal waveform 4136, the element 4118, and the element 4104 using the following equations:

$$\begin{matrix}Corr ( i ) = {max}_{k} {Corr}_{k} ( i ) & Equation 23 \\ and & \\ Arms ( i ) = {mean}_{k} {Arms}_{k} ( i ) & Equation 24 \\ where & \\ {Corr}_{k} ( i ) = \frac{{mean}_{t} {s}_{k} ( t ) {{h}_{i} ( t )}^{*}}{\sqrt{{mean }_{t} {{s}_{k} ( t )}^{2}} \sqrt{{mean}_{t} {( {{h}_{i} ( t )}^{*} )}^{2}}} \\ and & Equation 25 \\ {Arms}_{k} ( i ) = \sqrt{{mean }_{t} {( {s}_{k} ( t ) - {{m}_{k , i} ( t )}^{*} )}^{2}} & Equation 26\end{matrix}$$

for k=1, 2, . . . , n. In Equation 23, Corrₖ(i) is the sampled correlation computed between the waveform 4136 and each data stream “k” in the element 4104, and in Equation 24, Armsₖ (i) is the sampled root mean square accuracy computed between each data stream “k” in the element 4104 and each data stream “k” in the element 4118. The sampled means are taken over the finite discrete-time data sets in the elements 4104, 4118, and the waveform 4136 (e.g., 250 samples) indexed by variable “t”. In this example, for a given “i”, Corr(i) is the maximum in k over all Corrₖ(i) values (n values), and Arms(i) is the sampled mean in kover all Armsₖ(i) values (n values). The power, sampled correlation, and sampled root mean square accuracy arrays are computed for each epoch, comprised of a finite and sequential number of samples in “t”, according to the expressions in Equations 22, 23, and 24, where the elements 4104, 4118, and the waveform 4136 are calculated and/or updated at each epoch according to the embodiments described in conjunction with FIGS. 41A-41D.

2. The parameters in the element 4182 are a set of parameters from the current reference model 4172. Because the current reference model 4172 is an instance from the series of indexed optimal reference models 4154, the parameters in the element 4182 have the same description and size as the parameters in the elements 4144 and 4156. The parameters in the elements 4144 and 4182 are used in the classification process 4176 to compute the metric arrays that relate to each indexed optimal model “i” 4116 with the current reference model 4172. The parameters in the element 4144 can be obtained from the embodiment shown in FIG. 41E or in FIG. 41G. An example embodiment for a metric can be the computation of the root means square accuracy array,

$$Arms ( i ) = \sqrt{{\sum }_{k = 1}^{q} \frac{{( {p}_{k , i}^{*} )}^{2}}{2} + \frac{{( {r}_{k} )}^{2}}{2} - ( {p}_{k , i}^{*} ) ( {r}_{k} )} \qquad Equation 27$$

In this example, the parameters in the elements 4144 and 4182 can be obtained from weighted sinusoidal models, such as the weighted sinusoidal models in the element 4116 and the indexed reference model 4140 (e.g., for q=6).

3. The current search limits 4174 are provided by the current reference model 4172. The current search limits 4174 are sub-ranges where the classification process 4176 is most likely to find the best estimates for the measurements (e.g., heart rate, respiration rate, body and/or skin temperature, blood pressure, oxygen saturation, pulse amplitude, perfusion index, glucose concentration in blood, water in blood, etc.) and parameters (e.g., relation and functional relation values used in the computation of measurements such as optical ratios, higher-order fractional and other normalized values) based on prior information acquired and computed in past epochs. Consider the example embodiment where a pulse oximeter measures pulse rate (PR), pulse amplitude (PA), oxygen saturation (SpO2), and/or perfusion index (PI). If the current PR, SpO2, PA, and/or PI of the user have current values of 100 BPM, 95%, and 1% respectively, then most likely within a short period of time thereafter the PR, SpO2, and the PI measurement values do not change by more than certain upper and lower bounds defined by relative or absolute threshold values. For instance, the PR of the user typically does not change more than +/−25% from the current PR baseline within the next few seconds. This has to do with the fact that the human heart has a maximum frequency bandwidth defined by its size and constitution that prevents the human heart from changing frequency abruptly under typical clinical, fitness, and wellness scenarios. In the case of SpO2 measurements, the current SpO2 baseline typically does not change more than +25% and −35% within the next few seconds. The human body requires a specific balance of oxygen in the blood, and as a result, the human body has mechanisms in place that prevent the oxygen levels in the blood from abruptly dropping or increasing. This implies that SpO2 as a measurement signal has a maximum bandwidth that prevents instantaneous or abrupt changes under typical clinical, fitness, and wellness scenarios. Similar rationale can be applied to the PI as a measurement signal. Typically, given the governing dynamics of peripheral blood circulation and perfusion, the current PI baseline does not change more than +1000% and −90% within the next few seconds. The perfusion index signal typically has a frequency bandwidth that is larger than the oxygen saturation and pulse rate measurement signals. However, the PI is still bounded by the intrinsic dynamics of the peripheral blood circulation. The concept of search limits in the context of biosensing measurements, parameters, and functionalities can be usefully applied to any clinical, fitness, and wellness setting given that physiology is intertwined with biofeedback systems that stimulate, regulate, and control parameter dynamics around equilibria. In this example embodiment of the pulse oximeter, the current search limits 4174 can be employed by the classification process 4176 using the following inequalities,

m₁PR(t)<PR(i)<M₁PR(t), Equation 28

m₂SpO2(t)<SpO2(i)<M₂SpO2(t), Equation 29

m₃PI(t)<PI(i)<M₃PI(t), Equation 30

where M₁, M₂, and M₃ are greater than one, and m₁, m₂, and m₃ are less than one. The values of M₁, M₂, M₃, m₁, m₂, and m₃ are defined by the application. In the pulse oximeter example, M₁=1.25, M₂=1.25, M₃=10, m₁=0.75, m₂=0.65, and m₃=0.1. The interpretation of the inequalities in the Equations 27, 28, and 29 is the candidate measurements of SpO2(i), PR(i), and PI(i) in the next epoch “(t+1)” and are obtained from a series of indexed optimal models 4116. The inequalities in the Equations 27, 28, and 29 can be bounded by the upper and lower relative limits defined by the current epoch “t” measurements of SpO2(t), PR(t), and PI(t).

4. The classification process 4176 can be implemented through multi-criteria and multi-variate techniques that estimate the measurements and the parameters that best fit the performance criteria. In an example embodiment, the performance criteria can be defined by Equations 22, 23, 24, and 27 when tested against thresholds during the classification process 4176. Measurements can be obtained from the information input into the classification process 4176. For instance, if the series of indexed optimal models “i” 4116 are indexed by PR values, then the index “i” can be directly mapped into PR values via an affine transformation,

PR(i)=PR_{MIN}+ci Equation 31

where PR_{MIN} and c are constants defined by the application. For example, if “i” varies from 1 to 226 in unit steps, PR_{MIN}=24 BPM, and c=1, then PR will vary from 25 BPM to 250 BPM in steps of 1 BPM. SpO2 values can be estimated from the amplitudes of the photoplethysmographs expressed in a functional ratiometric form. The optimal weights represented by the element 4138 are input into the classification process 4176 through the element 4120 and can be used to compute the SpO2 value for each “i” in the following manner,

$$Sp O 2 ( i ) = cal ( \frac{{α}_{0} + {\sum }_{l = 1}^{L} {α}_{l} \frac{{a}_{l , 0} + {\sum }_{k = 1}^{n} {a}_{l , k} {g}_{k , i}^{*}}{{b}_{l , 0} + {\sum }_{k = 1}^{n} {b}_{l , k} {g}_{k , i}^{*}}}{{β}_{0} + {\sum }_{l = 1}^{L} {β}_{l} \frac{{a}_{l , 0} + {Σ}_{k = 1}^{n} {a}_{l , k} {g}_{k , i}^{*}}{{b}_{l , 0} + {\sum }_{k = 1}^{n} {b}_{l , k} {g}_{k , i}^{*}}} ) \qquad Equation 32$$

where a_{l,k}, b_{l,k}, αₗ, βₗ, α_{l,0}, b_{l,0} (for k=1, 2, . . . , n, and l=1, 2, . . . , L), α₀, and β₀ are constants obtained experimentally from clinical data and fitting techniques, through optical modeling techniques such as Boltzmann's Transport Equations (or photon diffusion modeling approximations), or a combination of data fitting and simulation techniques. The function “cal” maps (i.e., translates) the optical data lumped together in a ratiometric quantity into measurement units (e.g., % for SpO2). The function “cal” can also be a polynomial, linear or affine, piecewise linear, piecewise nonlinear, transcendental, or discrete mapping through a table of values, or a combination thereof. Equation 32 can also be used to calculate (estimate) the measurement values of other blood or body constituents and rates, such as glucose, total hemoglobin, dyshemoglobins, water, proteins, cholesterol, bone density, body fat, calorie consumption, calorie intake, calorie balance over a period of time, etc., depending on the number of optical wavelength signals probing a measurement site and collected by means of the data streams in the element 4104. The PI values can be computed by a weighted sum of the amplitudes of the photoplethysmographs. The optimal waveform 4136 and the optimal weights in element 4138 are input into the classification process 4176 through the element 4120 and can be used to compute the PI value for each “i” in the following manner,

$$\begin{matrix}PI ( i ) = {max}_{t} A m {p}_{i} ( t ) - {min}_{t} A m {p}_{i} ( t ) \\ where & Equation 33 \\ {Amp}_{i} ( t ) = {{h}_{i} ( t )}^{*} {\sum }_{k = 1}^{n} {d}_{k} {g}_{k , i}^{*} & Equation 34\end{matrix}$$

dₖ are constants obtained experimentally from the clinical data an fitting techniques, through optical modeling techniques such as Boltzmann's Transport Equations (or photon diffusion modeling approximations), or a combination of data fitting and simulation techniques. Equation 33 computes the peak-to-peak amplitude of Ampᵢ(t), a weighted sum of the data streams in the element 4118 (obtained from the optimal waveform 4136 and the optimal weights 4138 and the dₖ constants), over a certain period of time capable of capturing at least one heart rate cycle.

5. The measurement and parameter selection that occurs in the classification process 4176 aims at applying classification techniques such as Linear, Nonlinear, Radial Basis, Bayes, Decision Tree, Neural Networks, k-Nearest Neighbor, Ruled-Based and Experts Systems in general to estimate the measurement and parameter values that best reflect the real-time data and information collected and processed by the monitoring device and the host device. In an example embodiment, using Equations 21, 26, 27, 28, and 29 can be described by the following ruled-based description,

$$\begin{matrix} {i}^{+} = {arg max}_{i ∈ φ} Power ( i ) & Equation 35 \\ where & \\ φ = \{ i \text{:} ( {Power}_{Norm} ( i ) > {Power}_{MIN} ) \& ( Arms ( i ) < {Arms}_{MAX} \& ( {m}_{1} PR ( t ) < PR ( i ) < {M}_{1} PR ( t ) ) \& ( {m}_{2} Sp O 2 ( t ) < Sp O 2 ( i ) < {M}_{2} Sp O 2 ( t ) ) \& ( {m}_{3} PI ( t ) < PI ( i ) < {M}_{3} PI ( t ) ) \} & Equation 36 \\ and & \\ {Power}_{NORM} ( i ) = \frac{Power ( i )}{{max}_{i} Power ( i )} , & Equation 37\end{matrix}$$

where ϕ represents the set of allowed values for index “i”, “&” is the AND logical operator, Power_{MIN} corresponds to the minimum normalized power threshold level, and ArmS_{MAX} corresponds to the maximum allowed root mean square error between the optimal model 4116 and the current reference model 4172. The optimal model 4116 has a normalized energy or power level (Equation 37) that is greater than a certain value (i.e., Power_{MIN}), and a root mean square accuracy (Equation 27) when compared to the current reference model 4172, less than a certain value (i.e., Arms_{MAX}) to be considered valid. The result of the ruled-based classification process 4176 defined by Equations 35, 36, and 37 is the determination of an index i=i⁺∈ϕ, where Power(i⁺) is maximum. This enables the selection or computation of the measurement values (e.g., SpO2(i⁺), PR(i⁺), PI(i⁺)), the selection index “j(i*)”, and the selection of indexed parameters and metrics, which are all represented by the outputs 4184, 4186, 4188 of the classification process 4176. Signals and/or values in the output 4184 are used in the computation of the output measurements 4170. Signals and/or values in the output 4188 are used in the computation of a new reference model, and signals and/or values in the output 4186 are used in the computation of new search limits. Depending on the application, some of the signals and/or values in the output 4184 can also be part of elements 4186 and 4188. The classification result “i⁺” indexes the optimal model 4116, the optimal reference model 4154, and all of the indexed measurements and parameters computed from the optimal model 4116 and the optimal reference model 4154. The measurement values SpO2(i⁺), PR(i⁺), and PI(i⁺) can be used, for instance, to update the search limits 4180 at block 4190, to update the measurements SpO2(t+1), PR(t+1), PI(t+1) in the next epoch “(t+1)” at block 4192, to calculate a new reference model at block 4194, and to update the “j(i+)” index that can be used to update the current reference model for the next epoch “(t+1)” at block 4196.

6. The output 4184 of the classification process 4176 can be used to calculate or to update new output measurements 4170. This is done in an outlier removal and averaging process shown in block 4178. Block 4178 receives the output 4184 (e.g., SpO2(i⁺), PR(i⁺), and PI(i⁺)) and calculates the next epoch “(t+1)” output measurements 4170 (e.g., SpO2(t+1), PR(t+1), PI(t+1)). Block 4178 uses the current and the past outputs 4184 to compute the next epoch output measurements 4170. The current and the past outputs 4184 can be stored in arrays or matrices that can be used by the outlier removal and averaging process 4178 to extract the best estimates for next epoch output measurements 4170. In one embodiment, the technique and/or system disclosed in U.S. patent application Ser. No. ______, entitled “Method and System for Processing Data Streams” can be used to remove outliers and perform averaging in block 4178 to calculate the next epoch output measurements 4170. This technique produces by design the efficient numerical calculation of the global optimal solutions for the next epoch output measurements 4170 using the current and the past outputs 4184.

7. The calculation of the new search limits occurs in block 4190. Block 4190 uses the outputs 4186 from the classification process 4176, the outputs from block 4178 to calculate the new search limits. In the example embodiment defined by Equations 28, 29, and 30, the calculation of the new search limits occur by using current and past epoch measurements 4198 and relative limits. Depending on the application, other information 4199 (e.g., model parameters, metrics, etc.) from block 4178 can be used to define the search limits that are a function of the current and past selected optimal models' parameters and metrics and the current and past selected optimal reference models' parameters and metrics obtained from the classification process 4176. It is also possible to have multiple search intervals for a particular candidate measurement or parameter. In an example embodiment, Equation 28 for the measurement values of PR can be generalized as,

m_{1,k}PR(t)<PR(i)<M_{1,k}PR(t) Equation 38

where each pair, m_{1,k}, and M_{1,k}, for k=1, 2, . . . , define a search limit interval. The search limit intervals can be disjointed or not, depending on the application. The search limit intervals can also be multi-dimensional where the search limits become multi-dimensional regions such as,

(x(i)=x₀(t))^{T}Aₖ(x(i)−x₀(t))<1 Equation 39,

where the variable x is a vector representing the candidate measurements and/or the parameters (e.g., x(i)=[PR(i) SpO2(i) PI(i) . . . ]^{T}), the variable x₀ is a vector representing the current epoch t measurements and or/parameters (e.g., x₀(t)=[PR(t) SpO2(t) PI(t) . . . ]^{T}) and Aₖ for k=1, 2, . . . , are positive definite matrices that have dimensions implied by the context (i.e., vector×dimension). Each matrix Aₖ defines a search region. Equation 39 represents multi-dimensional search regions that are enclosed by ellipsoids defined by x₀(t) and Aₖ pairs. The search regions can also be defined by Voronoi diagrams, or a set of non-overlapping multi-dimensional regions that form a partition of the entire search regions. In this context, each non-overlapping multi-dimensional region is a search region that can be used as the current search limits (region) 4174 (with realization 4180) by the classification process 4176 in a particular epoch. Once the new search limits are obtained in block 4190, the current search limits 4174 that will be used in the next epoch by the classification process 4176 are updated at block 4192. The current search limits 4174 can be stored in a particular configuration to save memory and/or improve data retrieving. The current search limits represented by the element 4174 outputs the current search limits in a realization represented by element 4180 that can be more efficiently used by the classification process 4176. For instance, each non-overlapping multi-dimensional region of a Voronoi diagram can be stored in the element 4174 as a set of points (seeds, generators, sites). Depending on the current search limits (region), the element 4174 can map the non-overlapping multi-dimensional region computed from a seed into a set of inequalities available through the element 4180 that can be more easily evaluated by the classification process 4176.

8. The calculation of the reference model occurs in block 4194. Block 4194 uses the output 4188 from the classification process 4176 and the current and past epoch measurements and relative limits 4198 from block 4178 to calculate the new reference model. In an example embodiment, a new reference model can be calculated by using the current and past epoch values 4198 for “j(i⁺)”, where each optimal model index “i*” is selected by the classification process 4176 as shown in Equations 35, 36, and 37, and “j(i⁺)” indexes the corresponding optimal reference model 4154. The current and past epoch values 4198 for “j(i+)” determine (e.g., select) instances of reference models. As a result, in some embodiments, the current and past epoch values 4198 can be fed back into block 4178 to calculate an optimal average index for the new reference model. The new reference model can be fed into block 4194 via the other information 4199 for the determination of the new reference model. Optionally, the current and past epoch instances of the reference models selected from the current and past epoch values for “j(i⁺)” can be fed into block 4194 via the output 4188 to provide additional information for the determination of the new reference model. Depending on the application, the output 4188 can be used in the determination or calculation of a new reference model at each epoch, such as the current and past selected optimal models' parameters and metrics, and the current and past selected optimal reference models' parameters and metrics obtained from the classification process 4176. Once the new reference model is obtained at block 4194, the current reference model 4172 that will be used in the next epoch by the classification process 4176 is updated at block 4198. The current reference model 4172 can be stored in a particular configuration to save memory and/or improve data retrieving. The current reference model 4172 outputs the parameters in the element 4182 in a realization that can be more efficiently used by the classification process 4176. For instance, the parameters in the element 4182 can be output in a format that enables a direct comparison with the set of normalized parameters in the element 4144 as shown in Equation 27.

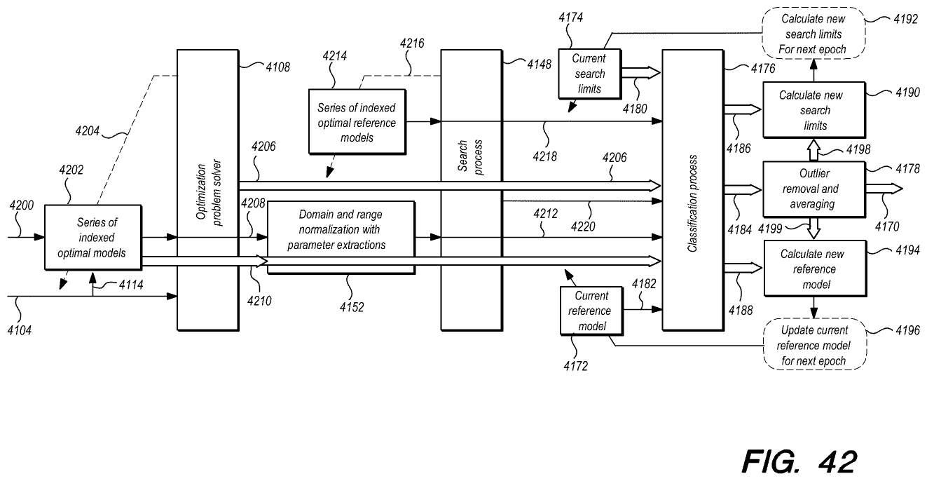

FIG. 42 is a block diagram that illustrates how the example data flow diagrams shown in FIGS. 41A-41H can be connected together and processed at each epoch for a series of indexed models (i.e., i=1, 2, . . . , I), and a series of reference models (i.e., j=1, 2, . . . , J). The input 4200 represents “I” instances of the functions in the element 4102, one for each value of index “i”. The input 4200 and the different operating regions represented by the element 4114 are input into the series of indexed optimal models 4202. The series of indexed optimal models 4202 comprises “I” instances of an indexed optimal model 4116, one for each value of index “i”. The series of indexed optimal models 4202 is calculated for each epoch by the optimization problem solver 4108. In one embodiment, the series of indexed optimal models 4202 are determined according to the example embodiments depicted in FIGS. 41A-41D.

For each epoch, the element 4204 represents the process of applying the optimization problem solver 4108 “I” times over the series of indexed models 4100 to obtain the series of indexed optimal models 4202. The optimization problem solver 4108 outputs the element 4206, which comprises “I” instances of the element 4122, one for each value of index “i”. As discussed previously, in an example embodiment, the element 4122 can be a structure or a container that stores, for each indexed optimal model “i” 4116, the power, sampled correlation, and sampled root mean square accuracy arrays.

The elements 4208 and 4210 are produced by the element 4202 and comprise of “I” instances of the elements 4118 (e.g., the data set or data streams) and the element 4120, one for each value of index “i”. As discussed earlier, the element 4120 represents all parameters (e.g., optimal values for the optimization variables or the model's parameters, etc.) and internal/intermediate signals and variables other than the “i” data sets or “i” sequences of “n” real or complex data streams represented by the element 4118. The elements 4118 and 4120 are input into the element 4152 for domain and range normalization with parameter extractions. Element 4152 outputs the element 4212, which comprises “I” instances of the set of normalized parameters represented by the element 4144, one for each value of index “i”.

A series of indexed optimal reference models 4214 is determined for each epoch by the search process 4148. In one embodiment, the series of indexed optimal reference models 4214 is calculated according to the embodiments shown in FIGS. 41E-41G. For each epoch, the element 4216 represents the process of applying the search process 4148 “I” times over the series of indexed optimal models 4116 and the series of indexed reference models 4140 to obtain the series of indexed optimal reference models 4214. The series of indexed optimal reference models 4214 comprises “I” instances of the indexed optimal reference models indexed by “j(i)” 4154, one for each value of index “j(i)”, where j(i)∈{1, 2, . . . , J} for i=1, 2, . . . , I.

The series of indexed optimal reference models 4214 outputs the element 4218, which comprise “I” instances of the optimal set of parameters represented by the element 4156. The search process 4148 outputs the element 4220, which comprises “I” instances of an index pair “(i, j(i))” represented by the element 4146, one for each value of index “i”.

The elements 4180, 4182, 4206, 4210, 4212, 4218, and 4220 are input into the classification process 4176, which in turn determines the outputs 4184, 4186, and 4188. In one embodiment, the outputs 4184, 4186, 4188 are computed according to the embodiment depicted in FIG. 41H. The outputs 4184, 4186, and 4188 are used by the blocks 4178, 4190, and 4194 to calculate the output measurements 4170, the new search limits at block 4190, and the new reference model (block 4194). The new search limits and the new reference model are used by the blocks 4192 and 4196 to update the current search limits 4174, 4180, the current reference model 4172, and the parameters in the element 4182 that are used in the next epoch.

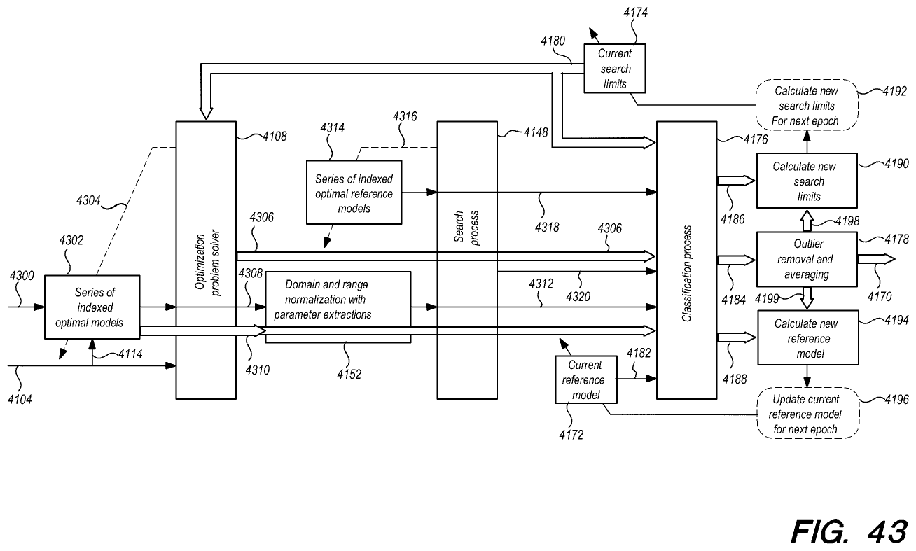

FIG. 43 depicts a block diagram of an alternate embodiment of how the example data flow diagrams shown in FIGS. 41A-41H can be connected together and processed at each epoch for a series of indexed models (i.e., i=1, 2, . . . , I), and a series of reference models (i.e., j=1, 2, . . . , J). The illustrated embodiment can be more efficient by using a series of indexed models with a reduced (and variable) number of instances. The use of the series of indexed models with the reduced (and variable) number of instances is accomplished by inputting into the optimization problem solver 4108 the current search limits 4180 received from the element 4174 to reduce the number indexed model instances to a number “I_{R}” less than (or equal to) “I”.

In the initial or first epochs, the number of instances can be equal to “I”. As data from more epochs are processed, and output measurements and related information are known or stabilized, the search limits can be narrowed along with the number of model instances “I_{R}”. If the output measurements and the related information are subject to discontinuity, such as the removal of the monitoring device from a user or an interrupt and a restart in the wired or wireless connection, or if noise, interference, and/or motion artifacts present in the data streams or data set 4104 increase to higher levels, leading to measurement trends and related information that have statistically less confidence, then the search limits can be increased along with the number of model instances “I_{R}”.

In general, the number of model instances “I_{R}” are updated at each epoch depending on the current search limits 4180. For example, the following equation,

I_{R}−|ϕ| =Equation 40

where

ϕ{i_{ALLOWED}: (m₁PR(t)−PR_{MIN})≤ci_{ALLOWED}≤(M₁PR(t)−PR_{MIN}),1≤i_{ALLOWED}≤I} Equation 41

can be adopted by the optimization problem solver 4108 to select model instances at each epoch. Operator |ϕ| is the cardinality of set ϕ, and PR_{MIN}, c, m₁, and M₁ are constants defined by the application. In one example, m₁PR(t) and M₁PR(t) represent the current search limits (i.e., lower and upper limits) from pulse rate measurements available from the elements 4174, 4180. Argument “t” represents the time epoch when the current search limits 4180 were calculated, and hence are available for computing measurements and related information in the next time epoch “(t+1)”.

The input 4300 in FIG. 43 represents “I_{R}” instances of the functions represented by the element 4102, one for each value of index “i_{ALLOWED}”. The input 4300 and the different operating regions represented by the element 4114 are input into the series of indexed optimal models 4302. The element 4302 comprises “I_{R}” instances of the indexed optimal models represented by the element 4116, one for each value of index “i_{ALLOWED}”. The series of indexed optimal models 4302 is calculated for each epoch by the optimization problem solver 4108. In one embodiment, the series of indexed optimal models 4302 is determined according to the example embodiments depicted in FIGS. 41A-41D.

For each epoch, the element 4304 represents the process of applying the optimization problem solver 4108 “I_{R}” times over a series of indexed models to obtain the series of indexed optimal models 4302. The optimization problem solver 4108 outputs the element 4306. The element 4306 comprises of “I_{R}” instances of the element 4122, one for each value of index “i_{ALLOWED}”. As discussed previously, in an example embodiment, the element 4122 can be a structure or a container that stores, for each indexed optimal model “i” 4116, the power, sampled correlation, and sampled root mean square accuracy arrays.

The elements 4308, 4310 are produced by the element 4302 and comprise “I_{R}” instances of elements 4118 (e.g., the data set or data streams) and the element 4120, one for each value of index “i_{ALLOWED}”. As discussed earlier, the element 4120 represents all parameters (e.g., optimal values for the optimization variables or the model's parameters, etc.) and internal/intermediate signals and variables other than the “i” data sets or “i” sequences of “n” real or complex data streams represented by the element 4118. The elements 4118, 4120 are input into the element 4152 for domain and range normalization with parameter extractions. Element 4152 outputs the element 4312. The element 4312 comprises “I_{R}” instances of the set of normalized parameters represented by the element 4144, one for each value of index “i_{ALLOWED}”.

The series of indexed optimal reference models 4314 is determined for each epoch by the search process 4148. In one embodiment, the series of indexed optimal reference models 4314 is computed according to the example embodiments depicted in FIGS. 41E-41G. For each epoch, the element 4316 represents the process of applying the search process 4148 “I_{R}” times over the series of indexed optimal models 4116 and the series of indexed reference models 4140 to obtain the series of indexed optimal reference models 4314. The element 4314 comprises “I_{R}” instances of the series of indexed optimal reference models indexed by “j(i)” 4154, one for each value of index “j(i_{ALLOWED})”, where j(i_{ALLOWED})∈{1, 2, . . . , J} for i_{ALLOWED} ∈ϕ. The element 4314 outputs the element 4318, which comprise of “I_{R}” instances of the optimal set of parameters represented by the element 4156.

The element 4320 is output by the search process 4148. The element 4320 comprises “I_{R}” instances of the index pair “(i, j(i))”, one for each value of index “i_{ALLOWED}”. The elements 4180, 4182, 4306, 4310, 4312, 4318, and 4320 are input into the classification process 4176, which in turn determines the outputs 4184, 4186, 4188. In one embodiment, the outputs 4184, 4186, 4188 are computed according to the example embodiment depicted in FIG. 41H. The outputs 4184, 4186, 4188 are used by blocks 4178, 4190, and 4194 to calculate the output measurements 4170, the new search limits at block 4192, and the new reference model at block 4194. The new search limits and the new reference model are used by the blocks 4192 and 4196 to update the current search limits 4174, 4180, the current reference model 4172, and the parameters in the element 4182 that are used in the next epoch.

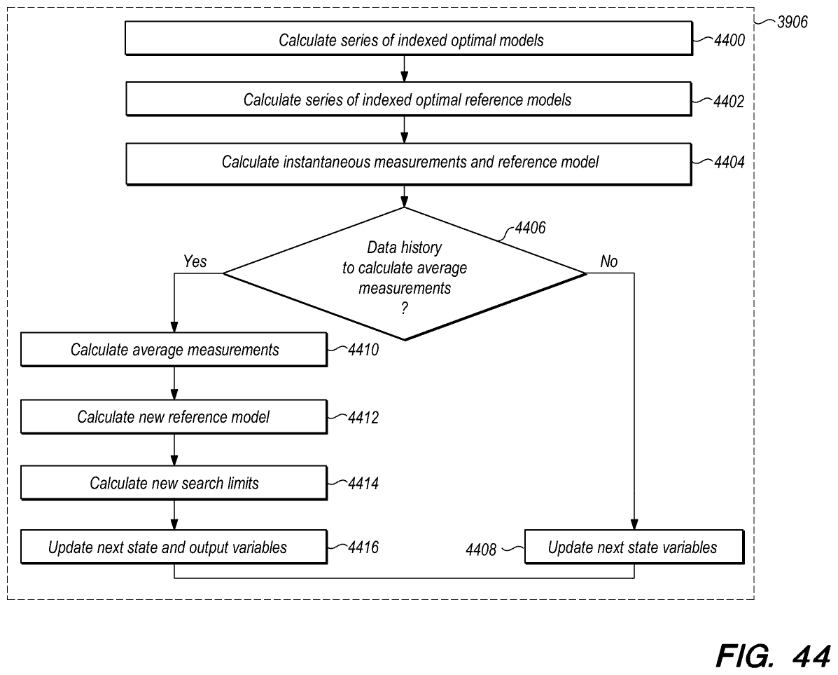

FIG. 44 illustrates a flowchart of example operations that are suitable for use in block 3906 shown in FIG. 39. In block 4400, the host device (e.g., via the host software) calculates a series of indexed optimal models at each time epoch. In one embodiment, the series of indexed optimal models are determined by an optimization problem solver using the example embodiments shown in FIGS. 41A-41D, 42, and 43.

Next, as shown in block 4402, the host device calculates a series of indexed optimal reference models at each epoch. In one embodiment, the series of indexed optimal reference models are determined by a search process according to the example embodiments shown in FIGS. 41E-41G, 42, and 43. The host device also calculates the instantaneous measurements (e.g., intermediate values and quantities, optical rations and other functionals, oxygen saturation, pulse rate, perfusion index, pulse amplitude, dyshemoglobin concentrations, glucose concentration in blood, hematocrit, water in blood, protein in blood, lipids in blood, blood or core temperature, blood pressure, etc.) and the reference model in block 4404. In one embodiment, the instantaneous measurements and the reference model are determined by a classification process according to the example embodiments shown in FIGS. 41H, 42, and 43.

A determination is made at block 4406 as to whether a sufficient amount of data history exists to calculate average measurements. Block 4406 ensures that enough instantaneous measurements, reference models, and related information (i.e., data history) have been successfully calculated by a classification process prior to the calculation of the average measurements. If a determination is made that a sufficient amount of data does not exist, the process passes to block 4408 where the next state variables in software 120 are updated using the results from blocks 4400, 4402, and 4404 (e.g., the results from the optimization problem solver, the search process, the classification process, and the related information).

When a determination is made at block 4406 that a sufficient amount of data exists, the method continues at block 4410 where the average measurements are calculated from the instantaneous measurement data history. In one embodiment, the calculation of the average measurements is performed via block 4178 shown in FIG. 41H, 42, and 43. As described earlier, the block 4178 uses the output 4184 (e.g., instantaneous measurement data history, etc.) output by the classification process 4176, and the related information to perform outlier removal, and averaging of the instantaneous measurement data history. Block 4178 produces the information in the elements 4198, 4199 and the output measurements 4170.

Next, as shown in block 4412, the new reference model is calculated. In one embodiment, calculation of the new reference model occurs in block 4194 and is discussed in conjunction with the example embodiments depicted in FIG. 41H, 42, and 43. The block 4194 receives the information in the output 4188 (e.g., the reference model data history, etc.) and the element 4199 produced by the classification process 4176 and the element 4178. The block 4194 calculates the new reference model to be used in the next epoch.

Block 4414 calculates the new search limits using the results from blocks 4400, 4402, and 4404. In one embodiment, calculation of the new search limits is performed by block 4190 as shown in FIGS. 41H, 42, and 43. The block 4190 receives the information in the output 4186 and in the element 4198 that are produced by the classification process 4176 and the block 4178, respectively, to calculate the new search limits to be used in the next epoch.

Block 4416 updates the next state and the output variables using the results from blocks 4400, 4402, and 4404. In one embodiment, the results that are used to update the next state and the output variables include the results from the optimization problem solver, the search process, the classification process 766, the average measurements from block 4410, the new reference model from block 4412, the new search limits from block 4414, and related information.

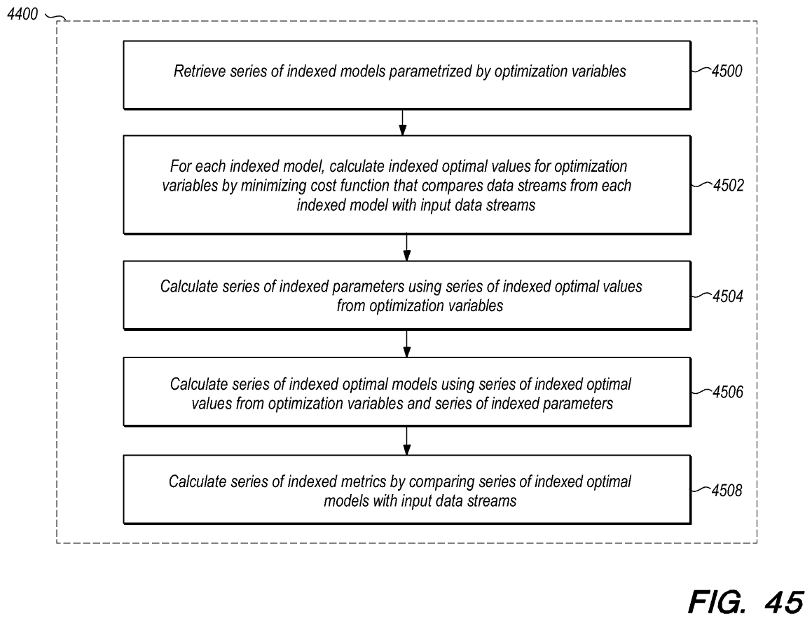

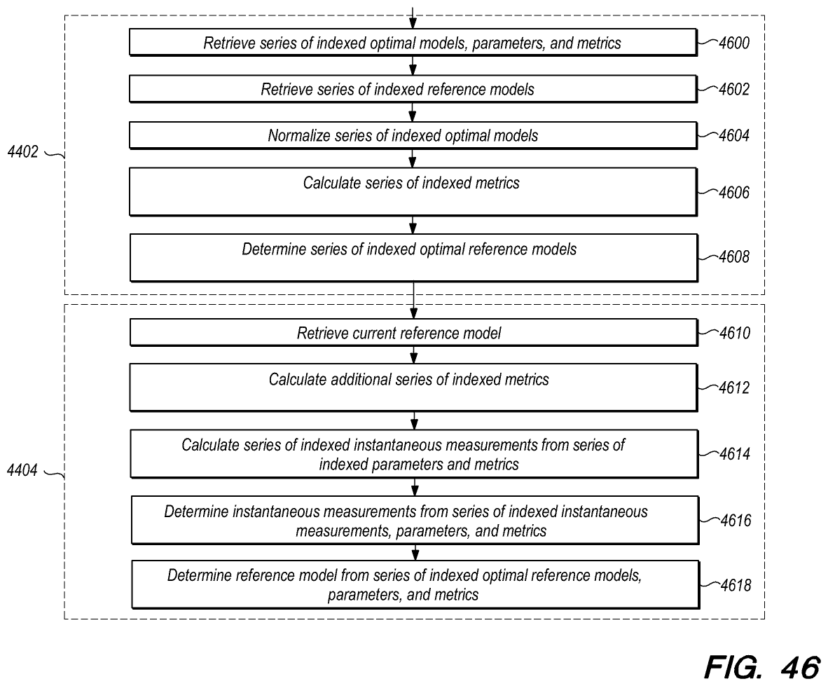

FIG. 45 depicts a flowchart of example operations that are suitable for use in block 4400 shown in FIG. 44. Initially, as shown in block 4500, a series of indexed models parametrized by optimization variables are retrieved from the state variables in the host software. FIGS. 41A-41D, 42, and 43 describe example embodiments for a series of indexed (optimal) models. Next, as shown in block 4502, the indexed optimal values for the optimization variables are calculated for each indexed model. In one embodiment, the calculation of the indexed optimal values is performed by an optimization problem solver that minimizes a cost function that compares each indexed model (e.g., indexed model 4100 in FIG. 41A) via the data streams produced by the series of indexed models (e.g., element 4106 in FIG. 41A) with the normalized data streams (i.e., element 4104 in FIG. 41A) stored in the input variables. The data streams that are produced by the series of indexed models are generated by inputting the function and the different operating regions represented by the elements 4102 and 4114 in FIG. 41A into each indexed model (e.g., indexed model 4100 in FIG. 41A).

Next, as shown in block 4504, a series of indexed parameters is calculated from the indexed optimal values for optimization variables. The series of indexed parameters and the series of optimal values for the optimization variables are used to calculate a series of indexed morphological and time-frequency measures (e.g., amplitudes, phases, power, energy, distributions, harmonic content, etc.) that characterize the normalized data streams represented in element 4104 at each epoch. In block 4506, the resulting series of indexed optimal models are calculated from a series of indexed parameters and the series of optimal values for the optimization variables. In one embodiment, the series of indexed optimal models (e.g., series of indexed optimal models 4116 in FIG. 41B, 4202 in FIG. 42, and 4302 in FIG. 43) are obtained using the example embodiments depicted in FIGS. 41A-41D, 42, and 43.

A series of indexed metrics (e.g., root mean square accuracies, correlations, variances, absolute errors, minimum-, maximum-, mean-, and median-based metrics, etc.) is then calculated by comparing the series of indexed optimal models with the normalized data streams represented in element 4104 in FIG. 41 (block 4508). The results from blocks 4504, 4506, and 4508 and the series of indexed optimal models are output. In one embodiment, the results from blocks 4504, 4506, and 4508 are produced by an optimization problem solver 4108 (FIG. 41A) and the series of indexed optimal models (e.g., series of indexed optimal models 4116 in FIG. 41B, 4202 in FIG. 42, and 4302 in FIG. 43) are represented by elements 4118, 4120, and 4122 in FIGS. 41A-41D, elements 4206, 4208, 4210 in FIG. 42, or elements 4306, 4308, and 4310 in FIG. 43.

FIG. 46 illustrates a flowchart of example operations that are suitable for use in blocks 4402 and 4404 shown in FIG. 44. Blocks 4402 and 4404 describe example processes to calculate a series of indexed optimal reference models, instantaneous measurements, and a series of reference models to assemble a data history through multiple epochs to enable the calculation of average measurements in block 4410 (FIG. 44), a new reference model in block 4412, and new search limits in block 4414. In one embodiment, the new reference model is a model instance selected from a series of indexed optimal reference models. The selection of the new reference model can be the reference model itself or an index (e.g., “j(i+))” as described in FIG. 41H). The calculation of the new reference model in block 4412 can involve calculations with the selected reference models and/or their selected index values that are obtained through multiple epochs (data history). For instance, a new reference model can be determined by performing outlier removal, averaging, and rounding directly on the selected index values and picking an instance from the series of indexed optimal reference models that is indexed by the averaged and rounded index result.

Referring to FIG. 46, a series of indexed optimal models, parameters, and metrics, as well as a series of indexed reference models, are retrieved from the state variables in the host device (e.g., the host software) (blocks 4600, 4602). Next, as shown in block 4604, a series of indexed optimal models are normalized. In one embodiment, the series of indexed optimal models are normalized using the domain and range normalization with parameter extractions represented in element 4152 in FIGS. 41E, 41G, 42, and 43.

A series of indexed metrics that compare a series of indexed reference models with the series of (normalized) indexed optimal models are determined at block 4606. Non-limiting examples of the indexed metrics include root mean square accuracies, correlations, variances, absolute errors, minimum-based metrics, maximum-based metrics, mean-based metrics, and median-based metrics. A series of indexed optimal reference models is then computed at block 4608 using the series of indexed metrics calculated in block 4606. In one embodiment, the calculations of the series of indexed metrics and optimal reference models are performed by the search process 4148 (FIG. 41E) detailed in the example embodiments depicted in FIGS. 41E-42G, 42, and 43.

Blocks 4610, 4612, 4614, 4616, and 4618 describe example operations that are performed by the classification process 4176 to calculate the (optimal) instantaneous measurements and the reference model at each epoch to assemble the data history. In block 4610, the parameters of the current reference model (e.g., parameters 4182 of the current reference model 4172) are retrieved from the state variables in the host device (e.g., the host software). An additional series of indexed metrics that enable a comparison of the series of (normalized) indexed optimal models with the current reference model is computed at block 4612. In one embodiment, the series of (normalized) indexed optimal models are represented by elements 4116, 4242, and 4302 in FIGS. 41E, 41G, 42, and 43, respectively and the parameters of the current reference model are represented by element 4182 in FIG. 41H.

Next, as shown in block 4614, a series of indexed instantaneous measurements (e.g., SpO2, PR, PI, etc.) are calculated from the series of indexed optimal parameters and metrics. A selection of instantaneous measurements from the series of indexed instantaneous measurements, parameters, and metrics is performed in block 4616. In one embodiment, the selection of the instantaneous measurements is performed by the classification process 4176 in FIGS. 41H, 42, and 43.

A reference model (i.e., index “j(i⁺)”) is selected from a series of indexed optimal reference models, parameters and metrics at block 4618. In one embodiment, the selection of the reference model (i.e., index “j(i⁺)”) is performed by the classification process 4176 in FIGS. 41H, 42, and 43. The classification process 4176 calculates the reference model (e.g., the current and past epoch values for “j(i⁺)”), instantaneous measurements, and related parameters through multiple epochs to enable the data history to be assembled. The data history is input into blocks 4178, 4194, and 4190 via the outputs 4184, 4188, and 4186, respectively, to compute the average measurements (block 4178), the new reference model (block 4194), and the new search limits (block 4190).

In some embodiments, the host software in the host device and the device firmware in the monitoring device are operable to implement a distributed fuel gauge system to measure the battery life of the monitoring device. The distributed fuel gauge system can reduce the number of components, the hardware complexity, and/or the manufacturing costs of the monitoring device. The distributed fuel gauge system relies on measurements of the battery and circuitry (e.g., temperatures, clock frequencies, currents, voltages, loads, etc.) that are taken by the monitoring device and transmitted to the host device. In addition, the monitoring device can maintain one or more non-volatile counters that log the energy consumed by the monitoring device in each of the states of the monitoring device (e.g., the states shown in FIG. 26). In one example embodiment, the monitoring device can have a standby counter that is active whenever the monitoring device is in standby and is not connected to the host device (e.g., state 0 in FIG. 26). In standby, the monitoring device consumes less power because the monitoring device is not connected to the host device and is not performing any biosensing functionalities.

Additionally or alternatively, the monitoring device may have a usage counter that is active whenever the monitoring device (not in state 0 in FIG. 26) is running and/or is connected to the host device. When the monitoring device is running and/or is connected to the host device, the power consumption of the monitoring device increases because of the wired or wireless connection and/or the execution of one or more biosensing functionalities.

The rate of change for the standby counter may be constant (assuming standby power consumption is always the same) or variable (assuming standby power consumption changes depending on observable variables, such as temperatures, clock frequencies, currents, voltages, loads, etc.). The same rational can be applied to the usage counter. The rate of change for the usage counter may be constant (assuming running power consumption is always the same) or variable (assuming running power consumption changes depending on observable variables, such as temperatures, clock frequencies, currents, voltages, loads, etc.). In one embodiment, the rate of change for both the standby counter and the usage counter is increments or decrements in discrete-time intervals.

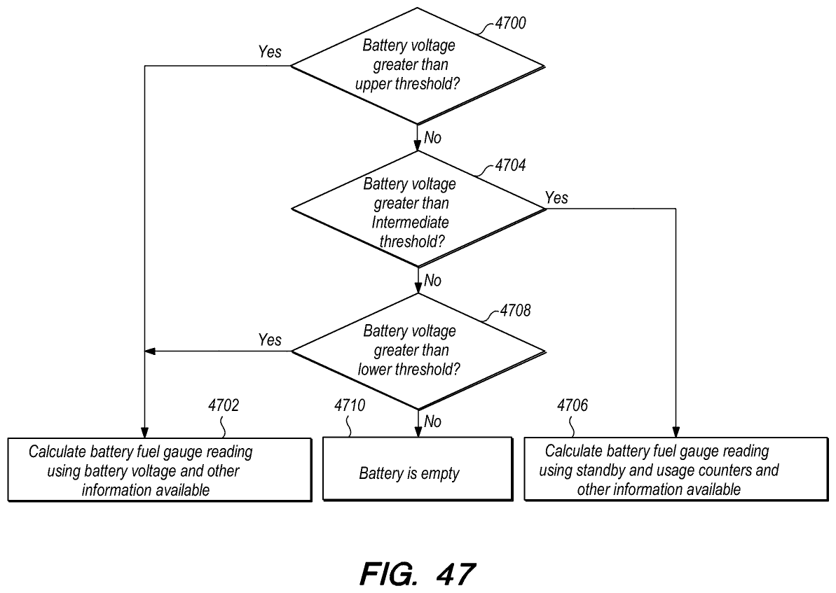

FIG. 47 depicts a flowchart of a method of operating a distributed fuel gauge system that measures the battery life of the monitoring device. The process is performed by the host device and divides the battery life measurement into four regions:

1. The first region is associated with battery voltages that are greater than a pre-defined upper threshold. In the first region, the battery is charged fully and changes (decreases) in the battery energy cause the battery voltage to change (decrease). As a result of the battery voltage decreases in the first region, the battery voltage can be mapped into the energy remaining in the battery (i.e., the battery energy that is left).

2. The second region is associated with battery voltages that are less than or equal to the upper threshold and greater than a predefined intermediate threshold. In the second region, changes (decreases) in the battery energy do not cause the battery voltage to change. This lack of change in the battery voltage enables the battery voltage to be mapped into the energy remaining in the battery. In the second region, the battery energy consumption is accounted for by one or more non-volatile counters implemented in the monitoring device that log the energy spent by the monitoring device.

3. The third region is associated with battery voltages that are less than or equal to the intermediate threshold and greater than a pre-defined lower threshold. In the third region, the battery is low in charge and changes (decreases) in the battery energy cause the battery voltage to change (decrease). As a result of the decreases in the battery voltage, the battery voltage can be mapped into the energy remaining in the battery.

4. The fourth region is associated with battery values that are less than the lower threshold. In the fourth region, the energy left in the battery is considered to have reached a level where the battery is considered fully discharged (empty).

Referring to block 4700 in FIG. 47, a determination is made as to whether the battery voltage is greater than an upper threshold. If so, the process passes to block 4702 where the battery fuel gauge reading (i.e., battery's normalized energy left) is computed by an equation such as,

E=min(100,e₀+e₁V_{BAT})V_{BAT}>V_{H} Equation 42

where E is the battery's normalized energy from 0 to 100%, e₀ and e₁ are constants, V_{H} is the battery voltage's upper threshold, and V_{BAT} is the device's battery voltage read by the monitoring device and sent to the host device. The “min” function and number “100” in Equation 42 are to prevent the computation of normalized energy values that are higher than 100%. Equation 42 can also be a higher order polynomial function of V_{BAT}, or some other function that maps V_{BAT} and optionally other measurable parameters (e.g., battery's temperature, environmental pressure, temperature and humidity, device's circuitry current consumptions, etc.) into the normalized energy value E.

If a determination is made at block 4700 that the battery voltage is not greater than the upper threshold, the method continues at block 4704 where a determination is made as to whether the battery voltage is less than or equal to the upper threshold and greater than an intermediate threshold. If so, the process passes to block 4706 where the battery's normalized energy can be computed by an equation such as,

E=max(E_{L},100−e₂C_{STANDBY}−e₃C_{USAGE}),V_{H}≥V_{BAT}>V_{I} Equation 43

where e₂ and e₃ are constants, V_{I} is the intermediate threshold of the battery voltage, C_{STANDBY} and C_{USAGE} are the standby and the usage counter values read by the monitoring device and sent to the host device. Constant E_{L} is the minimum normalized energy threshold (e.g., 8%, 10%, 15%, etc.) value calculated by Equation 43. Equation 43 can also be a higher order polynomial function of C_{STANDBY} and C_{USAGE}, or some other function that maps C_{STANDBY} andC_{USAGE} and optionally other measurable parameters (e.g., battery's temperature, environmental pressure, temperature and humidity, device's circuitry current consumptions, etc.) into the normalized energy value E.

If a determination is made at block 4704 that the battery voltage is less than or equal to the upper threshold but is not greater than the intermediate threshold, the process continues at block 4708 where a determination is made as to whether the battery voltage is less than or equal to the intermediate threshold and greater than a lower threshold. If so, the method passes to block 4702 and the normalized energy of the battery can be computed by an equation such as,

E=max(0,e₄+e₅V_{BAT}),V_{I}≥V_{BAT}>V_{L} Equation 44

where e₄ and e₅ are constants and V_{L} is the lower threshold of the battery voltage. The “max” function and number “0” in Equation 44 is to prevent the computation of normalized energy values that are lower than 0%. Equation 44 can also be a higher order polynomial function of V_{BAT}, or some other function that maps V_{BAT} and optionally other measurable parameters (e.g., battery's temperature, environmental pressure, temperature and humidity, device's circuitry current consumptions, etc.) into the normalized energy value E.

If a determination is made at block 4704 that the battery voltage is less than or equal to the intermediate threshold and is less than the lower threshold, the process continues at block 4710 where the normalized energy is set to zero to signifies the battery is discharged fully (e.g., is empty).

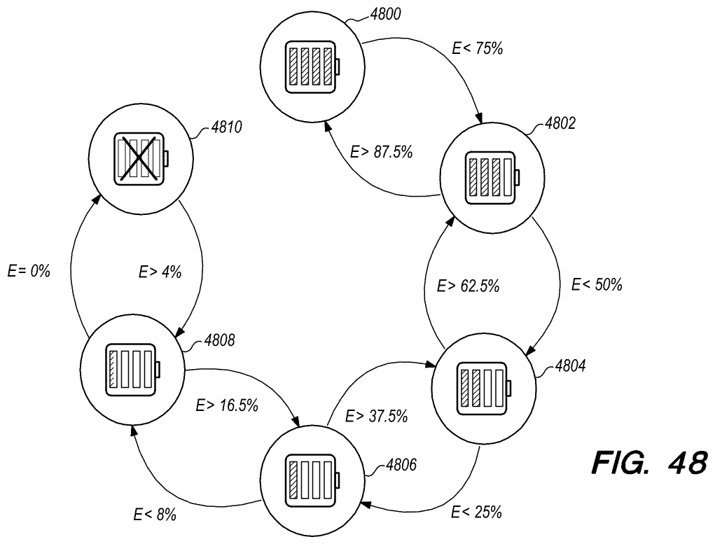

In FIG. 47, the measurement of the battery's energy involves multiple equations that utilize voltage and other measurements that are subjected to inherent noise, as well as the one or more counters. As a result, when translating the battery's continuous normalized energy values from Equations 42, 43, and 44 into discrete values in fuel gauge indicator for the battery, hysteresis can be added to the logic to enable the transition from one discrete level to another, in order to prevent jittering. FIG. 48 illustrates an example fuel gauge indicator that considers hysteresis to prevent jittering. FIG. 48 depicts six states for the battery and a battery icon for each state, although other embodiments are not limited to this configuration. In other embodiments, a fuel gauge indicator for a battery can have any number of states and/or any type of a graphic representation of a battery or the battery energy.

The fuel gauge indicator begins at state 4800 with the battery charged fully. When the battery's normalized energy is less than 75%, the fuel gauge indicator transitions to state 4802 where battery energy is ¾ full. In the illustrated embodiment, the state 4802 only returns to the state 4800 when the battery's normalized energy is greater than 87.5%. The hysteresis in 75%-87.5% prevents jittering when the fuel gauge indicator transitions from the state 4800 to the state 4802.

When the battery's normalized energy is less than 50%, the fuel gauge indicator transitions from state 4802 to state 4804 where the battery energy is ½ full. In the example embodiment, the state 4804 only returns to the state 4802 if the battery's normalized energy is greater than 62.5%. The hysteresis in 50%-62.5% prevents jittering when the fuel gauge indicator transitions from state 4802 to state 4804.

When the battery's normalized energy is less than 25%, the fuel gauge indicator transitions from state 4804 to state 4806 where the battery energy is ¼ full. In the illustrated embodiment, the state 4806 only returns to the state 4804 if the battery's normalized energy is greater than 37.5%. The hysteresis in 25%-37.5% prevents jittering when the fuel gauge indicator transitions from the state 4804 to the state 4806.

When the battery's normalized energy is less than 8%, the fuel gauge indicator transitions from the state 4806 to the state 4808 where the battery energy is low. In the example embodiment, the state 4808 only returns to the state 4806 if the battery's normalized energy is greater than 16.5%. The hysteresis in 8%-16.5% prevents jittering when the fuel gauge indicator transitions from state 4806 to the state 4808.

When the battery's normalized energy is equal or substantially equal to 0%, the fuel gauge indicator transitions from the state 4808 to the state 4810 where the battery is empty. In the illustrated embodiment, the state 4810 only returns to the state 4808 if the battery's normalized energy is greater than 4%. The hysteresis in 0%-4% prevents jittering when the fuel gauge indicator transitions from the state 4808 to the state 4810.

FIG. 49A depicts a flowchart of a method of initializing and restarting a monitoring device based on one or more counters associated with battery energy. In one embodiment, the method is performed by a monitoring device. Initially, as shown in block 4900, a determination is made as to whether a count of a standby counter is at a maximum count. If so, the process passes to block 4902 where the monitoring device is turned off and restarted.

If a determination is made at block 4900 that the count of the standby counter is not at a maximum count, the method continues at block 4904 where a determination is made as to whether a count of a usage counter is at a maximum count. If so, the process transitions to block 4902. When the standby counter or the usage counter reach a maximum count, the monitoring device shuts down and is restarted at block 4902. Once the count on the standby counter or on the usage counter has maxed out, the monitoring device is no longer connected to the host device and does not perform any biosensing functionalities. In one embodiment, the standby counter and the usage counter are non-volatile and cannot be reset once the standby counter or the usage counter reaches the maximum count. Thus, the standby and the usage counters can prevent unauthorized users from extending the use of the monitoring device. Preventing the extension of the use of the monitoring device is especially useful in single-use applications where the monitoring device is disposable and has a non-rechargeable battery that cannot be replaced. For example, in some situations the battery cannot be replaced due to safety and/or regulatory reasons after the battery has been discharged fully.

If a determination is made at block 4904 that the count of the usage counter is not at a maximum count, the method continues at block 4906 where the standby counter data structure is updated with an increment to reflect a decrease in the battery's energy. In block 4908, the firmware data structures in the device firmware in the monitoring device are initialized. The initialization includes, but is not limited to, data pointers, structures, unions, arrays, and/or multi-dimensional arrays.

Next, as shown in block 4910, one or more wireless and/or communication services are initialized. In an example embodiment, the wireless service for a Bluetooth Low Energy (BLE) connection can be a collection of data and associated behaviors and functions to enable wireless functionalities (pairing and connection management, data transfer, interruptions, error handling, etc.) used by the device firmware via a BLE software stack and main application. In block 4912, a data collection interruption service is initialized. The data collection interruption service enables the device firmware to store and process real-time data acquired periodically by the frontend circuitry in the monitoring device. The data collection service managed by interruption, which can be trigged by a number of data samples collected or by an elapsed time, reduces power consumption in the monitoring device and frees up resources that can be used by other services (e.g., supervisory, communication, etc.).

In block 4914, a supervisory interruption service is initialized. The supervisory interruption function is activated periodically to monitor and control the states of the monitoring device. For example, the states of the device firmware are the states depicted in FIG. 26. A watchdog timer interruption service is initialized at block 4916. The watchdog timer interruption occurs whenever a watchdog timer timeout occurs because of a software or hardware failure, or a network disconnect between the monitoring device and the host device, after a certain amount of time has passed (e.g., a watchdog timer timeout period). For example, the watchdog timer timeout period may be 5 or ten seconds. The watchdog timer interruption service is particular useful in embodiments where the monitoring device is low-cost, single use, and disposable monitoring device that does not include a reset switch or interface that enables reinitialization and recovery from a hardware or software fault.

FIG. 49B illustrates a flowchart of a method of operating a watchdog timer. In one embodiment, the process shown in FIG. 49B is executed by the monitoring device and operates concurrently with the method shown in FIG. 49A. Whenever the monitoring device is disconnected from the host device, the watchdog timer is not refreshed by the supervisory interruption service or the data collection interruption service. At block 4918, a determination is made as to whether the watchdog timer is timed out (e.g., the timeout period is reached). If so, the method passes to block 4902 where the monitoring device is shut down and restarted. The shutdown and reinitialization process occurs periodically while the monitoring device is turned on but is not connected to the host device. The shutdown and reinitialization process happens at a frequency that is defined by the watchdog timer timeout period, and optionally by other factors such as the bootup time for the microprocessor and peripherals in the monitoring device.

When a determination is made at block 4918 that the watchdog timer has not timed out, the method waits at block 4918. The watchdog timer ensures that faults are detected and handled while the monitoring device is connected to the host device. Additionally, the watchdog timer also ensures that the monitoring device is always ready for operation and fault-free (whenever possible) when the monitoring device is disconnected from the host device.

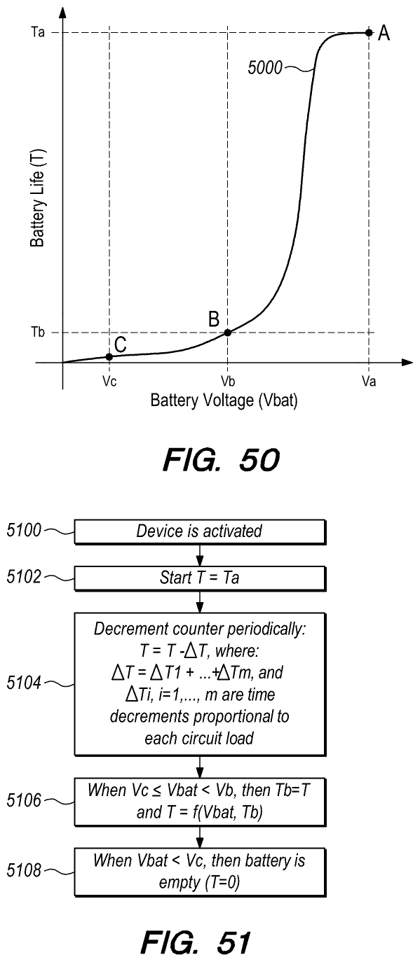

In some embodiments, the battery is a non-rechargeable battery. Information about the charge on the non-rechargeable battery and/or the usage of the monitoring device is provided to the user at select times or continuously to enable the user to predict when the monitoring device is to be replaced. FIG. 50 and FIG. 51 illustrate a method to compute the life of the battery in the monitoring device. The method does not use specialized circuitry that can increase the manufacturing costs of the monitoring device. The challenge to accurately estimate the battery life directly from the battery voltage is that the battery voltage changes very little throughout most of its discharge curve, which can make mapping from battery voltage to battery life (or time left) unreliable.

FIG. 50 depicts an example battery discharge curve. The battery voltage (x axis) starts from Va (fully charged) and ends in Vc (fully discharged). From Va to Vb, the battery discharge curve 5000 is too vertical to produce an accurate estimate for battery life (y axis) from the battery voltage. The y-axis can represent time, percentage, joules, or another suitable value. The challenge of estimating the battery life is addressed herein by a hybrid method of estimating the battery life. The hybrid method uses one or more counters in combination with battery voltage measurements to more accurately estimate the battery life.

FIG. 51 illustrates a flowchart of a method of estimating the life of the battery in the monitoring device based on the battery discharge curve shown in FIG. 50. The monitoring device is activated and the processing device in the monitoring device (e.g., 102 in FIG. 1) starts a usage counter T (blocks 5100 and 5102). Ta represents the time it takes to completely discharge the battery completely when the monitoring device is under operation. For instance, if the battery life has typical value, a minimum value, a maximum value or an average value of 24 hours, then Ta is set to a value that corresponds to 24 hours of battery life.

In one embodiment, the usage counter is implemented in non-volatile memory (e.g., a flash memory) so as not to lose the counter value in the event of a power transient. The usage counter is decremented periodically by the processing device at block 5104 based on the electrical loads in the circuitry of the monitoring device. The higher the power consumption of a particular load, the larger the corresponding decrement (ΔTi). Some loads are constant over time, and some tend to vary depending on known factors. For instance, the required current of a light source (LED) may vary depending on the measurement site's optical opaqueness. As a result, the corresponding ΔTi of a particular LED should be adjusted by its set current. The higher the current, the higher the ΔTi value.

When the battery voltage Vbat is less than Vb and greater than or equal to Vc (FIG. 50), the amount of battery life that remains (e.g., the amount of charge on the battery) is estimated through the function ƒ(Vbat, Tb) at block 5106. The function ƒ(Vbat, Tb) can be implemented in several ways. One implementation for ƒ(Vbat, Tb) is to be the minimum value between the battery life estimate from the battery voltage curve (as depicted in FIG. 50) and the last (Tb) battery life estimate from the usage counter, with ƒ(Vbat,Tb)=Tb for Vbat=Vb. This ensures functional continuity for the measurements of battery life, even if the battery voltage increases momentarily because of changes in ambient temperature, circuitry loads, etc. The battery is considered fully discharged (empty) when the battery voltage drops below Vc (FIG. 50) at block 5108. The different stages of the battery life estimate shown in FIG. 50 can be used to inform the user via any suitable technique or techniques. For example, a graphic representation (e.g., a battery icon) can be displayed on a host device. Additionally or alternatively, the monitoring device and/or the host device can produce an alert, such as an audible alert or a haptic alert. The stages of the battery line may also be used to trigger a battery empty alarm/warning when the battery is fully discharged.

The usage counter implemented in block 5102 of FIG. 51 can also be used to monitor the usage time of the monitoring device. In some embodiments, an additional counter (e.g., a standby counter) can be used to account for the amount of time the monitoring device is in a standby mode (i.e., activated but not connected to the host device). The standby counter can be implemented in the same non-volatile memory (e.g., flash memory) with the usage counter. The usage and the standby times are calculated based on the aforementioned usage and standby counters, which are implemented in the non-volatile memory. In an example embodiment, the values of the usage and the standby counters are sent wirelessly at select times (e.g., periodically) to the host device. Additionally, in the example embodiment, because the usage and the standby counters cannot be deleted from the outside, or by an unauthorized user without deleting the entire firmware, thereby rending the monitoring device nonoperational, the usage and the standby counters can be used to disable the monitoring device once the usage and/or the standby counter reach a certain value. As discussed previously, the standby and the usage counters prevent unauthorized users from replacing the non-rechargeable battery (tampering) to enable the monitoring device to be used for an extended period of time. The usage and the standby counters can increase the safety of the monitoring device because the monitoring device cannot be repaired or disassembled without compromising the circuitry in the monitoring device.

Another safety feature can be implemented by starting a voltage timer or counter (“voltage timer”) whenever the voltage of the battery drops below a certain threshold. If the battery voltage stays below the threshold until the voltage timer reaches a pre-defined value, the battery voltage cannot increase past the threshold given that the battery is non-rechargeable, and a flag is set in the non-volatile memory indicating the battery is fully discharged. Later, in the event the battery voltage becomes greater than the threshold, with the usage and/or the standby counters expired, an inference can be made that an unauthorized user (or third-party) has tempered with monitoring device. In this case, the device firmware of the monitoring device may reset the processing device (e.g., processing device 102 in FIG. 1) and go to an idle state in order to prevent the unauthorized use or repurposing of the monitoring device.

FIG. 52 illustrates a flowchart of an anti-tampering method for the monitoring device. Initially, as shown in block 5200, a determination is made as to whether the monitoring device is active and in use. If the monitoring device is in use, the process passes to block 5202 where the usage counter is incremented. A determination is made at block 5204 as to whether the usage counter has reached its maximum value. If so, the method continues at block 5206 where the monitoring device stops operating and cannot be used. In an alternate embodiment, if the usage counter reaches its maximum value, then block 5206 can be executed only after the connection (e.g., wireless connection) between the monitoring device and the host device is lost to stop operation. This ensures that the user is not put at risk due to the lack of operation of the monitoring device because the usage counter has reached its limit (maximum value).

Returning to block 5200, if a determination is made that the monitoring device is not in use, it is assumed the monitoring device is in standby mode and the standby counter is incremented at block 5208. A determination is made at block 5210 as to whether the standby counter has reached its maximum value. If so, the process passes to block 5212 where the monitoring device stops operating and cannot be used. Again, in an alternate embodiment, if the standby counter reaches its maximum value, then block 5212 can be executed only after the connection (e.g., wireless connection) between the monitoring device and the host device is lost to stop operation. This ensures that the user is not put at risk due to the lack of operation of the monitoring device because the standby counter has reached its limit (maximum value).

When the standby counter and the usage counter have not reached the maximum values, the method continues at block 5214 where a determination is made as to whether the battery voltage is below a pre-defined voltage threshold. If so, a threshold counter is incremented at block 5216. Otherwise, the threshold counter is reset at block 5218.

A determination is made at block 5220 as to whether the threshold counter has reached its maximum value. If so, the method passes to block 5222 where the monitoring device either stops operation (e.g., immediately), continues normal operation until the standby or usage counters expire, or stops operation after the battery voltage increases higher than the pre-defined voltage threshold. The method returns to block 5200 when a determination is made at block 5220 that the threshold counter has not reached its maximum value.

In some embodiments, the standby, usage and threshold counters are implemented in the non-volatile memory in the monitoring device. Alternatively, the threshold counter can be implemented in the volatile memory in the monitoring device, or in the volatile memory of the host device, given that the threshold counter is typically used to measure time intervals much smaller than typical usage or standby time intervals. Implementing the counters in the non-volatile memory decreases the chances of an occurrence of power outage, failure, or reset that may cause the counters to lose their current values

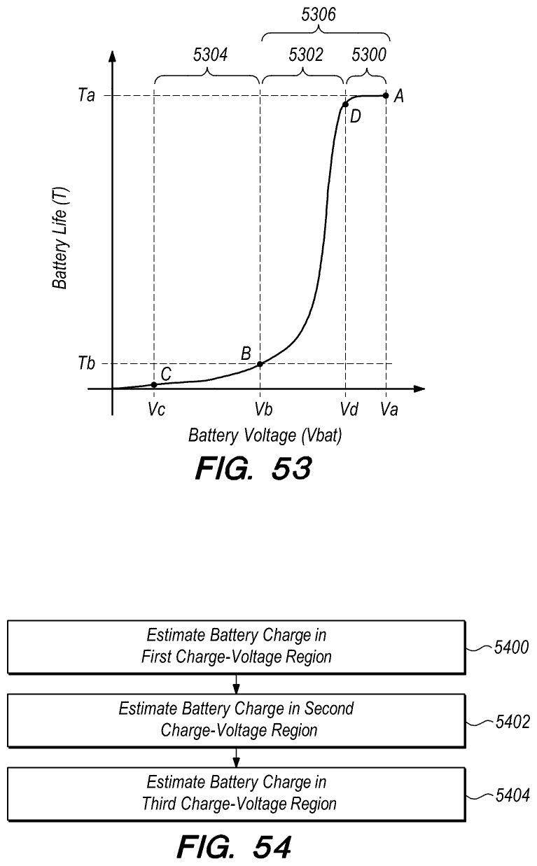

FIG. 53 depicts the example battery discharge curve that is shown in FIG. 50. The x-axis represents the battery voltage and the y-axis can represent time, percentage, joules, voltage or other values. The example battery discharge curve is divided into three charge-voltage regions 5300, 5302, and 5304. In the first charge-voltage region 5300, the battery is charged fully or almost fully. In region 5300, small changes (drops) in the battery charge create relatively large changes (drops) in the battery voltage, which are observable. As a result, it is possible to determine the battery charge (e.g., battery life) from the battery voltage and/or other available parameters of interest (e.g., ambient temperature, circuitry loads, etc.).

In the second charge-voltage region 5302, changes in battery charge do not affect (or substantially affect) the battery voltage. As a result, in the second charge-voltage region 5302, the battery charge is accounted for via one or more counters (or timers). In other words, region 5302 does not include observable or measurable parameters that can be used to estimate the battery charge directly. Thus, the one or more counters and/or information about other available parameters of interest (e.g., ambient temperature, circuitry loads, etc.) is used to estimate the battery charge.

As the battery discharges, the battery enters the third charge-voltage region 5304 where the battery charge can again be estimated from the observable battery voltage and/or other available parameters of interest (e.g., ambient temperature, circuitry loads, etc.). In an alternate embodiment, the first and the second regions 5300, 5302 can be combined into a single first/second non-observable charge-voltage region 5306 and the calculations are performed as described for region 5302.

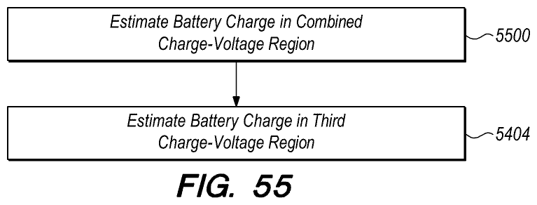

FIGS. 54 and 55 illustrate example methods of estimating the battery life of the battery in the monitoring device. The computations in each method are performed in both a closed loop and an open loop via different functions. In the closed loop, the battery voltage and/or other available parameters of interest (e.g., ambient temperature, circuitry loads, etc.) is used to estimate the battery charge directly. In the open loop, the battery charge is estimated indirectly using one or more counters and/or other available parameters of interest (e.g., ambient temperature, circuitry loads, etc.).

In FIG. 54, the battery in in the monitoring device is operating in the first charge-voltage region at block 5400 (e.g., 5300 in FIG. 53). In an example embodiment, the battery charge is calculated by a function that maps the battery voltage and other parameters of interest (i.e., voltage, temperature, circuitry loads, etc.) into normalized battery charge values. In one embodiment, the monitoring device measures the battery voltage and the other parameters of interest and transmits the measurements to the host device. The host device determines the normalized battery charge values.

In block 5402, the battery charge reaches the second charge-voltage region (e.g., 5302 in FIG. 53) and the battery charge is estimated using one or more counters that are implemented in the monitoring device (e.g., in non-volatile memory). The counter value(s) are transmitted to the host device for processing and the host device decreases the battery charge over time as a function of the counter value(s). In one embodiment, the one or more counters represent different operating modes in the monitoring device. In an example embodiment, a first counter and a second counter can be implemented in the monitoring device. The first counter (e.g., the standby counter) is incremented whenever the monitoring device is in standby mode (e.g., not connected to the host device). The second counter (e.g., the usage counter) is incremented whenever the monitoring device is connected to the host device and in operation. The standby and usage modes can each have a different power consumption level, and as a result, the different standby and usage counters are used to record values that are proportional to the energy consumption of the monitoring device. The standby and usage counters can be incremented (or decremented) based on elapsed time and/or other available parameters of interest (e.g., ambient temperature, circuitry loads, etc.) to account for the battery discharging over time. The values of the standby and usage counters are mapped by the host device into normalized battery energy values whenever the battery is operating in the second charge-voltage region 5302.

Next, as shown in block 5404, the battery is operating in the third charge-voltage region (e.g., region 5304 in FIG. 53), and similar to the first charge-voltage region, the battery charge is calculated by a function that maps the battery voltage and other parameters of interest (i.e., voltage, temperature, circuitry loads, etc.). The battery remains in the third charge-voltage region until the battery discharges completely. When the battery is discharged fully, the host device can produce warnings and notification that the charge on the battery in the monitoring device is low or empty.

FIG. 55 depicts a flowchart of an alternate example method of estimating the battery life of the battery in the monitoring device. In FIG. 55, the first and the second charge-voltage regions (e.g., regions 5300 and 5302 in FIG. 53) are combine into a combined charge-voltage region (5306 in FIG. 53). The combined region reduces the number of charge-voltage regions and simplifies the operations of estimating the battery life. Typically, the battery is in the first charge-voltage region (e.g., 5300 in FIG. 53) for a shorter period of time than in the second charge-voltage region (e.g., 5302). Thus, combining the first and the second charge-voltage regions into one combined region does not affect significantly the accuracy of the estimations.

Initially, as shown in block 5500, the battery in the monitoring device is in the combined (e.g., first+second) charge-voltage region and the battery charge is estimated using the one or more counters that are implemented in the monitoring device. The counter value(s) are transmitted to the host device for processing. The host device decreases the battery charge over time as a function of the counter value(s). In a non-limiting embodiment, the counter(s) are implemented as the usage and the standby counters. The values of the standby and usage counters are mapped by the host device into normalized battery energy values whenever the battery is operating in the combined region.

Next, as shown in block 5404, the battery is operating in the third charge-voltage region (e.g., 5304 in FIG. 53) and the battery charge is calculated by a function that maps the battery voltage and other parameters of interest (i.e., voltage, temperature, circuitry loads, etc.) into normalized battery energy values. The battery remains in the third charge-voltage region until the battery discharges completely. When the battery is discharged fully, the host device can produce warnings and notification that the charge on the battery in the monitoring device is low or empty.

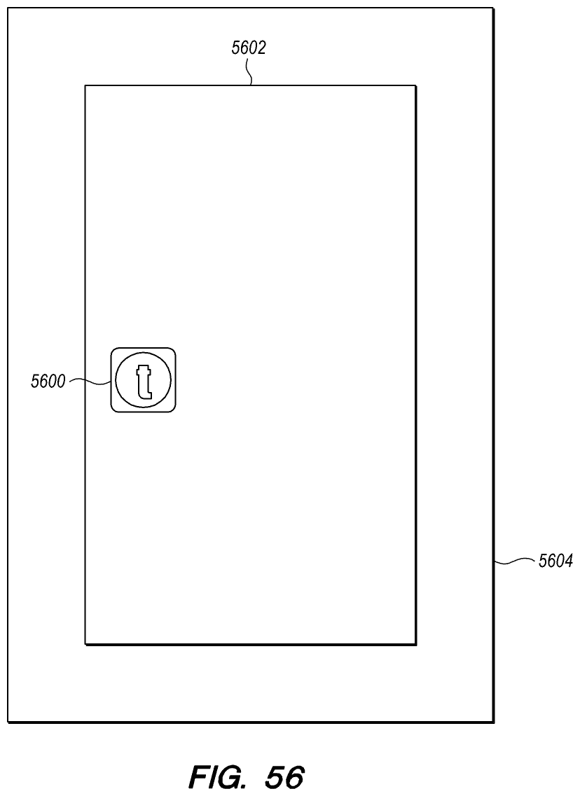

FIG. 56 illustrates a graphic representation of the host application installed in a host device that works with a monitoring device. In one embodiment, the host application is downloaded from an online application program site or the website of the provider of the host application and installed in the host device. The graphic representation 5600 is an icon that represents the host application. The graphic representation 5600 can be displayed on a user interface screen 5602 on a display 5604 of the host device. For example, the monitoring device is an OXXIOM pulse oximeter and the host application is the OXXIOM Application by True Wearables, Inc. Example host devices include, but are not limited to, a smart watch, a desktop computer, a laptop computer, and a cell phone or other mobile computing device.

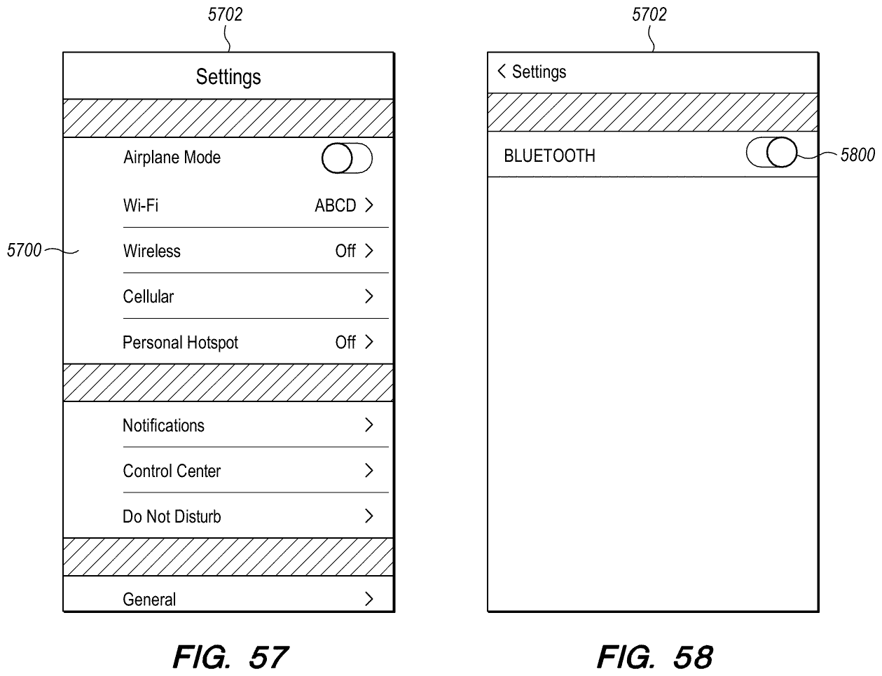

FIGS. 57-58 depict example operations to start the host application in a host device. As shown in FIGS. 57 and 58, a wireless communication device in the host device (e.g., 116 in FIG. 1) is enabled via a setting 5700 in a settings user interface screen 5702 on the host device. For example, in one embodiment, the BLUETOOTH wireless communication device is enabled using the switch 5800 (see FIG. 58).

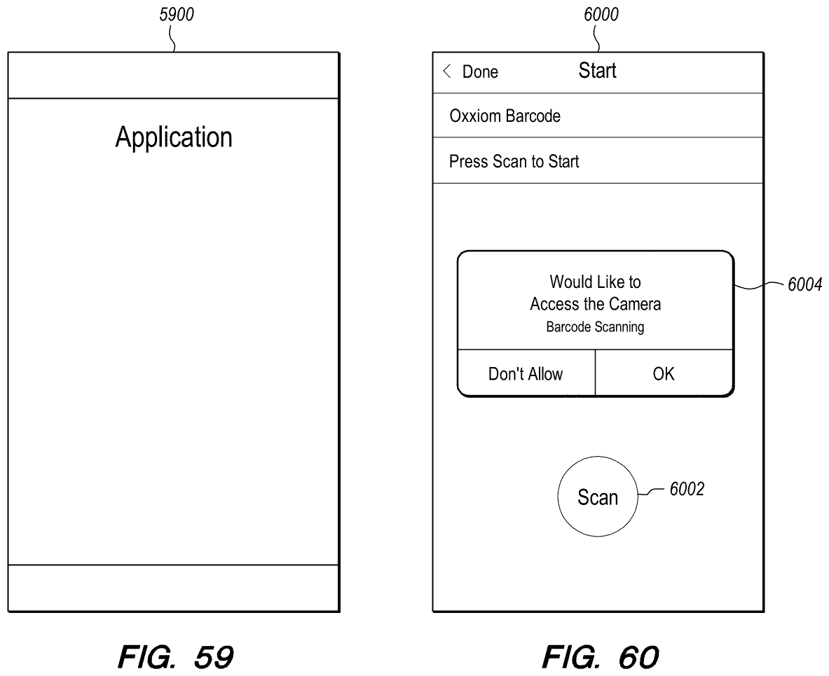

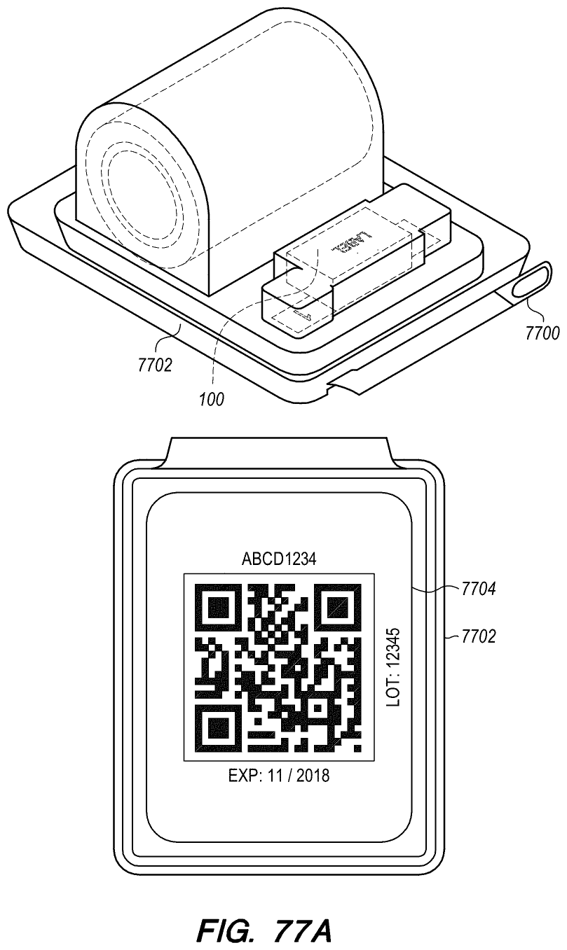

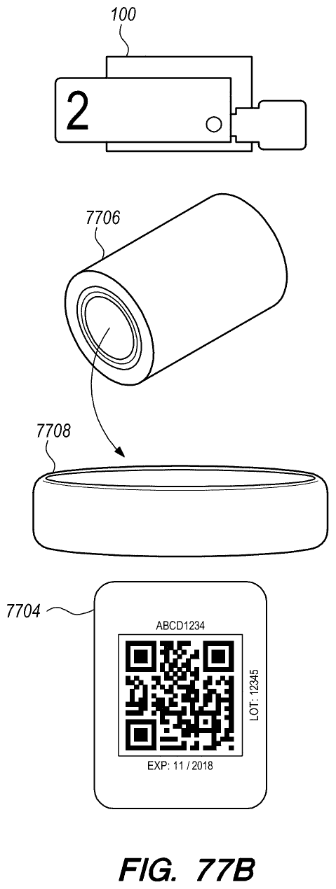

FIGS. 59-61 illustrate example operations to connect the monitoring device to the host device. As shown in FIG. 59, the user launches the host application that interacts with the monitoring device, which can optionally cause a startup screen 5900 with information about the host application to be displayed (e.g., information such as the application program name, manufacturer's name, copyright, etc.). A screen 6000 with a selectable element 6002 is displayed (FIG. 60). In FIG. 60, the selectable element 6002 is a button labeled “scan”, although other embodiments are not limited to this implementation. When the user launches the host application for the first time (e.g., the host application has not been used before), in some embodiments the host application is locked into the screen 6000 until the user scans a valid barcode from a product label (e.g., product label 7704 in FIG. 77A).

When the user selects the selectable element 6002 (e.g., the scan button), an optional notification 6004 is presented stating the host application is requesting access to an imaging device (e.g., the camera) on the host device (FIG. 60). The imaging device is used to scan the barcode on the product label. FIG. 61 illustrates an example screen 6100 displaying an image 6102 of the scanned product label. In some embodiments, an indicator (e.g., a colored square; not shown) is produced on the screen of the host device around the image 6102 of the scanned barcode. Additionally or alternatively, an audio sound, such as a beep, is produced to indicate the barcode has been scanned and is valid. If the scanned barcode is not valid, then a different indicator or an indicator having a different color (e.g., a red square) is displayed adjacent to or around the invalid barcode. In some instances, a different audio sound (e.g., a bell) is produced in addition to, or as an alternative to, the indicator. The host device produces haptic feedback (e.g., vibrations produced by a haptic transducer) to indicate the barcode is invalid in other embodiments.

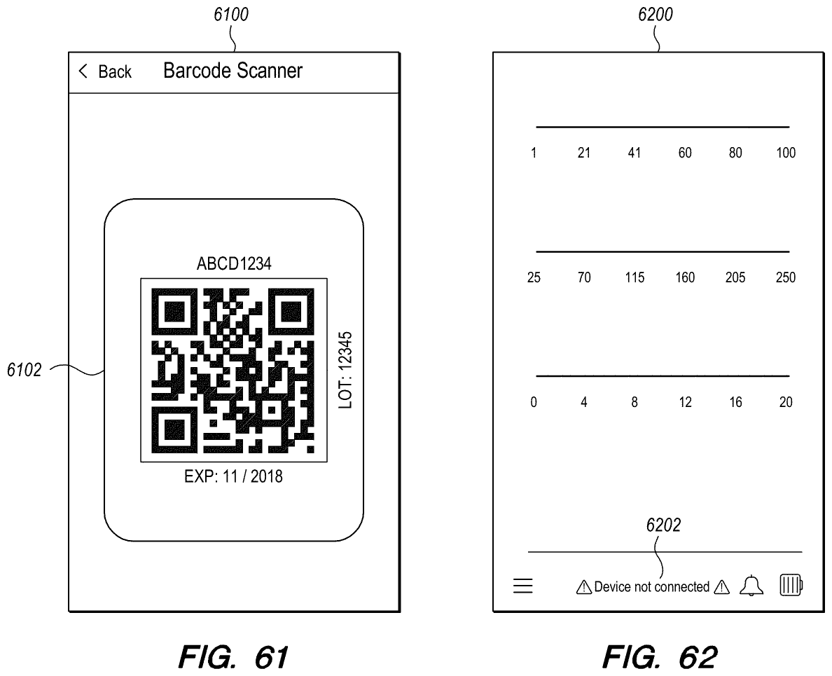

FIGS. 62-64 depict an example user interface screen that may be displayed at a host device. The user interface screen 6200 can be presented in a portrait mode or in a landscape mode. In FIG. 62, the user interface screen 6200 can display (or cause to be displayed) a notification 6202 that the monitoring device is not connected to the host device (e.g., the host application). The measurements and the waveforms are presented when the monitoring device is operating and connected to the host device.

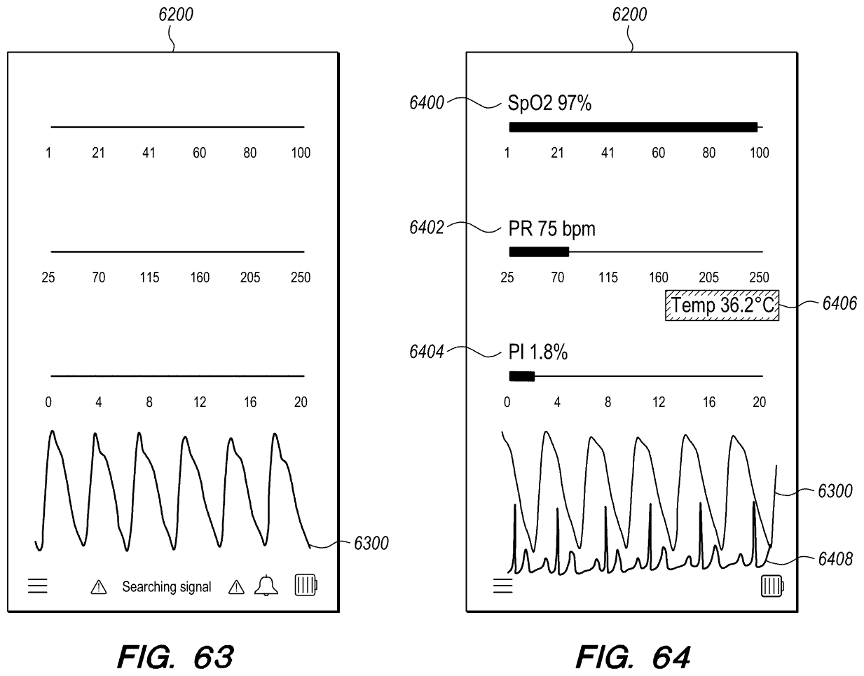

As shown in FIG. 63, a waveform 6300 (photoplethysmograph) is displayed in the user interface screen 6200. In FIG. 64, the SpO2, PR, and PI measurement gauges 6400, 6402, 6404, respectively, the body core temperature gauge 6406, and the waveform 6300 (photoplethysmographs) are displayed in the user interface screen 6200. When the monitoring device includes the first and the second electrical contact sensors (e.g., 707, 708 in FIG. 7D), and the user touches the second electrical contact sensor with a body part (e.g., a finger), a spot-check ECG waveform 6408 is displayed in the User interface screen 6200. The spot-check ECG waveform 6408 may be updated in real-time on the User interface screen 6200 until the body part (e.g., the finger) is no longer in contact with the second electrical contact sensor. When the user removes the body part from the second electrical contact sensor, the ECG waveform 6408 may be frozen on the User interface screen 6200 and/or may disappear from the User interface screen 6200. In some embodiments, the user can set a setting for the ECG waveform 6408 to remain frozen or to be removed.

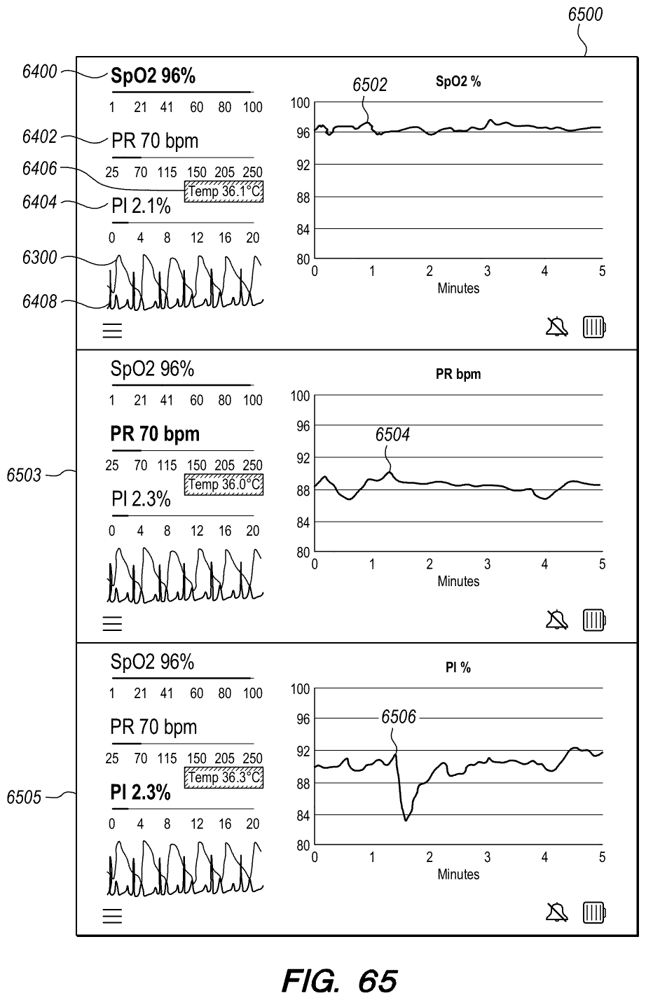

FIG. 65 depicts several user interface screen that can be displayed on a host device. The user interface screen 6500 displays the SpO2 trend 6502 in addition to the gauges 6400, 6402, 6404, 6406 and the waveforms 6300, 6408 shown in FIG. 64. The user interface screen 6503 displays the PR trend 6504 in addition to the gauges and the waveforms shown in FIG. 64. The user interface screen 6505 displays the PI trend 6506 in addition to the gauges and the waveforms shown in FIG. 64. Fewer gauges and/or waveforms can be presented in the user interface screens 6500, 6503, 6505 in other embodiments. In some embodiments, the user can select which of the trends 6502, 6504, 6506, gauges 6400, 6402, 6404, 6406 and waveforms 6300, 6408 to display on a user interface screen. For example, the user can touch a particular gauge on the user interface screen 6200 to display a corresponding trend in the user interface screen 6500. Additionally or alternatively, a user can select which of the trends 6502, 6504, 6506, gauges 6400, 6402, 6404, 6406 and waveforms 6300, 6408 to display using a settings user interface screen.

FIGS. 66A-66B depict a workflow of a host device sharing the data received from the monitoring device. FIG. 66A illustrates a screen 6600 that is displayed by the host application on the host device (e.g., host device 105 in FIG. 1). In one embodiment, the host application can share reports 6602, trends 6604, and waveforms 6606 with another computing device based on a selection of the selectable element 6608. In FIG. 66A, the selectable element 6608 is a button labeled “Share”, although other embodiments are not limited to this implementation.

The reports 6602 can be a combination of analyses and charts shared as a file (i.e., PDF, PS, HTML, etc.) or designed to be interactive with the user. The trends 6604 may be a spreadsheet or database (interactive or shared as a file) with data trend measurements over time. The waveforms 6606 can be a database or spreadsheet file that logs the waveforms collected, or the raw data from where the waveforms and other variables of interest can be extracted.

In FIG. 66B, a screen 6610 depicts example data sharing methods, such as Wi-Fi, Transfer 6612, Message 6614, Mail 6616, Cloud 6618, Print 6620, etc. One example of a Transfer 6612 data sharing method is AIRDROP by Apple. Available data sharing methods may vary depending on the configuration of the host device and/or the type(s) of data being shared.

In one embodiment, after the monitoring device is connected to the host device and the host device is displaying one or more measurement gauges, one or more waveforms and/or one or more trends (e.g., SpO2, PR, PA, and/or PI, and temperature measurements, photoplethysmograph and/or ECG waveforms) received over a given period of time, the user can share the data via the Reports 6602. FIGS. 66C-66L illustrate example reports with analyses from data collected over a given period of time (e.g., overnight or 569 minutes) by a user wearing the monitoring device. The example reports including particular values, and other embodiments can present different values. The example reports include, but are not limited to:

1. SpO2 Measurements Over Time—The report 6622 in FIG. 66C shows an example of a SpO2 trend. The report 6622 can be created by the host application on the host device. Normal SpO2 levels are typically between 94% and 100%. However, during sleep, exercise, or situations of high stress, or in high altitude places, the SpO2 readings may reach lower values (i.e., lower than 94%). The example report 6622 includes an optional footer 6624 with information about the data collected (i.e., identification 6626 of monitoring device used in the data collection, the period of data collection 6628, and data and time the report was created 6630). Although not shown in FIGS. 66D-66L, the embodiments shown in FIGS. 66D-66L can also include the footer 6624.

2. PR Measurements Over Time—The report 6632 in FIG. 66D shows an example of PR trend. The report 6632 can be created by the host application on the host device. Normal resting PR for adults typically ranges from 60 bpm 6634 to 100 bpm 6636. However, during sleep, exercise, or situations of high stress, or in high altitude places, PR readings may reach higher values 6638. A report having different values can be produced in other embodiments.

3. PI Measurements Over Time—The report 6640 in FIG. 66E shows an example of PI trend. The example report 6640 can be created by the host application on the host device. PI values typically range from 0.02% (very weak pulsatile signal) 6642 to 20% (very strong pulsatile signal). Extreme PI values 6644 may indicate a situation of discomfort (e.g., cold, hot, stress, etc.). A report having different values can be produced in other embodiments.

4. SpO2 Distribution—The report 6646 in FIG. 66F shows an example of SpO2 distribution. The example report 6646 can be created by the host application on the host device. As shown in the example report 6646, in 87.4% of the time 6648 (i.e., 497 minutes), the SpO2 values were within 94% and 100%, and in 12% of the time 6650 (i.e., 68 minutes), the SpO2 values were between 88% and 93%. A report having different values can be produced in other embodiments.

5. PI Distribution—The report 6652 in FIG. 66G shows an example of PI distribution. The example report 6652 can be created by the host application on the host device. As shown in the example report 6652, in 58.1% of the time 6654 (i.e., 331 minutes), the PR values were within 60 bpm and 80 bpm, and in 39.1% of the time 6656 (i.e., 222 minutes), the PR values were between 50 bpm and 59 bpm. A report having different values can be produced in other embodiments.

6. PI Distribution—The report 6658 in FIG. 66H shows an example of PI distribution. The example report 6658 can be created by the host application on the host device. As shown in the example report, in 64.2% of the time 6660 (i.e., 365 minutes), the PI values were within 0.1% and 0.5%, and in 35% of the time 6662 (i.e., 199 minutes), the PI values were between 0.01% and 0.1%. A report having different values can be produced in other embodiments.

7. SpO2 Desaturations per Hour—The report 6664 in FIG. 66I shows the number of desaturations per hour from baseline. The example report 6664 can be created by the host application on the host device. According to the example report 6664, the user had 1.9 desaturations per hour 6666 with an amplitude greater than 4%. A healthy person will typically have less than five or so desaturations per hour with an amplitude greater than 4%. The values of the desaturations per hour may increase during exercise, in situations of high stress, or at high altitude locations. A report having different values can be produced in other embodiments.

8. Cumulative Time Percentage with SpO2 Less than Threshold—The report 6668 in FIG. 66J shows the cumulative time percentages with SpO2 less than threshold. The example report 6668 can be created by the host application on the host device. According to the example report 6668, in 1.5% (i.e., 8.5 minutes) of the time, the user's SpO2 values 6670 were less than 90%. A healthy person will typically have an SpO2 less than 90% for a small time percentage. The time percentage may increase during exercise, in situations of high stress, or at high altitude locations. A report having different values can be produced in other embodiments.

9. PR Volatility Distribution—The PR Volatility expresses the heart rate short-term variability. In one embodiment, the PR volatility is calculated following the example operations described in conjunction with FIG. 35, where the measurement data in block 3502 is a series of instantaneous and sequential measurements of PR and the corresponding distribution is one of the statistics computed in block 3508. Typically, for healthy adults, higher PR Volatility values are better. Increasing the PR Volatility trends are positive and indicative of positive adaptation and/or increase in fitness. The report 6672 in FIG. 66K shows an example of PR Volatility distribution. The report 6672 can be created by the host application on the host device. As shown in the example report, over 50% of the time 6674 (i.e., 286 minutes), the PR Volatility values were between 0.46 BPM and 1 BPM. A report having different values can be produced in other embodiments.

10. PI Log-Volatility Distribution—The PI Log-Volatility expresses the perfusion short-term variability. In one embodiment, the PI Log-Volatility is calculated following the example operations described in the embodiment depicted in FIG. 35, where the measurement data in block 3500 is a series of instantaneous and sequential measurements of PI and the corresponding distribution is one of the statistics computed in block 3508. Typically, for healthy adults, lower PI Log-Volatility values are better. Decreasing PI Log-Volatility trends are positive and indicative of positive adaptation and/or decrease in overall stress levels. The report 6676 in FIG. 66L shows an example of PI Log-Volatility distribution. The report 6676 can be created by the host application on the host device. As shown in the example report, over 52% of the time 6678 (i.e., 286 minutes), the PI Log-Volatility values were within 9.4% and 15%. A report having different values can be produced in other embodiments.

Additionally or alternatively, a user may share trends in SpO2, PR, PA, PI, and/or temperature measurements in a file at any time through the host application on the host device (e.g., Trends 6604 in FIG. 66A). One example of a file is a Comma-Separated Values (CSV) file. The file can be opened and displayed by the host device directly or by most presentation or spreadsheet software, such as EXCEL and NUMBERS. In FIG. 66M, the example CSV file 6680 contains 6 columns. Other embodiments can display different values, a different number of rows, and/or a different number of columns. The illustrated columns in the example file 6680 are:

1. Date/Time—The column 6682 stores the date and the time of each measurement taken. Measurements can be stored once a second (for 12 h trend storage), once every 2 seconds (for 24 h trend storage), once every 3 seconds (for 36 h trend storage), and once every 4 seconds (for 48 h trend storage). Other embodiments can store the measurements at different times.

2. Product Label Barcode. The column 6684 stores the 8-digit hexadecimal number that identifies the monitoring device that is used at the corresponding time and date.

3. SpO2(%)—The column 6686 records the SpO2 measurements.

4. PR (BPM)—The column 6688 stores the PR measurements.

5. PI (%)—The column 6690 records the PI measurements.

6. Temperature (C)—The column 6692 stores the core body (or skin) temperature measurements.

In some instances, the user may share waveforms (e.g., using Waveforms 6606 in FIG. 66A) through the host application on the host device. In one embodiment, the waveforms are shared in the form of raw data, auxiliary variables, and parameters collected by the monitoring device (up to a certain number of hours) and stored in the host device. Other embodiments can share the waveforms in a different form. The data can be stored into a database file (e.g., WaveformsDB.db) using an encoded data format. The data can be used for in-depth technical analysis of potential problems detected by users, or for off-line computations of other parameters of interest, such as heart rate variability, respiration rate, etc., or to recalculate off-line the values of SpO2, PR, PI, temperature, photoplethysmographs, and ECG waveforms using custom algorithms that may be more suitable for a particular off-line application.

FIG. 66N depicts an example format of a file 6694. The file 6694 includes three columns: Index 6696, Time Stamp 6697, and Data 6698. The Index column 6696 indexes the rows of the file 6694. The Time Stamp column 6697 has an example format YYYY-MM-DD hh:mm:ss.uuu, where YYYY-MM-DD is the year, month, and day, hh:mm:ss.uuu is the time, in hours, minutes, seconds, and milliseconds, and is the date and time the measurement data was saved to the file 6694. Each time stamp value is an approximation of the time the measurement data was actually collected, since the measurement data is saved by the host device to the file 6694 in real-time. The time stamp values can be used to synchronize in time the measurement data stored in the file 6694 with other measurement systems so as to perform analyses where such a time synchronization is used.

The Data column 6698 is the actual data saved to the file 6694. In FIG. 66N, the Data column 6698 has three arrays of example data separated by square brackets ([first data array][second data array] [third data array]. Each of the three arrays has forty samples per row. The samples of each data array are collected synchronously by the monitoring device at every twenty milliseconds (50 Hz sampling frequency) and transmitted wirelessly to the host device for data storage. In one embodiment, the host device stores the data in real-time to temporary buffers to reduce latency requirements and then saves the data in the buffers to the file 6694 every N seconds, where N is a whole or a fractional number greater than zero. For example, the data can be stored to the file 6694 every three-fourths of a second. The data arrays contain the raw waveform data from the one or more light sources in the optical sensor (e.g., red, infrared, and green light sources), and the monitoring device hardware diagnostics variables and parameters, such as, for example, the battery voltage, SoC temperature, current settings for the one or more light sources of the optical sensor, gains for the optical, temperature, and ECG frontends, SoC usage time counters, SoC standby timer counters, ambient light intensity detected by the optical frontend, device identification number, and the like.

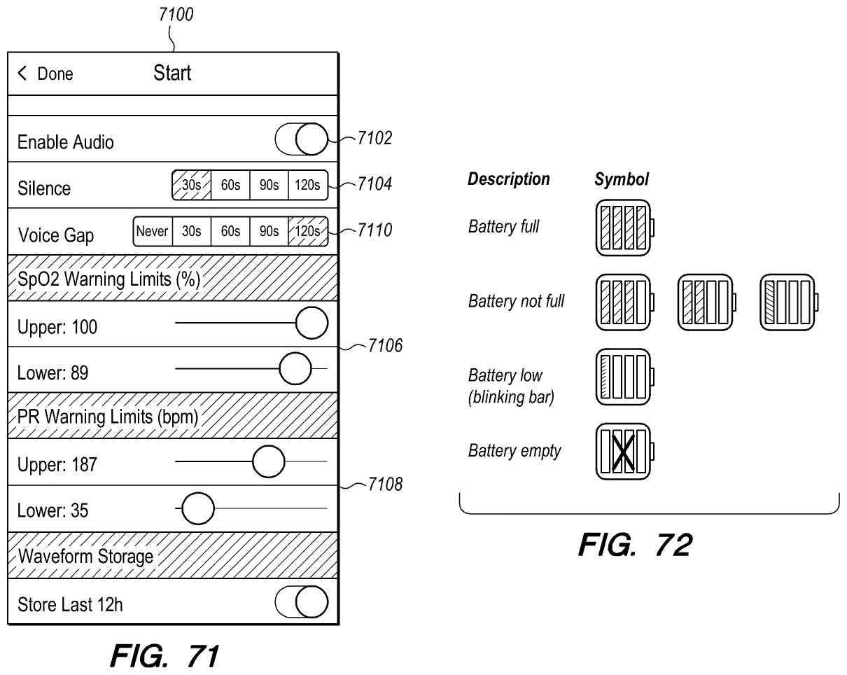

In some embodiments, to increase the battery life of the host device (e.g., a smart watch or smart phone), the waveforms (e.g., Waveforms 6606 in FIG. 66A) may not be stored on the host device. In this situation, the user may disable the storage of the waveforms. In one embodiment, the storage of the waveforms can be disabled using a user settings interface (see e.g., FIG. 71).

There are situations where it is desirable to take screenshots of the measurement gauges and the trend data for the user's records or for sharing with third parties. Typically, the host device offers this functionality as a standard feature. For instance, in the case of an iOS device from Apple, Inc., the user presses and holds the top (or side) button and one of the volume buttons concurrently to capture a screenshot of the display. The user interface screenshot can be displayed on the display of the iOS device and/or shared by the iOS device.

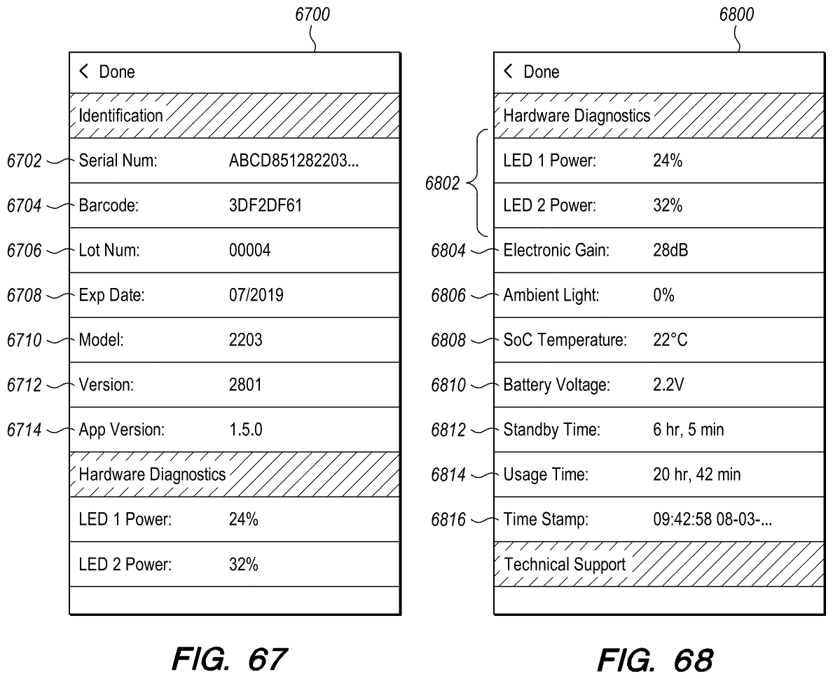

FIG. 67 illustrates an example screen of identification parameters produced by the host application on the host device. The example screen 6700 can include one or more of the following identification parameters:

1. Serial Num 6702—When the monitoring device is connected to the host device, the host device can display an M-digit hexadecimal number in the screen 6700, where M is a number greater than zero. For example, the M-digit hexadecimal number is a 16-digit hexadecimal number. In one embodiment, the M-digit hexadecimal number is the serial number of the monitoring device.

2. Barcode 6704—When the monitoring device is connected to the host device, the host device may display a P-digit hexadecimal number that identifies the monitoring device in the screen 6700, where P is a number greater than zero. For example, the P-digit hexadecimal number is the barcode from the product label (e.g., product label 7704 in FIG. 77A). Additionally, the host device can display the barcode in a start screen of the host application and/or in the file that is shared by the host device (e.g., data file 6694 in FIG. 66N). In a non-limiting example, the P-digit hexadecimal number is an 8-digit hexadecimal number.

3. Lot Num 6706—When the monitoring device is connected to the host device, the host device can display the manufacturing lot number of the monitoring device in the screen 6700.

4. Exp Date 6708—When the monitoring device is connected to the host device, the host device may display a date after which the monitoring device should not be sold or used in the screen 6700.

5. Model 6710—When the monitoring device is connected to the host device, the host device can display a Q-digit hexadecimal number of the model number of the monitoring device in the screen 6700. Q is a number greater than zero. For example, the Q-digit hexadecimal number is a 4-digit hexadecimal number.

6. Version 6712—When the monitoring device is connected to the host device, the host device may display an R-digit hexadecimal number of the version number of the monitoring device. For example, the R-digit hexadecimal number is a 4-digit hexadecimal number.

7. App Version 6714—When the monitoring device is connected to the host device, the host device can display an S-digit number of the software version. In a non-limiting example, the S-digit number is a three digit number.

FIG. 68 depicts an example screen of hardware diagnostics parameters produced by the host application. The example screen 6800 can include one or more of the following parameters:

1. LED Power 6802—The LED power parameter in the screen 6800 displays the power levels (reported at a Time Stamp) for each light source of the optical sensor in the monitoring device. For example, the power levels of light source one (LED1) and light source two (LED2) are presented. The power level of each light source varies between 0 and 100%.

2. Electronic Gain 6804—The electronic gain parameter in the screen 6800 represents the analog frontend electronic gain (reported at Time Stamp) of the monitoring device. The electronic gain varies between 0 and 40 dB.

3. Ambient Light 6806—The ambient light parameter in the screen 6800 represents the ambient light intensity detected by the one or more photodetectors in the optical sensor in the monitoring device (reported at Time Stamp). It varies between 0 and 100%.

4. SoC Temperature 6808—The SoC parameter in the screen 6800 represents the System on Chip (SoC) temperature in the monitoring device. The SoC temperature can be displayed in Celsius (reported at Time Stamp) or Fahrenheit.

5. Battery Voltage 6810—The battery voltage parameter in the screen 6800 represents the battery voltage in Volts (reported at Time Stamp) of the battery in the monitoring device.

6. Standby Time 6812—The standby time parameter in the screen 6800 represents the amount of time the monitoring device has been activate and disconnected from the host device (reported at Time Stamp).

7. Usage Time 6814—The usage time parameter in the screen 6800 represents the amount of time the monitoring device has been activate and connected to the host device (reported at Time Stamp).

8. Time Stamp 6816—The time stamp parameter in the screen 6800 is the date and time when the one or more hardware diagnostic parameters are reported.

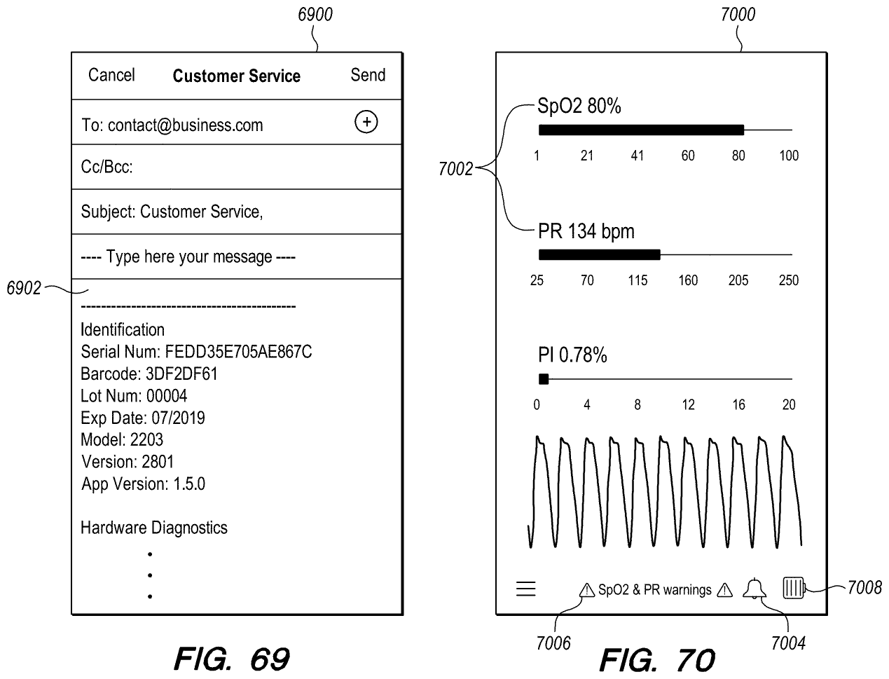

FIG. 69 depicts an example embodiment on sharing the measurement data with a technical support team of the monitoring device. When the user wants to share the measurement data, the host device displays the screen 6900. In this example embodiment, the user shares the Identification and Hardware Diagnostics parameters and variables via an electronic mail message, although other embodiments are not limited to electronic mail. The user can also add comments regarding the specific problem or issue found in section 6902. Several other methods can be used to share such information with the manufacturer. In addition, depending on the issue, the user may elect to share the file of the waveform data (e.g., file 6694 in FIG. 66N) with the technical support team for in-depth technical analysis of a potential problem detected by the user.

In some applications, the system that includes the monitoring device, the host device, and the host application can include a built-in an alarm/warning system to keep the user (e.g., a patient) informed and/or safe. FIG. 70 depicts an example embodiment of an alarm/warning system. FIG. 70 illustrates an example screen 7000, where the measurement gauges 7002 can blink in a color (e.g., red) whenever a measurement value crosses an upper or lower preset limit, or whenever the monitoring device stops providing measurement data. Additionally or alternatively, an audible alarm/warning 7004 (e.g., voice, sound) is produced and/or a written notification 7006 is displayed whenever the measurement value crosses an upper or lower preset limit, or whenever the monitoring device stops providing measurement data.

Example alarm/warning are now described. The example alarms are listed in descending order of priority, although other embodiments are not limited to the illustrated priority order.

1. Monitoring device is not connected to host device—This alarm is issued whenever the monitoring device is not connected to host device. A visual warning can include causing one or more gauges to blink and/or be displayed in a dashed line. An audible warning may include a beep and/or an audible notification such as “Device not connected.” This alarm/warning has the highest priority in one embodiment, given that the warning indicates the monitoring device is not connected to the host device, may be out-of-range, defective, or turned off.

2. Battery is empty—The warning is issued whenever the battery in the monitoring device is low or empty. A visual warning can include causing a graphic representation (e.g., battery icon 7008) to blink and/or be displayed in a dashed line. An audible warning may include a beep and/or an audible notification such as “battery is empty or low.” This alarm/warning has the second highest priority in one embodiment. In some embodiments, the measurement gauges are no longer displayed on the host device when the battery on the monitoring device is low. When the battery on the monitoring device is empty, the measurements are not displayed on the host device.

3. Monitoring device is searching for a valid signal—The warning is issued whenever the monitoring device is connected to the host device but measurements are not yet displayed on the host device because the monitoring device is either not correctly placed on subject, or the data collected is within a transient time where there is not enough collected data to produce a reliable set of measurements. This alarm/warning has the third highest priority in one embodiment. A visual warning can include causing one or more measurement gauges to blink and/or be displayed in a dashed line. An audible warning may include a beep and/or an audible notification such as “searching for signal.”

4. Wireless connection with the host device is not reliable—This warning is issued whenever the monitoring device has an unreliable wireless or wired connection with the host device. In one embodiment, a visual warning causes all of the measurement gauges blink in a color, such as red. An audible warning may include a beep and/or an audible notification such as “poor connection.”

5. SpO2 measurement has crossed either upper or lower limits—This alarm is issued whenever the SpO2 value is outside the preset normal limits. In one embodiment, a visual warning causes the SpO2 measurement gauge to blink in a color, such as red and/or be displayed in a different color. An audible warning may include a beep and/or an audible notification such as “saturation warning.” In some instances, the upper and/or lower limits of the SpO2 measurements can be defined by the user in a settings screen, as will be described in more detail in conjunction with FIG. 71.

6. PR measurement has crossed either upper or lower limits—This alarm is issued whenever the PR value is outside the preset normal limits. In one embodiment, a visual warning causes the PR measurement gauge to blink in a color, such as red and/or be displayed in a different color. An audible warning may include a beep and/or an audible notification such as “pulse rate warning.” In some instances, the upper and/or lower limits of the PR measurements can be defined by the user in a settings screen, as will be described in more detail in conjunction with FIG. 71.

7. SpO2 and PR measurements have crossed either upper or lower limits—This alarm is issued whenever both the SpO2 and the PR values are outside the preset normal limits. In one embodiment, a visual warning causes the SpO2 and PR measurement gauges to blink in a color, such as red and/or be displayed in a different color. An audible warning may include a beep and/or an audible notification such as “saturation and pulse rate warning.”

8. Battery in host device is low—This warning is issued whenever the battery in the host device is low or empty. In one embodiment, a visual warning causes a message to be displayed, such as “charge host device.” An audible warning may include a beep and/or an audible notification such as “charge host device.” In a non-limiting example, the warning is issued when the charge on the battery in the host device is less than 23%, although other embodiments are not limited to this percentage value.

FIG. 71 depicts an example settings screen of the host application executing on the host device. The screen 7100 enables the user to set the alarm/warning limits and audio settings. The selectable control 7102 (e.g., switch) enables/disables audible warnings. The selectable options 7104 enables the user to set a silence time interval (i.e., 30, 60, 90, or 120 seconds). The silence time interval is used to silence a warning for a given period of time and will be activated when an alarm/warning is active and the user touches one of the measurement gauges (i.e., SpO2, PR, PI, or temperature). The warning/alarm will be paused during the silence time interval and then resume automatically after the silence time interval expires. The selectable elements 7106 and 7108 (e.g., slide control elements) enable the user to set the alarm limits for particular measurement gauges. For example, in the illustrated embodiment, the alarm limits can be set for the SpO2, and PR measurement gauges and the selected values are displayed for each upper and lower limit. In one embodiment, the SpO2 and PR alarm/warning limits are initially set to their default values.

In FIG. 71, the selectable option 7110 (labeled Voice Gap) enables the user to define the periodicity of voice-based measurements. In the example embodiment depicted in FIG. 71, the periodicity options are every 30, 60, 120 seconds or “never”. For instance, if the user selects the 30-second option, then every 30 seconds the host device will speak to the user through its speech synthesizer, informing the current measurement values. The host device through its audio system, or through an audio system wirelessly or wired connected to the host device (i.e., headphones, speakers, car sound system, etc.), may speak to the user informing, for example, that “SpO2 is one hundred percent, PR is sixty five beats per minute, PI and/or PA is one percent, and Temperature is thirty six Celsius”. This functionality enables users to shut-off the screen of the host device, or be driving, exercising, etc. while wearing the monitoring device and not having direct access to the display screen on the host device and still able to hear their current measurement values periodically.

If the user does not want to hear the measurement values, then the option “never” in the selectable option 7110 disables the voice-based measurements. In some embodiments, it is possible to set the host device to provide only voice-based measurements whenever one or some of the measurement values change more than a certain threshold value (absolute or relative). For instance, the host device can audibly output the current measurements whenever the value of SpO2 changes more than +/−2 points, or the value of PR changes more than +/−5 bpm, or the value of PI changes more than +/−10%, or the value of temperature changes more than +/−0.3 Celsius, from their last spoken measurement values. Spoken measurements can also be triggered depending on how the rate of change (first derivative), or the rate of change of the rate of change (second derivative), etc. of a particular measurement variable varies over time.

Additionally or alternatively, the user is informed about the measurement tendency (or rate of change) over time since the last spoken measurement. For example, the host device could speak to the user messages such as “SpO2 is ninetyfour percent and decreasing”, “PR is one hundred beats per minute and stable”, “PI and/or PA is zero point two percent and increasing”, “Temperature is thirty six Celsius and stable”. The rate of change can also be specified numerically or qualitatively, such as “increasing slowly”, “increasingfast”, “decreasing slowly”, or “decreasingfast”, etc. The type and content of the measurement message, the triggering rule, and the information contained therein depends on the monitoring application and its particular requirements.

In one aspect, the host device is restricted to the application (a single application) with the hardware buttons and access to the host application menu are disabled and protected by a password (or some other form of authentication) to prevent unauthorized users from changing the settings or disabling the application program. In one example, the host application is compliant with the iOS Guided Access mode when the host device is an iOS device. The Guided Access mode temporarily restricts the iOS device to a single application and lets the user control which features of the application are available. The default behavior of the host application during a Guided Access section can be: (1) The application termination is disabled; (2) The application menu is disabled; (3) Portrait and landscape views are enabled; (4) Hardware buttons are disabled (e.g., volume, sleep/wake, etc.); and (5) The user can silence audible warnings (if enabled and active) for a period of time by taping on any measurement gauge. However, Audible warnings will resume automatically after the silence duration (i.e., 30, 60, 90, or 120 seconds) expires. If required, audible warnings can be permanently disabled before starting a Guided Access section.

The alarm/warning system described in FIGS. 70 and 71 can also relay information about an active alarm/warning to a third party via the host device. The host device can send notifications (wirelessly or wired) directly to third parties. Example third parties include, but are not limited to nurses, medical doctors, and caretakers. Additionally or alternatively, the notifications are sent to a central alarm/warning system that in turn, relays the notifications to the appropriate receiver.

As discussed previously, the battery in the monitoring device is non-rechargeable battery. Information about the device usage and/or the charge on the battery are provided to the user at select times or continuously to enable the user to predict when monitoring device is to be replaced. The battery icon 7008 shown in FIG. 70 is shown in detail in FIG. 72 (see also FIG. 48). FIG. 72 depicts a battery icon and example battery states (e.g., battery full, battery not full, battery low, and battery empty).

In some embodiments, the host device can compute SpO2 values higher than 100%, and/or other extended measurements of interest. FIG. 73 illustrates a flowchart of an example method of providing SpO2 measurement values that exceed one hundred percent. The process is described with reference to SpO2 values, but the method can be used with other parameters or measurements of interest. Non-limiting examples of the other parameters or measurements of interest are PR, PI, non-invasive blood glucose, total hemoglobin, carboxyhemoglobin, methemoglobin, body fat, and body hydration levels.

Initially, a determination is made as to whether the “SpO2 Out-of-Range Suppression” is disabled (see 7502 in FIG. 75). If suppression is enabled (e.g., “SpO2 Out-of-Range Suppression” is enabled) the process passes to block 7302 where the computational range(s) for the optimization variables, parameters, and metrics in an optimization problem solver (e.g., optimization problem solver 4108 in FIG. 41C) are set or reduced to non-extended ranges to determine SpO2 values that do not exceed one hundred percent. Next, as shown in block 7304, the allowed search ranges for the current search limits (e.g., current search limits 4174 in FIG. 41H) are set or decreased to non-extended values to be compatible with the settings in the optimization problem solver and the non-extended ranges of the associated optimization metrics, variables, and parameters. Depending on the application, the allowed search ranges can also be changed to reflect absolute (i.e., expressed in a particular unit) and/or relative (i.e., expressed in percentage) specifications.

The non-extended (e.g., not extrapolated) curve(s) that are used to determine the SpO2 values that do not exceed one hundred percent are selected at block 7306. The SpO2 values are then computed and provided to an output device at block 7308. For example, the embodiments shown in FIG. 42 or 43 can be used compute the SpO2 values in a non-extended range (e.g., not over 100%). For instance, any SpO2 values that exceed 100% may be rounded down to 100% and provided to an output device (e.g., a display) as an integer number that is equal to or less than 100%.

Returning to block 7300, when a determination is made that the “SpO2 Out-of-Range Suppression” is disabled (e.g., suppression disabled), the method continues at block 7310 where the computational range(s) for the optimization variables, parameters, and metrics in an optimization problem solver are set or extended to extended ranges to determine the SpO2 values that may exceed one hundred percent. Next, as shown in block 7312, the allowed search ranges for the current search limits (e.g., current search limits 4174 in FIG. 41H) are set or increased to extended values to be compatible with the settings in the optimization problem solver and the non-extended ranges of the associated optimization metrics, variables, and parameters. Depending on the application, the allowed search ranges can also be changed to reflect absolute (i.e., expressed in a particular unit) and/or relative (i.e., expressed in percentage) specifications.

The extended (e.g., extrapolated) curve(s) that are used to determine the SpO2 values that exceed one hundred percent are selected at block 7314. An example extrapolated curve is shown in FIG. 31. The SpO2 values are then computed and provided to an output device at block 7316. The SpO2 values may include one or more SpO2 values that exceed one hundred percent. In a non-limiting example, the SpO2 values that exceed 100% may be represented by a whole number and (optionally) one or more decimal place values (e.g., tenths place value, hundredths place value, etc.).

For example, the embodiments shown in FIG. 42 or 43 can be used compute the SpO2 values in an extended range (e.g., over 100%). Equation 32, as well as other polynomial (i.e., Taylor, Volterra, etc.), transcendental, and/or table-based (i.e., interpolation/extrapolation grid defined by a Voronoi diagram stored as a set of points such as seeds, generators, sites, etc.) representations can also implement extended (extrapolated) and non-extended (non-extrapolated) single-dimensional or multi-dimensional curves that can be used to compute the extended values for SpO2.

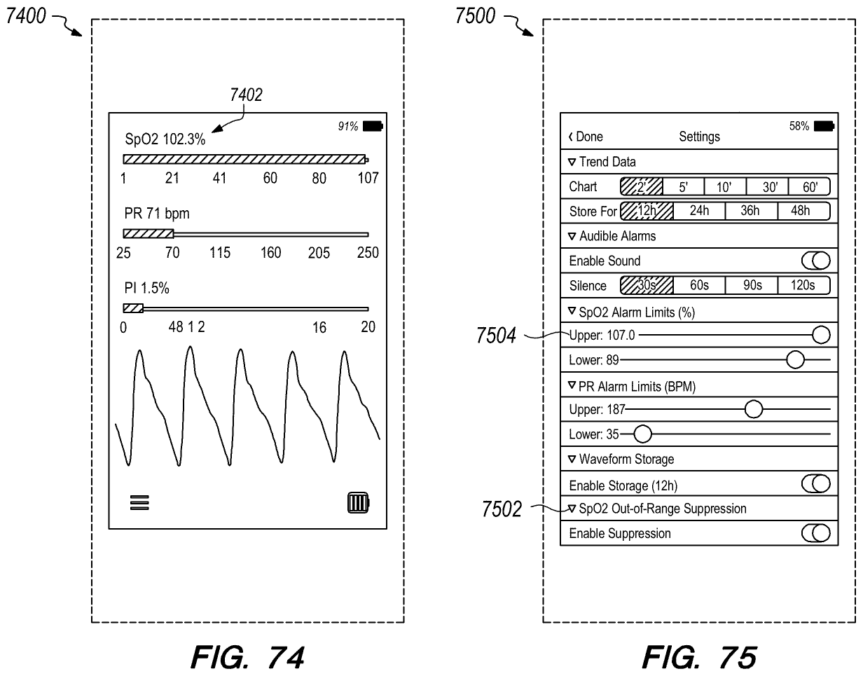

FIG. 74 illustrates a user interface screen of the host application that is suitable to use for displaying one or more measurement values over one hundred percent. The user interface screen 7400 displays a SpO2 numerical value 7402 of 102.3%. Note that a tenth place value can be added to the numerical value 7402 whenever the SpO2 is greater than 100% to give clinicians and users a higher measurement granularity, and to compensate for a smaller dynamic range when the SpO2 values are above 100%. In addition, the overall accuracy of the SpO2 can get better as the SpO2 measurement values increase, and as a result, the added decimal place (tenth place) displays additional and meaningful measurement precision.

FIG. 75 illustrates an example settings user interface screen of the host application that can be to display extended measurement values over one hundred percent. The user interface screen 7500 includes a setting 7502 for “SpO2 out-of-range suppression”. The setting 7502 provides a clinician with the option to display (or not display) SpO2 values above 100%. The dashed line in FIGS. 74 and 75 represents a display operably connected to a host device that displays the user interface screens 7400, 7500.

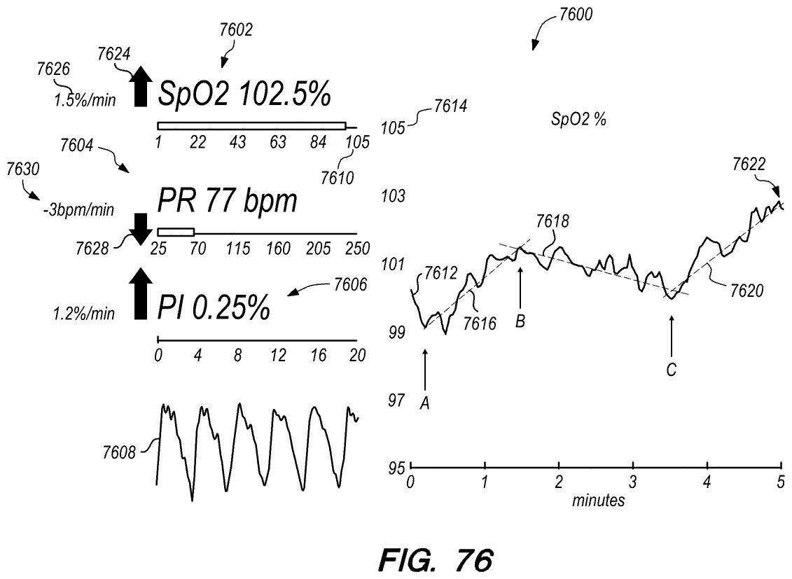

FIG. 76 depicts an example user interface screen of a host application. One section of the user interface screen 7600 (e.g., the left side) displays measurement gauges 7602, 7604, 7606 for SpO2, PR, and PI, respectively, and waveforms 7608 (photoplethysmographs). In other embodiments, a measurement gauge for PA can be displayed in addition to, or as an alternative to, the SpO2, PR, and PI measurement gauges 7602, 7604, 7606. The SpO2 measurement gauge 7602 is capable of displaying SpO2 values from 1% to 105.0%. In the illustrated embodiment, the SpO2 scale upper limit 7610 is 105% whenever the “Sp2 Out-Of-Range Suppression” is disabled (see e.g., 7502 in FIG. 75). The SpO2 scale upper limit 7610 can be defined and adjusted (i.e., increased or decreased) based on the application and/or the subject population (adults, neonatal, animal patients, etc.). In an example embodiment that uses the calibration curve in Equation 12, experimental clinical data has shown that users under oxygen therapy are very unlikely to produce a red-over-infrared ratios (i.e., x values) less than 0.136 (which corresponds to SpO2 values higher than 105%). As a result, the SpO2 scale upper limit 7610 of the SpO2 measurement gauge 7602 is kept at 105%.

Another section of the user interface screen 7600 (e.g., the right side) shows a SpO2 data trend 7612. In one embodiment, other data trends (e.g., PR, PI, etc.) are displayed whenever the user selects the corresponding measurement gauge on a display device (e.g., a touchscreen) in the host device. The SpO2 data trend 7612 has an upper limit scale 7614 of 105% because the SpO2 values reached levels higher than 100%.

The SpO2 data trend 7612 contains three labels (A, B, and C) which tag events/actions that happened during the continuous monitoring of SpO2, PR, and PI (or PA in an alternate embodiment). Label A indicates a point in time (with SpO2 around 99%) where a user was asked to inhale and exhale at a higher frequency so as to hyperventilate. This caused the SpO2 levels of the user to increase at a rate defined by the dashed line 7616. Label B indicates the point in time (with SpO2 around 101.5%) where the user was asked to return to a normal breathing pattern. The SpO2 levels of the user returned gradually to the previous baseline at a rate defined by dashed line 7618. Label C indicates a point in time (with SpO2 around 100.3%) where the user was given oxygen via an oxygen mask. As a result, the SpO2 levels of the user started increasing again at a rate defined by dashed line 7620. The last value 7622 displayed in the SpO2 data trend 7612 is around 102.5% after about 1 and ½ minutes of giving oxygen to the user.

The graphic element 7624 is a gauge with an arrow pointing upwards. An indicator 7626 displays the current SpO2 rate of change (i.e. 1.5%/min), which is defined by the slope of the dashed line 7620. The graphic element 7624 has a length that is proportional to the SpO2 rate of change displayed in the indicator 7626. The graphic element 7624 will point downwards whenever the rate of change is negative, as it is depicted in the graphic element 7628 (i.e.,−3 bpm/min) for the PR rate of change (as indicated by the indicator 7630).

The slopes of the dashed lines 7616, 7618, and 7620 can be calculated in real-time using the estimation technique and/or system disclosed in U.S. patent application Ser. No. ______, entitled “Method and System for Processing Data Streams”. This estimation technique and/or system can be used to efficiently calculate global, optimal, numerical solutions for the slopes that are insensitive to outliers that can be present in the SpO2 data trend due to physiological changes, motion artifacts, interferences, and noise. The dashed lines 7616, 7618, and 7620 represent an affine (or linear) relationship between SpO2 and time. Depending on the application, the dashed lines 7616, 7618, and 7620 can be replaced with second-order or higher-order polynomial curves that reflect a non-linear relationship between SpO2 and time. The estimation technique can also be applied to second-order or higher-order polynomials. In one embodiment, the polynomial coefficients provide additional information related to the higher-order rate of changes (second, third, etc. derivatives) that can also be displayed to the user via the measurement gauges and/or the data trends in a user interface screen of a host application.

FIGS. 77A and 77B illustrate a package that includes a monitoring device and example accessories. To prevent tampering with the monitoring device during storage and/or transport, an anti-tamper tab 7700 seals the package 7702 until the monitoring device is ready for use by the user (FIG. 77A). When the user is ready to use the monitoring device 100, the user tears off the anti-tamper tab 7700 and removes the product label 7704 with the barcode.

In FIG. 77B, the monitoring device 100 and the accessories are shown as removed from the package 7702. In this example, in addition to the monitoring device 100 and the product label 7704, the package 7702 contains a roll of a tape 7706 and a headband 7708. In one embodiment, the tape 7706 is a self-adhering gentle breathable tape. The headband 7708 can be stored inside the roll of tape 7706 and removed when the roll of tape 7706 is removed from the package 7702) (removal of headband 908 represented by arrow).

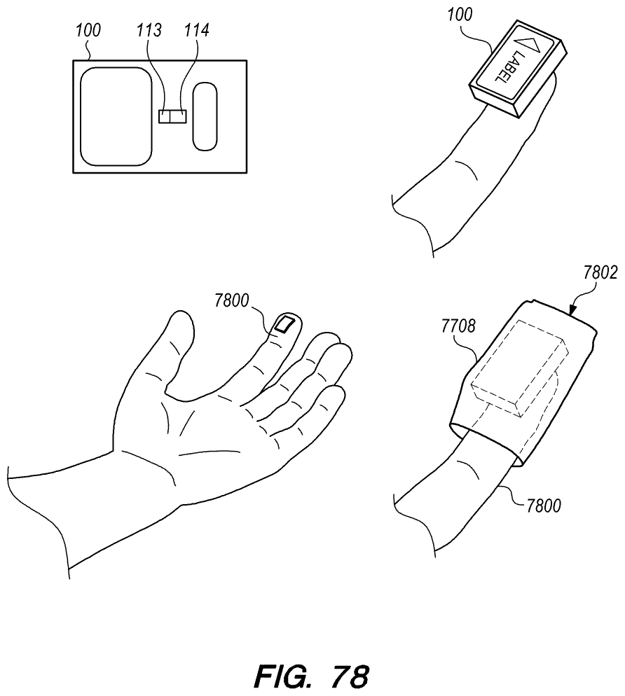

FIG. 78 depicts a workflow showing processes for attaching a monitoring device to a measurement site on a digit of a user. In an example embodiment, the operations shown in FIG. 20 to activate the monitoring device 100 have been performed prior to attaching the monitoring device 100 to a measurement site. The monitoring device 100 is attached to the measurement site 7800 (e.g., the fingertip) with the light source(s) 113 and the photodetector(s) 114 (and the temperature sensor 801 and/or the first electrical contact sensor 802 if included in the monitoring device 100) placed on, and in contact with, the measurement site 7800. In some embodiments, the monitoring device 100 is wrapped by the tape 7708 to improve the optical, electrical, and/or thermal coupling between the monitoring device 100 and the measurement site 7800. The tape 7708 can also protect the monitoring device 100 against a variety of events, including, but not limited to, mechanical shocks, dislodging, and direct sunlight exposure.

However, in embodiments where the monitoring device 100 includes the first and the second electrical contact sensors (e.g., 707, 708 in FIG. 7D), the tape 7708 may cover the second electrical contact sensor (e.g., 708 in FIG. 7D) depending on how the tape 7708 is wrapped around the measurement site 7800 and the monitoring device 100. Accordingly, conductive fibers (not shown) can be included in the tape 7708 to produce a tape with conductive properties. The conductive fibers in the tape 7708 create an electrical contact between the second electrical contact sensor and the tape 7708. A closed electrical path to the subject's heart (e.g., 710 in FIG. 7D) is created whenever the subject touches the tape 7708 with the conductive fibers with another body part (e.g., the opposite hand 709 in FIG. 7D). In some instances, the conductive fibers are arranged so as not to block the wave frequencies in the applicable wireless frequency range (for instance, for Bluetooth Low Energy, the frequency range is between 2400 and 2483.5 MHz) to enable the monitoring device 100 to communicate wirelessly with a host device.

In another embodiment, the tape 7708 with the conductive fibers can be made part of the monitoring device 100 so as to perform the functions of boosting the gain of the wireless antenna in the monitoring device 100. To boost the wireless efficiency, when using the conductive tape 7708 with the conductive fibers, the wrapping of the tape 907 around the measurement site 7800 can be done in a way to leave a cavity (or aperture, gap) 7802 at the fingertip extremity. The cavity 7802 can be created based on how the tape 7708 is wrapped around the monitoring device 100 and the measurement site 7800 (e.g., the fingertip) and/or by the way the tape 7708 is designed and/or attached to the monitoring device 100.

The conductive fibers in the tape 7708 can improve the optical, electrical and/or thermal coupling (contact) with the measurement site 7800 when pressure is applied to the monitoring device 100 at the measurement site 7800. Additionally or alternatively, the conductive fibers may serve as the second electrical contact sensor (e.g., 708 in FIG. 7D) that enables the user to create a closed electrical path to the heart when the subject touches the tape 7708 with the conductive fibers.

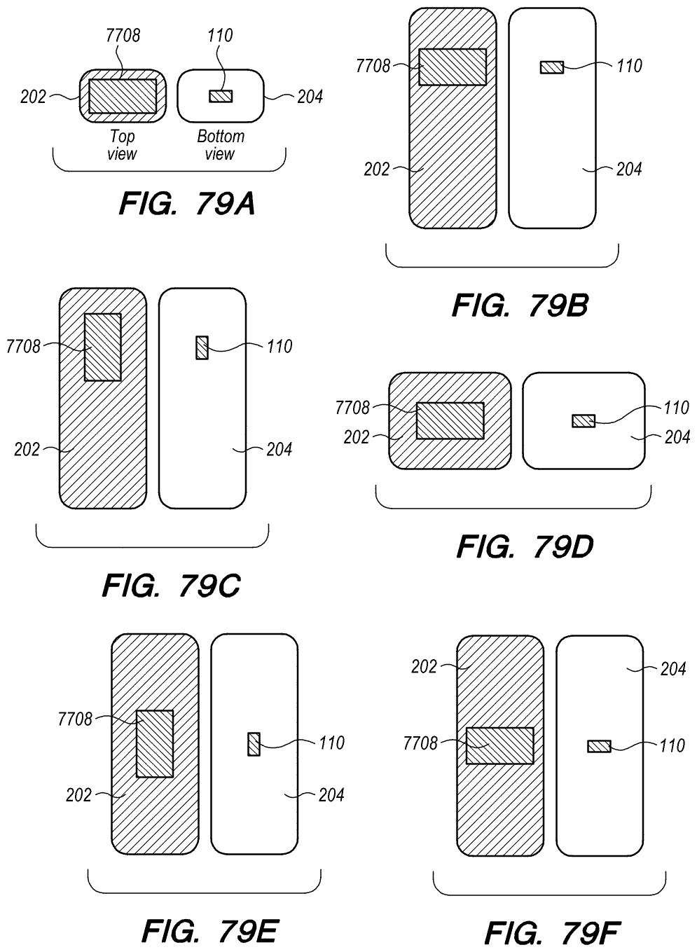

FIGS. 79A-79F illustrate example tape layouts suitable for use with a monitoring device. In the figures, each monitoring device uses a different tape layout, such as those depicted in FIGS. 2A and 2B, respectively. Specifically, in the embodiment shown in FIG. 2A, a monitoring device 100 is secured to the fingertip 200 using a flat adhesive bandage 202. The monitoring device is encapsulated in a polytetrafluoroethylene (PTFE) pocket or in an origami made out of the tape 7708. As shown in FIG. 79A, the optical sensor 110 of the monitoring device 100 is positioned on the underside 204 to contact the user's skin when the underside 204 is adhered to the user's skin. In some of the many alternative configurations, such as those shown in FIGS. 79B through 79F, the bandage or tape 202 may be used to attach the monitoring device 100 to a measurement site. The tape 202 can have different shapes with the monitoring device 100 positioned in different locations and/or orientations.

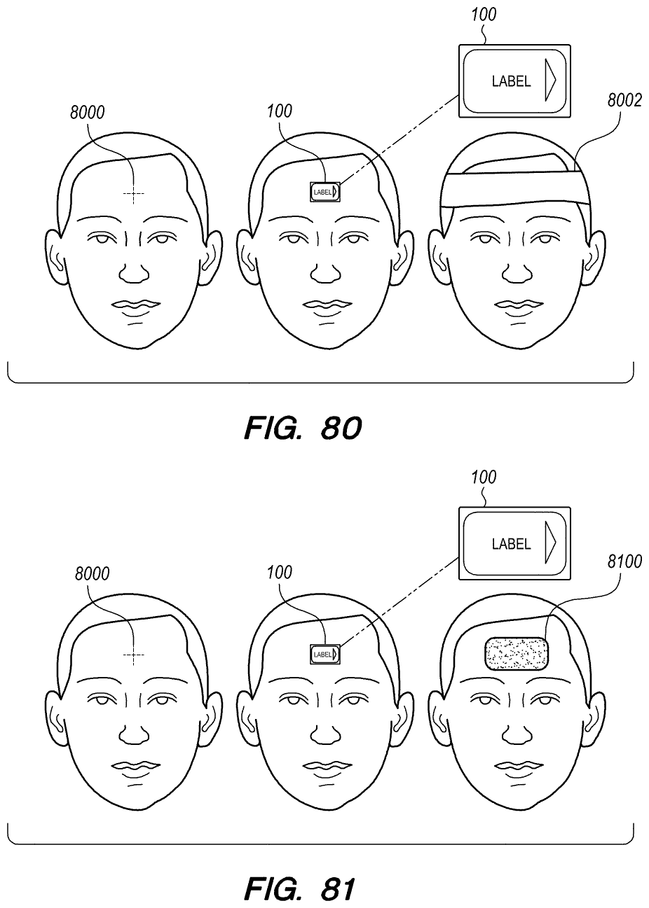

Although the measurement site in FIG. 78 is described as a digit of a user, other embodiments are not limited to that configuration. The monitoring device can be used with other types of measurement sites, such as the measurement sites shown in FIGS. 2B-2I. FIG. 80 depicts a monitoring device attached to the forehead of a user. In an example embodiment, the operations shown in FIG. 20 to activate the monitoring device 100 are performed prior to attaching the monitoring device 100 to the forehead (the measurement site). The monitoring device 100 is attached to the measurement site 8000 (e.g., the forehead) with the light source(s) and photodetector(s) of the optical sensor (and the temperature sensor and/or the first electrical contact sensor if included in the monitoring device 100) placed on, and in contact with, the measurement site 8000.

In some embodiments, the use of the forehead as a measurement site 8000 can result in improved measurements for SpO2, PR, PA, and/or PI. The perfused tissue in the forehead region is a few millimeters thick and is suitable for a reflective pulse oximeter that has a relatively small separation (gap) between the one or more light sources and the one or more photodetectors (e.g., light source(s) 113 and photodetector(s) 114 in FIG. 1). In some embodiments, the monitoring device 100 has a separation gap between 2.5 mm and 7 mm. However, depending on the optical sensor design, separation gaps smaller than 2.5 mm can be attained. The monitoring device 100 can probe the shallow and perfused blood tissue from the forehead, providing waveforms with relatively high signal-to-noise ratio, and enabling the accurate detection of SpO2, PR, PI, and/or PI, even at very low perfusion levels.

The forehead as a measurement site 8000 may also be advantageous during physical activity. The measurement site 8000 is less prone to motion artifacts given that it is located on the subject's head. As a mechanism of self-protection against brain injuries, the body's motion effects are always minimized at the subject's head, through motion dumping enabled by the skeleton and muscles. In addition, the forehead is subjected to shorter transport delays to physiological changes (i.e., SpO2, PR, PA, and/or PI changes. etc.), and also shows less vasoconstrictor response to cold, given its proximity to the brain. Optionally, a headband 8002 (e.g., headband 7708 in FIG. 77B) can be used to protect the monitoring device 100 against various events, such as mechanical shocks, dislodging, and direct sunlight exposure. The headband 8002 can also improve optical, thermal and/or electrical coupling with the measurement site 8000.

However, in embodiments where the monitoring device 100 includes the first and the second electrical contact sensors (e.g., 707, 708 in FIG. 7D), the headband 8002 may cover the second electrical contact sensor (e.g., 708 in FIG. 7D). Accordingly, conductive fibers (not shown) can be included in the headband 8002 to produce a fabric or headband with conductive properties. The conductive fibers in the headband 8002 create an electrical contact between the second electrical contact sensor and the headband 8002. A closed electrical path to the subject's heart (e.g., 710 in FIG. 7D) is created whenever the user places a body part (e.g., fingertip 709 in FIG. 7D) on the headband 8002 with the conductive fibers.

To enable the monitoring device 100 to communicate wirelessly with the host device (e.g., host device 105 in FIG. 1), the conductive fibers in the headband 8002 can be arranged so as not to block the wave frequencies in the applicable wireless frequency range. In another embodiment, the headband 8002 with the conductive fibers can be made part of the monitoring device 100 so as to perform the functions of boosting the gain of the wireless antenna in the monitoring device 100 through the conductive fibers, and/or improving the optical, electrical, and thermal coupling (contact) with the measurement site 8000 (e.g., forehead) when pressure is applied to the monitoring device 100 at the measurement site 8000. Additionally or alternatively, the conductive fibers may serve as the second electrical contact sensor (e.g., 708 in FIG. 7D) that enables the user to create a closed electrical path to the heart whenever the user places a body part (e.g., fingertip 709 in FIG. 7D) on the headband 8002 with the conductive fibers.

FIG. 81 depicts a monitoring device attached to the forehead of a user. The light source(s) and the photodetector(s) of the optical sensor (and the temperature sensor and/or the first electrical contact sensor if included in the monitoring device 100) are placed on, and in contact with, the measurement site 8000. In this embodiment, an adhesive bandage 8100 is used to protect the monitoring device 100 against various events, including mechanical shocks, dislodging, and direct sunlight exposure. The adhesive bandage 8100 can also improve the optical, thermal and/or electrical coupling with the measurement site 8000 (e.g., forehead).

However, in embodiments where the monitoring device 100 includes the first and the second electrical contact sensors (e.g., 707, 708 in FIG. 7D), the adhesive bandage 8100 may cover the second electrical contact sensor (e.g., 708 in FIG. 7D). Accordingly, conductive fibers (not shown) can be included in the adhesive bandage 8100 to produce a bandage with conductive properties. The conductive fibers in the bandage 8100 create an electrical contact between the second electrical contact sensor (e.g., 708 in FIG. 7D) and the bandage 8100. A closed electrical path to the user's heart (e.g., 710 in FIG. 7D) is created whenever the user touches the bandage 8100 with the conductive fibers with a body part (e.g., a fingertip 709 in FIG. 7D).

To enable the monitoring device 100 to communicate wirelessly with a host device (e.g., host device 105 in FIG. 1), the conductive fibers in the bandage 8100 can be arranged so as not to block wave frequencies in the applicable wireless frequency range. In another embodiment, the bandage 8100 with the conductive fibers can be made part of the monitoring device 100 so as to perform the functions of boosting the gain of the wireless antenna in the monitoring device 100 through the conductive fibers, and/or improving the optical, electrical, and thermal coupling (contact) with the measurement site 8000 (e.g., forehead) when pressure is applied to the monitoring device 100 at the measurement site 8000. Additionally or alternatively, the conductive fibers may serve as the second electrical contact sensor (e.g., 708 in FIG. 7D) that enables the user to create a closed electrical path to the heart whenever the user touches the bandage 8100 with conductive fibers using a body part (e.g., fingertip 709 in FIG. 7D).

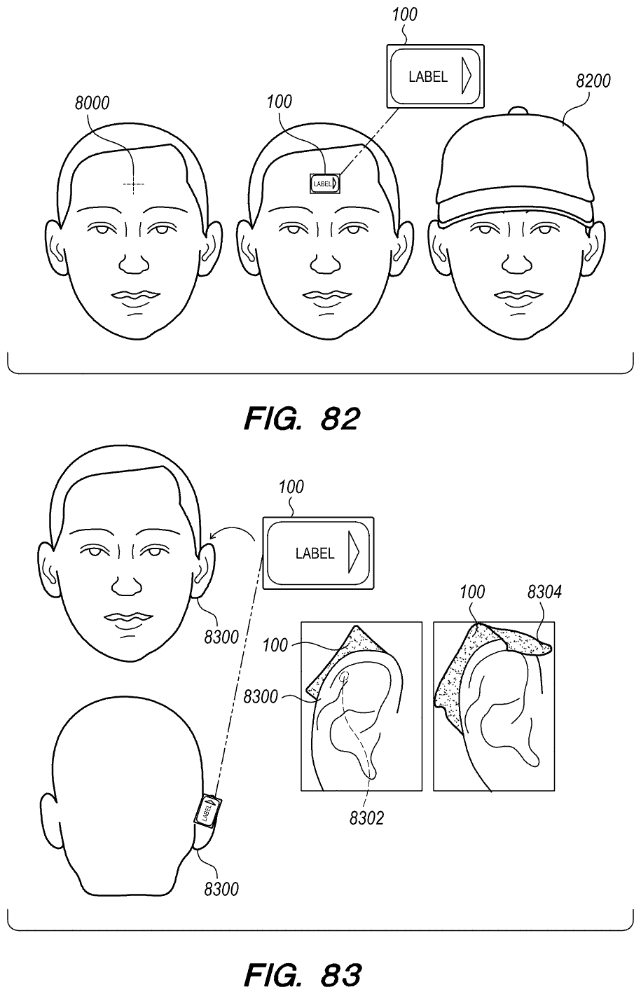

FIG. 82 depicts a monitoring device attached to the forehead of a user. The monitoring device 100 is attached to the measurement site 8000 (e.g., the forehead) with the light source(s) and the photodetector(s) of the optical sensor (and the temperature sensor and/or the first electrical contact sensor if included in the monitoring device 100) placed on, and in contact with, the measurement site 8000. In this embodiment, a hat 8200 can be used to protect the monitoring device 100 against various events, including mechanical shocks, dislodging, and direct sunlight exposure. The hat 8200 can also improve optical, thermal and/or electrical coupling with the measurement site 8000.

However, in embodiments where the monitoring device 100 includes the first and the second electrical contact sensors (e.g., 707, 708 in FIG. 7D), the hat 8200 may cover the second electrical contact sensor (e.g., 708 in FIG. 7D). Accordingly, conductive fibers (not shown) can be included in the hat 8200 to produce a fabric or a hat 8200 with conductive properties. The conductive fibers in the hat 8200 create an electrical contact between the second electrical contact sensor and the hat 8200. A closed electrical path to the user's heart (e.g., 710 in FIG. 7D) is created whenever the user places a body part (e.g., a fingertip 709 in FIG. 7D) on the hat 8200 with the conductive fibers.

To enable the monitoring device 100 to communicate wirelessly with a host device (e.g., host device 105 in FIG. 1), the conductive fibers in the hat 8200 can be arranged so as not to block wave frequencies in the applicable wireless frequency range. In another embodiment, the hat 8200 with the conductive fibers can be made part of the monitoring device 100 so as to perform the functions of boosting the gain of the wireless antenna in the monitoring device 100 through the conductive fibers, and/or improving the optical, electrical, and thermal coupling (contact) with the measurement site 8000 when pressure is applied to the monitoring device 100 at the measurement site 8000. Additionally or alternatively, the conductive fibers may serve as the second electrical contact sensor (e.g., 708 in FIG. 7D) that enables the user to create a closed electrical path to the heart whenever the user touches the hat 8200 with conductive fibers using a body part (i.e., fingertip 709 in FIG. 7D).

FIG. 83 illustrates a monitoring device attached to the posterior auricle (ear) of a user. The monitoring device 100 is attached to the measurement site 8300 (e.g., the ear) with the light source(s) and the photodetector(s) of the optical sensor (and the temperature sensor and/or the first electrical contact sensor if included in the monitoring device 100) placed on, and in contact with, the measurement site 8300. In some instances, depending on the intensity of the light and the skin color of the user, the light emitted by the one or more light source(s) may be visible through the ear's cartilage (see region 8302).

In one aspect, the use of the posterior auricle as a measurement site 8300 can result in improved measurements for SpO2, PR and PI. The thickness of the ear cartilage in this region of the ear is typically only a few millimeters thick. Thus, the measurement site 8300 is suitable for a reflective pulse oximeter that has a small separation (gap) between the one or more light sources and the one or more photodetectors of the optical sensor (e.g., 113, 114 in FIG. 1).

The measurement site 8300 enables the monitoring device 100 to probe the shallow and perfused blood tissue from the posterior auricle, providing waveforms with high signal-to-noise ratio and enabling the accurate detection of SpO2, PR, PA, and/or PI, even at very low perfusion levels. The posterior auricle as a measurement site 8300 is also advantageous during physical activity. The measurement site 8300 is less prone to motion artifacts given that it is located on the user's head. As a mechanism of self-protection against brain injuries, the body's motion effects are always minimized at the user's head through motion dumping enabled by the skeleton and muscles. In addition, the posterior auricle is subjected to shorter transport delays to physiological changes (i.e., SpO2, PR, PA, and/or PI changes. etc.), and also shows less vasoconstrictor response to cold, given its proximity to the brain.

In some embodiments, an adhesive tape 8304 can be used to protect the monitoring device 100 against various events, including mechanical shocks, dislodging, and direct sunlight exposure. The adhesive tape 8304 may also improve optical, thermal and/or electrical coupling with the measurement site 8300 (posterior auricle). However, in embodiments where the monitoring device 100 includes the first and the second electrical contact sensors (e.g., 707, 708 in FIG. 7D), the adhesive tape 8304 may cover the second electrical contact sensor (e.g., 708 in FIG. 7D). Accordingly, conductive fibers (not shown) can be included in the adhesive tape 8304 to produce an adhesive tape 8304 with conductive properties. The conductive fibers in the adhesive tape 8304 create an electrical contact between the second electrical contact sensor and the adhesive tape 8304. A closed electrical path to the user's heart (e.g., 710 in FIG. 7D) is created whenever the user touches the adhesive tape 8304 with the conductive fibers with a body part (e.g., a fingertip 709 in FIG. 7D).

To enable the monitoring device 100 to communicate wirelessly with a host device (e.g., host device 105 in FIG. 1), the conductive fibers in the adhesive tape 8304 can be arranged so as not to block wave frequencies in the applicable wireless frequency range. In another embodiment, the adhesive tape 8304 with the conductive fibers can be made part of the monitoring device 100 so as to perform the functions of boosting the gain of the wireless antenna in the monitoring device 100 through the conductive fibers, and/or improving the optical, electrical, and thermal coupling (contact) with the measurement site 8300 when pressure is applied to the monitoring device 100 at the measurement site 8300. Additionally or alternatively, the conductive fibers may serve as the second electrical contact sensor (e.g., 708 in FIG. 7D) that enables the user to create a closed electrical path to the heart whenever the user touches the adhesive tape 8304 with conductive fibers using a body part (e.g., fingertip 709 in FIG. 7D).

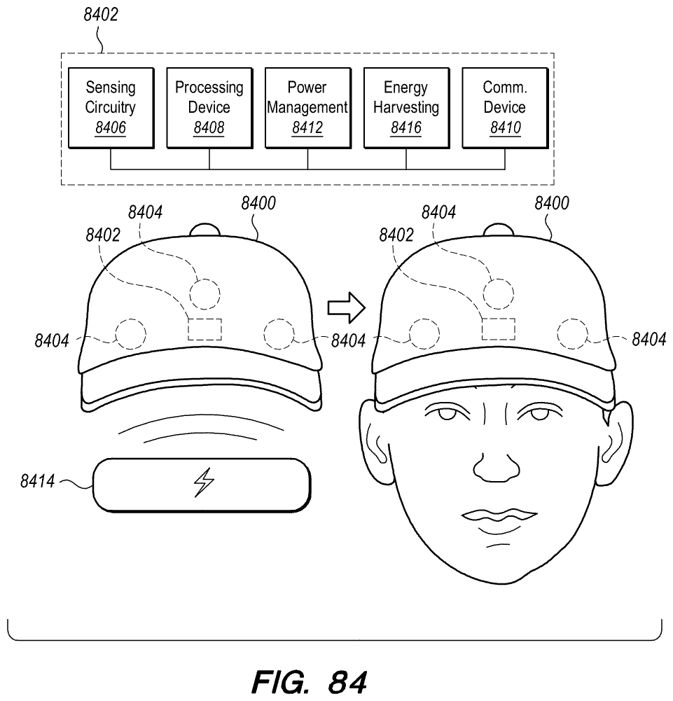

FIG. 84 depicts an example wearable hat that includes a monitoring device. The wearable hat 8400 includes built-in hat circuitry 8402 that enables the hat 8400 to be a wearable device for medical, fitness, and/or wellness applications. The hat 8400 can further include one or more built-in electroencephalogram (EEG) electrodes 8404. The EEG electrodes 8404 can be part of the fabric of the hat 8400 or attached to the hat 8400, depending on the application. Other optical, temperature, electrical contact, electrical capacitive contact, ultrasound, etc. sensors can be part of the hat circuitry 8402 or can be distributed around the hat layout (as the EEG electrodes) and/or part of the fabric of the hat 8400, depending on the application.

The hat circuitry 8402 may or may not include some or all of the circuitry in the monitoring device 100 shown in FIG. 1. The hat circuitry 8402 senses one or more physiological measurements and/or environmental data, processes and conditions the physiological and/or environmental data and sends the data wirelessly to a host device (e.g., host device 105 in FIG. 1). In one embodiment, the data is transmitted to the host device in real time. For example, the signals from the photodetector(s), the EEG electrodes, and/or the temperature sensor are transmitted to the host device.

The host device processes and analyzes the data, displays one or more measurements and trends, generates an alarm or warning, stores the data, shares the data, and the like. In the illustrated embodiment, the hat circuitry 8402 has the following functionalities:

1. Sensing Circuitry—The hat circuitry 8402 includes sensing circuitry 8406 such as electronics and sensors, as discrete and/or distributed components, standard integrated circuits, and/or Application-Specific Integrated Circuit (ASIC), to sense continuously or at select times the body temperature, environmental temperature, environmental pressure, environmental ultraviolet (A, B, and/or C bands), body hydration, non-invasive blood total hemoglobin, carboxyhemoglobin and methemoglobin, SpO2, PR, PA, and/or PI, plethysmographs, non-invasive blood glucose levels, respiration rate, user's activity and calorie consumption, EEG, etc., and spot-check measurements of ECG waveforms.

2. Processing Device—A processing device 8408, such as a low-power processing device (e.g., ARM-based, ASIC, etc.) receives the data from the sensing circuitry 8406, processes the data, and transmits (e.g., wirelessly) the data to a host device using the communication device 8410. In some embodiments, a processing device, such as ASIC circuitry, performs specialized signal processing functions to reduce overall power consumption and the footprint of the hat circuitry 8402. In a non-limiting example, the processing device 8408 performs functions having a low complexity and low latency (i.e., hard real-time processing) and the host device processes data having a higher complexity and latency (i.e., soft real-time processing). The processing device 8408 can be part of the hat circuitry 8402 or can be distributed around the hat layout and/or part of the hat's fabric, depending on the application.

3. Power Management Circuitry—The power management circuitry 8412 includes a battery and manages the voltages and loads, battery life, and charging of the battery. In one embodiment, the battery in the hat 8400 is charged through a wireless charger 8414. The wireless charger 8414 minimizes circuitry isolation requirements and eliminates the need for cables and/or connectors. It may also be more convenient for the user given that to charge the battery in the hat 8400, the user can place the hat on top or near the wireless charger 8414. The voltages can be regulated through boost and buck converters that can be designed to provide voltage levels with maximum noise levels that match the requirements of the battery. The power management unit can be part of the hat circuitry 8402 or can be distributed around the hat layout and/or part of the fabric of the hat, depending on the application.

4. Energy Harvesting Circuitry—The energy harvesting circuitry 8416 can be composed of pyroelectric, piezoelectric, thermoelectric, photovoltaic, ambient-radiation, transducers and electronics that convert some or all forms of energy in the environment into electric power that can power the hart circuitry 8402 directly, or that can be used to recharge the battery in the power management circuitry 8412 to recharge or increase the battery life. The transducers and electronics can be part of the hat circuitry 8402 or can be distributed around the hat layout and/or part of the fabric of the hat, depending on the application.

5. Communication Device—Any suitable wireless communication device 8410 can be used. In one embodiment, the communication device 8410 is a wireless radio unit for receiving and sending data to a host device or to a network (e.g., network 120 in FIG. 1). In a non-limiting example, the hat 8400 can communicate wirelessly with a router using IPV6 (Internet protocol Version 6) over the BLUETOOTH Smart protocol. In one embodiment, the communication device 8410 supports multiple low-energy protocols such as BLUETOOTH Low Energy, ZIGBEE, ANT, or some custom/proprietary low-energy protocol. The communication device 8410, including one or more antennas, can be part of the hat circuitry 8402, or can be distributed around the hat layout and/or part of the fabric of the hat, depending on the application. In particular, the low-energy radio's antenna design can greatly benefit from the hat's layout, materials, and area, so as to enable distributed antenna layouts with very high intrinsic gains in the frequency bands of interest, thereby reducing significantly the radio's power consumption.

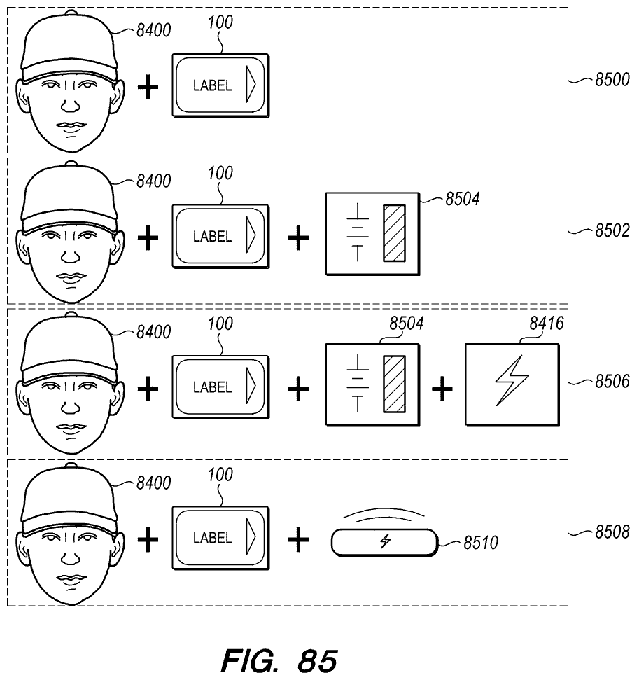

FIG. 85 illustrates several example configurations for the wearable hat shown in FIG. 84. Configuration 8500 includes the hat 8400 and the monitoring device 100. Configuration 8500 can be used in single-use disposable applications, where the hat 8400 and the hat circuitry 8402 are disposed of after the battery is empty of charge. In configuration 8500, the battery is a non-rechargeable battery and as a result, the power management circuitry 8412 and the energy harvesting circuitry 8416 may be omitted from the hat circuitry 8402. In one embodiment, the non-rechargeable battery can be part of the monitoring device 100.

Configuration 8502 includes the hat 8400, the monitoring device 100, and a removable battery 8504. Configuration 8502 can be used on multi-use disposable applications, where the hat 8400 and the hat circuitry 8402 are used multiple times. In this embodiment, whenever the amount of charge on the removable battery 8504 is low or empty, the removable battery 8504 is replaced with a new removable battery. The removable battery 8504 can be composed of standard non-rechargeable battery or can be a custom module with an anti-temper memory that prevents a user from using a battery that is not provided by the manufacturer of the hat 8400.

Configuration 8506 is similar to the configuration 8502, with the addition of the energy harvesting circuitry 8416. The energy harvesting circuitry 8416 can be used to increase the amount of charge on the removable battery 8504 and extend the amount of time the removable battery 8504 is used before replacement or recharging.

Configuration 8508 includes the hat 8400, the monitoring device 100, and a charger 8510 (e.g., wireless charger 8414). Configuration 8508 is used in reusable applications where the battery is rechargeable and the charger 8510 is used to charge the battery. In one embodiment, the rechargeable battery, fuel gauge, protection, and wireless charging receiver circuitries can be part of the monitoring device 100.

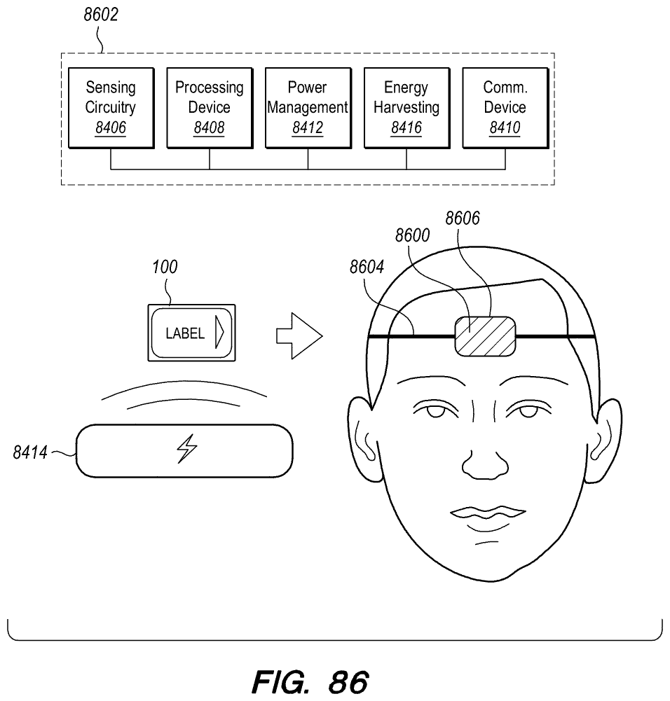

FIG. 86 depicts an example wearable patch positioned on a user. The wearable patch 8600 includes the monitoring device 100 and patch circuitry 8602 that senses one or more physiological and/or environmental data, processes and conditions the physiological and/or environmental data and sends the data wirelessly to a host device. In one embodiment, the data is transmitted to the host device in real time.

The host device processes and analyzes the data, displays one or more measurements and/or trends, generates one or more alarms or warnings, stores the data, and/or shares the data with another computing device. In one embodiment, the patch circuitry 8602 has similar functionalities as the hat circuitry 8402 shown in FIG. 84. However, given the relatively small size of the wearable patch 8600 when compared to the hat 8400, the patch circuitry 8602 can be integrated and lightweight.

The wearable patch 8600 may be a disposable, single-use patch, a multi-use patch, or a reusable patch. In the illustrated embodiment, instead of a headband or an adhesive tape, a monofilament 8604 is used to attach the wearable patch 8600 to the measurement site (e.g., the forehead). In one embodiment, the monofilament 8604 is a transparent or semi-transparent monofilament. The monofilament 8604 can be attached to a case 8606 with a cavity that fits the wearable patch 8600. The case 8606 can be made to match the user's skin color to create a discreet monitoring solution that works on the measurement site. The case 8606 with the monofilament 8604 can make the monitoring solution more discreet and improve the optical, electrical, and/or thermal coupling between the wearable patch 8600 and the measurement site. The monofilament 8604 may protect the wearable patch 8600 against various events, such as mechanical shocks, dislodging, and direct sunlight exposure.

In a reusable embodiment, the battery in the patch 8600 can be a rechargeable battery that is charged through a wireless charger 8414. The wireless charger 8414 can reduce or minimize the circuitry isolation requirements and/or reduce or eliminate the need for cables and/or connectors. Wireless charging can also be more convenient for the user given that to charge the battery in the wearable patch 8600, the user simply places the wearable patch 8600 on top or near the wireless charger 8414. The monitoring solution with the wearable patch 8600, the case 8606, and the monofilament 8604 can be applied to measurement sites other than the forehead. For example, measurement sites such as the arm, the leg, the wrist, the foot, the chest, and the neck, can be used. In addition, in some applications, the reusable monitoring solution with the wearable patch 8600, the case 8606, and the monofilament 8604 can be integrated into a single device to reduce costs and simplify usage workflow to make it more convenient for daily use.

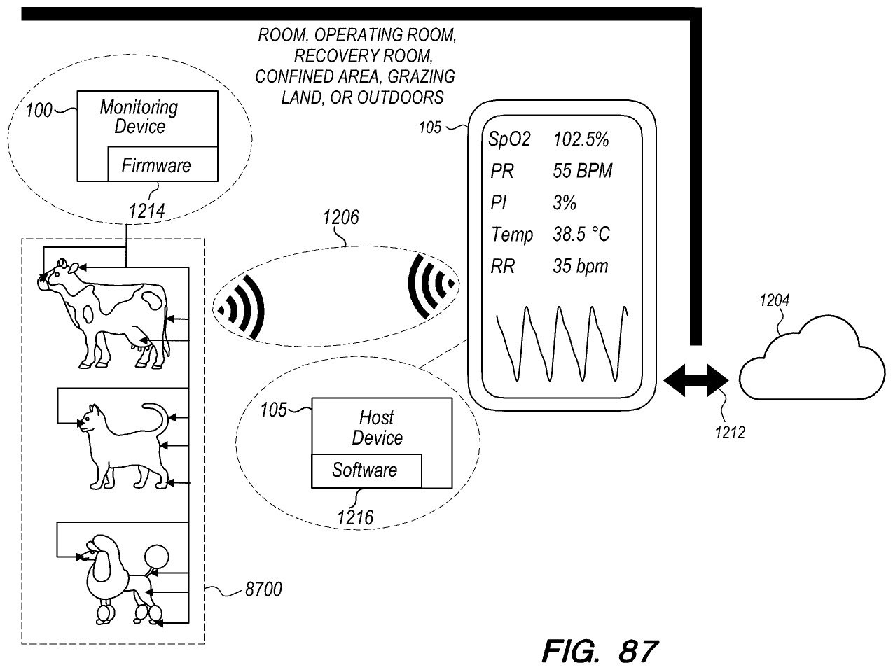

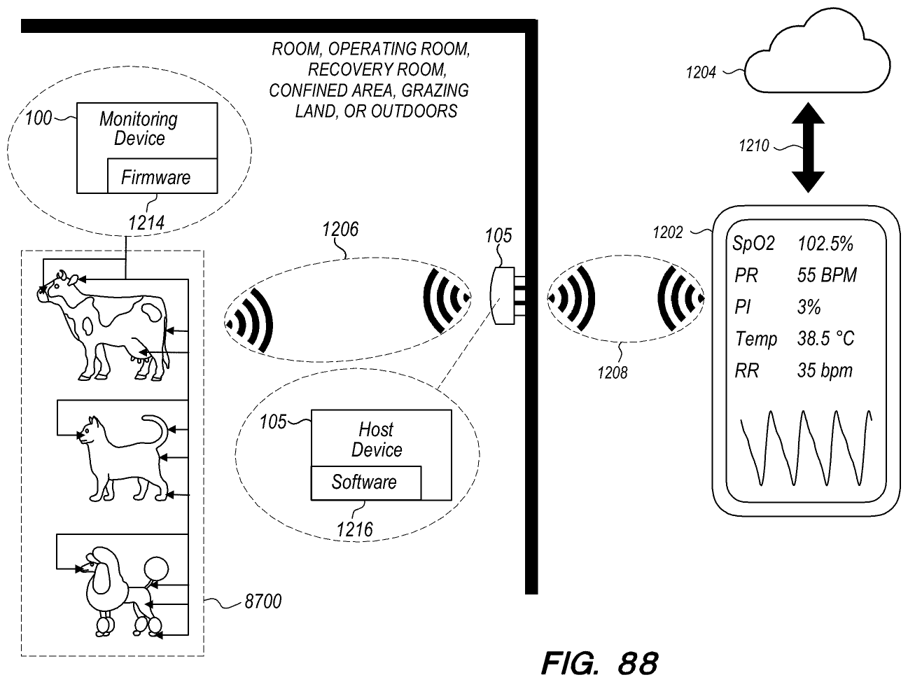

FIGS. 87-88 illustrate example embodiments for veterinary applications using a monitoring device, a host device, and a computing system as shown in FIG. 12A. The monitoring device 100 can be placed on measurement sites where perfusion and optical properties provide continuous, intense, stable, and repeatable signals. Depending on the type of animal 8700 (e.g., cattle, dog, cat, etc.), the tail, the nasal septum, an ear, the genital mucosa, the tongue, the lip, the vulva, the prepuce, the scrotum, a rear paw, a front paw, a lip, or some other measurement site can be more favorable than others for non-invasive measurements. In FIGS. 87 and 88, the host device can be any electronic device capable of running software. In FIG. 87, the host device 105 can be a multi-parameter monitor, mobile device, desktop, laptop, or tablet computer. In FIG. 88, the host device 105 is a wall appliance that is plugged into a power outlet on the wall (or some other place depending on the veterinary application) and is connected to the monitoring device 100 via the connection 1206. In the embodiment of FIG. 88, the software 1216 in the host device 105 does not provide interface functionalities. The interface functionalities are provided by a computing system 1202 in the form of a multi-parameter monitor that receives measurements and data from the host device 105 via the connection 1208. In FIGS. 87 and 88, the connection 1206 and the connection 1208 are wireless connections.

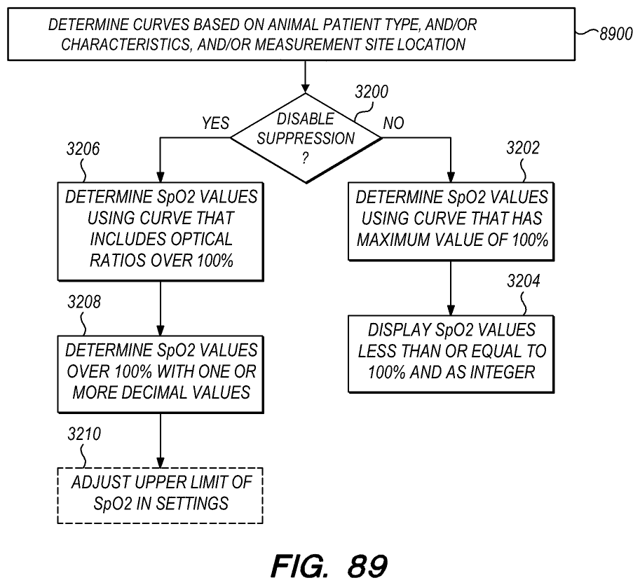

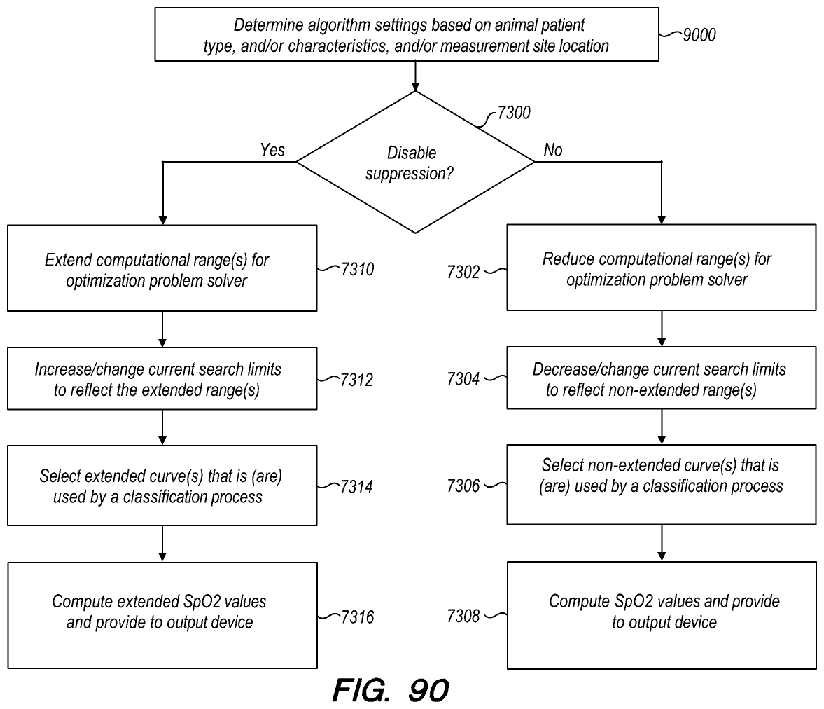

In some non-invasive, continuous monitoring embodiments for veterinary applications, the large variety of the types of animals, the characteristics, and the measurement sites may require different algorithms and/or calcurves to account for the wider range of physical (optical, mechanical, etc.) and physiological properties at the measurement site. The example flowcharts depicted in FIGS. 89-90 extend the embodiments shown in FIGS. 32 and 73 to account for the wider ranges of physical and physiological properties of the measurement sites on the animals. Like FIGS. 32 and 73, the embodiments of FIGS. 89 and 90 are described in conjunction with SpO2 measurements. However, in other embodiments, measurements of other parameters of interest can be used in the illustrated example methods.

FIG. 89 depicts a flowchart of an example first method of providing SpO2 values that exceed one hundred percent in veterinary uses. The process is described with reference to SpO2 values, but the method can be used with other parameters or measurements of interest. Non-limiting examples of the other parameters or measurements of interest are PR, PA, PI, non-invasive blood glucose, total hemoglobin, carboxyhemoglobin, methemoglobin, body fat, and/or body hydration levels.

Initially, as shown in block 8900, information about the type of animal (e.g., cattle, dog, cat, etc.), the animal's characteristics (including age, gender, body weight, height, etc.), and/or the measurement site location are obtained to determine and select one or more curves for SpO2 measurement values up to 100% and above 100% that best match the type of animal, the animal's characteristics, and/or the chosen measurement site location. Blocks 3200, 3202, 3204, 3206, 3208, 3210 are then performed using the selected curves.

FIG. 90 illustrates a flowchart of an example second method of providing SpO2 values that exceed one hundred percent in veterinary uses. The process is described with reference to SpO2 values, but the method can be used with other parameters or measurements of interest. Non-limiting examples of the other parameters or measurements of interest are PR, PA, PI, non-invasive blood glucose, total hemoglobin, carboxyhemoglobin, methemoglobin, body fat, and/or body hydration levels.

Initially, as shown in block 9000, information about the type of animal, the animal's characteristics (including age, gender, body weight, height, etc.), and/or the measurement site location are obtained to determine and select one or more algorithm settings (e.g., non-extended and extended ranges, curves, search limits, etc.) for SpO2 measurement values that best match the type of animal, the animal's characteristics, and/or the chosen measurement site location. Blocks 7300, 7302, 7304, 7306, 7308, 7310, 7312, 7314, and 7316 are then performed using the selected algorithm settings.

A wireless monitoring device that is small, lightweight, disposable (or reusable), with self-adhering adhesive tape that facilitates attachment to a measurement site can be very advantageous in veterinary applications. Site pigmentations, fur, and other non-ideal skin characteristics found in animal patients can restrict the available measurement site locations and areas where perfusion and optical properties provide continuous, intense, stable, and repeatable signals. A small, lightweight, wireless monitoring device with reflectance-based optical sensor can be easily attached to any part of the animal body without the need for transmittance-based signals applied and collected in opposite sides of a measurement site. Optical power is reduced in reflectance-based optical sensors given that the separation between the one or more light sources (e.g., 113 in FIG. 1) and the one or more photodetectors (e.g., 114 in FIG. 1) is much smaller than typical separations found in transmittance-based optical sensors. For example, using the monitoring device 100 shown in FIG. 7A, the monitoring device 100 is attached to a measurement site 700 to collect optical data through the optical sensor 110. The one or more light sources 113 emit light that penetrates the blood perfused dermis 704 and interacts with the heart's pulsatile signal (represented by dashed line) to create optical pulsatile signals. The optical pulsatile signals (photoplethysmographs) are captured by a frontend connected to the one or more photodetectors 114 and filtered, conditioned, and converted to digital signals. The photoplethysmographs are sent wirelessly to a host device for further processing and analysis, including real-time measurement display, warning (alarm) generation, and/or storage of the animal patient's SpO2, PR, PI, etc.

A monitoring device for veterinary use can also include a temperature sensor 705 (FIG. 7B) that is in contact with the measurement site 700. The temperature sensor 705 can be used to measure core body temperature of the animal patient. The monitoring device 100 can be attached to a location in the body of the animal patient with superficial temperature correlated to the body's core temperature. The monitoring device 100 collects and processes temperature sensor measurements and converts the temperature sensor measurements to digital signals. The digital signals can be transmitted wirelessly to a host device for further processing and analysis, including real-time measurement display, warning (alarm) generation, and/or storage of the animal patient body's core temperature.

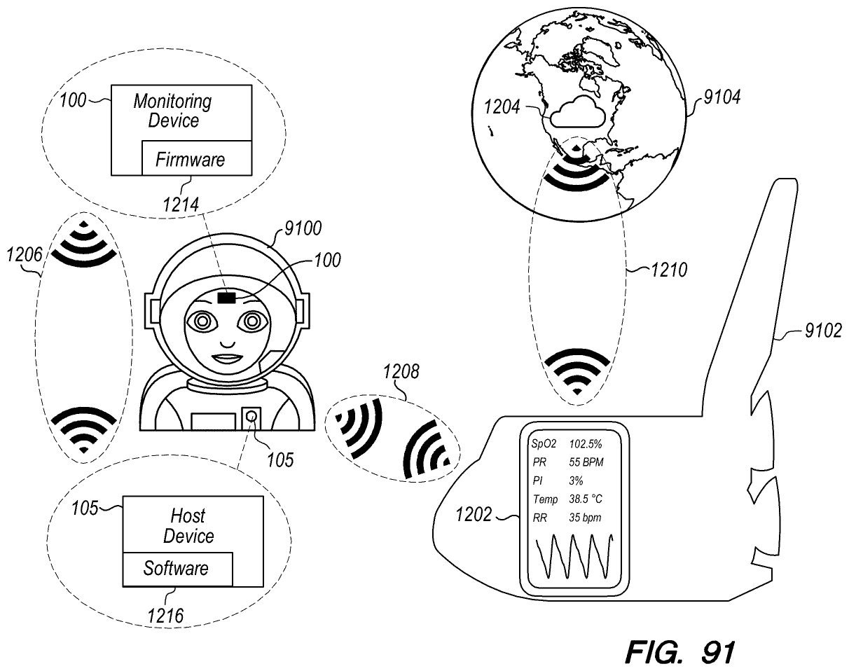

FIG. 91 depicts an example embodiment for space applications using a monitoring device, a host device, a computing system, and a cloud computing system as shown in FIG. 12A. A monitoring device 100 can be placed on one or more measurement sites on the body of the astronaut where perfusion and optical properties provide continuous, intense, stable, and repeatable signals (i.e., forehead, finger, hand, ear, chest, wrist, etc.). The monitoring device 100 can be disposable or reusable, built-in in the space suit 9100, or as a separate unit that is attached to the astronaut's body. The host device 105 can be any computing device that is capable of running software 1216 and being part of the space suit 9100 or a wearable device worn by the astronaut (e.g., wristwatch, etc.).

The host device 105 transfers information to the computing system 1202 in a spacecraft 9102 for analysis, display, warnings, storage, etc. The computing system 1202 transmits information to the cloud computing system 1204 for analysis, display, warnings, storage, etc. located on a planet (e.g., earth), a moon, or another location 9104 where the astronaut's physiological information is accessed by others (e.g., a mission control center). In FIG. 91, the connections 1206, 1208, and 1210 are wireless connections (e.g., Deep Space Network).

The embodiment in FIG. 91 can be applied to the continuous monitoring of the astronaut's physiological measurements (i.e., SpO2, PR, PA, PI, RR, Temp, etc.) while the astronaut wears the space suit 9100 during a particular mission, or while the astronaut is in the spacecraft 9102 and not wearing the space suit 9100. This embodiment can be of vital importance to ensure that the astronaut's physiological conditions and/or vital signals are within acceptable ranges. For instance, the measurements of oxygen saturation above 100% can help to control oxygen consumption and avoid the effects of oxygen toxicity due to exposure for long periods of time to higher oxygen concentrations from the space suit 9100 and/or the spacecraft 9102.

FIG. 92 illustrates another example method of using a monitoring device. In the illustrated embodiment, the monitoring device 100 includes the firmware 1214 that is used purely for data collection. The hardware of the monitoring device 100 has an internal or external memory 9200 that is connected to a processing device (e.g., processing device 102 in FIG. 1) via a serial or a parallel interface. In one embodiment, the memory 9200 is a nonvolatile memory. In another embodiment, the memory 9200 can be embedded in the processing device in the monitoring device 100 to reduce the size and the cost of the overall monitoring device 100.

The monitoring device 100 collects optical and/or electrical and/or thermal and/or acoustic (including sound and ultrasound) data from the user and data from the environment where the user is located (i.e., environment temperature, pressure, humidity, ambient light, audio, etc.) as continuous and/or intermittent data streams. The monitoring device 100 optionally processes the collected data (i.e., demodulation, filtering, down-sampling, up-sampling, etc.) in real-time before storing the processed data streams 9202 in the memory 9200. The processed data streams 9202 are then processed by a data processing laboratory 9204. Several example techniques can be used for collecting, processing, storing, and transferring the processed data streams 9202 to the data processing laboratory 9204. In one example embodiment, the following workflow can be used:

1. The monitoring device 100 with the memory 9200 is placed on a measurement site of the user. View 9206 shows the monitoring device 100 placed on the forehead, although other embodiments are not limited to this implementation. Placement of the monitoring device 100 can be done by the user or by a caregiver, depending on the application. In a setting such as a hospital, a clinic, and a doctor's office, for instance, the monitoring device 100 is typically placed on the user by a caregiver. In home settings, the monitoring device 100 can be attached to a measurement site by the user, a family member, or an attending caregiver. Alternatively, the user can have an appointment in a doctor's office, outpatient clinic, or pharmacy where the monitoring device 100 with the memory 9200 can be placed on the user.

2. The monitoring device 100 with the memory 9200 operates for a period of time collecting, processing, and storing continuous or intermittent data streams from the user. View 9208 shows an example where data is being collected while the user is asleep, although other embodiments are not limited to this implementation. In one embodiment, the period of time can range from a few hours to several weeks depending on the application. For sleep disorders (e.g., insomnia, apnea, sleep movement disorder, etc.), for instance, an overnight data collection may suffice. For cardiovascular diagnosis (abnormal heart rhythms or arrhythmias, cardiomyopathy, etc.), one or more weeks of data collection may be required to provide assessment of the user's cardiovascular state with acceptable specificities and sensitivities.

3. The monitoring device 100 is then mailed to the data processing laboratory 9204 (view 9210), where the data collected and stored in the monitoring device 100 is read and processed by a computer system 9212 to produce a report 9214 with measurements, analysis, metrics, distributions, charts, and ranges used in the diagnosis of diseases for diagnostic care, prediction of potential future diseases for preventive care, and or general survey on the user's wellbeing (i.e., life style, mental stress, work environment condition and associated physical and/or metal stress, etc.).

4. The generated report 9214 can then be transmitted (e.g., mailed) to the user's caregiver (and optionally to the user) to aid in user assessment, diagnosis, and treatment.

In another embodiment, the manual data transfer 9210 is replaced by transmitting electronically 9216 the processed data streams 9202 to the computer system 9212 or a remote storage (e.g., a cloud-based storage) that is part of the data processing laboratory 9204 or is accessible by the data processing laboratory 9204 to enable the data processing laboratory 9204 to read, analyze, and process the data streams 9202.

In another embodiment, the data processing laboratory 9204 is replaced with the user's Electronic Health Record (EHR) 9218 and the generated report 9214 is transmitted electronically to the EHR 9218 via the computing system 9212, which can be a (distributed or centralized) cloud computing system without a specific physical location such as the data processing laboratory, and optionally mailed to the user and/or the user's caregiver 9220. In one embodiment, the electronic transmission of the processed data streams 9202 and/or the electronic transmission of the report 9214 is automatic (does not require any user input).

One advantage to the embodiment depicted in FIG. 92 is that only the monitoring device 100 is used during data collection. The data processing capabilities of a host device are replaced by the data processing laboratory 9204, which increases simplicity, reliability, and the costs associated with a host device. The embodiment shown in FIG. 92 can be used in clinical, fitness, and/or wellness applications where measurements, trends, and/or metrics are required to diagnosis a particular disease or state, and any real-time measurements or alarms need not be conveyed to a caregiver.

In some applications, a host device can be used to aid in the transfer of the processed data streams 9202 to the computer system 9212 of the data processing laboratory 9204. The processed data streams 9202 can be transferred after data collection is complete or while data collection is occurring. The host device can be a mobile device, a wall appliance, a desktop or laptop computer that can establish a wireless or wired connection with the monitoring device 100 for the purpose of transferring the processed data streams 9202 to the computer system 9212.

The types of algorithms that are run by the computer system 9212 are similar to the algorithms executed by the host device and described herein. However, because the processed data streams 9202 can be processed in batch mode by the computer system 9212 to produce the report 9214. Additionally or alternatively, non-causal algorithms with a higher complexity can be employed considering that the processing time and the power consumption requirements may not be impediments.

In another alternate embodiment, the monitoring device 100 does not include a communication device (e.g., communication device 103 in FIG. 1). Instead, the monitoring device 100 includes a connector or plug that can be used to enable a wired connection to the computer system 9212 to upload the processed data streams 9202 to the computer system 9212 or to a cloud-based system that can be accessed by the computer system 9212. Omitting the communication device 103 can simplify the monitoring device 100 and/or reduce the cost of the monitoring device 100. The lack of a wireless connection can reduce the processing requirements and the power consumption of the monitoring device 100. Alternatively, the connector or plug in the monitoring device 100 for the wired connection can be hidden from the user (internal) because it is not used during data collection, and is only used when the monitoring device 100 transfers the processed data streams 9202 to the computer system 9212.

The monitoring device 100 can be a single-user use device or a reusable device. The advantage of single-use implementations is that the monitoring device 100 can be recycled after the processed data streams 9202 are transferred to the computer system 9212 or to the EHR 9218. The single-use implementation can minimize the costs associated with shipping, handling, and sterilization, simplify the workflow and/or minimize the risks of cross contamination among users.

In reusable implementations, the overall cost of the monitoring device 100 can be reduced because the monitoring device 100 can be used multiple times. Hybrid implementations that include reusable, disposable, and/or removable parts or assemblies for the monitoring device 100 can be adopted to minimize the risks and maximize the benefits of such data collection process. In an example embodiment, the monitoring device 100 can be reusable with the one or more tapes that adhere the monitoring device 100 to the user being single-use and disposable tape(s). In another example embodiment, to eliminate the need for battery charging, the monitoring device 100 may use a non-rechargeable battery that is assembled together with the disposable tape(s), and together the battery and the tape(s) form a single disposable assembly. In such embodiments, the monitoring device 100 is reused multiple times and the tapes and non-rechargeable battery are replaced before each use. Several hybrid configurations with reusable, disposable and/or removable parts or assemblies can be designed to make the monitoring device 100 suitable for a particular clinical, fitness, or wellness application.

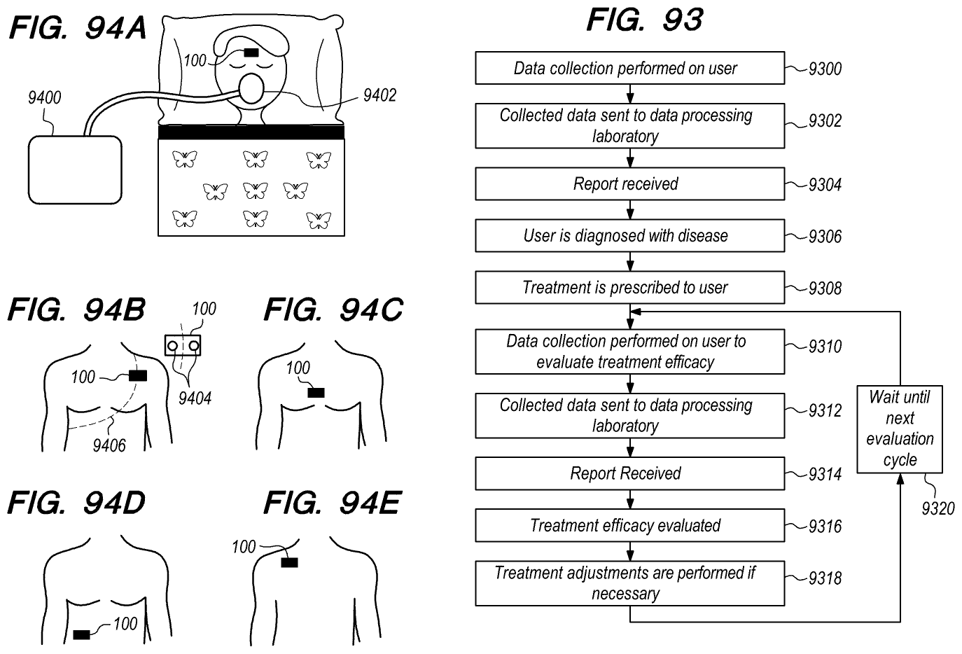

FIG. 93 depicts a flowchart of an example method for disease diagnosis and/or screening, and efficacy evaluation of disease treatment on human or veterinary users. The process describes data collections and report generations to aid in user diagnosis and/or screening as well as to evaluate treatment efficacy once a diagnosed user is under treatment. The data collections and transfers, and the report generations and transfers can be implemented per the embodiments described in conjunction with FIG. 92.

Initially, as shown in block 9300, data collection is performed on a user to aid in diagnosis and/or screening. In a non-limiting example, the data collection is performed according to some or all of the embodiments depicted in FIG. 92. In other embodiments, one or more other processes to collect data on an undiagnosed user is performed. In one aspect, the user is experiencing symptoms related to a sleep or respiratory disorder and the data collection relates to diagnosing the disorder.

The collected data is then transmitted to a data processing laboratory to create a report (block 9302). The caregiver, and optionally the user, receives the report at block 9304. The report is received at block 3312. For example, a caregiver receives the report and proceeds in block 9306 to perform disease diagnosis on the user. If the user is diagnosed with a disease, treatment is proscribed in block 9308. For example, if a user is diagnosed with sleep apnea after a sleep study, the caregiver may prescribe a Continuous Positive Air Pressure (CPAP) machine be worn during sleep, or an advanced home ventilator for users with neuromuscular diseases. An oxygen delivery system can also be prescribed for users (e.g., lung diseases, etc.) that need oxygen supplement. These types of machines are represented by machine 9400 and unit 9402 (e.g., a face mask) in FIG. 94. The machine 9400 and the unit 9402 reduce or eliminate respiratory complications that a user may experience. For example, in the case of users with untreated sleep apnea, the users are more likely to have an abnormal heart rhythm such as atrial fibrillation, which may increase the risks of cerebrovascular accident (CVA). In a non-limiting example, the machine 9400 can be a CPAP machine, an advanced home ventilator, or an oxygen delivery system connected via a tube to the unit 9402.

Once the diagnosed user begins treatment or therapy, the caregiver may prescribe data collections periodically or at select times to evaluate the efficacy of the treatment. Blocks 9310, 9312, 9314, 9316, 9318, and 9320 describe an example process for evaluating (periodically or at select times) the efficacy of the treatment. In block 9310, another data collection is performed on the user. The collected data is then transmitted to a data processing laboratory to create a report (block 9312). The report is received in block 9314 and the treatment efficacy is evaluated at block 9316. If needed, treatment adjustments are performed at block 9318. In block 9320, the user and/or the caregiver can be alerted periodically or nonperiodically (i.e., based on a schedule defined by the doctor as the treatment progresses) of upcoming or due data collections to evaluate the treatment efficacy. For example, a computer system, EHR system, or a third party can generate reminders and alerts for a caregiver.

FIGS. 94A-94E illustrate different placements of a monitoring device on a user. In FIG. 94A, the monitoring device 100 is placed on the forehead of the user. The forehead enables the measurement of electrical (e.g., electroencephalogram and bioimpedance), optical, mechanical, ultrasound, and core temperature signals. The monitoring device 100 can also be placed on another body part, such as a digit or an ear as shown in FIGS. 2A and 2G or 2I, respectively. FIGS. 94B-94E show example embodiments where the monitoring device 100 is placed on the upper left of the chest (FIG. 94B), on the center of the chest (FIG. 94C), on the lower right of the chest (FIG. 94D), and on the upper right of the back (FIG. 94E). The locations in FIGS. 94B-94E also enable the monitoring of electrical, mechanical, ultrasound, and optical signals that can be employed, for instance, for ECG, pulse oximetry, respiration rate, and pulse transit time data collections. In the case of ECG signals, the sensor (e.g., electrodes 9404 (shown in expanded drawing detail)) can be placed on any chest location (front and back) where the electrodes touch different electric equipotential (or isopotential) curves with measurable (detectable) difference of potential (voltage). The equipotential curves define continuous lines where the electric potential is the same. The electric equipotential curves enable the selection of locations and orientations for the monitoring device and electrodes that can maximize the voltage (difference of potential) measured so as to create ECG signals with acceptable signal-to-noise ratio.

FIG. 94B depicts an example of an electric equipotential curve 9406 (dashed line) that was used to select the location and orientation for the monitoring device 100. The two electrodes 9404 (shown in expanded drawing detail) in this example embodiment are placed orthogonally to the depicted equipotential curve 9406 to maximize or improve the ECG measured signals. Additionally, the monitoring device 100 can also have motion and audio sensors (not shown) that capture the user's activity and snoring during sleep to aid in the diagnosis and/or treatment efficacy. Depending on the number of light and/or ultrasound (mechanical) sources and detectors, data for quantifying body hydration, body fat, and metabolic and blood variables (e.g., blood glucose concentration, total hemoglobin, etc.) can also be acquired.

In one embodiment, the reports created in blocks 9302 and 9312 in FIG. 93 show some or all of oxygen saturation, pulse rate, perfusion index, respiration rate, and body and/or skin temperature and bioimpedance, trends and/or statistics such as oxygen desaturation index (i.e., number of desaturations per hour greater than a certain threshold, such as ODI 3%, ODI 4%, etc.), the cumulative time where oxygen saturation is below a certain threshold (e.g., time with oxygen saturation below 90% (T90)), pulse rate variability, pulse rate volatility index, perfusion index log-volatility, body temperature volatility, histogram measurements, charts showing time duration and intensity of respiratory events and important transition phases of measurement parameters and sleep modes (i.e., REM sleep and non-REM sleep), trends and statistics of metabolic variables and blood parameters, ECG and electroencephalogram trends and statistics, pulse transit time trends and statistics, blood pressure trends and statistics, etc. and automated disease diagnoses (e.g., respiratory, cardiovascular, cerebrovascular, metabolic, etc.). Algorithms and techniques described herein can be used to process the data required to create such reports.

In some situations, continuous thermometry can be an important feature in a medical and/or fitness wellness device (e.g., a monitoring device). Skin temperature closely correlates to core body temperature depending on the measurement site. As a result, sensors that measure directly or indirectly the skin temperature can be used to monitor the core body temperature. A continuous skin temperature measurement has several clinical and non-clinical applications. Example clinical applications include the monitoring of core body temperature, which can be accomplished if the temperature sensor(s) is placed on measurement sites such as, but not limited to, a user's forehead, head, ear, axilla, and near the axilla.

When a continuous temperature sensor measures the user's skin temperature at one or more extremities (e.g., wrist, arm, digit, leg, foot, etc.), the skin temperature can be used as a proxy for the user's “comfort level”. Other measurements can be used in combination with the continuous temperature measurements to determine the user's “comfort level”. For example, other physiological measurements such as heart rate, perfusion index, pulse amplitude, respiration rate, oxygen saturation, and the like can be combined with the continuous temperature measurements. For instance, when a user is exposed to low temperatures, the blood circulates mainly around the body's organs to ensure the temperatures of the organs are controlled and experience little changes. Maintaining the temperatures of the organs is possible because the blood circulation at the extremities is reduced, and as a result, the temperature at the extremities (e.g., wrist, arm, digit, leg, foot, etc.) tend to decrease.

The opposite happens when a user is exposed to high temperatures. Blood will circulate mainly at the body's extremities to improve heat exchange with the environment so as to control the temperatures of the organs such that the organs experience little changes. As a result, the temperature at the extremities tends to increase. The measure of the user's “comfort level” is obtained by comparing the temperature(s) from an extremity (or extremities) to the temperature from a measurement site that closely correlates to the core body temperature (e.g., head, forehead, ear, axilla, etc.), or to a certain predefined core body temperature value if the user is in normal health conditions (i.e., no fever or infections) and no significant change in core body temperature is observed. Depending on the difference between the temperature of an extremity and the core body temperature, it is possible to create a mapping that quantifies the user's “comfort level”.

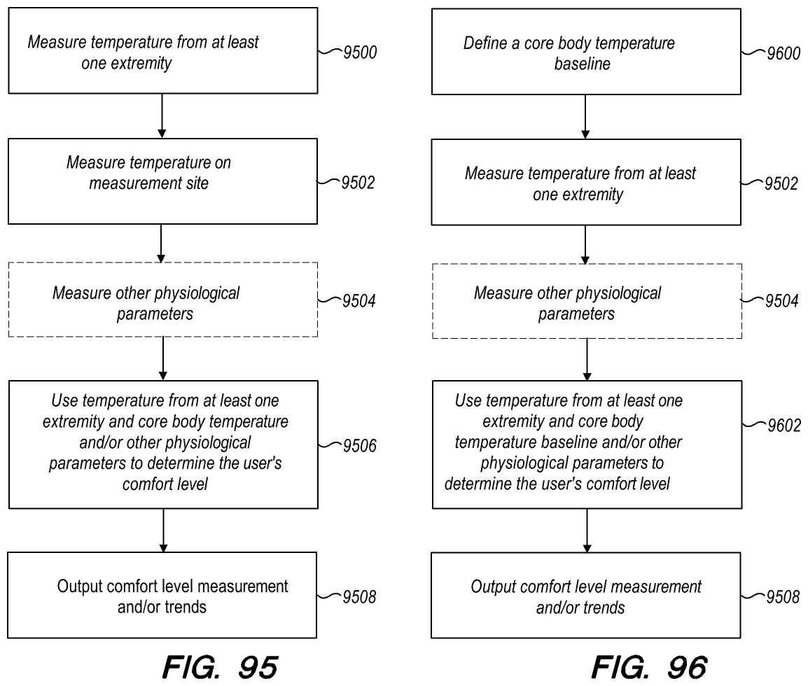

FIG. 95 depicts an example first method of determining a comfort level of a user. The method can be used on human beings or in veterinarian applications. In block 9500, the skin temperature of at least one extremity is measured. In some embodiments, multiple temperature sensors positioned at different extremities are used to obtain more accurate information about the average temperature of the extremities. A skin temperature measurement is taken at block 9502 at a measurement site that closely correlates to the core body temperature. Measurements of other physiological parameters, such as heart rate, perfusion, oxygen saturation, ECG, and respiration rate, can optionally be measured at block 9504.

Next, as shown in block 9506, the measurements obtained at blocks 9500 and 9502 (and optionally block 9504) are used to determine the “comfort level” of the user. In one example technique for determining the “comfort level”, the differences between the core body temperature and the temperatures at one or more extremities are mapped through a single- or multi-variate function (or calcurve) to values that indicate “comfort level”. The comfort level measurement and/or trend data is output in a suitable scale (block 9508). An example scale would be 0-100%, where 100% means very comfortable, and 0% indicates very uncomfortable. Optionally, a qualitative scale can be adopted such as LOW, MEDIUM, and HIGH “comfort levels”. In one embodiment, the comfort level measurement and/or trend data is displayed on a display device.

FIG. 96 illustrates an example second method of determining a comfort level of a user. In this process, the core body temperature is assumed to be a constant baseline value that is defined for a user, a population, or for a period of time depending on exogenous factors. The exogenous factors include, but are not limited to, a location, a time of day, the weather conditions, and/or the user's stress levels.

Initially, as shown in block 9600, a core body temperature baseline is determined. The skin temperature of at least one extremity is measured at block 9502. Measurements of other physiological parameters, such as heart rate, perfusion, oxygen saturation, ECG, and respiration rate, can optionally be measured at block 9504. Next, as shown in block 9602, the measurements obtained at blocks 9600 and 9502 (and optionally block 9504) are used to determine the “comfort level” of the user. The comfort level measurement and/or trend data is then output in a suitable scale (block 9508).

Another application for continuous temperature measurements at the extremities relates to corrections in calorie consumption calculations (e.g., calorie counting). Typical calorie counting meters rely on heart rate measurements, demographic data (e.g., height, weight, gender, age, body area, ethnicity, etc.), and motion sensors (e.g., accelerometers, optical sensors, etc.) to estimate the calorie consumption of a user during his or her daily activities. Calorie consumption, however, is highly dependent on the comfort level of the user. If one assumes that the core temperature is virtually unchanged when the user is in healthy conditions, then the temperature at the body's extremities can be used to correct calorie consumption calculations. A monitoring device, such as a wristwatch, can have a built-in temperature sensor that measures continuously or at select times the skin temperature at the wrist and corrects the calorie count (e.g., obtained from the heart rate and the motion sensor measurements) based on the temperature readings. For instance, assume that a temperature around 33 Celsius at the wrist is considered an equilibrium or baseline temperature for a particular user when the user is at rest and comfortable. A temperature increase or decrease can indicate that the body is expending more energy either by warming up or cooling down. The body warms up or cools down to substantially maintain the temperature of the internal organs at an almost constant temperature (e.g., 36.5-37.5 Celsius). The increase or decrease in the metabolism based on the temperature change may not be detected by changes in the heart rate, and thus, a calorie meter may not be able to detect or compensate for the change in metabolism if the calorie meter does not measure the extremity temperature. Not compensating for the changes in metabolism can produce errors in the estimates of the calorie consumption.

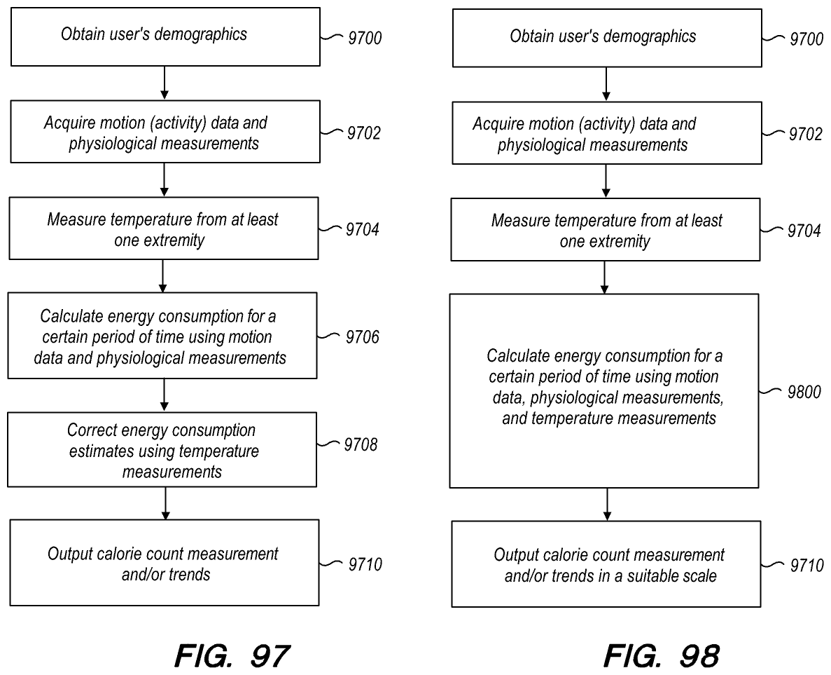

FIG. 97 depicts an example first method of determining a calorie count using temperature measured at least one extremity of a user. The process shown in FIG. 97 can be more suitable in redesigns of existing calorie count meters, where the redesigns include temperature measurements. Initially, demographic data associated a user is obtained at block 9700. Example demographic data includes, but is not limited to, height, weight, gender, age, body area, and ethnicity. In one embodiment, the demographic data is used to select one or more equations or set of equations that best estimate a calorie count for a particular user or population. Depending on the type of equations that are selected, all, some, or none of the demographic data may be used to estimate the calorie count.

Next, as shown in block 9702, motion sensor data, physiological parameters (e.g., heart rate, respiration rate, perfusion index, pulse amplitude, etc.) and/or physiological data (i.e., optical, electrical, mechanical, sound, ultrasound, photoplethysmographs, ECG, EEG, etc.) are acquired or calculated for a given period of time. Again, depending on the type of equations selected to estimate the calorie count, all, some, or none of the motion sensor data, the physiological parameters, and/or the physiological data may be used to estimate the calorie count.

The temperature at one or more extremities of the user is measured during the given period of time (block 9704). An estimate of the energy consumption during the given period of time is then determined using the motion data, the physiological data, the physiological parameters, and/or the temperature measurements (block 9706). A correction to the energy consumption estimate is determined at block 9708 using the temperature measurements acquired in block 9704. In one embodiment, the correction is calculated by convolving data streams of temperature at one or more extremities with linear or nonlinear filter(s) or kernel(s) that weight(s), differentiate(s), and/or integrate(s) temperature functionals over time to produce the required correction to the energy consumption estimate determined in block 9706. The corrected calorie count for the given period of time is then output (block 9710). In anon-limiting example, the corrected calorie count is displayed on a display device.

FIG. 98 illustrates an example second method of determining a calorie count using temperature measured at least one extremity of a user. The process shown in FIG. 98 is similar to the method shown in FIG. 97 except that the process of FIG. 98 does not include block 9708. Initially, demographic data associated a user is obtained at block 9700. Next, as shown in block 9702, motion sensor data, physiological parameters (e.g., heart rate, respiration rate, perfusion index, pulse amplitude, etc.) and/or physiological data (i.e., optical, electrical, mechanical, sound, ultrasound, photoplethysmographs, ECG, EEG, etc.) are acquired or calculated for a given period of time.

The temperature at one or more extremities of the user is measured during the given period of time (block 9704). An estimate of the energy consumption during the given period of time is then determined using the temperature data and one or more of the motion data, the physiological data, or the physiological parameters at block 9800. A calorie count for the given period of time is then output at block 9802.

In the method of FIG. 98, the temperature measurement obtained at block 9704 is used directly to determine the energy consumption. The method shown in FIG. 98 can be more suitable in designs for new calorie count meters that do not make use of existing calorie count equations. Additionally or alternatively, the temperature measurements can be incorporated directly as variables in the equations that are used to estimate the calorie count and validated through clinical tests and/or data collections.

Another application of a continuous temperature measurement is to use the continuous temperature measurement in combination with a pulse oximeter. For example, with pandemics such as the coronavirus disease 2019 (Covid-19) and other viruses, continuous pulse oximetry and thermometry can be an invaluable tool in the early detection of symptoms, such as decreases in oxygen saturation (desaturations) and changes in the core body temperature of a user over time. The continuous monitoring of temperature and oxygen saturation is used on Covid-19 patients (and patients with other respiratory diseases) under treatment.

One of the advantages of integrating thermometry and pulse oximetry in the same monitoring device relates to the ability to detect whether the measurement site is from a real and live user and whether the sensor or sensors in the monitoring device is positioned correctly on the measurement site. With a temperature sensor, information about the temperature alone may not be sufficient to detect if the temperature sensor has been applied to a valid measurement site. However, a strong indication that a measurement site is a valid measurement site (e.g., from a human or an animal) is provided when optical signals (and electrical signals in some multi-parameter monitoring applications) are measured at the same measurement site and when valid measurements are received, such as valid plethysmographs, saturation, heart rate, perfusion index, pulse amplitude, respiration rate, electrical impedance, ECG, and/or EEG measurements. Alternatively, depending on the measurement site, if the temperature is too low, this can indicate that the measurement site needs to be warmed to improve the performance of the monitoring device (e.g., a pulse oximeter). A notification regarding the low temperature can be conveyed to the user or a caregiver via a visible, tactile, and/or audible notification. Abnormally high and low values of temperature, when combined with the inability to obtain pulse oximetry readings, are strong indications that either the sensor(s) in the monitoring device is misplaced or that the measurement site is not from a live human being or live animal. During extended periods of monitoring, sensors tend to be misplaced due to the activity of the user. Additionally or alternatively, with disposable sensors, the adhesives tend to wear out over time, which increases the chances of a misplaced sensor. If a contact temperature sensor is used in combination with a contact pulse oximeter, any sensor dislodgment from the measurement site will affect the readings from the pulse oximeter and from temperature sensor, thereby increasing the possibilities of detecting sensor misplacement and providing alerts/alarms to the user or the caregiver.

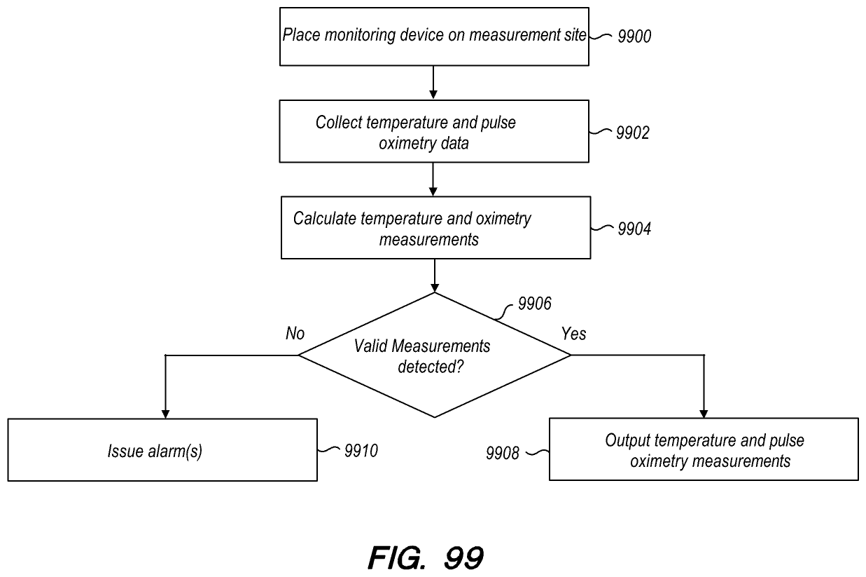

FIG. 99 depicts an example first method of using pulse oximetry and thermometry data to determine a sensor is placed correctly on a valid measurement site. Initially, as shown in block 9900, a monitoring device that includes one or more sensors is placed on a measurement site. The one or more sensors include a temperature sensor and an optical sensor for pulse oximetry. Next, as shown in block 9902, temperature data and pulse oximetry data are received or collected. Based on the temperature data and the pulse oximetry data, thermometry measurements and oximetry measurements are calculated (block 9904).

A determination is made at block 9906 as to whether the thermometry measurements and the oximetry measurements are valid. If so, the process continues at block 9908 where the thermometry measurements and the oximetry measurements are output. For example, the thermometry measurements and the oximetry measurements can be displayed on a display device and/or output to a speaker as audible thermometry measurements and oximetry measurements. If a determination is made at block 9906 that valid measurements are not detected, the method passes to block 9910 where one or more alarm(s) are provided to alert a user and/or a caregiver. Non-limiting example of detecting valid measurements can be oxygen saturation in a valid range, a pulse rate in a valid range, pulse amplitude in a valid range, a perfusion index in valid range, valid photoplethysmographs (i.e., produced from the heart activity of a human or animal), a temperature in a valid clinical range depending on measurement site, and/or a respiration rate in a valid range.

FIG. 100 illustrates an example second method of using pulse oximetry and thermometry data to determine a sensor is placed correctly on a valid measurement site. Initially, as shown in block 10000, a monitoring device that includes one or more sensors is placed on a measurement site. The one or more sensors include a temperature sensor and an optical sensor for pulse oximetry. Next, as shown in block 10002, temperature data and optionally other physiological data and/or non-physiological data are received or collected. Physiological data can include, but is not limited to, pulse oximetry data, blood pressure, ECG, EEG, physical activity (accelerometers), optical data, ultrasound data. Non-physiological data may include, but is not limited to, environment temperature, pressure, and altitude, location, velocity, and acceleration, data received from devices or third parties.

Based on the temperature data and the optional physiological data and/or non-physiological data, thermometry and other physiological and/or non-physiological measurements are calculated (block 10004). A determination is made at block 10006 as to whether the thermometry measurements and the oximetry measurements are valid. If so, the process continues at block 10008 where the thermometry measurements and the optional other physiological and/or non-physiological measurements are output. For example, the thermometry measurements and the and other physiological and/or non-physiological measurements can be displayed on a display device and/or output to a speaker as audible measurements. If a determination is made at block 10006 that valid measurements are not detected, the method passes to block 10010 where one or more alarm(s) are provided to alert a user and/or a caregiver.

FIG. 10A depicts example user interface screens that are suitable for display on a host device. In one embodiment, the user interface screens are displayed at host device. FIG. 101 illustrates optical data and thermometry data collected by a monitoring device. The optical data 10126 is produced by an optical sensor in the monitoring device 100 (e.g., the optical sensor 110 shown in FIG. 1). The thermometry data 10128 is output from one or more temperature sensors in the monitoring device 100 (e.g., temperature sensor 705 shown in FIG. 7B). As described previously, the optical data 10126 and the thermometry data 10128 are sent via wireless connection 1206 to a host device 105 for processing and storage, and for the display of corresponding measurement data and/or waveform data. FIG. 101A and FIG. 101B are described together.

The user interface screen 10102 is created based on data collection that occurs under normal operations, where valid optical data 10126 and thermometry data 10128 are detected and received. Measurement data for SpO2, PR, PI, and temperature (Temp) are shown in the screen 10102. Unlike the user interface screen 10102, the user interface screen 10104 is displayed when valid optical data is not received. Thus, the SpO2, PR, PI, and Temp measurements are displayed as dashed 10106, and a warning message 10108 “Searching signal” is displayed. The warning message 10108 may indicate that the monitoring device 100 is misplaced, placed on an invalid measurement site (i.e., not from a human or animal measurement site) or is placed on a measurement site incapable of producing valid pulsatile signals (photoplethysmographs).

The user interface screen 10110 and the user interface screen 10114 depict scenarios where valid optical data and thermometry data are collected but the thermometry data is outside a normal clinical range for the user. The thermometry data can be greater than or less than the normal clinical range. When the thermometry data is greater than the normal clinical range (high temperature), an alarm is triggered (see alarm 10112 in user interface screen 10110). When the thermometry data is less than the normal clinical range (low temperature), an alarm can be produced (see alarm 10116 in user interface screen 10114). The normal clinical range can be defined by a temperature upper limit 10130 and a temperature lower limit 10132.

The user interface screen 10118 exemplifies a situation where the temperature measurements have dropped below the lowest allowed clinical value 10120, which may indicate the sensor(s) is misplaced or placed on an invalid measurement site. As a result, the SpO2, PR, PI, and Temp measurements are not displayed (e.g., displayed as dashed) and a warning message 10122 of “Temperature too low” is displayed. In one embodiment, the warning message 10122 is presented even when valid optical data is being acquired.

The user interface screen 10124 depicts a host device resuming normal operations after the temperature measurements that were below the lowest allowed clinical value 10120 have increased and reached the normal clinical range. As shown in FIG. 101B, motion artifacts and interferences 10134 are present in the acquired optical signals 10126. The motion artifacts and interferences 10134 are removed via algorithms described herein (e.g., FIGS. 39-46). As a result, even when the collected optical signals are subject to interferences (e.g., electrical, mechanical, optical, etc.) and subject to motion caused by the physical activities of the user, the host device is capable of outputting valid measurements.

FIGS. 102 and 103 illustrate various aspects to consider when designing an accurate thermometry system. The aspects include heat exchange with the measurement site and/or the environment and changes in the internal temperatures and the effects of the temperature estimates at the measurement site. FIGS. 102 and 103 are described in conjunction with the design of a multi-parameter monitoring device that includes one or more temperature sensors, and with the correct estimation of the temperature(s) at the measurement site in light of the internal temperature of the multi-parameter monitoring device.

In FIG. 102, initially a multi-parameter monitoring device with a built-in skin temperature sensor(s) and other internal temperature sensor(s) is designed at block 10202. The thermal design is such that the built-in skin temperature sensor(s) is placed in the multi-parameter monitoring device where the thermal resistance between the multi-parameter monitoring device and the user's skin is lowest or substantially lowest (block 10204). This ensures that the temperature at the measurement site is as close as possible to the temperature(s) measured by the built-in skin temperature sensor(s). In block 10206, the design of the low power electronics for the multi-parameter monitoring device is performed and an encapsulation for the multi-parameter monitoring device is designed so as to increase or maximize the heat exchange with the user's skin at the measurement site and to decrease or minimize the heat exchange with the surrounding environment. A low power electronics design reduces or minimizes the effects that the heat dissipation by the electronics in the multi-parameter monitoring device has on the temperature at the measurements site.

FIG. 103 depicts a method for correcting skin temperature measurements and trends based on temperature measurements or trends. The process shown in FIG. 103 uses temperature measurements and/or trends that are received from one or more temperature sensors in a multi-parameter monitoring device. As noted earlier, embodiments of the monitoring device described herein comprise a multi-parameter monitoring device.

Initially, at block 10302, temperature data is received from a built-in temperature sensor and other internal and/or external temperature sensor(s). In one embodiment, the built-in temperature sensor and the other internal and/or external temperature sensor(s) measure the skin temperature at a measurement site. Using the temperature data received from the built-in temperature sensor, temperature measurements and trends are calculated at block 10304. Using the temperature data received from the other internal and/or external temperature sensors, internal and/or external temperature measurements and trends are calculated at block 10306. Next, as shown in block 10308, the temperature measurements and trends that were determined with the temperature data received from the built-in temperature sensor are corrected using the internal and/or external temperature measurements and trends. In one embodiment, the temperature measured by the built-in temperature sensor is a function of the measurement site temperature (i.e., the skin), the environment temperature, the internal temperature(s) of the monitoring device, the thermal resistance between the built-in temperature sensor and the skin, the device internal circuitry and components, and the environment. Assuming that the thermal resistances are fairly constant for the same monitoring device design when applied to a subject population, then the temperature at the measurement site can be expressed as a function,

\hat{T}ₛᵢₜₑ=ƒ(Tₛₑₙ,Tᵢₙₜ,Tₑₙᵥ,p) Equation 45

where \hat{T}ₛᵢₜₑ is a vector representing temperature estimates at the measurement site's single location and/or time interval (epoch), or at the measurement site's multiple locations and/or time intervals (epochs). Tₛₑₙ is a vector representing temperature readings near or at the measurement site's single location and/or time interval (epoch), or near or at the measurement site's multiple locations and/or time intervals (epochs). Tᵢₙₜ is a vector representing temperature readings at the monitoring device's internal single location and/or time interval (epoch), or at the monitoring device's internal multiple locations and/or time intervals (epochs). Tₑₙᵥ is a vector representing temperature readings at the monitoring device's external (environment) single location and/or time interval (epoch), or at the monitoring device's external multiple locations and/or time intervals (epochs). Parameter p is a vector representing design parameters. In an example embodiment, the function ƒ can have a functional form defined by modeling via the laws of physics (thermodynamics, thermostatic, etc.), and the vector of parameters p can be determined though experimental data where readings from gold standard sensor(s) (calibrated sensor(s) placed on the measurement site(s)) are correlated through multivariate analysis with Tₛₑₙ, Tᵢₙₜ, and Tₑₙᵥ readings, while the measurement site and the environment are subjected to changes in temperature over time. This can be expressed by the optimization problem,

$${p}^{*} = {arg min}_{p} {\sum }_{t = 0}^{n - 1} J ( f ( {T}_{sen , t} , {T}_{int , t} , {T}_{env , t} , p ) , {T}_{site , t} ) \qquad Equation 46$$

where Tₛᵢₜₑ is a vector representing temperature readings from the gold standard sensor(s) at the measurement site's single location and/or time interval (epoch), or at the measurement site's multiple locations and/or time intervals (epochs). J is a sampled cost function (e.g., absolute (L1), least-squares (L2), absolute supremum (L∞), etc.). The index t denotes data samples (n sampled data sets, {T_{sen,t}, T_{int,t}, T_{env,t}, T_{site,t}}) obtained at different points in time (epochs), and environment and measurement site temperatures.

For some designs of monitoring devices, only the readings from Tₛₑₙ are sufficient to obtain accurate \hat{T}ₛᵢₜₑ estimates. For other monitoring device designs, the readings from Tₛₑₙ and Tᵢₙₜ can be sufficient to obtain accurate \hat{T}ₛᵢₜₑ estimates. Changes in the optimization problem (Equation 46) can be made in terms of dropping the temperature vectors that are not applicable to a particular design.

FIGS. 104A-104C illustrate an example monitoring device that includes built-in thermometry and pulse oximetry. FIG. 104A depicts a monitoring device 100 (cross-sectional view) attached to a measurement site 700. The monitoring device 100 includes an optical sensor 110 that has one or more light sources 113 and one or more photodetectors 114, a built-in temperature sensor (T) 705 that measures the temperature (e.g., skin temperature) at the measurement site 700, and a built-in temperature sensor Ti 10400 that measures the internal temperature of the monitoring device 100. At least one surface of the built-in temperature sensor 705 is covered with a rounded thermally conductive material 10402 to prevent the built-in temperature sensor 705 from contacting the skin directly. The size and shape of thermally conductive material 10402 depends on the thermal design of the monitoring device 100. For instance, almost the entire edge of the monitoring device 100 that is in contact with the measurement site 700 can be made of the same thermally conductive material 10402 to minimize the thermal time constants for temperature equalization (steady-state equilibrium) of the measurement site 700 and the internal components in the monitoring device 100. The material 10402 can be an adhesive tape (such as the 3M™ Thermally Conductive Adhesive Transfer Tapes 8805, 8810, 8815 and 8820, etc.) or some other material that is biocompatible and with high thermal conductive and low electrical conductivity (insulator) to reduce the thermal resistance between the built-in temperature sensor 705 and the skin at the measurement site, while preventing the built-in temperature sensor 705 from touching the skin directly to minimize the risks of skin irritation (due to biocompatibility issues) and electrical shocks and/or electrical discharges on the user.

FIG. 104B depicts a top view of the monitoring device and FIG. 104C depicts a bottom view of the monitoring device. The top view shows the edge of the monitoring device that is attached to the measurement site. The outer shell 10406 of the monitoring device 100 is a thermal (and electrical) insulator to minimize the heat exchange with the environment. Measurement(s) from the built-in temperature sensor 705 can be represented by vector Tₛₑₙ, and measurement(s) from the built-in temperature sensor 10400 can be represented by vector Tᵢₙₜ in Equations 45 and 46. In this example embodiment, the external (environment) temperatures around the monitoring device 100 are not measured. The monitoring device 100 in this example embodiment is assumed to be small enough and low power enough to collect, process, and transmit data from the built-in temperature sensors 705, 10400 and from the optical sensor 110 without creating any significant internal heat. As a result, and given the relative small size of the monitoring device 100 and the low thermal resistance between built-in temperature sensor 705 and the skin at the measurement site, the small thermal mass combined with the low internal power dissipation and thermally (and electrically) insulator outer shell 10406 enable accurate estimates of the temperature at the measurement site 700 (\hat{T}ₛᵢₜₑ in Equation 45) using only the built-in temperature sensors 705, 10400. The utilization of the internal temperature sensor 10400 can improve the accuracy of \hat{T}ₛᵢₜₑ during thermal transients and large excursions in environmental temperature.

FIG. 105 illustrates example circuitry that is suitable for use as a built-in temperature sensor in a monitoring device. The built-in temperature sensor 705 is based on the TMP117, a high-accuracy, low-power, digital temperature sensor with I2C-compatible interface integrated circuit (element 10510) from Texas Instruments Incorporated. The TMP117 10510 communicates via I2C with a processing device (e.g., 501 in FIG. 5B-1) via signal lines SDA_I2C_TMP 10508 (data) and SCL_I2C_TMP 10506 (clock), with current levels defined by two pull-up resistors 10500, 10502 (R5 and R6) connected to the power voltage 522 (VCC_TMP) and capacitor 10006 (C19). In one embodiment, the resistors 10500, 10502 are five thousand (5000) Ohms (or 5 k Ohms) resistors and the capacitor 10506 is a 0.1 microfarad decoupling capacitor. The temperature probe for the built-in temperature sensor 705 is the SMD package and leads of the integrated circuit 10510.

An example temperature sensor for the built-in internal temperature sensor 10400 can be a built-in temperature sensor that measures the die temperature of the processing device (e.g., 501 in FIG. 5B-1).

FIG. 106 depicts an example embodiment that is used to describe how to apply the embodiments described herein to implement a distributed biosensing system with a wristwatch and a forehead patch both worn by a user. The forehead patch is a monitoring device 100. The wristwatch 1702 in this example has the dual function of a host device (e.g., host device 105 in FIG. 1) and of an integrated device (e.g., integrated device 1218 in FIG. 12B). The monitoring device 100 includes an additional adhesive tape 10602 (similar to embodiments shown in FIGS. 2C, 79A-F) that can be part of the monitoring device 100 or separate to increase the adhesion area on the forehead to prevent the monitoring device 100 from detaching from the forehead, which enables the prolonged use of the monitoring device 100. The monitoring device 100 and the wristwatch 1702 are connected wirelessly through connection 1206. The wristwatch 1702 is also wirelessly connected to the cloud computing server(s) 1204 by means of the wireless connection 1212. The temperature and optical data are collected, processed, and sent wirelessly from the monitoring device 100 to the wristwatch 1702 (e.g., the host device). The wristwatch 1702 processes the received data and stores and outputs measurements such as Temperature 10604, SpO2 10606, PR 10608, and PI 10610 (or PA in an alternate embodiment) and alarms (not shown). The temperature, motion, and optical data collected by the sensors in the wristwatch 1702 related to calories and comfort measurements are processed, stored, and output by the wristwatch 1702 as Calories 10614 and Comfort 10616 measurements. For the Calorie and Comfort measurements 10614, 10616, the wristwatch 1702 acts as an integrated device (e.g., integrated device 1218 in FIG. 12B). The wristwatch 1702 has circuitry 10620 that includes optical sensor(s), motion sensor(s), and built-in skin temperature sensor(s) to sense the user's activity, heart rate, and wrist temperature and enable accurate calculations of the calorie count and the comfort level that are presented (e.g., displayed continuously or at select times) by the wristwatch 1702. It is also possible to use the temperature and optical data received from the monitoring device 100 to improve the accuracies of the calorie count and/or the comfort level. Likewise, the wristwatch 1702 can use the data received from the monitoring device 100 (e.g., the data received from the optical sensor(s) in the monitoring device 100) to estimate time delays (or phase shifts) between the measured forehead and wrist photoplethysmographs (waveforms). These time delays can be used to estimate (or serve as a proxy for) the user's arterial blood pressure level, which can be output continuously or at select times on the wristwatch 1702 (e.g., BP 10612). The time delays can also be used to improve the accuracy of the Comfort 10616 measurements given that the time delays are also a proxy for stress level. The shorter the time delays (or phase shifts) between the forehead (or head, temple, etc.) and the wrist (or digit, foot, leg, arm, etc.) photoplethysmographs, the higher the user's stress level or blood pressure, which in turn can affect the user's comfort level. Depending on the user's activity, the wristwatch 1702 can have built-in algorithms that either display the waveforms 10618 from the optical data received from the monitoring device 100, or directly from the data collected by the optical sensor(s) in the wristwatch 1702, or as a combination of both. All these different configurations are possible because of the distributed nature of the system depicted in FIG. 106.

Battery technology is one aspect of wearable devices (including garments). Wearable devices typically employ battery and/or energy harvesting technologies to power the circuitries in the wearable devices. Batteries require recharging or replacement, and energy harvesting technologies (i.e., mechanical, optical, electromagnetic, thermal, etc.) do not always guarantee that energy is available in all possible operating conditions.

Disclosed herein are several embodiments where wearable monitoring devices and garments can be made so that the biological tissue of a user acts as the electrolytes of a battery, and the circuitry in a wearable monitoring device or a garment provides the electrodes (i.e., cathode(s) and anode(s)) of the battery. As used herein, the term “wearable monitoring device” is to be interpreted broadly to include wearable electronic devices, garments, eyewear, jewelry, and other technologies that are worn on or in the body of a user. When the electrodes (or plates) in the wearable monitoring device are in contact with the biological tissue (i.e., skin) of the user, a bio-battery is formed where the energy is generated from the electrolytes of the user (skin, biological tissue) and the plates or the electrodes (cathode(s) and anode(s)) in the circuitry of the wearable monitoring device.

Several materials and technologies may be adopted or adapted to create electrodes or plates that are suitable for a particular application. For instance, a wearable monitoring device can be built with circuitry that has an anode plate of magnesium and a cathode plate of copper. The magnesium and copper plates form a bio-battery when the magnesium and copper plates are in contact with the skin of the user. The bio-battery formed by the magnesium and the copper plates can be used to power the circuitry in the wearable monitoring device. Several materials and compounds can be used as cathode and anodes. Examples of materials for a cathode electrode or plate include copper, iron, graphite, etc., and example materials for an anode electrode or plate include zinc, magnesium, aluminum, etc. The materials for the cathode and the anode plates/electrodes can be in pure form or as alloys and compounds. The voltage, current, and energy level of a bio-battery are functions of the materials and the dimensions of the cathode and the anode electrodes, the metabolic state of the user's biological tissue (epidermis hydration, skin sweet levels, electrolyte levels, etc.), and the form of contact between cathode and the anode electrodes and the user's biological tissue. For instance, a bio-battery is formed by attaching a copper foil electrode (cathode electrode) to a user's biological tissue and a thin magnesium electrode (anode electrode), both with a few square inches of contact surface area (with the electrodes separated by a gap of a few millimeters). This type of a bio-battery can produce short-circuit currents of a few microamps to dozens of microamps and an open terminal voltage around one and a half (1.5) volts. The bio-battery leads to a Thevenin equivalent voltage source circuit with a sufficiently low impedance to provide energy to power a wearable monitoring device 100 with one or more thermometry, Ph, humidity, interstitial lactate and glucose, and/or heart rate sensor(s) that acquire measurements from the measurement site every thirty (30) or sixty (60) seconds and transmits the measurements wirelessly to a host device 105 for display, alarms, and/or storage. Depending on the materials, the effective contact area of the cathode and the anode electrodes with the user's skin, and a low-power circuitry designs, bio-batteries can be used to power wearable monitoring devices or patches that are pulse oximeters, continuous non-invasive or minimally invasive glucometers, vital signs patches and/or monitors, ECG and/or EEG patches, bioimpedance-based devices, ultra-sound based devices, smart garments (i.e., garments with circuitries and/or sensing functionalities) such as shirts, t-shirts, socks, underwear, dresses, pants, and/or the like.

FIG. 107 illustrates a flowchart of an example first method of operating a wearable monitoring device that includes one or more bio-batteries. Initially, as shown in block 10700, a wearable monitoring device with circuitry, cathode electrode(s), and anode electrode(s) is attached to the biological tissue (e.g. skin) of a user. After attachment, the cathode electrode(s) and the anode electrode(s) form one or more bio-batteries, where the biological tissue of the user serves as the electrolyte for the bio-battery or bio-batteries (block 10702).

FIG. 108 depicts a flowchart of an example second method of operating a wearable monitoring device that includes one or more bio-batteries. In one embodiment, the frequency of operation (interval between operations) is defined by the one or more bio-batteries and the power levels of the circuitry in the wearable monitoring device. In the illustrated embodiment, the one or more bio-batteries has a current capacity compatible with the measurement frequency of the wearable monitoring device to ensure the measurement data and/or trend data of interest is captured.

Initially, the energy produced by the one or more bio-batteries accumulates in one or more electrical accumulators (e.g., one or more capacitors, etc.) at block 10800. A determination is made at block 10802 as to whether a sufficient amount of energy has accumulated. For example, a determination may be made as to whether the accumulated energy equals a pre-defined threshold (e.g., defined by the voltage levels of the one or more capacitors). If not, the process returns to block 10800.

When a determination is made at block 10802 that there is a sufficient amount of accumulated energy, the method continues at block 10804 where the accumulated electrical energy is converted to appropriate voltage and/or current levels to power on (turn on) the circuitry in the wearable monitoring device. In block 10806, the circuitry in the wearable monitoring device acquires or receives data and processes the data. The circuitry in the wearable monitoring device then stores, transmits, receives, and/or outputs the data (block 10808). The data can be measurement data and/or trend data that are transmitted to a host device, to a computing system (e.g., computing system 1202 in FIG. 12A), and/or to a cloud computing system (e.g., cloud computing system 1204 in FIG. 12A). The process shown in FIG. 108 is suitable for applications where the maximum power capacity of the one or more bio-batteries is not sufficient to ensure the continuous operation of the circuitry in the wearable monitoring device, but is adequate to ensure periodic or aperiodic spot-check operations of the wearable monitoring device.

FIG. 109 illustrates a flowchart of an example third method of operating a wearable monitoring device that includes one or more bio-batteries. The process shown in FIG. 109 can be used when the power capacity of a bio-battery is large enough to enable the continuous operation of a wearable monitoring device 100. Initially, the wearable monitoring device (with circuitry, cathode electrode(s), and anode electrode(s)) is attached to the biological tissue (e.g., skin associated with measurement site) of the user to form one or more bio-batteries that can continuously power the circuitry of the wearable monitoring device (blocks 10700, 10702). The circuitry of the wearable monitoring device is powered on in block 10900, which may also implement the processes of authentication and pairing with a host device, or connecting with a router or appliance that connects the wearable monitoring device to a computing system or to a cloud computing system (e.g., computing system 1202 or cloud computing system 1204 in FIG. 12A).

Next, as shown in block 10902, the circuitry of the wearable monitoring device (e.g., the power management modules) continuously accumulates energy produced by the one or more bio-batteries and converts the accumulated energy into the appropriate voltage and current levels that are used to power the circuitry of the wearable monitoring device. The circuitry of the wearable monitoring device acquires/receives and processes data continuously (block 10806), and continuously stores, transmits, receives, and/or outputs the data (block 10808). For example, the data can be measurements, trends, parameters, and/or waveforms that are transmitted to a host device continuously or at select times, or directly to a computing system, or directly to a cloud computing system.

FIG. 110 depicts a flowchart of an example method of operating a wearable monitoring device that includes reusable circuitry and disposable component(s) with at one least bio-battery. Initially, the reusable circuitry in the wearable monitoring device is attached to one or more disposable components, including at least one cathode electrode and/or at least one anode electrode (block 11000). The wearable monitoring device with the reusable circuitry and the disposable component(s) is attached to the biological tissue (e.g., skin) of a user and begins operating at block 11002. After a period of time, at least one disposable component of the wearable monitoring device is replaced due to, for example, wear and tear of an adhesive tape or tapes, oxidation of the anode electrode, and/or reduction of the cathode electrode. The wearable monitoring device is then detached from the biological tissue and the at least one disposable component is replaced, including at least one cathode electrode and/or at least one anode electrode (blocks 11004, 11006). In some embodiments, block 11004 is optional because at least some of the disposable component(s) can be replaced without detaching the wearable monitoring device from the biological tissue. The process shown in FIG. 110 can be repeated at select times when one or more disposable components are to be replaced.

In FIG. 110, the disposable component(s) can be adhesive tapes with different metal foils and/or plates and/or chemical elements/compounds suitable for bio-battery electrode applications. In some embodiments, a disposable component can have the dual role of housing the reusable circuitry and the cathode and anode electrodes of the wearable monitoring device, and attaching the wearable monitoring device (with circuitry, adhesive tapes, and the cathode and anode electrodes) to the biological tissue that is the measurement site (e.g., skin). A wearable monitoring device in the embodiment shown in FIG. 110 can be implement as a vital signs monitor, a continuous invasive or minimally invasive glucometer, a pulse oximeter, and the like. The disposable component(s) can have an anti-tamper secure memory to provide secure authentication with the circuitry of the wearable monitoring device to minimize the risks for the user by limiting the total usage time and preventing counterfeiting.

FIG. 111 illustrates a cross-section of a measurement site and cross-section and top views of an example wearable monitoring device that is powered by a bio-battery. In the illustrated embodiment, the wearable monitoring device 100 can be a single use and fully disposable wearable monitoring device or a reusable wearable monitoring device. The example wearable monitoring device 100 includes a temperature sensor 705, a cathode electrode 11100, an anode electrode 11102, and an optical sensor 110 comprising one or more light sources 113 and one or more photodetectors 114. The wearable monitoring device 100 is powered by a bio-battery formed by the cathode electrode 11100 and the anode electrode 11102 in contact with the measurement site 700 (user's skin).

The wearable monitoring device 100 (powered by the bio-battery) is capable of sensing the temperature though the temperature sensor 705 and optical signals using the optical sensor 110. The one or more light sources 113 emit light 702 towards the measurement site 700 that penetrates the epidermis 703 and the blood perfused dermis 704 and interacts with the heart's pulsatile signal to create optical pulsatile signals. FIG. 112 depicts the bio-battery of the wearable monitoring device shown in FIG. 111. The bio-battery 11200 includes the cathode electrode 11100 and the anode electrode 11102 that interact with the measurement site 700, where the measurement site 700 acts as the electrolyte of the bio-battery 11200. The bio-battery 11200 is operably connected to the circuitry 11202 of the wearable monitoring device 100. To simplify the description of the operation of the wearable monitoring device 100, the circuitry 11202 is shown as divided into two components, the electrical capacitor 11204 and a circuitry box 11606 that contains all other components and systems such as power management integrated circuits, microprocessor(s), ratio(s), frontends, sensors, etc. of the circuitry of the wearable monitoring device 100. For example, the circuitry 11606 can include some or all of the components shown in FIG. 3.

FIG. 113 is a chart illustrating an example operation of the wearable monitoring device shown in FIG. 111. The example wearable monitoring device 100 includes an operation mode that is suitable for applications where the maximum power capacity of the bio-battery is not sufficient to ensure the continuous operation of the wearable monitoring device but is adequate to ensure periodic or aperiodic spot-check operations. The chart 11300 depicts the voltage of the capacitor 11204 (FIG. 112) during a charging phase 11302 of the wearable monitoring device 100 (curve 11304) and an ON phase 11306 (curve 11308) of the wearable monitoring device 100. For a given set of cathode and anode electrodes 11100, 11102 (FIG. 111), the duration of the charging phase 11302 is defined by the electrical capacity (capacitance) of the capacitor 11204 (FIG. 112), the electrical impedance of the bio-battery 11200 (FIG. 112), and the voltage that is used to start normal operations (e.g., sensing operations) of the wearable monitoring device 100. The capacitance of the capacitor 11204 is a function of the minimum energy level needed for the wearable monitoring device 100 to operate and to execute a full sequence of spot-check operations in the ON phase 11306, and the electrical capacitor voltage required for the wearable monitoring device 100 to start and safely end a full sequence of spot-check operations. The impedance of the bio-battery 11200 is function of the electrical and physiological properties of the measurement site, the dimensions and the composition of the cathode and anode electrodes 11100, 11102, and the contact impedance between the cathode and the anode electrodes 11100, 11102 and the measurement site 700.

The electrical energy stored in the capacitor 11204 can be converted into the appropriate voltage and/or current levels that is used for the wearable monitoring device 100 to operate during the ON phase 11306. The curve 11310 is a typical voltage profile generated by a power management system (part of the circuitry 11202) that converts the voltage level of the capacitor 11204 to a higher/lower and regulated voltage suitable for operation of the wearable monitoring device 100. In FIG. 113, during the ON phase 11306, the power management system can have a boost and/or buck converter that is capable of maintaining the required voltage (curve 11310) to ensure the correct operation of the wearable monitoring device 100 whenever the capacitor 11204 is discharging and the voltage of the capacitor 11204 is decreasing from a maximum voltage value 11312 to a minimum voltage value 11314. When the voltage of the capacitor 11204 reaches the minimum voltage value 11314, the circuitry 11202 is disabled or enters a low-power mode (with no or reduced number of functionalities) to enable the capacitor 11204 to be recharged by the bio-battery 11200. In the ON phase 11306, the wearable monitoring device 100 executes spot-check operations such as, for example, the operations discussed in conjunction with blocks 10806 and 10808 in FIG. 108.

The cathode and the anode electrodes 11100, 11102 shown in FIG. 111 can have any shape or form that is suitable for a particular application. In addition, multiple electrodes (i.e., multiple cathodes and anodes) may be connected in series and/or in parallel to optimize or to increase/decrease the equivalent open circuit voltage and impedance characteristics of a bio-battery.

FIGS. 114-128 illustrate embodiments where a bio-battery is implemented in smart garments that include one or more monitoring devices. There are several manufacturing techniques that can be used to add the cathode and the anode electrodes of a bio-battery into garments, where the garments are made of, or include, electrically conductive fabrics or fibers of different materials. Conductive fabrics can be produced by coating nonconductive fabrics made of cotton, nylon, wool, polyester, etc. with conductive materials or compounds via a number of different coating processes. The coating processes include, but are not limited to, electroless plating, plasma treatment, chemical vapor deposition, and textile printing. Conductive fabrics can also be made by blending nonconductive fibers with conductive metals and alloys such as gold, silver, carbon, nickel, titanium, copper, and/or stainless steel. Other new and more efficient processes to manufacture conductive fabrics with electrical, thermal, optical, and mechanical propertied that are more suitable for biosensing applications will be developed as the technologies for creating conductive fabrics or fibers evolve.

FIG. 114 depicts a flowchart of an example method of operating a garment that includes one or more bio-batteries. Initially, a user wears a garment that includes circuitry, one or more cathode electrodes, and one or more anode electrodes that are partially or in full contact with the biological tissue of the user (block 11400). In block 11402, the cathode electrode(s), the anode electrode(s), and the biological tissue of the user form one or more bio-batteries. As discussed previously, the biological tissue acts as the electrolyte of the bio-battery or bio-batteries.

FIG. 115 illustrates a flowchart of an example first method of operating a garment that includes one or more bio-batteries and one or more monitoring devices. In the representative process, the frequency of operation of the one or more monitoring devices in the smart garment (interval between operations) is defined by the one or more bio-batteries and the power levels of the circuitry of the smart garment (including the one or more monitoring devices). In this example application, the one or more bio-batteries has a current capacity compatible with the operation frequency of the smart garment to ensure the measurement data and/or trend data of interest is captured by the monitoring device(s).

The energy produced by the one or more bio-batteries accumulates in one or more electrical accumulators (i.e., capacitors, etc.) at block 11500. A determination is made at block 11502 as to whether a sufficient amount of energy has accumulated. For example, a determination may be made as to whether the accumulated energy equals a pre-defined threshold (e.g., defined by the voltage levels of the one or more capacitors). If not, the process returns to block 11500.

When a determination is made at block 11502 that there is a sufficient amount of accumulated energy, the method continues at block 11504 where the accumulated electrical energy is converted to appropriate voltage and/or current levels to power on (turn on) the circuitry in the smart garment. In block 11506, the one or more monitoring device(s) in the smart garment acquires or receives data and processes the data. The circuitry in the smart garment then stores, transmits, receives, and/or outputs the data (block 11508). The data can be measurements that are transmitted to a host device, to a computing system (e.g., computing system 1202 in FIG. 12A), and/or to a cloud computing system (e.g., cloud computing system 1204 in FIG. 12A). The process shown in FIG. 115 is suitable for applications where the maximum power capacity of the one or more bio-batteries is not sufficient to ensure the continuous operation of the smart garment, but is adequate to ensure periodic or aperiodic spot-check operations by the monitoring device(s) in the smart garment.

FIG. 116 depicts a flowchart of an example second method of operating a garment that includes one or more bio-batteries and one or more monitoring devices. The process shown in FIG. 116 can be used when the power capacity of the one or more bio-batteries is large enough to enable the continuous operation of the smart garment. Initially, the smart garment is worn with the circuitry, cathode electrode(s), and anode electrode(s) in partial or complete contact with the biological tissue (e.g., skin) of the user to form one or more bio-batteries that can continuously power the smart garment (blocks 11400, 11402). The circuitry of the smart garment, including the one or more monitoring devices, is powered on in block 11600, which may also implement the processes of authentication and pairing with a host device, or connecting with a router or appliance that connects the circuitry of the smart garment to a computing system or to a cloud computing system (e.g., computing system 1202 or cloud computing system 1204 in FIG. 12A).

Next, as shown in block 11602, the circuitry of the smart garment (e.g., the power management modules) continuously accumulates the energy produced by the one or more bio-batteries and converts the accumulated energy into the appropriate voltage and/or current levels that is used to power the circuitry of the smart garment (including the one or more monitoring devices). The one or more monitoring devices in the smart garment acquire/receive and process data continuously (block 11506), and the smart garment (e.g., the one or more monitoring devices) stores, transmits, receives, and/or outputs the data (block 11508). For example, the data can be measurements, trends, parameters, and/or waveforms that are transmitted to a host device, a computing system, and/or a cloud computing system continuously or at select times.

FIG. 117 illustrates a flowchart of an example method of operating a disposable smart garment that includes reusable circuitry, at least one bio-battery, and at least one monitoring device. Initially, the reusable circuitry is attached to a new disposable smart garment (block 11700). In one embodiment, the reusable circuitry includes the at least one monitoring device and the disposable smart garment includes the cathode electrode and the anode electrode of the at least one bio-battery. A user wears the disposable smart garment and the circuitry of the disposable smart garment (including the at least one monitoring device) begins operating at block 11702 (powered by the at least one bio-battery). The user wears the smart garment for a period of time with the cathode electrode and the anode electrode of the at least one bio-battery in partial or complete contact with the biological tissue of the user (block 11704). After the period of time, the disposable smart garment is replaced due to, for example, wear and tear, oxidation of the anode electrode, and/or reduction of the cathode electrode. The reusable circuitry is detached from the disposable smart garment, and the disposable smart garment is disposed of or recycled (block 11706). The process can return to block 11700 since the reusable circuitry is now available for attachment to another disposable smart garment. The process shown in FIG. 117 can be repeated at select times when a disposable smart garment is to be replaced.

In non-limiting examples, the one or more monitoring devices in the embodiment shown in FIG. 117 may be implemented as a vital signs monitor, a continuous invasive or minimally invasive glucometer, a pulse oximeter, and the like. The disposable smart garment can also have built-in circuitry with anti-tamper secure memory to provide secure authentication with the reusable circuitry to minimize the risks for the user by limiting the total usage time and preventing counterfeiting.

FIG. 118 depicts a flowchart of an example first method of operating a washable smart garment. The process shown in FIG. 118 is suitable for a washable smart garment that includes detachable non-washable circuitry. In one embodiment, the non-washable circuitry includes a cathode electrode and an anode electrode of at least one bio-battery and at least one monitoring device.

Initially, as shown in block 11800, the non-washable circuitry is attached to the washable smart garment. The user wears the washable smart garment for a period of time with the non-washable circuitry operating during the period of time (block 11802). To power the non-washable circuitry, the cathode electrode and the anode electrode of the at least one bio-battery are in partial or complete contact with the biological tissue of the user while the user wears the disposable smart garment, and the at least one monitoring device is operational while the user wears the washable smart garment (block 11804). Next, as shown block 11806, the non-washable circuitry is detached from the washable smart garment so that washable smart garment can be washed. The process shown in FIG. 118 can be repeated whenever the washable smart garment is to be worn by the user.

FIG. 119 illustrates a flowchart of an example second method of operating a washable smart garment. The process shown in FIG. 119 is suitable for a washable smart garment that includes washable circuitry. In one embodiment, the washable circuitry is built into the washable smart garment and includes at least one bio-battery and at least one monitoring device.

Initially, the user wears the washable smart garment for a period of time with the washable circuitry operating during the period of time (blocks 11900, 11902). To power the washable circuitry, the cathode electrode and the anode electrode of the at least one bio-battery are in partial or complete contact with the biological tissue of the user while the user wears the disposable smart garment, and the at least one monitoring device is operational while the user wears the washable smart garment (block 11904). Next, as shown block 11906, the washable smart garment with the washable circuitry is washed. The process shown in FIG. 119 can be repeated whenever the washable smart garment is to be worn by the user.

FIGS. 120-122 depict example smart garments that have the cathode and anode electrodes of bio-batteries distributed throughout the smart garment. The garments in FIGS. 120-122 are shown as transparent to enable viewing of the cathode electrodes and the anode electrodes. However, the garments described herein can be transparent, opaque, implemented in any color, texture, and suitable shape, loose, tight, or custom fit, and made of conductive, semiconductive, and/or nonconductive materials suitable for a particular application.

FIG. 120 shows a shirt 12000 that has the anode electrodes 12002 and the cathode electrodes 12004 distributed across the internal surface of the shirt 12000. The anode and the cathode electrodes 12002, 12004 are on the internal surface of the shirt 12000 to enable the anode electrodes 12002 and the cathode electrodes 12004 to be in contact with the user's skin. FIG. 121 illustrates a pair of shorts 12100 that has the anode and the cathode electrodes 12002, 12004 distributed across the internal surface of the shorts 12100. Again, the anode and the cathode electrodes 12002, 12004 are on the internal surface of the shorts 12100 to enable the anode and the cathode electrodes 12002, 12004 to be in contact with the user's skin. FIG. 122 depicts a sock 12200 that has the anode and the cathode electrodes 12002, 12004 distributed across the internal surface of the sock 12200 to enable the anode and the cathode electrodes 12002, 12004 to be in contact with the user's skin. Other embodiments can use any suitable type of garment and are not limited to a shirt, shorts, or a sock.

The distribution, shape, and materials of the anode and the cathode electrodes 12002, 12004 depend on the functionalities, power, computing and wireless requirements, and the type of use (i.e., continuous, spot-check), of the one or more monitoring devices that are powered by the bio-batteries. The anode and the cathode electrodes 12002, 12004 are connected electrically via one or more internal layers of the garment so as to form a network 12300 of bio-batteries as shown in FIG. 123. Each node of the network 12300, represented by node 12302, has electrical impedances that are a function of the electrical connection, the anode and the cathode electrodes 12002, 12004, and the impedances of the user's skin. The layout of the network 12300 is typically chosen so as to maximize or increase the power capacity of the bio-batteries while allowing some or all of the cathode and the anode electrodes 12002, 12004 to act as sensor electrodes that measure the body's electrical activity (e.g., ECG), bioimpedance, and/or vital signs such as respiration rate and/or heart rate. This duality in the anode and the cathode electrodes 12002, 12004 creates tremendous flexibility in terms of biosensing functionalities.

The shirt 12000, the shorts 12100, and the sock 12200 (the garments) can have built-in circuitry 12400 shown in FIG. 124. In one embodiment, the circuitry 12400 is distributed around the garments. The anode and the cathode electrodes 12002, 12004 are input into the built-in circuitry 12400 to provide the electrical power and to measure the body's electrical activity, bioimpedance, and/or vital signs. The built-in circuitry 12400 can also have optical, thermal, motion, ultrasound, etc. sensors and frontends to enable the measurement of oxygen saturation, body temperature, blood glucose, total hemoglobin, dyshemoglobinemia, plethysmographs, pulse transit time (with shirt 12000), and the like. For example, the circuitry 12400 can include some or all of the components shown in FIG. 3.

In some embodiments, the garments 12000, 12100, 12200 can include the network 12300 and the built-in circuitry 12500 (FIG. 125) with the connections all lumped together into a single positive terminal 12502 (+Vbiobat) and a single negative terminal 12504 (−Vbiobat) that powers the built-in circuitry 12500. Like the circuitry 12400 in FIG. 124, the circuitry 12500 can include some or all of the components shown in FIG. 3.

Individual anode and cathode electrodes 12002, 12004 can be input into the circuitry 12500 via a capacitive coupling network 12506 that can also serve as a sensor electrode. The network 12300 of bio-batteries (FIG. 123) shares the same electrolyte (the user's skin), and as a result, battery parallel connections do not necessarily imply an increase in electrical current capacity, and battery serial connections do not necessarily imply an increase in overall electrical voltage. The net result of bio-battery parallel and serial connections, when sharing the same electrolyte, is a reduction in the overall electrical impedance of the bio-batteries and an increase in the overall capacity of the bio-batteries.

FIG. 126 illustrates a front and a back view of an example shirt, where the shirt includes two continuous areas that serve as the cathode and the anode electrodes of a bio-battery. The shirt 12600 is transparent to facilitate the visualization of the continuous areas that serve as the cathode and the anode electrodes of the bio-battery. However, the shirt can be transparent, opaque, implemented in any color, texture, and suitable shape, loose, tight, or custom fit, and made of conductive, semiconductive, and/or nonconductive materials suitable for a particular application.

The cathode electrode in the front view 12602 and in the back view 12604 is represented by a first region 12606 and the anode electrode by a second region 12608. The first and the second continuous electrically conductive regions 12606, 12608 touch the user's skin directly to create a bio-battery that powers the circuitry 12700 shown in FIG. 127. In one embodiment, the circuitry 12700 is distributed around the shirt 12600.

The cathode and the anode electrodes in the first and the second regions 12606, 12608 can also serve as sensor electrodes to enable sensing of the electrical activity of the heart. In some embodiments, the cathode and the anode electrodes in the first and the second regions 12606, 12608 can be input into the circuitry 12700 via capacitive coupling network 12702 to filter the electrical signals from the heart from the electrical currents and/or voltages of the bio-battery. It is also possible to apply frequency sweeps (voltage/current signals) from the circuitry 12700 to the cathode and the anode electrodes to perform Bioelectrical Impedance Analysis (BIA) and/or to estimate the body cell, fat, extracellular tissue, and fat-free concentrations and/or masses.

The isocurves 12610, 12612 represent lines of isoelectric potentials on the user that are created by the heart's electrical activity and can be used to optimally design the shape and the separation of the first and the continuous second regions 12606, 12608 that serve as both the cathode and the anode electrodes of the bio-battery and sensor electrodes. The illustrated isocurves 12610, 12612 on the user can be modeled via quasi-static Maxwell equations and/or generalized Laplace equation with boundary conditions. By using the quasi-static Maxwell and/or the generalized Laplace equations, it is possible to create shapes and locations for the cathode and the anode electrodes (e.g., first and second regions 12606, 12608) of the bio-battery that can also sense the electrical activity of the heart under the various planes (i.e., sagittal, coronal, and transverse), while providing electrical energy for the built-in circuitry 12700 (FIG. 127) of the shirt 12600.

The shirt 12600 depicted in FIG. 126 can be wirelessly connected to a host device, to a computing system, and/or to a cloud computing system for some or all of the operations described herein. The operations include, but are not limited to, display, storage, alarm generation, notifications, and real-time data sharing with third parties (e.g., remote caregivers in homecare settings). Other embodiments can use any suitable type of garment and are not limited to a shirt.

FIG. 128 illustrates an example user interface screen that is suitable for displaying measurements calculated from data received from the garments shown in FIGS. 120-122 and FIG. 126. In one embodiment, the user interface screen 12800 is displayed on a host device. The circuitry of the garment (e.g., circuitry 12700 of shirt 12600) can have optical and thermal sensors and frontends to acquire optical and thermal data that enables the calculation of, for example, temperature 12802, SpO2 12804, PR 12814, PI 12806, RR 12808, and photoplethysmographs 12810 measurements. For example, the circuitry shown in FIGS. 124, 125, and 127 can include some or all of the components shown in FIG. 3.

The cathode and the anode electrodes (e.g., cathode and anode electrodes 12606, 12608) can also sense the electrical activity waveforms of the heart and changes in the electrical impedances of the torso, which can be acquired by the circuitry in the garments (e.g., circuitry 12700 in shirt 12600) to measure, for example, ECG 12812, PR 12814, and RR 12808. The cathode and the anode electrodes (e.g., 12606, 12608) of the bio-battery or bio-batteries go through a process of oxidation and reduction while the user wears the garment (e.g., shirt 12600). Oxidation and reduction affect the capacity of the bio-battery or bio-batteries to provide energy to the circuitry of the garment (e.g., circuitry 12700 of the shirt 12600). It is therefore useful to calculate the internal impedance of the bio-battery or bio-batteries with usage and translate the calculated internal impedance into an equivalent measurement (e.g., battery life) as depicted by the battery symbol 12816 in the user interface screen 12800. For instance, a battery full icon can represent a pre-determined number of hours or days the garment (e.g., the shirt 12600) can continuously monitor the user.

Depending on the design and the application, the garments FIGS. 120-122 and 126 can be washable, disposable, with removal/non-removal, washable/non-washable, continuous/spot-check circuitry. The data acquired and processed by the garments in FIGS. 120-122 and 126 can be biosensing measurements, trends, parameters, and/or waveforms that can be continuously (or spot-check) and wirelessly transmitted to host device, to computing system, and/or to a cloud computing system for display, storage, alarm generation, notifications, real-time data sharing with third parties (e.g., remote caregivers in homecare settings), etc.

The descriptions and/or the figures of some of the embodiments disclosed herein include specific components, configurations of components, component values, user interface values and layouts, technical specifications, measurement values, trend data, waveforms, and other values. The specific components, configurations of components, component values, user interface values and layouts, technical specifications, measurement values, trend data, waveforms, and other values are intended to be non-limiting examples. Other embodiments are not limited to these specific components, configurations of components, component values, user interface values and layouts, technical specifications, measurement values, trend data, waveforms, and other values.

The description and illustration of one or more aspects provided in this application are not intended to limit or restrict the scope of the disclosure as claimed in any way. The aspects, examples, and details provided in this application are considered sufficient to convey possession and enable others to make and use the best mode of claimed disclosure. The claimed disclosure should not be construed as being limited to any aspect, example, or detail provided in this application. Regardless of whether shown and described in combination or separately, the various features (both structural and methodological) are intended to be selectively included or omitted to produce an embodiment with a particular set of features. Having been provided with the description and illustration of the present application, one skilled in the art may envision variations, modifications, and alternative aspects falling within the spirit of the broader aspects of the general inventive concept embodied in this application that do not depart from the broader scope of the claimed disclosure.

What is claimed is:

1. A monitoring device, comprising:
an optical sensor within an enclosure of the monitoring device and comprising a light source and a photodetector, wherein the light source is operable to emit light towards the measurement site and the photodetector is operable to receive light reflected from the measurement site when the monitoring device is in contact with a measurement site of a body part of a user;
an auxiliary sensor within the enclosure;
a wireless communication device;
a processing device operably connected to the optical sensor, the auxiliary sensor, and the wireless communication device; and
a memory operably connected to the processing device and storing instructions, that when executed by the processing device, cause operations to be performed, the operations comprising:
receiving a data stream from the optical sensor;
receiving an auxiliary data stream from the auxiliary sensor;
demodulating and demultiplexing the data stream to produce at least two individual data streams;
decimating the at least two individual data streams to produce at least two decimated individual data steams;
demodulating and demultiplexing the auxiliary data stream to produce a time-multiplexed data stream; and
transmitting, using the wireless communication device, the at least two decimated individual data streams and the time-multiplexed data stream to a computing device.

2. The monitoring device of claim 1, wherein the monitoring device is incorporated into a hat worn by the user.

3. The monitoring device of claim 1, wherein the computing device is incorporated into a space suit.

4. The monitoring device of claim 1, wherein the monitoring device comprises a wearable patch and a monofilament is used to attach the wearable patch to the measurement site.

5. The monitoring device of claim 1, further comprising an encapsulation assembly encapsulating the enclosure and configured to attach the monitoring device to the measurement site using an adhesive on a surface of the encapsulation assembly, wherein the encapsulation assembly comprises:
a sensor window positioned over the optical sensor;
a contact opening extending through an encapsulation component of the encapsulation assembly for receiving a first removable tab, the first removable tab including a liner layer portion disposed on a first surface of the encapsulation component covering at least one contact pad on a printed circuit board and a tab portion disposed on an opposite second surface of the encapsulation component; and
a second removable tab positioned on a surface of the encapsulation assembly, the second removable tab including an adhesive material that provides the adhesive layer on the surface of the encapsulation assembly when the second removable tab is removed from the encapsulation assembly.
